(19) United States
(12) Patent Application Publication
(10) Pub. No.: US 2023/0002361 A1
(43) Pub. Date: Jan. 5, 2023

(54) 2H-INDAZOLE DERIVATIVES AND THEIR USE IN THE TREATMENT OF DISEASE

(71) Applicant: BIOGEN MA INC., Cambridge, MA (US)

(72) Inventors: Emily Anne Peterson, Belmont, MA (US); Ryan Evans, Somerville, MA (US); Fang Gao, Somerville, MA (US); Philippe Bolduc, Weymouth, MA (US); Magnus Pfaffenbach, Chelsea, MA (US); Zhili Xin, Lexington, MA (US)

(21) Appl. No.: 17/623,182

(22) PCT Filed: Jun. 24, 2020
(86) PCT No.: PCT/US2020/039346

§ 371 Date: Dec. 27, 2021

Related U.S. Application Data
(60) Provisional application No. 62/867,521, filed on Jun. 27, 2019.

(51) Int. Cl. C07D 405/14; C07D 471/04; C07D 401/12; C07D 487/04; C07D 519/00

(52) U.S. Cl. CPC C07D 405/14; C07D 471/04; C07D 401/12; C07D 487/04; C07D 519/00

(57) ABSTRACT

This invention relates to 2H-indazole Derivatives of formula (I′), or pharmaceutically acceptable salts thereof, in which all of the variables are as defined in the specification, capable of modulating the activity of IRAK4. The invention further provides a method of manufacturing compounds of the invention, and methods for their therapeutic use. The invention further provides methods to their preparation, to their medical use, in particular to their use in the treatment and management of diseases or disorders including inflammatory disease, autoimmune disease, cancer, cardiovascular disease, a disease of the central nervous system, disease of the skin, an ophthalmic disease and condition, and a bone disease.

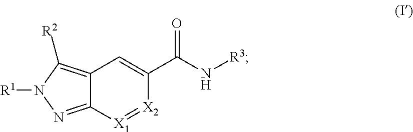

### RELATED APPLICATION

This application claims the benefit of the filing date under 35 U.S.C. § 119(e), of U.S. Provisional Patent Application No. 62/867,521, filed on Jun. 27, 2019, the entire content of which is hereby incorporated by reference.

### FIELD OF THE INVENTION

The present invention relates to 2H-indazole Derivatives and pharmaceutically acceptable salts thereof, compositions of these compounds, either alone or in combination with at least one additional therapeutic agent, processes for their preparation, their use in the treatment of diseases, their use, either alone or in combination with at least one additional therapeutic agent and optionally in combination with a pharmaceutically acceptable carrier, for the manufacture of pharmaceutical preparations, use of the pharmaceutical preparations for the treatment of diseases, and a method of treatment of said diseases, comprising administering the 2H-indazole Derivatives to a warm-blooded animal, especially a human.

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is the protein kinase family.

Kinases catalyze the phosphorylation of proteins, lipids, sugars, nucleosides and other cellular metabolites and play key roles in all aspects of eukaryotic cell physiology. Especially, protein kinases and lipid kinases participate in the signaling events which control the activation, growth, differentiation and survival of cells in response to extracellular mediators or stimuli such as growth factors, cytokines or chemokines. In general, protein kinases are classified in two groups, those that preferentially phosphorylate tyrosine residues and those that preferentially phosphorylate serine and/or threonine residues.

Kinases are important therapeutic targets for the development of anti-inflammatory drugs (Cohen, 2009. Current Opinion in Cell Biology 21, 1-8), for example kinases that are involved in the orchestration of adaptive and innate immune responses. Kinase targets of particular interest are members of the IRAK family.

The interleukin-1 receptor-associated kinases (IRAKs) are critically involved in the regulation of intracellular signaling networks controlling inflammation (Ringwood and Li, 2008. Cytokine 42, 1-7). IRAKs are expressed in many cell types and can mediate signals from various cell receptors including toll-like receptors (TLRs). IRAK4 is thought to be the initial protein kinase activated downstream of the interleukin-1 (IL-1) receptor and all toll-like-receptors (TLRs) except TLR3, and initiates signaling in the innate immune system via the rapid activation of IRAK1 and slower activation of IRAK2. IRAK1 was first identified through biochemical purification of the IL-1 dependent kinase activity that co-immunoprecipitates with the IL-1 type 1 receptor (Cao et al., 1996. Science 271(5252): 1128-31). IRAK2 was identified by the search of the human expressed sequence tag (EST) database for sequences homologous to IRAK1 (Muzio et al., 1997. Science 278(5343): 1612-5). IRAK3 (also called IRAKM) was identified using a murine EST sequence encoding a polypeptide with significant homology to IRAK1 to screen a human phytohemagglutinin-activated peripheral blood leukocyte (PBL) cDNA library (Wesche et al., 1999. J. Biol. Chem. 274(27): 19403-10). IRAK4 was identified by database searching for IRAK-like sequences and PCR of a universal cDNA library (Li et al., 2002. Proc. Natl. Acad. Sci. USA 99(8):5567-5572). Many diseases are associated with abnormal cellular responses triggered by kinase-mediated events.

Many diseases and/or disorders are associated with abnormal cellular responses triggered by kinase-mediated events. These diseases and/or disorders include, but are not limited to, cancers, allergic diseases, autoimmune diseases, inflammatory diseases and/or disorders and/or conditions associated with inflammation and pain, proliferative diseases, hematopoietic disorders, hematological malignancies, bone disorders, fibrosis diseases and/or disorders, metabolic disorders, muscle diseases and/or disorders, respiratory diseases, pulmonary disorders, genetic development diseases, neurological and neurodegenerative diseases and/or disorders, chronic inflammatory demyelinating neuropathies, cardiovascular, vascular or heart diseases, epilepsy, Ischemic stroke, ophthalmic diseases, ocular diseases, asthma, Alzheimer's disease, Amyotrophic Lateral Sclerosis, Parkinson's disease, traumatic brain injury, Chronic Traumatic Encephalopathy and hormone-related diseases.

In view of the above, IRAK4 inhibitors are considered to be of value in the treatment and/or prevention for multiple therapeutic indications over a wide range of unmet needs.

### SUMMARY OF THE INVENTION

In a first aspect, the invention relates to a compound of formula (I′):

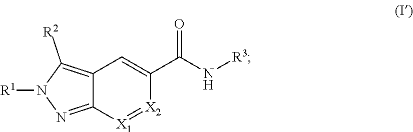

or a pharmaceutically acceptable salt thereof, wherein:

R¹ is selected from the group consisting of C₁₋₅ alkyl, C₃₋₆ cycloalkyl, —C₁₋₂ alkyl-C₃₋₆ cycloalkyl, a fully saturated 4 to 7 membered heterocycle containing 1 to 2 heteroatoms independently selected from nitrogen, sulfur and oxygen, —C₁₋₂ alkyl-C₄₋₇ heterocycle, wherein the C₄₋₇ heterocycle may be fully or partially saturated and contains 1 to 2 heteroatoms independently selected from nitrogen, sulfur and oxygen, —C₁₋₄ alkyl-O—C₁₋₂ alkyl, a fully saturated 5 to 8 membered bridged-carbocyclic ring, a fully saturated 5 to 8 membered bridged-heterocyclic ring system having 1 to 2 heteroatoms independently selected from nitrogen and oxygen, a 5 to 10 membered fused heterobicyclic ring system having 1 to 2 heteroatoms independently selected from nitrogen and oxygen and a 5 to 10 membered spiro heterobicyclic ring system having 1 to 2 heteroatoms independently selected from nitrogen and oxygen, wherein R¹ may be optionally substituted with 1, 2 or 3 substituents which are independently selected from halo, nitrile, oxo, halo-substitutedC₁₋₄ alkyl, hydroxy-substitutedC₁₋₄ alkyl, C₁₋₄ alkyl, C₄₋₇ heterocycle containing 1 to 2 heteroatoms independently selected from nitrogen and oxygen, a fully saturated 5 to 8 membered bridged-heterocyclic ring system having 1 to 2 heteroatoms independently selected from nitrogen and oxygen, C₁₋₄ alkyl-O—C₁₋₂ alkyl, hydroxyl and C₁₋₄ alkoxy;

R² is hydrogen, C₁₋₄ alkyl or halogen;

R³ is selected from the group consisting of
i. a 5 or 6 membered heteroaryl having 1 to 3 (e.g. 1 to 2) heteroatoms independently selected from nitrogen, oxygen and sulfur, said heteroaryl is optionally substituted with 1 to 3 R⁴;
ii. Phenyl optionally substituted with 1 to 3 R⁴,
iii. a 5-6 membered partially or fully saturated heterocycle having 1 to 2 heteroatoms independently selected from oxygen and nitrogen, said heterocycle may be optionally substituted with 1 to 3 R⁴;
iv. a partially or fully saturated C₃₋₆ cycloalkyl which may be optionally substituted with 1 to 3 R⁴;
v. a 7 to 10 membered fused heterobicyclic ring system having 1, 2 or 3 heteroatoms independently selected from nitrogen and oxygen, said ring system is optionally substituted with 1 to 3 R⁴; and
vi. a 7 to 10 membered fused bicyclic ring system optionally having 1, 2 or 3 heteroatoms independently selected from nitrogen and oxygen, said ring system is optionally substituted with 1 to 3 R⁴;

X₁ and X₂ are independently selected from N, CH and CR⁵, wherein only one of X₁ or X₂ may be N;

R⁵ is selected from halogen, C₁₋₄ alkyl, nitrile and —OR⁶, wherein the C₁₋₄ alkyl is optionally substituted with C₁₋₄ alkoxy;

R⁶ is hydrogen, C₁₋₅ alkyl, C₃₋₆ cycloalkyl, a 4 to 7 membered partially or fully saturated heterocycle containing 1 or 2 heteroatoms selected from nitrogen and oxygen, a 5 to 10 membered spiro carbocyclic ring and a 5 to 10 membered spiro heterobicyclic ring system having 1 to 2 heteroatoms independently selected from nitrogen and oxygen, wherein the C₁₋₅ alkyl represented by R⁶ is optionally substituted with 1 to 3 substituents R^{6a} independently selected from halogen, hydroxyl, C₁₋₄ alkoxy, halo-substitutedC₁₋₄ alkoxy, C₃₋₆ cycloalkyl, phenyl, a 4 to 7 membered partially or fully saturated heterocycle containing 1 or 2 heteroatoms selected from nitrogen and oxygen, an a fully saturated 5 to 8 membered bridged-heterocyclic ring system having 1 to 2 heteroatoms independently selected from nitrogen and oxygen; the C₃₋₆ cycloalkyl represented by R⁶ is optionally substituted with 1 to 3 substituents R^{6b} independently selected from halo, C₁₋₄ alky, halo-substitutedC₁₋₄ alkyl, and C₁₋₄ alkoxy; the 4 to 7 membered partially or fully saturated heterocycle, the 5 to 10 membered spiro carbocyclic ring and 5 to 10 membered spiro heterobicyclic ring system represented by R⁶ is optionally substituted with 1 to 3 substituents R⁶, independently selected from C₁₋₄ alky and oxo, and wherein said C₃₋₆ cycloalkyl, phenyl, 4 to 7 membered partially or fully saturated heterocycle represented by R^{6a} are optionally substituted with 1 to 3 R⁷;

each R⁷ is independently selected from oxo, halo, halo-substitutedC₁₋₄ alkyl and C₁₋₄ alkyl;

R⁴ for each occurrence, is independently selected from CN, hydroxyl, C₁₋₄ alkyl, CN-substitutedC₁₋₄ alkyl, oxo, halo, halo-substitutedC₁₋₄ alkyl, C₁₋₄ alkoxy-C₁₋₄ alkyl, —NR⁸R⁹, C₁₋₄ alkoxy, C₁₋₄ alkoxy-C₁₋₄ alkoxy, hydroxy-substituted C₁₋₄ alkyl, halo-substitutedC₁₋₄ alkoxy, C₃₋₆ cycloalkyl, —C₁₋₄ alkyl-C₃₋₆ cycloalkyl, C(O)NR¹⁰R¹¹, a C₄₋₇ heterocycle, and a 5 or 6 membered heteroaryl having 1 to 2 heteroatoms independently selected from nitrogen, oxygen and sulfur, said C₃₋₆ cycloalkyl and heteroaryl may be optionally substituted with 1 to 2 substituents independently selected from the group consisting of C₁₋₄ alkyl, hydroxyl and halogen; or two R⁴ groups on the same atom may form a C₃₋₆ cycloalkyl, or two R⁴ groups on adjacent ring atoms may form phenyl, C₄₋₆ carbocycle, C₄₋₆ heterocycle, or a 7 membered bridged ring system optionally having 1 heteroatom selected from nitrogen and oxygen, wherein said phenyl, C₃₋₆ cycloalkyl C₄₋₆ carbocycle and C₄₋₆ heterocycle may be optionally substituted with 1 to 2 C₁₋₄ alkyl, halo or halo-substitutedC₁₋₄ alkyl;

R⁸ and R⁹ are each independently selected from hydrogen, —C(O)C₁₋₄ alkyl and C₁₋₄ alkyl; or R⁸ and R⁹ may combine to form a 4 to 6 membered saturated ring optionally containing one additional heteroatom selected from nitrogen or oxygen wherein said additional nitrogen may be optionally substituted with C₁₋₄ alkyl; and

R¹⁰ and R¹¹ are each independently selected from hydrogen and C₁₋₄ alkyl.

In some embodiments, the invention relates to compounds of formula (I′) described above, wherein:

R⁵ is selected from halogen, C₁₋₄ alkyl, nitrile and —OR⁶;

R⁶ is hydrogen, a C₁₋₅ alkyl, a C₃₋₆ cycloalkyl or a fully saturated 4 to 7 membered heterocycle containing 1 or 2 heteroatoms selected from nitrogen and oxygen, wherein the C₁₋₅ alkyl represented by R⁶ is optionally substituted with 1 to 3 substituents R^{6a} independently selected from halogen, hydroxyl, C₁₋₄ alkoxy, C₃₋₆ cycloalkyl, phenyl and a 4 to 7 membered partially or fully saturated heterocycle containing 1 or 2 heteroatoms selected from nitrogen and oxygen, the C₃₋₆ cycloalkyl represented by R⁶ is optionally substituted with 1-3 substituent R^{6b} independently selected from halogen, C₁₋₄ alkyl, halo-substitutedC₁₋₄ alkyl and C₁₋₄ alkoxy; wherein said C₃₋₆ cycloalkyl and phenyl represented by R^{6a} may be optionally substituted with 1 to 3 R⁷;

R⁴ for each occurrence, is independently selected from CN, hydroxyl, C₁₋₄ alkyl, CN-substitutedC₁₋₄ alkyl, oxo, halo, halo-substitutedC₁₋₄ alkyl, —NR⁸R⁹, C₁₋₄ alkoxy, C₁₋₄ alkoxy-C₁₋₄ alkoxy, hydroxy-substituted C₁₋₄ alkyl, halo-substitutedC₁₋₄ alkoxy, C₃₋₆ cycloalkyl, C(O)NR¹⁰R¹¹ and a 5 or 6 membered heteroaryl having 1 to 2 heteroatoms independently selected from nitrogen, oxygen and sulfur, said C₃₋₆ cycloalkyl and heteroaryl may be optionally substituted with 1 to 2 substituents independently selected from the group consisting of C₁₋₄ alkyl, hydroxyl and halogen; or two R⁴ groups on the same atom may form a C₃₋₆ cycloalkyl, or two R⁴ groups on adjacent ring atoms may form phenyl, C₄₋₆ carbocycle, C₄₋₆ heterocycle, or a 7 membered bridged ring system optionally having 1 heteroatom selected from nitrogen and oxygen, wherein said phenyl, C₃₋₆ cycloalkyl C₄₋₆ carbocycle and C₄₋₆ heterocycle may be optionally substituted with 1 to 2 C₁₋₄ alkyl, halo or halo-substitutedC₁₋₄ alkyl; and the remaining variables are as described above in the first aspect.

Also in the first aspect, the invention relates to a compound of formula (I):

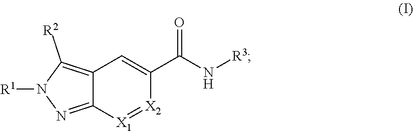

or a pharmaceutically acceptable salt thereof, wherein:

R¹ is selected from the group consisting of C₁₋₅ alkyl, C₃₋₆ cycloalkyl, —C₁₋₂ alkyl-C₃₋₆ cycloalkyl, a fully saturated 4 to 7 membered heterocycle containing 1 to 2 heteroatoms independently selected from nitrogen, sulfur and oxygen, —C₁₋₂ alkyl-C₄₋₇ heterocycle, wherein the C₄₋₇ heterocycle may be fully or partially saturated and contains 1 to 2 heteroatoms independently selected from nitrogen, sulfur and oxygen, —C₁₋₄ alkyl-O—C₁₋₂ alkyl, a fully saturated 5 to 8 membered bridged-carbocyclic ring, a fully saturated 5 to 8 membered bridged-heterocyclic ring system having 1 to 2 heteroatoms independently selected from nitrogen and oxygen, a 5 to 10 membered fused heterobicyclic ring system having 1 to 2 heteroatoms independently selected from nitrogen and oxygen and a 5 to 10 membered spiro heterobicyclic ring system having 1 to 2 heteroatoms independently selected from nitrogen and oxygen, wherein R¹ may be optionally substituted with 1, 2 or 3 substituents which are independently selected from halo, nitrile, oxo, halo-substitutedC₁₋₄ alkyl, hydroxy-substitutedC₁₋₄ alkyl, C₁₋₄ alkyl, C₄₋₇ heterocycle containing 1 to 2 heteroatoms independently selected from nitrogen and oxygen, C₁₋₄ alkyl-O—C₁₋₂ alkyl, hydroxyl and C₁₋₄ alkoxy;

R² is hydrogen, C₁₋₄ alkyl or halogen;

R³ is selected from the group consisting of
i. a 5 or 6 membered heteroaryl having 1 to 2 heteroatoms independently selected from nitrogen, oxygen and sulfur, said heteroaryl is optionally substituted with 1 to 3 R⁴;
ii. Phenyl optionally substituted with 1 to 3 R⁴,
iii. a 5-6 membered partially or fully saturated heterocycle having 1 to 2 heteroatoms independently selected from oxygen and nitrogen, said heterocycle may be optionally substituted with 1 to 3 R⁴;
iv. a partially or fully saturated C₃₋₆ cycloalkyl which may be optionally substituted with 1 to 3 R⁴;
v. a 7 to 10 membered fused heterobicyclic ring system having 1, 2 or 3 heteroatoms independently selected from nitrogen and oxygen, said ring system is optionally substituted with 1 to 3 R⁴; and
vi. a 7 to 10 membered fused bicyclic ring system optionally having 1, 2 or 3 heteroatoms independently selected from nitrogen and oxygen, said ring system is optionally substituted with 1 to 3 R⁴;

X₁ and X₂ are independently selected from N, CH and CR⁵, wherein only one of X₁ or X₂ may be N;

R⁵ is selected from halogen, C₁₋₄ alkyl, nitrile and —OR⁶;

R⁶ is hydrogen or an optionally substituted C₁₋₅ alkyl having 1 to 3 substituents independently selected from halogen, hydroxyl, C₁₋₄ alkoxy, C₃₋₆ cycloalkyl, phenyl and a 4 to 7 membered partially or fully saturated heterocycle containing 1 or 2 heteroatoms selected from nitrogen and oxygen, wherein said C₃₋₆ cycloalkyl and phenyl may be optionally substituted with 1 to 3 R⁷;

each R⁷ is independently selected from oxo, halo, halo-substitutedC₁₋₄ alkyl and C₁₋₄ alkyl;

R⁴ for each occurrence, is independently selected from CN, hydroxyl, C₁₋₄ alkyl, CN-substitutedC₁₋₄ alkyl, oxo, halo, halo-substitutedC₁₋₄ alkyl, —NR⁸R⁹, C₁₋₄ alkoxy, C₁₋₄ alkoxy-C₁₋₄ alkoxy, hydroxy-substituted C₁₋₄ alkyl, halo-substitutedC₁₋₄ alkoxy, C₃₋₆ cycloalkyl, C(O)NR¹⁰R¹¹ and a 5 or 6 membered heteroaryl having 1 to 2 heteroatoms independently selected from nitrogen, oxygen and sulfur, said C₃₋₆ cycloalkyl and heteroaryl may be optionally substituted with 1 to 2 substituents independently selected from the group consisting of C₁₋₄ alkyl, hydroxyl and halogen; or two R⁴ groups on the same atom may form a C₃₋₆ cycloalkyl, or two R⁴ groups on adjacent ring atoms may form phenyl, C₄₋₆ carbocycle, C₄₋₆ heterocycle, or a 7 membered bridged ring system optionally having 1 heteroatom selected from nitrogen and oxygen, wherein said phenyl, C₃₋₆ cycloalkyl C₄₋₆ carbocycle and C₄₋₆ heterocycle may be optionally substituted with 1 to 2 C₁₋₄ alkyl, halo or halo-substitutedC₁₋₄ alkyl;

R⁸ and R⁹ are each independently selected from hydrogen, —C(O)C₁₋₄ alkyl and C₁₋₄ alkyl; or R⁸ and R⁹ may combine to form a 4 to 6 membered saturated ring optionally containing one additional heteroatom selected from nitrogen or oxygen wherein said additional nitrogen may be optionally substituted with C₁₋₄ alkyl; and R¹⁰ and R¹¹ are each independently selected from hydrogen and C₁₋₄ alkyl.

Another aspect of the invention relates to pharmaceutical compositions comprising compounds of (I′) or (I) or pharmaceutically acceptable salts thereof, and a pharmaceutical carrier. Such compositions can be administered in accordance with a method of the invention, typically as part of a therapeutic regimen for the treatment or prevention of conditions and disorders related to interleukin-1 receptor-associated kinases activity. In a particular aspect, the pharmaceutical compositions may additionally comprise further one or more therapeutically active ingredients suitable for the use in combination with the compounds of the invention. In a more particular aspect, the further therapeutically active ingredient is an agent for the treatment of autoimmune diseases, inflammatory diseases, bone diseases, metabolic diseases, neurological and neurodegenerative diseases, cancer, cardiovascular diseases, allergies, asthma, Alzheimer's disease, and hormone-related diseases.

Another aspect of the invention relates to the pharmaceutical combinations comprising compounds of the invention and other therapeutic agents for the use as a medicament in the treatment of patients having disorders related to interleukin-1 receptor-associated kinases activity. Such combinations can be administered in accordance with a method of the invention, typically as part of a therapeutic regiment for the treatment or prevention of autoimmune diseases, inflammatory diseases, bone diseases, metabolic diseases, neurological and neurodegenerative diseases, cancer, cardiovascular diseases, allergies, asthma, Alzheimer's disease, and hormone-related diseases. Accordingly, there remains a need to find protein kinase inhibitors useful as therapeutic agents.

### DETAILED DESCRIPTION OF THE INVENTION

The present invention provides compounds and pharmaceutical formulations thereof that may be useful in the treatment or prevention of conditions and/or disorders through mediation of IRAK4 function, such as neurological and neurodegenerative diseases, Alzheimer's disease, Ischemic stroke, Cerebral Ischemia, hypoxia, TBI (Traumatic Brain Injury), CTE (Chronic Traumatic Encephalopathy), epilepsy, Parkinson's disease (PD), Multiple Sclerosis (MS) and Amyotrophic Lateral Sclerosis (ALS).

In a first embodiment, the invention provides a compound of formula (I′) or a pharmaceutically acceptable salt thereof, wherein the variables in formula (I′) are as defined above in the first aspect.

In a second embodiment, the invention provides a compound of formula (I):

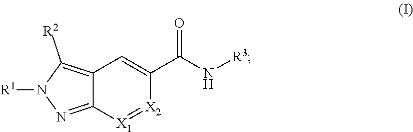

or a pharmaceutically acceptable salt thereof, wherein:

R¹ is selected from the group consisting of C₁₋₅ alkyl, C₃₋₆ cycloalkyl, —C₁₋₂ alkyl-C₃₋₆ cycloalkyl, a fully saturated 4 to 7 membered heterocycle containing 1 to 2 heteroatoms independently selected from nitrogen, sulfur and oxygen, —C₁₋₂ alkyl-C₄₋₇ heterocycle, wherein the C₄₋₇ heterocycle may be fully or partially saturated and contains 1 to 2 heteroatoms independently selected from nitrogen, sulfur and oxygen, —C₁₋₄ alkyl-O—C₁₋₂ alkyl, a fully saturated 5 to 8 membered bridged-carbocyclic ring, a fully saturated 5 to 8 membered bridged-heterocyclic ring system having 1 to 2 heteroatoms independently selected from nitrogen and oxygen, a 5 to 10 membered fused heterobicyclic ring system having 1 to 2 heteroatoms independently selected from nitrogen and oxygen and a 5 to 10 membered spiro heterobicyclic ring system having 1 to 2 heteroatoms independently selected from nitrogen and oxygen, wherein R¹ may be optionally substituted with 1, 2 or 3 substituents which are independently selected from halo, nitrile, oxo, halo-substitutedC₁₋₄ alkyl, hydroxy-substitutedC₁₋₄ alkyl, C₁₋₄ alkyl, C₄₋₇ heterocycle containing 1 to 2 heteroatoms independently selected from nitrogen and oxygen, C₁₋₄ alkyl-O—C₁₋₂ alkyl, hydroxyl and C₁₋₄ alkoxy;

R² is hydrogen, C₁₋₄ alkyl or halogen;

R³ is selected from the group consisting of
i. a 5 or 6 membered heteroaryl having 1 to 2 heteroatoms independently selected from nitrogen, oxygen and sulfur, said heteroaryl is optionally substituted with 1 to 3 R⁴;
ii. Phenyl optionally substituted with 1 to 3 R⁴,
iii. a 5-6 membered partially or fully saturated heterocycle having 1 to 2 heteroatoms independently selected from oxygen and nitrogen, said heterocycle may be optionally substituted with 1 to 3 R⁴;
iv. a partially or fully saturated C₃₋₆ cycloalkyl which may be optionally substituted with 1 to 3 R⁴;
v. a 7 to 10 membered fused heterobicyclic ring system having 1, 2 or 3 heteroatoms independently selected from nitrogen and oxygen, said ring system is optionally substituted with 1 to 3 R⁴; and
vi. a 7 to 10 membered fused bicyclic ring system optionally having 1, 2 or 3 heteroatoms independently selected from nitrogen and oxygen, said ring system is optionally substituted with 1 to 3 R⁴;

X₁ and X₂ are independently selected from N, CH and CR⁵, wherein only one of X₁ or X₂ may be N;

R⁵ is selected from halogen, C₁₋₄ alkyl, nitrile and —OR⁶;

R⁶ is hydrogen or an optionally substituted C₁₋₅ alkyl having 1 to 3 substituents independently selected from halogen, hydroxyl, C₁₋₄ alkoxy, C₃₋₆ cycloalkyl, phenyl and a 4 to 7 membered partially or fully saturated heterocycle containing 1 or 2 heteroatoms selected from nitrogen and oxygen, wherein said C₃₋₆ cycloalkyl and phenyl may be optionally substituted with 1 to 3 R⁷;

each R⁷ is independently selected from oxo, halo, halo-substitutedC₁₋₄ alkyl and C₁₋₄ alkyl;

R⁴ for each occurrence, is independently selected from CN, hydroxyl, C₁₋₄ alkyl, CN-substitutedC₁₋₄ alkyl, oxo, halo, halo-substitutedC₁₋₄ alkyl, —NR⁸R⁹, C₁₋₄ alkoxy, C₁₋₄ alkoxy-C₁₋₄ alkoxy, hydroxy-substituted C₁₋₄ alkyl, halo-substitutedC₁₋₄ alkoxy, C₃₋₆ cycloalkyl, C(O)NR¹⁰R¹¹ and a 5 or 6 membered heteroaryl having 1 to 2 heteroatoms independently selected from nitrogen, oxygen and sulfur, said C₃₋₆ cycloalkyl and heteroaryl may be optionally substituted with 1 to 2 substituents independently selected from the group consisting of C₁₋₄ alkyl, hydroxyl and halogen; or two R⁴ groups on the same atom may form a C₃₋₆ cycloalkyl, or two R⁴ groups on adjacent ring atoms may form phenyl, C₄₋₆ carbocycle, C₄₋₆ heterocycle, or a 7 membered bridged ring system optionally having 1 heteroatom selected from nitrogen and oxygen, wherein said phenyl, C₃₋₆ cycloalkyl C₄₋₆ carbocycle and C₄₋₆ heterocycle may be optionally substituted with 1 to 2 C₁₋₄ alkyl, halo or halo-substitutedC₁₋₄ alkyl;

R⁸ and R⁹ are each independently selected from hydrogen, —C(O)C₁₋₄ alkyl and C₁₋₄ alkyl; or R⁸ and R⁹ may combine to form a 4 to 6 membered saturated ring optionally containing one additional heteroatom selected from nitrogen or oxygen wherein said additional nitrogen may be optionally substituted with C₁₋₄ alkyl; and

R¹⁰ and R¹¹ are each independently selected from hydrogen and C₁₋₄ alkyl.

In a third embodiment, the invention provides a compound of the first or second embodiment of formula (I):

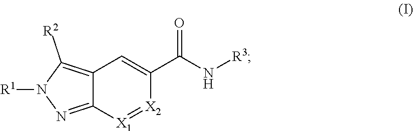

or a pharmaceutically acceptable salt thereof, wherein:

R² is H; and

X₁ is N or CH; and X² is CR⁵; and the remaining variables are as defined in the first or second embodiment.

In a fourth embodiment, the invention provides a compound of the first or second embodiment of formula (I):

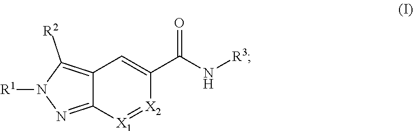

or a pharmaceutically acceptable salt thereof, wherein:

R² is H; and

X₁ is CR⁵ and X² is N or CH; and the remaining variables are as defined in the first or second embodiment.

In a fifth embodiment, the invention provides a compound of the first or second embodiment of formula (Ia):

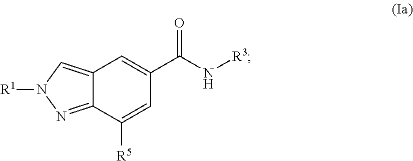

or a pharmaceutically acceptable salt thereof; wherein the variables are as defined in the first or second embodiment.

In a sixth embodiment, the invention provides a compound of the first or second embodiment of formula (Ib):

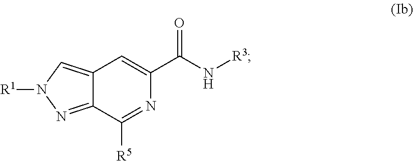

or a pharmaceutically acceptable salt thereof, wherein the variables are as defined in the first or second embodiment.

In a seventh embodiment, the invention provides a compound of the first or second embodiment of formula (Ic):

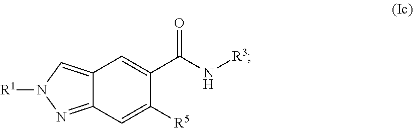

or a pharmaceutically acceptable salt thereof, wherein the variables are as defined in the first or second embodiment.

In an eighth embodiment, the invention provides a compound of the first or second embodiment of formula (Id):

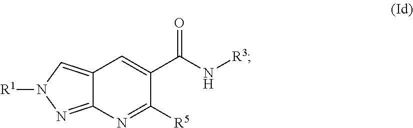

or a pharmaceutically acceptable salt thereof, wherein the variables are as defined in the first or second embodiment.

A ninth embodiment of the invention provides a compound according to any of the preceding embodiments or a pharmaceutically acceptable salt thereof, wherein:

R³ is selected from the group consisting of
i. a 5 or 6 membered heteroaryl having 1 to 2 heteroatoms independently selected from nitrogen, oxygen and sulfur, said heteroaryl is optionally substituted with 1 to 3 R⁴;
ii. Phenyl optionally substituted with 1 to 3 R⁴,
iii. a 5-6 membered partially or fully saturated heterocycle having 1 to 2 heteroatoms independently selected from oxygen and nitrogen, said heterocycle may be optionally substituted with 1 to 3 R⁴;
iv. a partially or fully saturated C₃₋₆ cycloalkyl which may be optionally substituted with 1 to 3 R⁴;
v. a 7 to 10 membered fused heterobicyclic ring system having 1, 2 or 3 heteroatoms independently selected from nitrogen and oxygen, said ring system is optionally substituted with 1 to 3 R⁴; and
vi. a 7 to 10 membered fused bicyclic ring system optionally having 1, 2 or 3 heteroatoms independently selected from nitrogen and oxygen, said ring system is optionally substituted with 1 to 3 R⁴; and

the remaining variables are as defined in the first, second, third, fourth, fifth, sixth, seventh or eighth embodiment described above.

In a tenth embodiment, the invention provides a compound of any one of the first to eighth embodiments or a pharmaceutically acceptable salt thereof, wherein:

R³ is phenyl, a 5 or 6 membered monocyclic heteroaryl having 1 to 3 heteroatoms independently selected from nitrogen and oxygen, pyridinyl-2 (1H)-one or a 9 to 10 membered bicyclic heteroaryl having 1 to 3 heteroatoms independently selected from nitrogen and oxygen, wherein the monocyclic heteroaryl, pyridinyl-2 (1H)-one or the bicyclic heteroaryl are each optionally substituted with 1 to 3 (e.g. 1 or 2) R⁴; and the remaining variables are as defined in the first, second, third, fourth, fifth, sixth, seventh or eighth embodiment described above.

In an eleventh embodiment, the invention provides a compound of any one of the first to eighth embodiments or a pharmaceutically acceptable salt thereof, wherein:

R³ is phenyl, a 5 or 6 membered monocyclic heteroaryl having 1 to 2 nitrogen atoms, pyridinyl-2 (1H)-one or a 9 to 10 membered bicyclic heteroaryl having 2 to 3 nitrogen atoms, wherein the monocyclic heteroaryl, pyridinyl-2 (1H)-one or the bicyclic heteroaryl are each optionally substituted with 1 to 3 (e.g. 1 or 2) R⁴; and the remaining variables are as defined in the first, second, third, fourth, fifth, sixth, seventh or eighth embodiment described above.

In a twelfth embodiment, the invention provides a compound of any one of the first to eighth embodiments or a pharmaceutically acceptable salt thereof, wherein:

R³ is selected from cyclopropyl, cyclobutyl, cyclohexyl, bicyclo[3.1.0]hexane, bicyclo[4.1.0]heptane, tetrahydrofuran, 4-oxaspiro[bicyclo[3.2.0]heptane-6,1′-cyclobutane], oxaspirobicyclo[3.2.0]heptane, spiro[2.5]octane, phenyl, 2H-1,2,3-triazole, isoxazole, isothiazole, thiazole, pyrazole, pyridine, pyridinyl-2 (1H)-one, 6,7-dihydro-5H-cyclopenta[b]pyridine, pyrazolo[1,5-a]pyridine, [1,2,4]triazolo[4,3-a]pyridine, isothiazolo[4,3-b]pyridine, pyrimidine, pyrimidin-4 (3H)-one, pyrazolo[1,5-a]pyrimidine, pyrido[3,2-d]pyrimidine, imidazo[1,2-b]pyridazine, thieno[2,3-b]pyrazine, 1H-benzo[d]imidazole, benzo[d]thiazole, 2,3-dihydrobenzofuran, indane, 2,3-dihydro-1H-indene, 1,6-naphthyridine, 1,5-naphthyridine, 5,6,7,8-tetrahydronaphthalene, 2H-indazole, 6,7-dihydro-5H-pyrazolo[5,1-b][1,3]oxazine, thiophene, chromane and isochromane, wherein said R³ is optionally substituted with 1 to 3 (e.g., 1 or 2) R⁴; and the remaining variables are as defined in the first, second, third, fourth, fifth, sixth, seventh or eighth embodiment.

In some embodiments, for the compounds of the twelfth embodiment or a pharmaceutically acceptable salt thereof, R³ is selected from phenyl, pyrazole, pyridine, pyridinyl-2 (1H)-one, pyrimidine, pyrazolo[1,5-a]pyridine, pyrazolo[1,5-a]pyrimidine, and 2,3-dihydrobenzofuran, wherein R³ is optionally substituted with 1 to 3 (e.g., 1 or 2) R⁴; and the remaining variables are as defined in the twelfth embodiment.

In a thirteenth embodiment, the invention provides a compound of any one of the first to twelfth embodiments or a pharmaceutically acceptable salt thereof, wherein R⁴, for each occurrence, is independently selected from hydroxyl, halo, halo-substitutedC₁₋₄ alkyl, —NR⁸R⁹, C₁₋₄ alkoxy, C₃₋₆ cycloalkyl, and C₁₋₄ alkyl; and the remaining variables are as defined in the first, second, third, fourth, fifth, sixth, seventh, eighth, ninth, tenth or eleventh embodiment described above. In some embodiments, for compounds of the thirteenth embodiment or a pharmaceutically acceptable salt thereof, R⁴, for each occurrence, is independently selected from hydroxyl, halo, halo-substitutedC₁₋₄ alkyl, —NR⁸R⁹, and C₁₋₄ alkyl.

In a fourteenth embodiment, the invention provides a compound of any one of the first to eighth embodiments or a pharmaceutically acceptable salt thereof, wherein:

R³ is selected from pyridyl, oxazolyl, pyrazinyl, oxadiazoyl, thiophenyl, thiazolyl, isothiazolyl, pyrazolyl, imidazolyl, said R³ is optionally substituted with 1 to 2 substituents independently selected from the group consisting of halo, halo-substitutedC₁₋₄ alkyl, —NR⁸R⁹, and C₁₋₄ alkyl; and the remaining variables are as defined in the first, second, third, fourth, fifth, sixth, seventh or eighth embodiment described above

In a fifteenth embodiment, the invention provides a compound of any one of the first to eighth embodiments or a pharmaceutically acceptable salt thereof, wherein:

R³ is pyridinyl-2 (1H)-one optionally substituted with 1 to 2 substituents independently selected from the group consisting of halo, halo-substitutedC₁₋₄ alkyl, —NR⁸R⁹, and C₁₋₄ alkyl; and the remaining variables are as defined in the first, second, third, fourth, fifth, sixth, seventh or eighth embodiment described above

In a sixteenth embodiment, the invention provides a compound of any one of the first to eighth embodiments or a pharmaceutically acceptable salt thereof, wherein:

R³ is phenyl, said phenyl is optionally substituted with 1 to 2 substituents independently selected from the group consisting of halo, halo-substitutedC₁₋₄ alkyl, —NR⁸R⁹, and C₁₋₄ alkyl; and the remaining variables are as defined in the first, second, third, fourth, fifth, sixth, seventh or eighth embodiment described above.

In a seventeenth embodiment, the invention provides a compound of any one of the first to eighth embodiments or a pharmaceutically acceptable salt thereof, wherein:

R³ is selected from the group consisting of 1,3-dihydroisobenzofuran, 2,3-dihydrobenzofuran, 4-oxaspiro[bicyclo[3.2.0]heptane-6,1′-cyclobutane], oxaspiro[bicyclo[3.2.0]heptane-6,1′-cyclobutane], bicyclo[3.1.0]hexane, cyclohexyl, spiro[2.5]octane, 1S,5R)-1-methylbicyclo[3.1.0]hexane, 2,3-dihydro-1H-indene, spiro[2.5]octane, 1,2,3,4-tetrahydronaphthalen, tetrahydrofuran, 2,3-dihydrobenzofuran, 2,3-dihydro-1H-indene, 4-methyl-3,4-dihydro-2H-benzo[b][1,4]oxazine, pyrido[3,2-d]pyrimidinyl, 1,2,3,4-tetrahydro-1,4-epoxynaphthalene, 5,6-dihydro-4H-pyrrolo[1,2-b]pyrazole, 6,7-dihydro-5H-cyclopenta[b]pyridine, 1,2,3,4-tetrahydronaphthalene, indolin-2-one, 2,3-dihydrobenzofuran, pyrazolo[1,5-a]pyrimidine, 1-methyl-2-oxo-1,2,3,4-tetrahydroquinoline, 3,4-dihydroquinolin-2 (1H)-one, chromane, and isochromane, wherein said R³ is optionally substituted with 1 to 2 substituents independently selected from the group consisting halo, halo-substitutedC₁₋₄ alkyl, —NR⁸R⁹, and C₁₋₄ alkyl; and the remaining variables are as defined in the first, second, third, fourth, fifth, sixth, seventh or eighth embodiment described above.

In some embodiments, for compounds of any one of first to eighth embodiments or a pharmaceutically acceptable salt thereof, wherein:

R³ is selected from the group consisting of 2-fluoro-3-methylphenyl, 1-(difluoromethyl)-1H-pyrazol-3-yl, 1-methyl-1H-pyrazol-3-yl, pyridin-2-yl, 2-methoxypyridin-3-yl, 6-methoxypyridin-2-yl, 6-(difluoromethyl)pyridin-2-yl, 2-(difluoromethoxy)pyridin-3-yl, 6-(trifluoromethyl)pyridin-2-yl, 1-methyl-2-oxo-1,2-dihydropyridin-3-yl, 5-fluoro-1-methyl-2-oxo-1,2-dihydropyridin-3-yl, 1-(difluoromethyl)-2-oxo-1,2-dihydropyridin-3-yl, 4-(difluoromethyl)pyrimidin-2-yl, pyrazolo[1,5-a]pyridin-4-yl, pyrazolo[1,5-a]pyridin-7-yl, pyrazolo[1,5-a]pyrimidin-3-yl, 5-methylpyrazolo[1,5-a]pyrimidin-3-yl, 6-methylpyrazolo[1,5-a]pyrimidin-3-yl, 6-chloropyrazolo[1,5-a]pyrimidin-3-yl, 6-fluoropyrazolo[1,5-a]pyrimidin-3-yl, 6-methoxypyrazolo[1,5-a]pyrimidin-3-yl, and 2,3-dihydrobenzofuran-7-yl; and the remaining variables are as defined in the first, second, third, fourth, fifth, sixth, seventh or eighth embodiment described above.

In an eighteenth embodiment, the invention provides a compound of any one of embodiments one, two, three or four of formula (II):

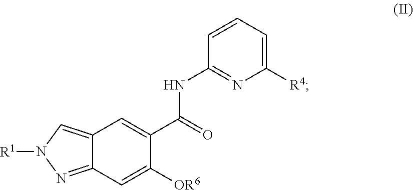

or a pharmaceutically acceptable salt thereof, wherein:

R⁶ is an optionally substituted C₁₋₅ alkyl having 1 to 3 substituents independently selected from halogen, hydroxyl, C₁₋₄ alkoxy, C₃₋₆ cycloalkyl, phenyl and a 4 to 7 membered partially or fully saturated heterocycle containing 1 or 2 heteroatoms selected from nitrogen and oxygen, wherein said C₃₋₆ cycloalkyl and phenyl may be optionally substituted with 1 to 3 R⁷; and the remaining variables are as defined in the first, second, third or fourth embodiment.

In a nineteenth embodiment, the invention provides a compound of any of one of embodiments one, two, three or four of formula (III):

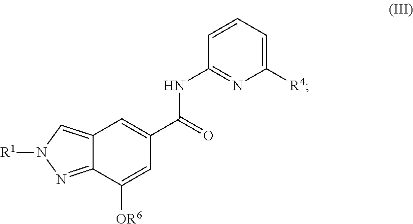

or a pharmaceutically acceptable salt thereof, wherein:

R⁶ is an optionally substituted C₁₋₅ alkyl having 1 to 3 substituents independently selected from halogen, hydroxyl, C₁₋₄ alkoxy, C₃₋₆ cycloalkyl, phenyl and a 4 to 7 membered partially or fully saturated heterocycle containing 1 or 2 heteroatoms selected from nitrogen and oxygen, wherein said C₃₋₆ cycloalkyl and phenyl may be optionally substituted with 1 to 3 R⁷; and the remaining variables are as defined in the first, second, third or fourth embodiment.

In a twentieth embodiment, the invention provides a compound of any of one of embodiments one, two, three or four of formula (IV):

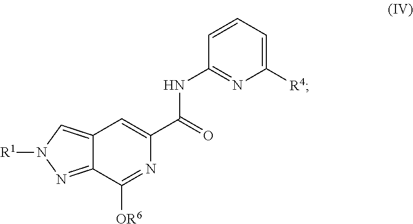

or a pharmaceutically acceptable salt thereof, wherein:

R⁶ is an optionally substituted C₁₋₅ alkyl having 1 to 3 substituents independently selected from halogen, hydroxyl, C₁₋₄ alkoxy, C₃₋₆ cycloalkyl, phenyl and a 4 to 7 membered partially or fully saturated heterocycle containing 1 or 2 heteroatoms selected from nitrogen and oxygen, wherein said C₃₋₆ cycloalkyl and phenyl may be optionally substituted with 1 to 3 R⁷; and the remaining variables are as defined in the first, second, third or fourth embodiment.

In a twenty-first embodiment, the invention provides a compound of any one of the preceding embodiments or a pharmaceutically acceptable salt thereof, wherein:

R¹ is a fully saturated C₄₋₇ heterocycle or a 5 to 8 membered bridged-heterocyclic ring system which contain 1 to 2 heteroatoms independently selected from nitrogen and oxygen, said C₄₋₇ heterocycle or a 5 to 8 membered bridged-heterocyclic ring system may be optionally substituted with 1 or 2 substituents independently selected from the group consisting of C₁₋₄ alkyl, halogen, halo-substitutedC₁₋₄ alkyl, hydroxyl and C₁₋₄ alkoxy; or R¹ is a C₁₋₅ alkyl which is optionally substituted with 1 or 3 substituents independently selected from the group consisting of halogen, halo-substitutedC₁₋₄ alkyl, hydroxy-substitutedC₁₋₄ alkyl, hydroxyl, C₁₋₄ alkoxy and C₃₋₆ cycloalkyl, wherein said C₃₋₆ cycloalkyl is optionally substituted with 1 or 2 substituents independently selected from the group consisting of halogen, halo-substitutedC₁₋₄ alkyl, hydroxyl and C₁₋₄ alkoxy; and the remaining variables are as defined in any one of the first to twentieth embodiments described above.

In a twenty-second embodiment of the invention provides a compound of any one of the preceding embodiments or a pharmaceutically acceptable salt thereof, wherein:

R¹ is a fully saturated C₄₋₇ heterocycle or a 5 to 8 membered bridged-heterocyclic ring system which contain 1 to 2 heteroatoms independently selected from nitrogen and oxygen, said C₄₋₇ heterocycle or a 5 to 8 membered bridged-heterocyclic ring system may be optionally substituted with 1 or 2 substituents independently selected from the group consisting of C₁₋₄ alkyl, halogen, halo-substitutedC₁₋₄ alkyl, hydroxyl and C₁₋₄ alkoxy; and the remaining variables are as defined in any one of the first to twenty-first embodiments described above.

In a twenty-third embodiment, the invention provides a compound of any one of embodiments one to twenty-first or a pharmaceutically acceptable salt thereof, wherein: R¹ is a C₁₋₅ alkyl which is optionally substituted with 1 or 3 substituents independently selected from the group consisting of halogen, halo-substitutedC₁₋₄ alkyl, hydroxyl, C₁₋₄ alkoxy and C₃₋₆ cycloalkyl, wherein said C₃₋₆ cycloalkyl is optionally substituted with 1 or 2 substituents independently selected from the group consisting of halogen, halo-substitutedC₁₋₄ alkyl, hydroxyl and C₁₋₄ alkoxy; and the remaining variables are as defined in any one of the first to twenty-first embodiments described above.

In a twenty-fourth embodiment, the invention provides a compound of any one of embodiments one to twenty or a pharmaceutically acceptable salt thereof, wherein:

R¹ is a C₄₋₇ heterocycle, —C₁₋₂ alkyl-C₄₋₇ heterocycle or a 5 to 8 membered bridged-heterocyclic ring system containing 1 to 2 heteroatoms independently selected from nitrogen and oxygen, wherein the C₄₋₇ heterocycle is fully saturated and contains 1 to 2 heteroatoms independently selected from nitrogen and oxygen and at least one of the heteroatoms is oxygen and wherein the C₄₋₇ heterocycle or the 5 to 8 membered bridged-heterocyclic ring system is optionally substituted with 1 or 2 substituents independently selected from the group consisting of C₁₋₄ alkyl, halogen, halo-substitutedC₁₋₄ alkyl, hydroxyl and C₁₋₄ alkoxy; or R¹ is a C₁₋₅ alkyl which is optionally substituted with 1 or 3 substituents independently selected from the group consisting of halogen, halo-substitutedC₁₋₄ alkyl, hydroxy-substitutedC₁₋₄ alkyl, hydroxyl, C₁₋₄ alkoxy and C₃₋₆ cycloalkyl, wherein said C₃₋₆ cycloalkyl is optionally substituted with 1 or 2 substituents independently selected from the group consisting of halogen, halo-substitutedC₁₋₄ alkyl, hydroxyl and C₁₋₄ alkoxy; and the remaining variables are as defined in any one of the first to twentieth embodiments described above.

In a twenty-fifth embodiment, the invention provides a compound of any one of embodiments one to twenty or a pharmaceutically acceptable salt thereof, wherein:

R¹ is a C₄₋₇ heterocycle, —C₁₋₂ alkyl-C₄₋₇ heterocycle or a 5 to 8 membered bridged-heterocyclic ring system containing 1 to 2 heteroatoms independently selected from nitrogen and oxygen, wherein the C₄₋₇ heterocycle is fully saturated and contains 1 to 2 heteroatoms independently selected from nitrogen and oxygen and at least one of heteroatom is oxygen and wherein the C₄₋₇ heterocycle or the 5 to 8 membered bridged-heterocyclic ring system may be optionally substituted with 1 or 2 substituents independently selected from the group consisting of C₁₋₄ alkyl, halogen, halo-substitutedC₁₋₄ alkyl, hydroxyl and C₁₋₄ alkoxy; and the remaining variables are as defined in any one of the first to twentieth embodiments described above.

In a twenty-sixth embodiment, the invention provides a compound of any one of embodiments one to twenty or a pharmaceutically acceptable salt thereof, wherein:

R¹ is a C₁₋₅ alkyl substituted with 1 or 3 substituents independently selected from the group consisting of halo-substitutedC₁₋₄ alkyl, hydroxyl, C₁₋₄ alkoxy and C₄₋₆ cycloalkyl, wherein said C₃₋₆ cycloalkyl is optionally substituted with 1 or 2 substituents independently selected from the group consisting of halogen, halo-substitutedC₁₋₄ alkyl, hydroxyl and C₁₋₄ alkoxy; and the remaining variables are as defined in any one of the first to twentieth embodiments described above.

In a twenty-seventh embodiment, the invention provides a compound of any one of embodiments one to twenty or a pharmaceutically acceptable salt thereof, wherein:

R¹ is a 5 to 8 membered bridged-heterocyclic ring system which contains 1 to 2 heteroatoms independently selected from nitrogen and oxygen, wherein the 5 to 8 membered bridged-heterocyclic ring system is optionally substituted with one or two substituents R^{1a} independently selected from C₁₋₄ alkyl, halogen, halo-substitutedC₁₋₄ alkyl, hydroxyl and C₁₋₄ alkoxy; and the remaining variables are as defined in any one of the first to twentieth embodiments described above. In one embodiment, R¹ is a 5 to 8 membered bridged-heterocyclic ring system containing one oxygen atom and wherein the 5 to 8 membered bridged-heterocyclic ring is optionally substituted with one or two substituents R^{1a} independently selected from C₁₋₄ alkyl, halogen, halo-substitutedC₁₋₄ alkyl, hydroxyl and C₁₋₄ alkoxy; and the remaining variables are as defined in the twenty-seventh embodiment. In one embodiment, R¹ is a 5 to 8 membered bridged-heterocyclic ring system selected from the group consisting of 3-oxabicyclo[3.1.0]hexane, 2-oxabicyclo[2.1.1]hexane, 3-oxabicyclo[2.1.1]hexane, 3-oxabicyclo[4.1.0]heptane, 2-oxabicyclo[2.2.1]heptane, 2-oxabicyclo[2.2.1]heptane, 2-oxabicyclo[3.1.1]heptane, 2-oxabicyclo[2.2.2]octane, 8-oxabicyclo[3.2.1]octane, and 2,6-dioxabicyclo[3.2.1]octane, wherein the 5 to 8 membered bridged-heterocyclic ring is optionally substituted with one or two substituents R^{1a} independently selected from C₁₋₄ alkyl, halogen, halo-substitutedC₁₋₄ alkyl, hydroxyl and C₁₋₄ alkoxy; and the remaining variables are as defined in the twenty-seventh embodiment.

In a twenty-eighth embodiment, the invention provides a compound of any one of embodiments one to twenty or a pharmaceutically acceptable salt thereof, wherein R¹ is a 5 to 8 membered bridged-heterocyclic ring system represented by the following formula:

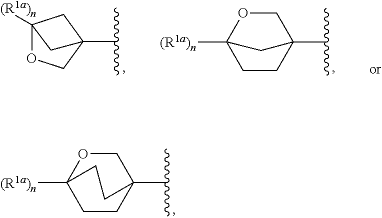

wherein R^{1a} is C₁₋₄ alkyl or halo-substitutedC₁₋₄ alkyl; and n is 0 or 1; and the remaining variables are as defined in any one of the first to twentieth embodiments described above. In one embodiment, R^{1a} is CH₃ or CH₂F.

In a twenty-ninth embodiment, the invention provides a compound of any one of embodiments one to twenty or a pharmaceutically acceptable salt thereof, wherein R¹ is selected from methyl, (tetrahydrofuran-3-yl)methyl, (R)-(tetrahydrofuran-3-yl)methy, (S)-(tetrahydrofuran-3-yl)methy, (1-methyl-2-oxabicyclo[2.1.1]hexan-4-yl)methyl, 2-methoxyethyl, 3-methoxypropyl, 4-methoxybutan-2-yl, 3-methoxy-3-methylbutyl, 3-hydroxy-3-methylbutyl, 3-methoxycyclobutyl, oxetan-3-yl, tetrahydrofuran-3-yl, (R)-tetrahydrofuran-3-yl, (S)-tetrahydrofuran-3-yl, tetrahydro-2H-pyran-3-yl, (R)-tetrahydro-2H-pyran-3-yl, (S)-tetrahydro-2H-pyran-3-yl, tetrahydro-2H-pyran-4-yl, 2,2-dimethyltetrahydro-2H-pyran-4-yl, (R)-2,2-dimethyltetrahydro-2H-pyran-4-yl, (S)-2,2-dimethyltetrahydro-2H-pyran-4-yl, 1-methyl-2-oxabicyclo[2.1.1]hexan-4-yl, (1R,4R)-1-methyl-2-oxabicyclo[2.2.1]heptan-4-yl, (1S,4S)-1-methyl-2-oxabicyclo[2.2.1]heptan-4-yl, 1-methyl-2-oxabicyclo[2.2.1]heptan-4-yl, and 1-methyl-2-oxabicyclo[2.2.2]octan-4-yl; and the remaining variables are as defined in any one of the first to twentieth embodiments described above.

In a thirtieth embodiment of the invention provides a compound of any one of embodiments one to eight or a pharmaceutically acceptable salt thereof, wherein:

R¹ is a C₁₋₅ alkyl which is optionally substituted with 1 or 3 substituents independently selected from the group consisting of halogen, halo-substitutedC₁₋₄ alkyl, hydroxyl, C₁₋₄ alkoxy and C₃₋₆ cycloalkyl, wherein said C₃₋₆ cycloalkyl is optionally substituted with 1 or 2 substituents independently selected from the group consisting of halogen, halo-substitutedCl₁₋₄ alkyl, hydroxyl and C₁₋₄ alkoxy; and

R³ is pyridinyl optionally substituted with 1 or 2 substituents independently selected from and C₁₋₄ alkyl and halo-substitutedC₁₋₄ alkyl; and the remaining variables are as defined in any one of the first to eighth embodiments described above.

In a thirty-first embodiment of the invention provides a compound of any one of embodiments one to eight or a pharmaceutically acceptable salt thereof, wherein:

R¹ is a fully saturated C₄₋₇ heterocycle or a 5 to 8 membered bridged-heterocyclic ring system which contain 1 to 2 heteroatoms independently selected from nitrogen and oxygen, said C₄₋₇ heterocycle or a 5 to 8 membered bridged-heterocyclic ring system may be optionally substituted with 1 or 2 substituents independently selected from the group consisting of C₁₋₄ alkyl, halogen, halo-substitutedC₁₋₄ alkyl, hydroxyl and C₁₋₄ alkoxy; and

R³ is pyridinyl optionally substituted with 1 or 2 substituents independently selected from and C₁₋₄ alkyl and halo-substitutedC₁₋₄ alkyl; and the remaining variables are as defined in any one of the first to eighth embodiments described above.

In a thirty-second embodiment, the invention provides a compound of any one of the first to thirty-first embodiments or a pharmaceutically acceptable salt thereof, wherein R⁶ is an optionally substituted C₁₋₅ alkyl or an optionally substituted C₃ 6 cycloalkyl, wherein the C₁₋₅ alkyl is optionally substituted with 1 to 3 substituents independently selected from halogen, hydroxyl and C₁₋₄ alkoxy and the C₃₋₆ cycloalkyl is optionally substituted with 1 to 3 substituents independently selected from halo, C₁₋₄ alkyl, halo-substitutedC₁₋₄ alkyl and C₁₋₄ alkoxy; and the remaining variables are as defined in any one of the first to thirty-first embodiments.

In a thirty-third embodiment, the invention provides a compound of any one of the first to thirty-first embodiments or a pharmaceutically acceptable salt thereof, wherein R⁶ is selected from methyl, (3,3-difluorocyclobutyl)methyl, ethyl, isopropyl, cyclobutyl, 3-(difluoromethyl)cyclobutyl, (1R,3R)-3-(difluoromethyl)cyclobutyl, 3-methoxycyclobutyl, (1R,3R)-3-methoxycyclobutyl, cyclopentyl, and tetrahydrofuran-3-yl; and the remaining variables are as defined in any one of the first to thirty-first embodiments.

In a thirty-fourth embodiment, the invention provides a compound of the first or second embodiment, wherein the compound is represented by formula (Ia), (Ib), (Ic) or (Id) or a pharmaceutically acceptable salt thereof, wherein:

R¹ is —C₁₋₂ alkyl-C₄₋₇ heterocycle or a 5 to 8 membered bridged-heterocyclic ring system containing 1 to 2 heteroatoms independently selected from nitrogen and oxygen, wherein the C₄₋₇ heterocycle is fully saturated and contains 1 to 2 heteroatoms independently selected from nitrogen, sulfur and oxygen and wherein the C₄₋₇ heterocycle and the 5 to 8 membered bridged-heterocyclic ring system is optionally substituted with one or two substituents R^{1a};

R^{1a}, for each occurrence, is independently selected from C₁₋₄ alkyl, halogen, halo-substitutedC₁₋₄ alkyl, hydroxyl and C₁₋₄ alkoxy;

R³ is phenyl, a 5 or 6 membered monocyclic heteroaryl having 1 to 2 heteroatoms independently selected from nitrogen and oxygen, pyridinyl-2 (1H)-one or a 8 to 10 membered bicyclic heteroaryl having 1 to 3 heteroatoms independently selected from nitrogen and oxygen, wherein the monocyclic heteroaryl, pyridinyl-2 (1H)-one or the bicyclic heteroaryl are each optionally substituted with 1 or 2 R⁴;

R⁴, for each occurrence, is independently selected from hydroxyl, halo, halo-substitutedC₁₋₄ alkyl, —NR⁸R⁹, and C₁₋₄ alkyl;

R⁵ is OR⁶; and

R⁶ is an optionally substituted C₁₋₅ alkyl or an optionally substituted C₃₋₆ cycloalkyl, wherein the C₁₋₅ alkyl is optionally substituted with 1 to 3 substituents independently selected from halogen, hydroxyl and C₁₋₄ alkoxy and the C₃₋₆ cycloalkyl is optionally substituted with 1 to 3 substituents independently selected from halo, C₁₋₄ alkyl, halo-substitutedC₁₋₄ alkyl and C₁₋₄ alkoxy.

In one embodiment, the compound of the thirty-fourth embodiment is represented by formula (Ic) or (Id) or a pharmaceutically acceptable salt thereof.

In a thirty-fifth embodiment, the invention provides a compound of the thirty-fourth embodiment, or a pharmaceutically acceptable salt thereof, wherein:

R¹ is —C₁₋₂ alkyl-C₄₋₇ heterocycle or a 5 to 8 membered bridged-heterocyclic ring system containing one oxygen atom, wherein the C₄₋₇ heterocycle contains one oxygen atom and wherein the C₄₋₇ heterocycle and the 5 to 8 membered bridged-heterocyclic ring system is optionally substituted with one substituent R^{1a};

R^{1a} is C₁₋₄ alkyl or halo-substitutedC₁₋₄ alkyl;

R³ is phenyl, a 5 or 6 membered monocyclic heteroaryl having 1 to 2 nitrogen atoms, pyridinyl-2 (1H)-one or a 8 to 10 membered bicyclic heteroaryl having 2 to 3 nitrogen atoms, wherein the monocyclic heteroaryl, pyridinyl-2 (1H)-one or the bicyclic heteroaryl are each optionally substituted with 1 or 2 R⁴;

R⁴, for each occurrence, is independently selected from hydroxyl, halo, C₁₋₄ alkoxy, halo-substitutedC₁₋₄ alkyl, and C₁₋₄ alkyl;

R⁵ is OR⁶; and

R⁶ is an optionally substituted C₁₋₅ alkyl or an optionally substituted C₃₋₆ cycloalkyl, wherein the C₁₋₅ alkyl is optionally substituted with 1 to 3 substituents independently selected from halogen and the C₃₋₆ cycloalkyl is optionally substituted with 1 to 3 substituents independently selected from C₁₋₄ alkyl, halo-substitutedC₁₋₄ alkyl and halogen.

In a thirty-sixth embodiment, the invention provides a compound of the thirty-fifth embodiment, or a pharmaceutically acceptable salt thereof, wherein:

R¹ is

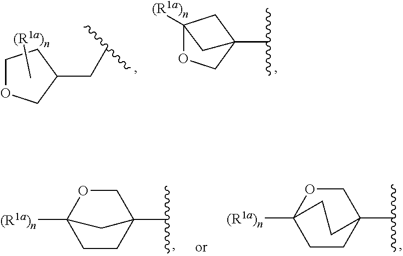

R^{1a} is C₁₋₄ alkyl or halo-substitutedC₁₋₄ alkyl;

n is 0 or 1;

R³ is

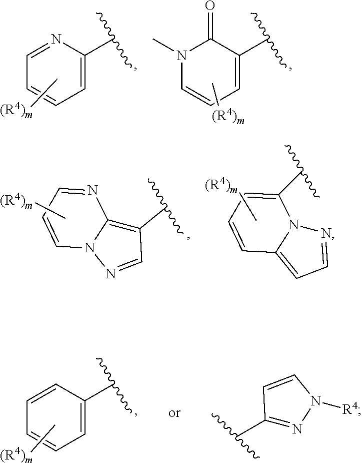

R⁴ is halo, C₁₋₄ alkoxy, C₁₋₄ alkyl or halo-substitutedC₁₋₄ alkyl;

m is 0 or 1;

R⁵ is OR⁶; and

R⁶ is C₁₋₄ alkyl or C₄₋₆ cycloalkyl.

In a thirty-seventh embodiment, the invention provides a compound of the thirty-sixth embodiment, or a pharmaceutically acceptable salt thereof, wherein:

R^{1a} is CH₃; R⁴ is CH₃, F, OMe, or CHF₂; and R⁶ is —CH(CH₃)₂, cyclobutyl, or cyclopentyl; and the remaining variables are as defined in the thirty-sixth embodiment.

In a thirty-eight embodiment, the invention provides a compound of formula (I′), (I), (Ia), (Ib), (Ic) or (Id), or a pharmaceutically acceptable salt thereof, wherein:

R¹ is a fully saturated C₄₋₇ heterocycle, —C₁₋₂ alkyl-C₄₋₇ heterocycle, or a fully saturated 5 to 8 membered bridged-heterocyclic ring system which contain 1 to 2 heteroatoms independently selected from nitrogen and oxygen, said C₄₋₇ heterocycle or said 5 to 8 membered bridged-heterocyclic ring system is optionally substituted with 1 or 2 substituents independently selected from the group consisting of C₁₋₄ alkyl, halogen, halo-substitutedC₁₋₄ alkyl, hydroxyl and C₁₋₄ alkoxy;

R³ is phenyl, 5 or 6 membered monocyclic heteroaryl having 1 to 3 heteroatoms independently selected from nitrogen and oxygen, pyridinyl-2 (1H)-one, pyrimidin-4 (3H)-one or a 9 to 10 membered bicyclic heteroaryl having 1 to 3 heteroatoms independently selected from nitrogen and oxygen, wherein the monocyclic heteroaryl, pyridinyl-2 (1H)-one, pyrimidin-4 (3H)-one or the bicyclic heteroaryl are each optionally substituted with 1 or 2 R⁴;

R⁴, for each occurrence, is independently selected from hydroxyl, halo, halo-substitutedC₁₋₄ alkyl, —NR⁸R⁹, C₁₋₄ alkoxy, C₃₋₆ cycloalkyl, and C₁₋₄ alkyl;

R⁵ is OR⁶; and

R⁶ is an optionally substituted C₁₋₅ alkyl or an optionally substituted C₃₋₆ cycloalkyl, wherein the C₁₋₅ alkyl is optionally substituted with 1 to 3 substituents independently selected from halogen, hydroxyl and C₁₋₄ alkoxy and the C₃₋₆ cycloalkyl is optionally substituted with 1 to 3 substituents independently selected from halo, C₁₋₄ alky, halo-substitutedC₁₋₄ alkyl and C₁₋₄ alkoxy.

In a thirty-ninth embodiment, the invention provides a compound of the thirty-eighth embodiment or a pharmaceutically acceptable salt thereof, wherein:

R¹ a fully saturated C₄₋₇ heterocycle, —C₁₋₂ alkyl-C₄₋₇ heterocycle, or a fully saturated 5 to 8 membered bridged-heterocyclic ring system, wherein the C₄₋₇ heterocycle is selected from the group consisting of tetrahydrofuran, tetrahydropyran, and 1,4-dioxane and the fully saturated 5 to 8 membered bridged-heterocyclic ring system is selected from the group consisting of 3-oxabicyclo[3.1.0]hexane, 2-oxabicyclo[2.1.1]hexane, 3-oxabicyclo[2.1.1]hexane, 3-oxabicyclo[4.1.0]heptane, 2-oxabicyclo[2.2.1]heptane, 2-oxabicyclo[2.2.1]heptane, 2-oxabicyclo[3.1.1]heptane, 2-oxabicyclo[2.2.2]octane, 8-oxabicyclo[3.2.1]octane, and 2,6-dioxabicyclo[3.2.1]octane, wherein the C₄₋₇ heterocycle or the 5 to 8 membered bridged-heterocyclic ring system is optionally substituted with 1 or 2 substituents independently selected from the group consisting of C₁₋₄ alkyl, halogen, halo-substitutedC₁₋₄ alkyl, hydroxyl and C₁₋₄ alkoxy;

R³ is phenyl, 5 or 6 membered monocyclic heteroaryl selected from the group consisting of pyridine, pyrimidine, 2H-1,2,3-triazole, isoxazole, isothiazole, thiazole, pyrazole and thiophene, pyridinyl-2 (1H)-one, pyrimidin-4 (3H)-one, or a 9 to 10 membered bicyclic heteroaryl selected from pyrazolo[1,5-a]pyridine, [1,2,4]triazolo[4,3-a]pyridine, isothiazolo[4,3-b]pyridine, pyrazolo[1,5-a]pyrimidine, pyrido[3,2-d]pyrimidine, imidazo[1,2-b]pyridazine, thieno[2,3-b]pyrazine, 1H-benzo[d]imidazole, benzo[d]thiazole, 1,6-naphthyridine, 1,5-naphthyridine, and 2H-indazole, wherein the monocyclic heteroaryl, pyridinyl-2 (1H)-one, pyrimidin-4 (3H)-one and the bicyclic heteroaryl are each optionally substituted with 1 or 2 R⁴; and the remaining variables are as defined above in the thirty-eighth embodiment.

In a fortieth embodiment, the invention provides a compound described herein (e.g., a compound of any one examples 1-140) or a pharmaceutically acceptable salt thereof.

In a forty-first embodiment of the invention provides a compound according embodiment one, selected from the group consisting of:
6-methoxy-N-(6-methoxypyridin-2-yl)-2-((tetrahydrofuran-3-yl)methyl)-2H-indazole-5-carboxamide;
6-methoxy-N-(pyridin-2-yl)-2-((tetrahydrofuran-3-yl)methyl)-2H-indazole-5-carboxamide;
6-methoxy-N-(6-methoxypyridin-2-yl)-2-((1-methyl-2-oxabicyclo[2.1.1]hexan-4-yl)methyl)-2H-indazole-5-carboxamide;
6-methoxy-N-(6-methoxypyridin-2-yl)-2-(tetrahydrofuran-3-yl)-2H-indazole-5-carboxamide;
6-methoxy-N-(6-methoxypyridin-2-yl)-2-(tetrahydro-2H-pyran-4-yl)-2H-indazole-5-carboxamide;
6-methoxy-N-(pyridin-2-yl)-2-(tetrahydro-2H-pyran-4-yl)-2H-indazole-5-carboxamide;
N-(6-methoxypyridin-2-yl)-2-((tetrahydrofuran-3-yl)methyl)-2H-pyrazolo[3,4-c]pyridine-5-carboxamide;
N-(6-(difluoromethyl)pyridin-2-yl)-2-((tetrahydrofuran-3-yl)methyl)-2H-pyrazolo[3,4-c]pyridine-5-carboxamide;
N-(6-methoxypyridin-2-yl)-7-methyl-2-((tetrahydrofuran-3-yl)methyl)-2H-indazole-5-carboxamide;
N-(6-(difluoromethyl)pyridin-2-yl)-6-methoxy-2-(2-methoxyethyl)-2H-indazole-5-carboxamide;
N-(6-(difluoromethyl)pyridin-2-yl)-6-methoxy-2-((tetrahydrofuran-3-yl)methyl)-2H-indazole-5-carboxamide;
N-(6-(difluoromethyl)pyridin-2-yl)-6-methoxy-2-(3-methoxy-3-methylbutyl)-2H-indazole-5-carboxamide;
N-(6-(difluoromethyl)pyridin-2-yl)-2-(3-hydroxy-3-methylbutyl)-6-isopropoxy-2H-indazole-5-carboxamide;
2-(3-hydroxy-3-methylbutyl)-7-methoxy-N-(6-methoxypyridin-2-yl)-2H-indazole-5-carboxamide;
7-methoxy-2-(3-methoxy-3-methylbutyl)-N-(6-methoxypyridin-2-yl)-2H-indazole-5-carboxamide;
7-methoxy-N-(6-methoxypyridin-2-yl)-2-(tetrahydro-2H-pyran-4-yl)-2H-indazole-5-carboxamide;
N-(6-(difluoromethyl)pyridin-2-yl)-6-methoxy-2-(3-methoxypropyl)-2H-indazole-5-carboxamide;
(R)—N-(6-(difluoromethyl)pyridin-2-yl)-6-methoxy-2-((tetrahydrofuran-3-yl)methyl)-2H-indazole-5-carboxamide;
(S)—N-(6-(difluoromethyl)pyridin-2-yl)-6-methoxy-2-((tetrahydrofuran-3-yl)methyl)-2H-indazole-5-carboxamide;
(R)—N-(6-(difluoromethyl)pyridin-2-yl)-6-isopropoxy-2-((tetrahydrofuran-3-yl)methyl)-2H-indazole-5-carboxamide;
(S)—N-(6-(difluoromethyl)pyridin-2-yl)-6-isopropoxy-2-((tetrahydrofuran-3-yl)methyl)-2H-indazole-5-carboxamide;
(S)-6-methoxy-N-(6-methoxypyridin-2-yl)-2-((tetrahydrofuran-3-yl)methyl)-2H-indazole-5-carboxamide; and
(R)-6-methoxy-N-(6-methoxypyridin-2-yl)-2-((tetrahydrofuran-3-yl)methyl)-2H-indazole-5-carboxamide;
   or a pharmaceutically acceptable salt thereof.

A forty-second embodiment of the invention provides a pharmaceutical composition comprising a compound according to any one of the preceding embodiments, or a pharmaceutically acceptable salt thereof.

A forty-third embodiment of the invention provides a pharmaceutical composition according to embodiment forty-second, or a pharmaceutically acceptable salt thereof and one or more pharmaceutically acceptable carriers, or diluents.

A forty-fourth embodiment of the invention provides a pharmaceutical composition according to embodiment forty-third, further comprising one or more additional pharmaceutical agent(s).

One embodiment of the invention includes a method of decreasing the expression or activity of IRAK4, or to otherwise affect the properties and/or behavior of IRAK4 polypeptides or polynucleotides comprising administering to said mammal an effective amount of at least one compound described herein, or a pharmaceutically acceptable salt thereof.

A forty-fifth embodiment of the invention is a method of treating an IRAK4 mediated disease in a subject comprising administering to the subject a compound or a pharmaceutically acceptable salt thereof of any one of embodiments one to forty-one or a pharmaceutical composition thereof of any one of embodiments forty-two to forty-four.

A forty-sixth embodiment, the invention provides the use of a compound according to any one of embodiments one to forty-one, for the treatment of a disorder or disease in a subject mediated by IRAK4.

A forty-seventh embodiment, the invention provides the use of a compound according to any one of embodiments one to forty-one in the manufacture of a medicament for the treatment of a disorder or disease in a subject mediated by IRAK4.

A forty-eighth embodiment of the invention comprising a method of treatment according to embodiment forty-five wherein the IRAK4 mediated disease is selected from an autoimmune disease, an inflammatory disease, bone diseases, metabolic diseases, neurological and neurodegenerative diseases and/or disorders, cancer, cardiovascular diseases, allergies, asthma, Alzheimer's disease, hormone-related diseases, Ischemic stroke, Cerebral Ischemia, hypoxia, TBI (Traumatic Brain Injury), CTE (Chronic Traumatic Encephalopathy), epilepsy, Parkinson's disease (PD), Multiple Sclerosis (MS) and Amyotrophic Lateral Sclerosis (ALS).

A forty-ninth embodiment of the invention comprising a method of treatment according to embodiment forty-five, wherein the IRAK4 mediated disease is selected from disorders and/or conditions associated with inflammation and pain, proliferative diseases, hematopoietic disorders, hematological malignancies, bone disorders, fibrosis diseases and/or disorders, metabolic disorders, muscle diseases and/or disorders, respiratory diseases, pulmonary disorders, genetic development diseases, chronic inflammatory demyelinating neuropathies, vascular or heart diseases, ophthalmic diseases and ocular diseases.

A fiftieth embodiment of the invention comprising a use of a compound according to embodiment forty-seven, wherein the IRAK4 mediated disease is selected from an autoimmune disease, an inflammatory disease, bone diseases, metabolic diseases, neurological and neurodegenerative diseases and/or disorders, cancer, cardiovascular diseases, allergies, asthma, Alzheimer's disease, hormone-related diseases, Ischemic stroke, Cerebral Ischemia, hypoxia, TBI (Traumatic Brain Injury), CTE (Chronic Traumatic Encephalopathy), epilepsy, Parkinson's disease (PD), Multiple Sclerosis (MS) and Amyotrophic Lateral Sclerosis (ALS).

A fifty-first embodiment of the invention comprising a use of a compound according to embodiment forty-seven, wherein the IRAK4 mediated disease is selected from disorders and/or conditions associated with inflammation and pain, proliferative diseases, hematopoietic disorders, hematological malignancies, bone disorders, fibrosis diseases and/or disorders, metabolic disorders, muscle diseases and/or disorders, respiratory diseases, pulmonary disorders, genetic development diseases, chronic inflammatory demyelinating neuropathies, vascular or heart diseases ophthalmic diseases and ocular diseases.

The compounds, or pharmaceutically acceptable salts thereof described herein may be used to decrease the expression or activity of IRAK4, or to otherwise affect the properties and/or behavior of IRAK4 polypeptides or polynucleotides, e.g., stability, phosphorylation, kinase activity, interactions with other proteins, etc.

One embodiment of the invention includes a method of decreasing the expression or activity of IRAK1, or to otherwise affect the properties and/or behavior of IRAK1 polypeptides or polynucleotides comprising administering to said mammal an effective amount of at least one compound described herein, or a pharmaceutically acceptable salt thereof.

In one embodiment, R¹ is elected from the group consisting of

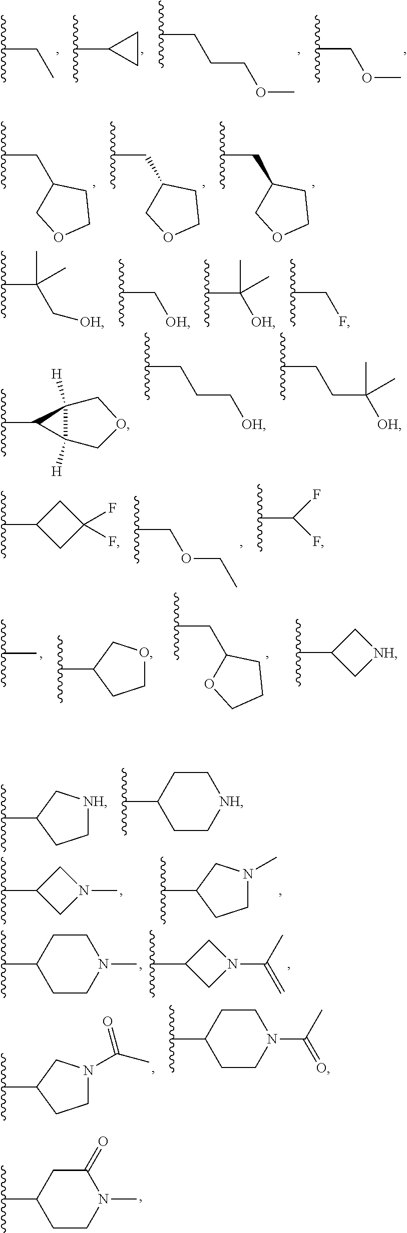

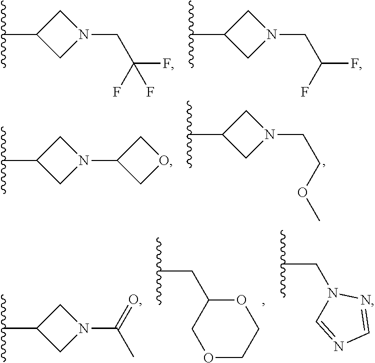

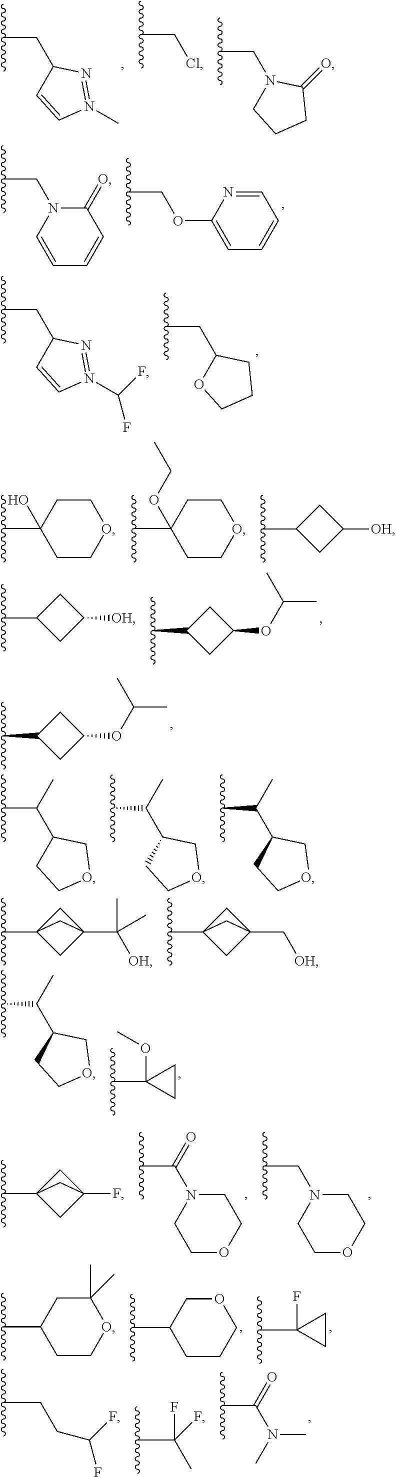

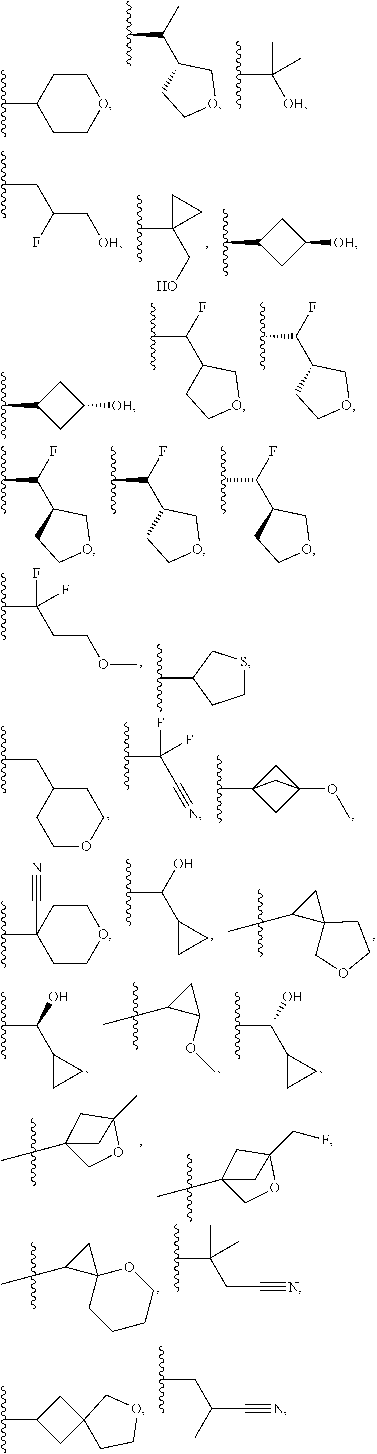

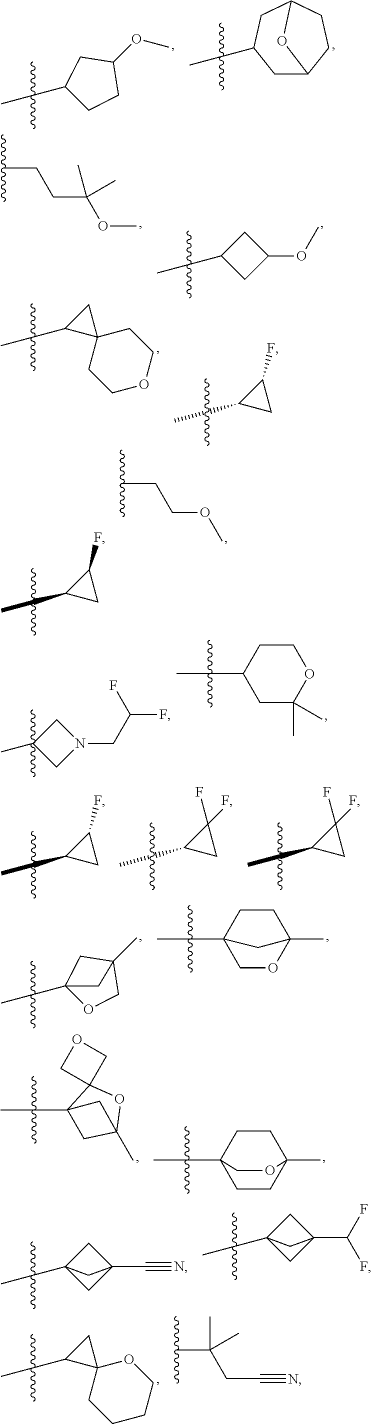

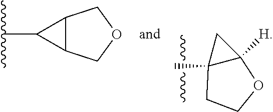

In one embodiment, R¹ is elected from the group consisting of

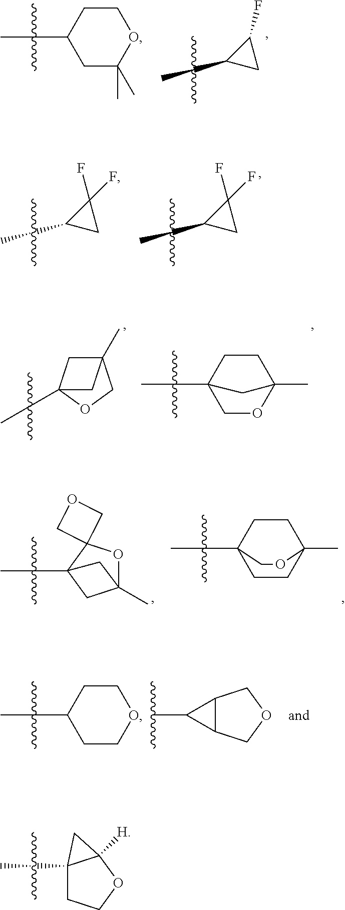

In one embodiment, R³ is elected from the group consisting of

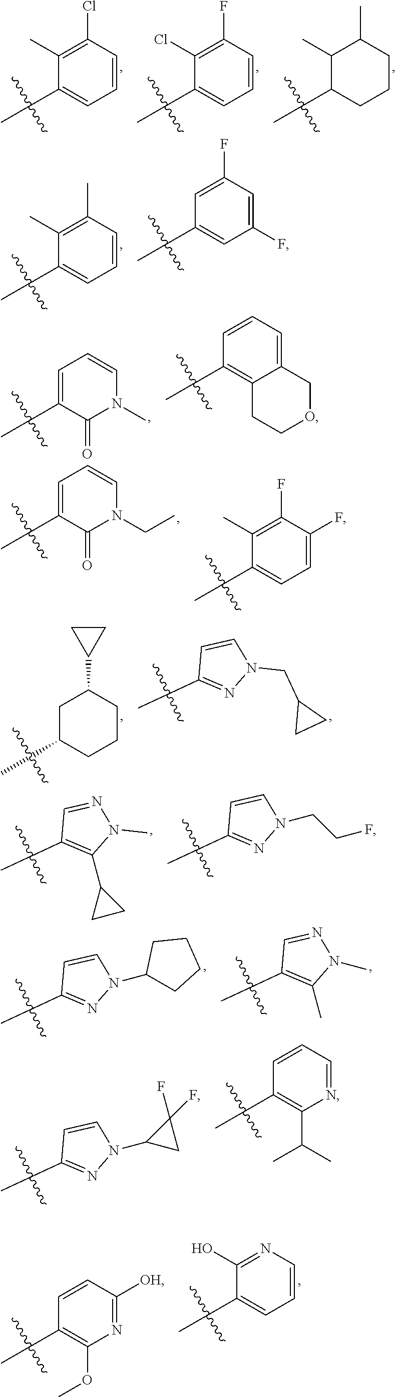

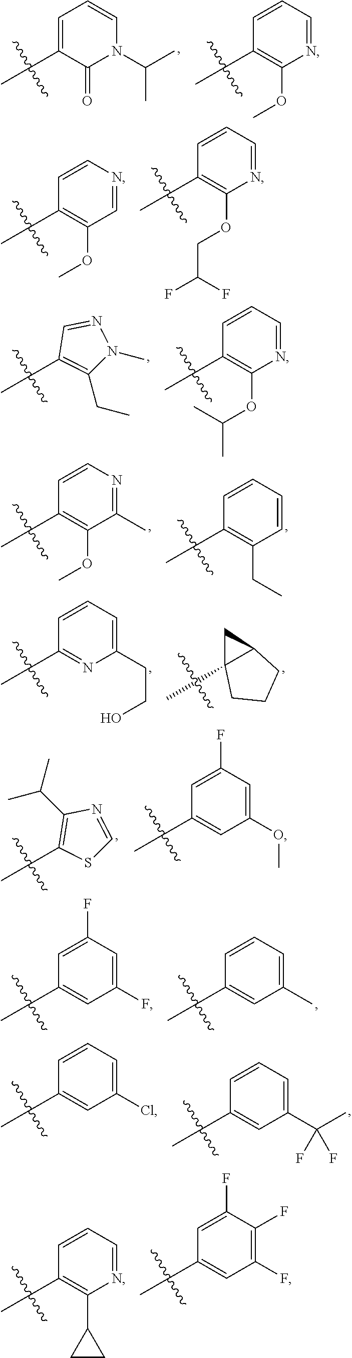

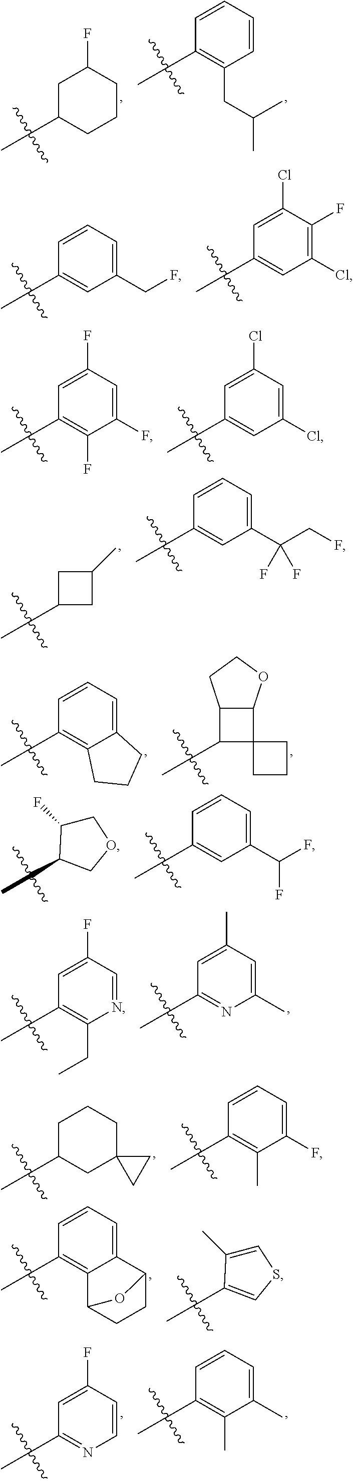

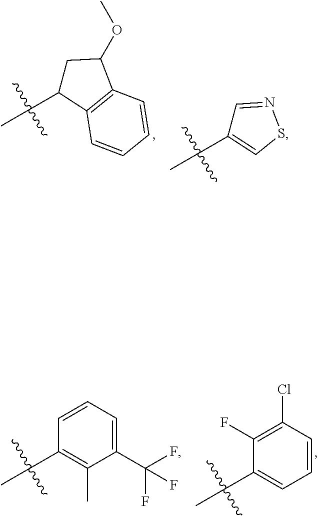 [SMILES: *C1CC(OC)c2ccccc21 ; *c1cnsc1 ; *c1cccc(C(F)(F)F)c1C ; *c1cccc(Cl)c1F]

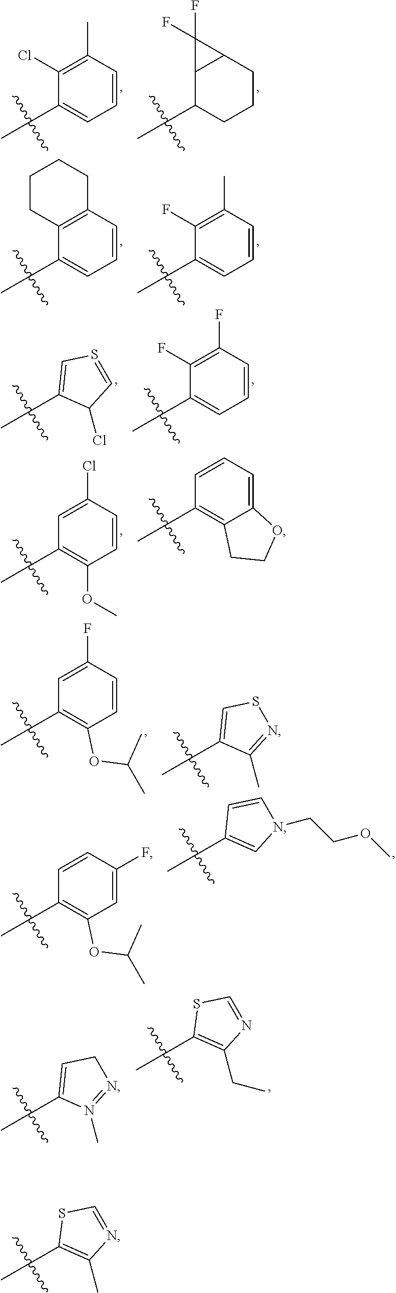

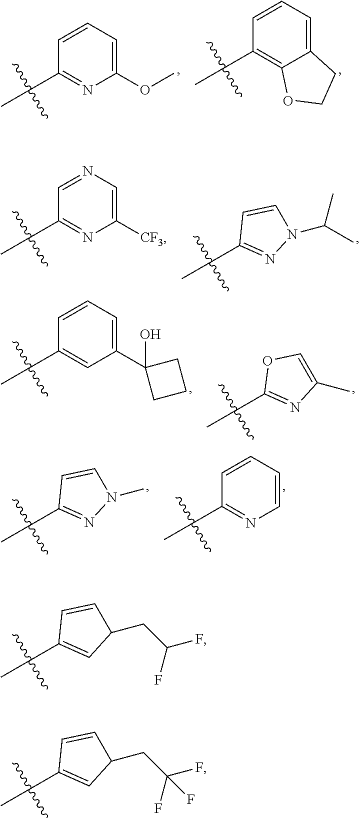

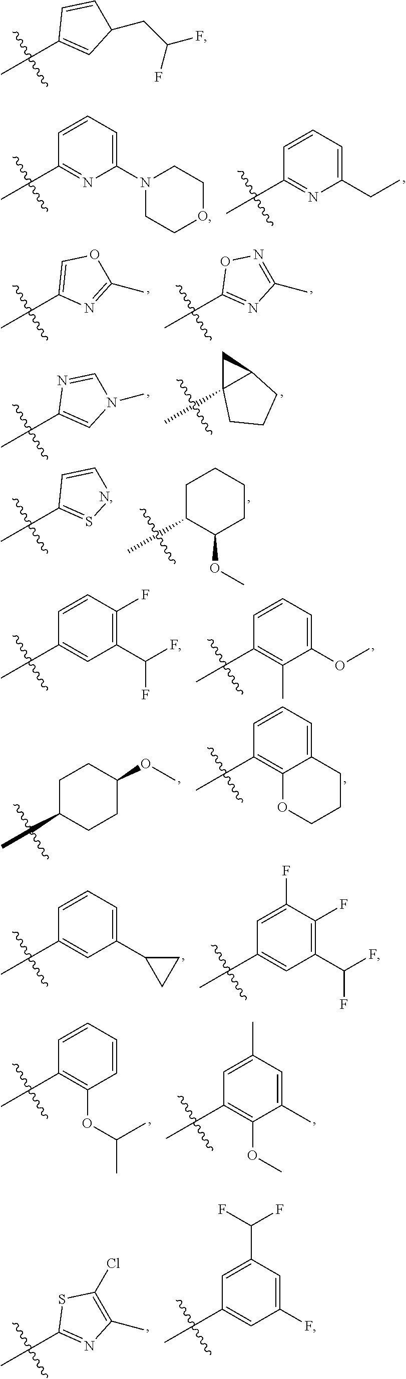

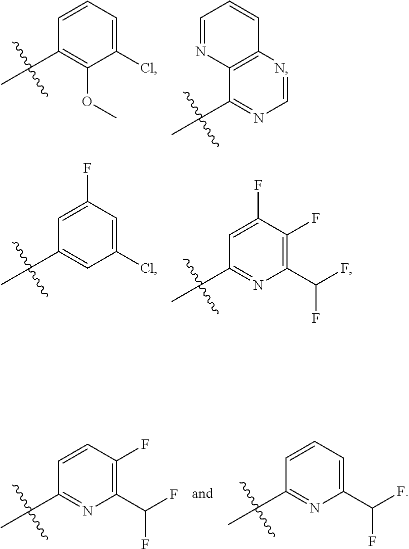

In one embodiment, R³ is elected from the group consisting of

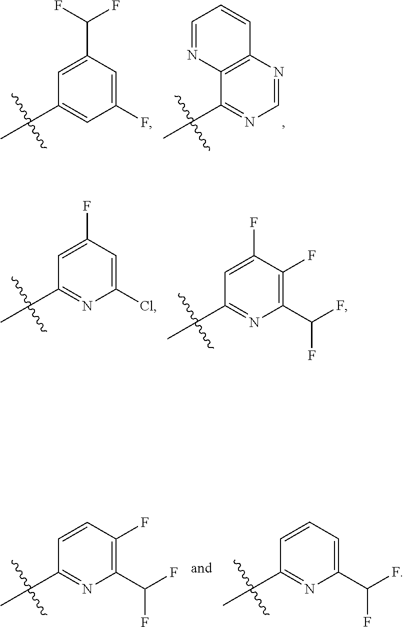 [SMILES: *c1cc(F)cc(C(F)F)c1 ; *c1ncnc2cccnc12 ; *c1cc(F)cc(Cl)n1 ; *c1cc(F)c(F)c(C(F)F)n1 ; *c1ccc(F)c(C(F)F)n1 ; *c1cccc(C(F)F)n1]

In one embodiment, R⁵ is elected from the group consisting of

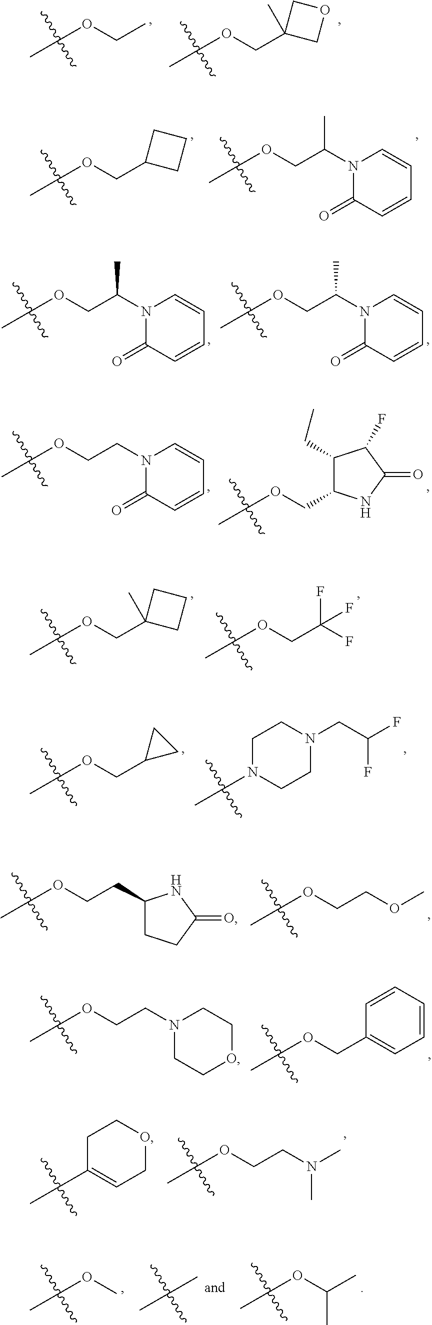

In one embodiment, R⁵ is elected from the group consisting of

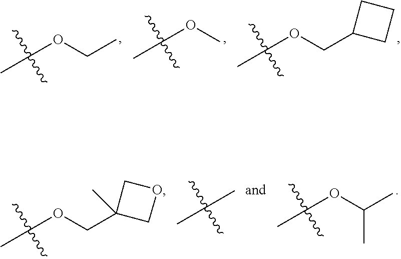

One embodiment of the invention includes a method of decreasing the expression or activity of IRAK4, or to otherwise affect the properties and/or behavior of IRAK4 polypeptides or polynucleotides comprising administering to said subject an effective amount of at least one compound described herein, or a pharmaceutically acceptable salt thereof.

One embodiment of the invention includes a method for treating an inflammatory disease in a subject, the method comprising administering to the patient a therapeutically effective amount of a compound described herein, or a pharmaceutically acceptable salt thereof, thereby treating the inflammatory disease in the subject.

In one embodiment, the inflammatory disease is a pulmonary disease or a disease of the airway.

In one embodiment, the pulmonary disease and disease of the airway is selected from Adult Respiratory Disease Syndrome (ARDS), Chronic Obstructive Pulmonary Disease (COPD), pulmonary fibrosis, interstitial lung disease, asthma, chronic cough, and allergic rhinitis.

In one embodiment, the inflammatory disease is selected from transplant rejection, CD14 mediated sepsis, non-CD14 mediated sepsis, inflammatory bowel disease, Behcet's syndrome, ankylosing spondylitis, sarcoidosis, and gout.

One embodiment of the invention includes a method for treating an autoimmune disease, cancer, cardiovascular disease, a disease of the central nervous system, a disease of the skin, an ophthalmic disease and condition, and bone disease in a subject, the method comprising administering to the patient a therapeutically effective amount of a compound disclosed herein, or a pharmaceutically acceptable salt thereof, thereby treating the autoimmune disease, cancer, cardiovascular disease, disease of the central nervous system, disease of the skin, ophthalmic disease and condition, and bone disease in the subject.

In one embodiment, the autoimmune disease is selected from rheumatoid arthritis, systemic lupus erythematosus, multiple sclerosis, diabetes, systemic sclerosis, and Sjogren's syndrome.

In one embodiment, the autoimmune disease is type 1 diabetes.

In one embodiment, the cancer is selected from Waldenstrim's macroglobulinemia, solid tumors, skin cancer, and lymphoma.

In one embodiment, the cardiovascular disease is selected from stroke and atherosclerosis.

In one embodiment, the disease of the central nervous system is a neurodegenerative disease.

In one embodiment, the disease of the skin is selected from rash, contact dermatitis, psoriasis, and atopic dermatitis.

In one embodiment, the bone disease is selected from osteoporosis and osteoarthritis.

In one embodiment, the inflammatory bowel disease is selected from Crohn's disease and ulcerative colitis.

One embodiment of the invention includes a method for treating an ischemic fibrotic disease, the method comprising administering to the patient a therapeutically effective amount of a compound described herein, or a pharmaceutically acceptable salt thereof, thereby treating the ischemic fibrotic disease in the subject. In one embodiment, the ischemic fibrotic disease is selected from stroke, acute lung injury, acute kidney injury, ischemic cardiac injury, acute liver injury, and ischemic skeletal muscle injury.

One embodiment of the invention includes a method for treating post-organ transplantation fibrosis, the method comprising administering to the patient a therapeutically effective amount of a compound described herein, or a pharmaceutically acceptable salt thereof, thereby treating post-organ transplantation fibrosis in the subject.

One embodiment of the invention includes a method for treating hypertensive or diabetic end organ disease, the method comprising administering to the patient a therapeutically effective amount of a compound described herein, or a pharmaceutically acceptable salt thereof, thereby treating hypertensive or diabetic end organ disease in the subject.

One embodiment of the invention includes a method for treating hypertensive kidney disease, the method comprising administering to the patient a therapeutically effective amount of a compound described herein, or a pharmaceutically acceptable salt thereof, thereby treating hypertensive kidney disease in the subject.

One embodiment of the invention includes a method for treating idiopathic pulmonary fibrosis (IPF), the method comprising administering to the patient a therapeutically effective amount of a compound described herein, or a pharmaceutically acceptable salt thereof, thereby treating IPF in the subject.

One embodiment of the invention includes a method for treating scleroderma or systemic sclerosis, the method comprising administering to the patient a therapeutically effective amount of a compound described herein, or a pharmaceutically acceptable salt thereof, thereby treating scleroderma or systemic sclerosis in the subject.

One embodiment of the invention includes a method for treating liver cirrhosis, the method comprising administering to the patient a therapeutically effective amount of a compound described herein, or a pharmaceutically acceptable salt thereof, thereby treating liver cirrhosis in the subject.

One embodiment of the invention includes a method for treating fibrotic diseases wherein tissue injury and/or inflammation are present, the method comprising administering to the patient a therapeutically effective amount of a compound described herein, or a pharmaceutically acceptable salt thereof, thereby treating fibrotic diseases where tissue injury and/or inflammation are present in the subject. The fibrotic diseases include, for example, pancreatitis, peritonitis, burns, glomerulonephritis, complications of drug toxicity, and scarring following infections.

Scarring of the internal organs is a major global health problem, which is the consequence of subclinical injury to the organ over a period of time or as the sequela of acute severe injury or inflammation. All organs may be affected by scarring and currently there are few therapies the specifically target the evolution of scarring. Increasing evidence indicates that scarring per se provokes further decline in organ function, inflammation and tissue ischemia. This may be directly due the deposition of the fibrotic matrix which impairs function such as in contractility and relaxation of the heart and vasculature or impaired inflation and deflation of lungs, or by increasing the space between microvasculature and vital cells of the organ that are deprived of nutrients and distorting normal tissue architecture.

However recent studies have shown that myofibroblasts themselves are inflammatory cells, generating cytokines, chemokines and radicals that promote injury; and myofibroblasts appear as a result of a transition from cells that normally nurse and maintain the microvasculature, known as pericytes. The consequence of this transition of phenotype is an unstable microvasculature that leads to aberrant angiogenesis, or rarefaction.

The present disclosure relates to methods and compositions for treating, preventing, and/or reducing scarring in organs. More particularly, the present disclosure relates to methods and composition for treating, preventing, and/or reducing scarring in kidneys.

It is contemplated that the present disclosure, methods and compositions described herein can be used as an antifibrotic, or used to treat, prevent, and/or reduce the severity and damage from fibrosis.

It is additionally contemplated that the present disclosure, methods and compositions described herein can be used to treat, prevent, and/or reduce the severity and damage from fibrosis.

It is further contemplated that the present disclosure, methods and compositions described herein can used as an anti-inflammatory, used to treat inflammation.

Some non-limiting examples of organs include: kidney, hearts, lungs, stomach, liver, pancreas, hypothalamus, stomach, uterus, bladder, diaphragm, pancreas, intestines, colon, and so forth.

In certain embodiments, the present invention relates to the aforementioned methods, wherein said compound is administered parenterally.

In certain embodiments, the present invention relates to the aforementioned methods, wherein said compound is administered intramuscularly, intravenously, subcutaneously, orally, pulmonary, rectally, intrathecally, topically or intranasally.

In certain embodiments, the present invention relates to the aforementioned methods, wherein said compound is administered systemically.

In certain embodiments, the present invention relates to the aforementioned methods, wherein said subject is a mammal.

In certain embodiments, the present invention relates to the aforementioned methods, wherein said subject is a primate.

In certain embodiments, the present invention relates to the aforementioned methods, wherein said subject is a human.

The compounds and intermediates described herein may be isolated and used as the compound per se. Alternatively, when a moiety is present that is capable of forming a salt, the compound or intermediate may be isolated and used as its corresponding salt. As used herein, the terms “salt” or “salts” refers to an acid addition or base addition salt of a compound of the invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The salts can be synthesized by conventional chemical methods from a compound containing a basic or acidic moiety. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two.

Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

It will be recognized by those skilled in the art that the compounds of the present invention may contain chiral centers and as such may exist in different stereoisomeric forms. As used herein, the term “an optical isomer” or “a stereoisomer” refers to any of the various stereo isomeric configurations which may exist for a given compound of the present invention. It is understood that a substituent may be attached at a chiral center of a carbon atom. Therefore, the invention includes enantiomers, diastereomers or racemates of the compound.

“Enantiomers” are a pair of stereoisomers that are non-superimposable mirror images of each other. A 1:1 mixture of a pair of enantiomers is a “racemic” mixture. The term is used to designate a racemic mixture where appropriate. When designating the stereochemistry for the compounds of the present invention, a single stereoisomer with known relative and absolute configuration of the two chiral centers is designated using the conventional RS system (e.g., (1S,2S)); a single stereoisomer with known relative configuration but unknown absolute configuration is designated with stars (e.g., (1R*,2R*)); and a racemate with two letters (e.g, (1RS,2RS) as a racemic mixture of (1R,2R) and (1S,2S); (1RS,2SR) as a racemic mixture of (1R,2S) and (1S,2R)). “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)- or (S)-.

Unless specified otherwise, the compounds of the present invention are meant to include all such possible stereoisomers, including racemic mixtures, optically pure forms and intermediate mixtures. Optically active (R)- and (S)-stereoisomers may be prepared using chiral synthons or chiral reagents, or resolved using conventional techniques (e.g., separated on chiral SFC or HPLC chromatography columns, such as CHIRALPAK® and CHIRALCEL® available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

### Pharmacology and Utility

Compounds of the present invention have been found to modulate IRAK4 activity and may be beneficial for the treatment of neurological, neurodegenerative and other additional diseases.

Another aspect of the invention provides a method for treating or lessening the severity of a disease, disorder, or condition associated with the modulation of IRAK4 in a subject, which comprises administering to the subject a compound of Formula (I′) or (I) or a pharmaceutically acceptable salt thereof.

In certain embodiments, the present invention provides a method of treating a condition, disease or disorder implicated by a deficiency of IRAK4 activity, the method comprising administering a composition comprising a compound of formula (I′) or (I) to a subject, preferably a mammal, in need of treatment thereof.

According to the invention an “effective dose” or an “effective amount” of the compound or pharmaceutical composition is that amount effective for treating or lessening the severity of one or more of the diseases, disorders or conditions as recited above.

The compounds and compositions, according to the methods of the present invention, may be administered using any amount and any route of administration effective for treating or lessening the severity of one or more of the diseases, disorders or conditions recited above.

The compounds of the present invention are typically used as a pharmaceutical composition (e.g., a compound of the present invention and at least one pharmaceutically acceptable carrier). As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. For purposes of this invention, solvates and hydrates are considered pharmaceutical compositions comprising a compound of the present invention and a solvent (i.e., solvate) or water (i.e., hydrate).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present invention is generally formulated for use as a parenteral or oral administration or alternatively suppositories.

For example, the pharmaceutical oral compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with

a) diluents, e.g., lactose, dextrose, sucrose, mannitol, sorbitol, cellulose and/or glycine;

b) lubricants, e.g., silica, talcum, stearic acid, its magnesium or calcium salt and/or polyethylene glycol; for tablets also

c) binders, e.g., magnesium aluminum silicate, starch paste, gelatin, tragacanth, methylcellulose, sodium carboxymethylcellulose and/or polyvinylpyrrolidone; if desired

d) disintegrants, e.g., starches, agar, alginic acid or its sodium salt, or effervescent mixtures; and/or

e) absorbents, colorants, flavors and sweeteners.

Tablets may be either film coated or enteric coated according to methods known in the art.

Suitable compositions for oral administration include a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compound of the present invention or pharmaceutical composition thereof for use in a subject (e.g., human) is typically administered orally or parenterally at a therapeutic dose of less than or equal to about 100 mg/kg, 75 mg/kg, 50 mg/kg, 25 mg/kg, 10 mg/kg, 7.5 mg/kg, 5.0 mg/kg, 3.0 mg/kg, 1.0 mg/kg, 0.5 mg/kg, 0.05 mg/kg or 0.01 mg/kg, but preferably not less than about 0.0001 mg/kg. When administered intravenously via infusion, the dosage may depend upon the infusion rate at which an IV formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations.

### Combination Therapy

The compounds of the present invention can be used, alone or in combination with other therapeutic agents, in the treatment of various conditions or disease states. The compound(s) of the present invention and other therapeutic agent(s) may be administered simultaneously (either in the same dosage form or in separate dosage forms) or sequentially.

Two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

The present invention includes the use of a combination of an IRAK inhibitor compound as provided in the compound of formula (I) and one or more additional pharmaceutically active agent(s). If a combination of active agents is administered, then they may be administered sequentially or simultaneously, in separate dosage forms or combined in a single dosage form. Accordingly, the present invention also includes pharmaceutical compositions comprising an amount of: (a) a first agent comprising a compound of formula (I) or a pharmaceutically acceptable salt of the compound; (b) a second pharmaceutically active agent; and (c) a pharmaceutically acceptable carrier, vehicle or diluent.

The compounds of the present invention can be administered alone or in combination with one or more additional therapeutic agents. By “administered in combination” or “combination therapy” it is meant that a compound of the present invention and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect. Thus, the methods of prevention and treatment described herein include use of combination agents.

The combination agents are administered to a mammal, including a human, in a therapeutically effective amount. By “therapeutically effective amount” it is meant an amount of a compound of the present invention that, when administered alone or in combination with an additional therapeutic agent to a mammal, is effective to treat the desired disease/condition e.g., inflammatory condition such as systemic lupus erythematosus. See also, T. Koutsokeras and T. Healy, Systemic lupus erythematosus and lupus nephritis, Nat Rev Drug Discov, 2014, 13(3), 173-174, for therapeutic agents useful treating lupus.

In particular, it is contemplated that the compounds of the invention may be administered with the following therapeutic agents: Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexyphenidyl, and amantadine; agents for treating Multiple Sclerosis (MS) such as Tecfidera® and beta interferon (e.g., Avonex® and Rebif®), Copaxone*, and mitoxantrone; treatments for asthma such as albuterol and Singulair®; agents for treating schizophrenia such as zyprexa, risperdal, seroquel, and haloperidol; anti-inflammatory agents such as corticosteroids, T F blockers, IL-1 RA, azathioprine, cyclophosphamide, and sulfasalazine; immunomodulatory and immunosuppressive agents such as cyclosporin, tacrolimus, apramycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3 A4 inhibitors (e.g., ketoconazole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

### Definitions

As used herein, a “patient,” “subject” or “individual” are used interchangeably and refer to either a human or non-human animal. The term includes mammals such as humans. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. Preferably, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder, refers to the management and care of a patient for the purpose of combating the disease, condition, or disorder and includes the administration of a compound of the present invention to prevent the onset of the symptoms or complications, alleviating the symptoms or complications, or eliminating the disease, condition or disorder.

As used herein the term “stroke” has the meaning normally accepted in the art. The term can broadly refer to the development of neurological deficits associated with the impaired blood flow regardless of cause. Potential causes include, but are not limited to, thrombosis, hemorrhage and embolism. The term “ischemic stroke” refers more specifically to a type of stroke that is of limited extent and caused due to a blockage of blood flow.

As used herein, a subject is “in need of” a treatment if such subject would benefit biologically, medically or in quality of life from such treatment (preferably, a human).

As used herein the term “co-administer” refers to the presence of two active agents in the blood of an individual. Active agents that are co-administered can be concurrently or sequentially delivered.

The term “combination therapy” or “in combination with” or “pharmaceutical combination” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent being administered prior to, concurrent with, or sequentially to each other with no specific time limits. In each case, the treatment regimen will provide beneficial effects of the drug combination in treating the conditions or disorders described herein.

As used herein, the phrase “optionally substituted” is used interchangeably with the phrase “substituted or unsubstituted.” In general the term “optionally substituted” refers to the replacement of hydrogen radicals in a given structure with the radical of a specified substituent. Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position.

As used herein, the term “C₁₋₅ alkyl” refers to a fully saturated branched or unbranched hydrocarbon moiety having 1 to 5 carbon atoms. The terms “C₁₋₄ alkyl”, “C₁₋₃ alkyl” and “C₁₋₂ alkyl” are to be construed accordingly. Representative examples of “C₁₋₅ alkyl” include, but are not limited to, methyl, ethyl, n-propyl, iso-propyl, n-butyl, sec-butyl, iso-butyl, tert-butyl, n-pentyl, isopentyl and neopentyl. Similarly, the alkyl portion (i.e., alkyl moiety) of an alkoxy have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls). “Halo-substituted alkyl” refers to an alkyl group having at least one halogen substitution.

As used herein, the term “C₁₋₄ alkoxy” refers to a fully saturated branched or unbranched alkyl moiety attached through an oxygen bridge (i.e. a —O—C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. Preferably, alkoxy groups have about 1-4 carbons, more preferably about 1-2 carbons. The term “C₁₋₂ alkoxy” is to be construed accordingly.

As used herein, the term “C₁₋₄ alkoxy-C₁₋₄ alkyl” refers to a C₁₋₄ alkyl group as defined herein, wherein at least of the hydrogen atoms is replaced by an C₁₋₄ alkoxy. The C₁₋₄ alkoxy-C₁₋₄ alkyl group is connected through the rest of the molecule described herein through the alkyl group.

“Halogen” or “halo” may be fluorine, chlorine, bromine or iodine (preferred halogens as substituents are fluorine and chlorine).

As used herein, the term “halo-substituted-C₁₋₄ alkyl” or “halo-C₁₋₄ alkyl” refers to a C₁₋₄ alkyl group as defined herein, wherein at least one of the hydrogen atoms is replaced by a halo atom. The halo-C₁₋₄ alkyl group can be monohalo-C₁₋₄ alkyl, dihalo-C₁₋₄ alkyl or polyhalo-C₁₋₄ alkyl including perhalo-C₁₋₄ alkyl. A monohalo-C₁₋₄ alkyl can have one iodo, bromo, chloro or fluoro within the alkyl group. Dihalo-C₁₋₄ alkyl and polyhalo-C₁₋₄ alkyl groups can have two or more of the same halo atoms or a combination of different halo groups within the alkyl. Typically the polyhalo-C₁₋₄ alkyl group contains up to 9, or 8, or 7, or 6, or 5, or 4, or 3, or 2 halo groups. Non-limiting examples of halo-C₁₋₄ alkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-C₁₋₄ alkyl group refers to a C₁₋₄ alkyl group having all hydrogen atoms replaced with halo atoms.

As used herein, the term “halo-substituted-C₁₋₄ alkoxy” or “halo-C₁₋₄ alkoxy” refers to C₁₋₄ alkoxy group as defined herein above wherein at least one of the hydrogen atoms is replaced by a halo atom. Non-limiting examples of halo-substituted-C₁₋₄ alkoxy include fluoromethoxy, difluoromethoxy, trifluoromethoxy, chloromethoxy, dichloromethoxy, trichloromethoxy, difluorochloromethoxy, dichlorofluoromethoxy, difluoroethoxy, difluoropropoxy, dichloroethoxy and dichloropropoxy and the like.

As used herein “Hydroxyl” or “Hydroxy” refers to the group —OH.

As used herein, the term “hydroxy-substituted-C₁₋₄ alkyl” refers to a C₁₋₄ alkyl group as defined herein, wherein at least one of the hydrogen atoms is replaced by a hydroxyl group. The hydroxy-substituted-C₁₋₄ alkyl group can be monohydroxy-C₁₋₄ alkyl, dihydroxy-C₁₋₄ alkyl or polyhydroxy-C₁₋₄ alkyl including perhydroxy-C₁₋₄ alkyl. A monohydroxy-C₁₋₄ alkyl can have one hydroxyl group within the alkyl group. Dihydroxy-C₁₋₄ alkyl and polyhydroxy-C₁₋₄ alkyl groups can have two or more of the same hydroxyl groups or a combination of different hydroxyl groups within the alkyl. Typically the polyhydroxy-C₁₋₄ alkyl group contains up to 9, or 8, or 7, or 6, or 5, or 4, or 3, or 2 hydroxy groups. Non-limiting examples of hydroxy substituted-C₁₋₄ alkyl include hydroxy-methyl, dihydroxy-methyl, pentahydroxy-ethyl, dihydroxyethyl, and dihydroxypropyl. A perhydroxy-C₁₋₄ alkyl group refers to a C₁₋₄ alkyl group having all hydrogen atoms replaced with hydroxy atoms.

The term “oxo” (═O) refers to an oxygen atom connected to a carbon or sulfur atom by a double bond. Examples include carbonyl, sulfinyl, or sulfonyl groups (—C(O)—, —S(O)— or —S(O)₂—) such as, a ketone, aldehyde, or part of an acid, ester, amide, lactone, or lactam group and the like.

The term “aryl or C₆₋₁₀ aryl” refers to 6- to 10-membered aromatic carbocyclic moieties having a single (e.g., phenyl) or a fused ring system (e.g., naphthalene). A typical aryl group is phenyl group.

The term “fully or partially saturated carbocyclic ring” refers to a nonaromatic hydrocarbon ring that is either partially or fully saturated and may exist as a single ring, bicyclic ring (including fused, spiral or bridged carbocyclic rings) or a spiral ring. Unless specified otherwise, the carbocyclic ring generally contains 4- to 7-ring members.

The term “C₃₋₆ cycloalkyl” refers to a carbocyclic ring which is fully saturated (e.g., cyclopropyl, cyclobutyl, cyclopentyl, and cyclohexyl).

The term “4 to 7 membered heterocycle” or “C₄₋₇ heterocycle” refers to a monocyclic ring which is fully saturated which has 4 to 7 ring atoms which contains 1 to 2 heteroatoms, independently selected from sulfur, oxygen and/or nitrogen. A typical “C₄₋₇ heterocycle” group includes oxetanyl, tetrahydrofuranyl, dihydrofuranyl, 1,4-dioxanyl, morpholinyl, 1,4-dithianyl, piperazinyl, piperidinyl, 1,3-dioxolanyl, pyrrolinyl, pyrrolidinyl, tetrahydropyranyl, oxathiolanyl, dithiolanyl, 1,3-dioxanyl, 1,3-dithianyl, oxathianyl, thiomorpholinyl, thiomorpholinyl 1,1 dioxide, tetrahydro-thiopyran 1,1-dioxide, 1,4-diazepanyl. In some embodiments, a “C₄₋₇ heterocycle” group contains at least one oxygen ring atom. In some embodiments, a “C₄₋₇ heterocycle” group is selected from oxetanyl, tetrahydrofuranyl, 1,4-dioxanyl and tetrahydropyranyl.

The term “fully or partially saturated heterocycle” or “fully or partially saturated 4 to 7 membered heterocycle” refers to a nonaromatic ring that is either partially or fully saturated and may exist as a single ring, bicyclic ring (including fused heterocyclic rings) or a spiral ring. Unless specified otherwise, the heterocyclic ring is generally a 4 to 7-membered ring containing 1 to 3 heteroatoms (preferably 1, 2 or 3 heteroatoms) independently selected from sulfur, oxygen and/or nitrogen. A partially saturated heterocyclic ring also includes groups wherein the heterocyclic ring is fused to an aryl or heteroaryl ring (e.g., 2,3-dihydrobenzofuranyl, indolinyl (or 2,3-dihydroindolyl), 2,3-dihydrobenzothiophenyl, 2,3-dihydrobenzothiazolyl, 1,2,3,4-tetrahydroquinolinyl, 1,2,3,4-tetrahydroisoquinolinyl, 5,6,7,8-tetrahydropyrido[3,4-b]pyrazinyl).

As used herein the term “spiral” or “spiro 5 to 10 membered heterobicyclic ring system” means a two-ring system wherein both rings share one common atom. Examples of spiral rings include oxaspiro[2.4]heptanyl, 5-oxaspiro[2.4]heptanyl, 4-oxaspiro[2.4]heptane, 4-oxaspiro[2.5]octanyl, 6-oxaspiro[2.5]octanyl, oxaspiro[2.5]octanyl, oxaspiro[3.4]octanyl, oxaspiro[bicyclo[2.1.1]hexane-2,3′-oxetan]-1-yl, oxaspiro[bicyclo[3.2.0]heptane-6,1′-cyclobutan]-7-yl, 2,6-diazaspiro[3.3]heptanyl, -oxa-6-azaspiro[3.3]heptane, 2,2,6-diazaspiro[3.3]heptane, 3-azaspiro[5.5]undecanyl, 3,9-diazaspiro[5.5]undecanyl, 7-azaspiro[3.5]nonane, 2,6-diazaspiro[3.4]octane, 8-azaspiro[4.5]decane, 1,6-diazaspiro[3.3]heptane, 5-azaspiro[2.5]octane, 4,7-diazaspiro[2.5]octane, 5-oxa-2-azaspiro[3.4]octane, 6-oxa-1-azaspiro[3.3]heptane, 3-azaspiro[5.5]undecanyl, 3,9-diazaspiro[5.5]undecanyl, and the like.

As used herein the term “spiro 3-8 membered cycloalkyl” means a two-ring system wherein both rings share one common carbon atom. Examples of spiro 3-8 membered cycloalkyl rings include spiro[2.5]octane, spiro[2.3]hexane, spiro[2.4]heptane, spiro[3.4]octane and the like.

Partially saturated or fully saturated heterocyclic rings include groups such as epoxy, aziridinyl, azetidinyl, tetrahydrofuranyl, dihydrofuranyl, dihydropyridinyl, pyrrolidinyl, imidazolidinyl, imidazolinyl, 1H-dihydroimidazolyl, hexahydropyrimidinyl, piperidinyl, piperazinyl, pyrazolidinyl, 2H-pyranyl, 4H-pyranyl, oxazinyl, morpholino, thiomorpholino, tetrahydrothienyl, tetrahydrothienyl 1,1-dioxide, oxazolidinyl, thiazolidinyl, 7-oxabicyclo[2.2.1]heptane, and the like.

The term “Fused heterocycle” or “7 to 10 membered fused heterobicyclic ring system” or “5 to 10 membered fused heterobicyclic ring system” refers to two ring systems share two adjacent ring atoms ad at least one the ring systems contain a ring atom that is a heteroatom selected from O, N and S. Examples of fused heterocycles include fully or partially saturated groups such as 1,3-dihydroisobenzofuran, 4-methyl-3,4-dihydro-2H-benzo[b][1,4]oxazine, pyrazolo[1,5-a]pyrimidine, 5,6-dihydro-4H-pyrrolo[1,2-b]pyrazole, 6,7-dihydro-5H-cyclopenta[b]pyridine, 2-oxabicyclo[2.1.0]pentane, indolin-2-one, 2,3-dihydrobenzofuran, 1-methyl-2-oxo-1,2,3,4-tetrahydroquinoline, 3,4-dihydroquinolin-2 (1H)-one, chromane, isochromane, 4,5,6,7-tetrahydro-3H-imidazo[4,5-c]pyridine, 8-azabicyclo[3.2.1]octan-3-ol, octahydropyrrolo[1,2-a]pyrazine, 5,6,7,8-tetrahydroimidazo[1,2-a]pyrazine, 3,8 diazabicyclo[3.2.1]octane, 8-oxa-3-azabicyclo[3.2.1]octane, 7-oxabicyclo[2.2.1]heptane, 1H-pyrazole, 2,5-diazabicyclo[2.2.1]heptane, 5,6,7,8-tetrahydro-[1,2,4]triazolo[4,3-a]pyrazine, 3-oxabicyclo[3.1.0]hexane, or 3-azabicyclo[3.1.0]hexane. A partially saturated heterocyclic ring also includes groups wherein the heterocyclic ring is fused to an aryl or heteroaryl ring (e.g., 2,3-dihydrobenzofuranyl, indolinyl (or 2,3-dihydroindolyl), 2,3-dihydrobenzothiophenyl, 2,3-dihydrobenzothiazolyl, 1,2,3,4-tetrahydroquinolinyl, 1,2,3,4-tetrahydroisoquinolinyl, 5,6,7,8-tetrahydropyrido[3,4-b]pyrazinyl, 6,7-dihydro-5H-pyrazolo[5,1-b][1,3]oxazine, and the like. In some embodiments, the “7 to 10 membered fused heterobicyclic ring system” is a 9 to 10 membered bicyclic heteroaryl, such as pyrazolo[1,5-a]pyrimidine, pyrazolo[1,5-a]pyridine, [1,2,4]triazolo[4,3-a]pyridine, [1,2,4]triazolo[1,5-a]pyridine, isothiazolo[4,3-b]pyridine, pyrrolo[1,2-a]pyrimidine, pyrido[3,2-d]pyrimidine, imidazo[1,2-b]pyridazine, thieno[2,3-b]pyrazine, 1H-benzo[d]imidazole, benzo[d]thiazole, 1,6-naphthyridine and 1,5-naphthyridine.

As used herein the term “7 to 10 membered fused bicyclic ring system” refers to a 7 to 10 membered carbocyclic moiety connected at two non-adjacent ring atoms of the carbocycle (e.g. 1,2,3,4-tetrahydronaphthalene, (1S,5R)-1-methylbicyclo[3.1.0]hexane, bicyclo[3.1.0]hexane, bicyclo[4.1.0]heptane and 2,3-dihydro-1H-indene.

As used herein the term “bridged-carbocyclic ring” refers to a 5 to 10 membered cyclic moiety connected at two non-adjacent ring atoms of the carbocycle (e.g. bicyclo[1.1.1]pentane, bicyclo[2.2.1]heptane and bicyclo[3.2.1]octane).

As used herein the term “bridged-heterocyclic ring” refers to a 5 to 10 membered heterobicyclic moiety connected at two non-adjacent ring atoms of the heterocycle containing at least one heteroatom (e.g., oxygen, sulfur, nitrogen or combinations thereof) within a 5 to 10 membered cyclic ring system. Examples of the “bridged-heterocyclic ring” include, but are not limited to, 2-oxabicyclo[2.1.1]hexane, 3-oxabicyclo[4.1.0]heptane, 2-oxabicyclo[2.2.1]heptane, 2-oxabicyclo[2.2.2]octane, 8-oxabicyclo[3.2.1]octane, and 2,6-dioxabicyclo[3.2.1]octane.

The term “heteroaryl” refers to aromatic moieties containing at least one heteroatom (e.g., oxygen, sulfur, nitrogen or combinations thereof) within a 5- to 6-membered aromatic ring system (e.g., pyrrolyl, pyridyl, pyrazolyl, thienyl, furanyl, oxazolyl, imidazolyl, tetrazolyl, triazinyl, pyrimidyl, pyrazinyl, thiazolyl, and the like) within a 9- to 10-membered aromatic ring system (e.g., indolyl, indazolyl, benzofuranyl, quinoxalinyl and the like.)

The term “5 to 6 membered heteroaryl” or “C₅₋₆ heteroaryl” refers to an aromatic moieties containing at least one heteroatom (e.g., oxygen, sulfur, nitrogen or combinations thereof) within a 5- to 6-membered monocyclic aromatic ring system. In some embodiments, a 5 to 6 membered heteroaryl is selected from pyrrolyl, pyridyl, pyrazolyl, thienyl, furanyl, oxazolyl, isoxazolyl, isothiazolyl, thiazolyl, imidazolyl, tetrazolyl, triazinyl, pyrimidyl, pyrazinyl, and thiazolyl. In some embodiments, a 5 to 6 membered heteroaryl is selected from pyridinyl, pyrimidinyl, 2H-1,2,3-triazolyl, isoxazolyl, isothiazolyl, thiazolyl, pyrazolyl and thienyl.

The term “9 to 10 membered heteroaryl” or “C₉₋₁₀ heteroaryl” refers to aromatic moieties containing at least one heteroatom (e.g., oxygen, sulfur, nitrogen or combinations thereof) within a 9- to 10-membered fused aromatic ring system. In some embodiments, a “9 to 10 membered heteroaryl” is selected from indolyl, indazolyl, benzofuranyl, quinoxalinyl, pyrazolo[1,5-a]pyridinyl, [1,2,4]triazolo[4,3-a]pyridinyl, isothiazolo[4,3-b]pyridinyl, pyrazolo[1,5-a]pyrimidinyl, pyrido[3,2-d]pyrimidinyl, imidazo[1,2-b]pyridazinyl, thieno[2,3-b]pyrazinyl, 1H-benzo[d]imidazolyl, benzo[d]thiazolyl, 1,6-naphthyridinyl, and 1,5-naphthyridinyl. In some embodiments, a “9 to 10 membered heteroaryl” is selected from pyrazolo[1,5-a]pyridinyl, [1,2,4]triazolo[4,3-a]pyridinyl, isothiazolo[4,3-b]pyridinyl, pyrazolo[1,5-a]pyrimidinyl, pyrido[3,2-d]pyrimidinyl, imidazo[1,2-b]pyridazinyl, thieno[2,3-b]pyrazinyl, 1H-benzo[d]imidazolyl, benzo[d]thiazolyl, 1,6-naphthyridinyl, 1,5-naphthyridinyl, and 2H-indazolyl.

The phrase “pharmaceutically acceptable” indicates that the substance, composition or dosage form must be compatible chemically and/or toxicologically, with the other ingredients comprising a formulation, and/or the mammal being treated therewith.

Unless specified otherwise, the term “compounds of the present invention” refers to compounds of formula (I′) or (I), as well as all stereoisomers (including diastereoisomers and enantiomers), rotamers, tautomers, isotopically labeled compounds (including deuterium substitutions), and inherently formed moieties (e.g., polymorphs, solvates and/or hydrates). When a moiety is present that is capable of forming a salt, then salts are included as well, in particular pharmaceutically acceptable salts.

As used herein, the term “a,” “an,” “the” and similar terms used in the context of the present invention (especially in the context of the claims) are to be construed to cover both the singular and plural unless otherwise indicated herein or clearly contradicted by the context. The use of any and all examples, or exemplary language (e.g. “such as”) provided herein is intended merely to better illuminate the invention and does not pose a limitation on the scope of the invention otherwise claimed.

In one Embodiment, there is provided a compound of the Examples as an isolated stereoisomer wherein the compound has one stereocenter and the stereoisomer is in the R configuration.

In one Embodiment, there is provided a compound of the Examples as an isolated stereoisomer wherein the compound has one stereocenter and the stereoisomer is in the S configuration.

In one Embodiment, there is provided a compound of the Examples as an isolated stereoisomer wherein the compound has two stereocenters and the stereoisomer is in the R R configuration.

In one Embodiment, there is provided a compound of the Examples as an isolated stereoisomer wherein the compound has two stereocenters and the stereoisomer is in the R S configuration.

In one Embodiment, there is provided a compound of the Examples as an isolated stereoisomer wherein the compound has two stereocenters and the stereoisomer is in the S R configuration.

In one Embodiment, there is provided a compound of the Examples as an isolated stereoisomer wherein the compound has two stereocenters and the stereoisomer is in the S S configuration.

In one Embodiment, there is provided a compound of the Examples, wherein the compound has one or two stereocenters, as a racemic mixture.

It is also possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

In one Embodiment, the invention relates to a compound of the formula (I′) or (I) as defined herein, in free form. In another Embodiment, the invention relates to a compound of the formula (I′) or (I) as defined herein, in salt form. In another Embodiment, the invention relates to a compound of the formula (I′) or (I) as defined herein, in acid addition salt form. In a further Embodiment, the invention relates to a compound of the formula (I′) or (I) as defined herein, in pharmaceutically acceptable salt form. In yet a further Embodiment, the invention relates to a compound of the formula (I′) or (I) as defined herein, in pharmaceutically acceptable acid addition salt form. In yet a further Embodiment, the invention relates to any one of the compounds of the Examples in free form. In yet a further Embodiment, the invention relates to any one of the compounds of the Examples in salt form. In yet a further Embodiment, the invention relates to any one of the compounds of the Examples in acid addition salt form. In yet a further Embodiment, the invention relates to any one of the compounds of the Examples in pharmaceutically acceptable salt form. In still another Embodiment, the invention relates to any one of the compounds of the Examples in pharmaceutically acceptable acid addition salt form.

Furthermore, the compounds of the present invention, including their salts, may also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

Compounds of the invention, i.e. compounds of formula (I′) or (I) that contain groups capable of acting as donors and/or acceptors for hydrogen bonds may be capable of forming co-crystals with suitable co-crystal formers. These co-crystals may be prepared from compounds of formula (I′) or (I) by known co-crystal forming procedures. Such procedures include grinding, heating, co-subliming, co-melting, or contacting in solution compounds of formula (I′) or (I) with the co-crystal former under crystallization conditions and isolating co-crystals thereby formed. Suitable co-crystal formers include those described in WO 2004/078163. Hence the invention further provides co-crystals comprising a compound of formula (I′) or (I).

The compounds of the present invention, including salts, hydrates and solvates thereof, may inherently or by design form polymorphs.

Compounds of the present invention may be synthesized by synthetic routes that include processes analogous to those well-known in the chemical arts, particularly in light of the description contained herein. The starting materials are generally available from commercial sources such as Sigma-Aldrich or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

The further optional reduction, oxidation or other functionalization of compounds of formula (I) may be carried out according to methods well known to those skilled in the art. Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, and in H. D. Jakubke and H. Jeschkeit, “Aminosauren, Peptide, Proteine” (Amino acids, Peptides, Proteins), Verlag Chemie, Weinheim, Deerfield Beach, and Basel 1982. A characteristic of protecting groups is that they can be removed readily (i.e. without the occurrence of undesired secondary reactions) for example by solvolysis, reduction, photolysis or alternatively under physiological conditions (e.g. by enzymatic cleavage).

Salts of compounds of the present invention having at least one salt-forming group may be prepared in a manner known to those skilled in the art. For example, acid addition salts of compounds of the present invention are obtained in customary manner, e.g. by treating the compounds with an acid or a suitable anion exchange reagent. Salts can be converted into the free compounds in accordance with methods known to those skilled in the art. Acid addition salts can be converted, for example, by treatment with a suitable basic agent.

Any resulting mixtures of isomers can be separated on the basis of the physicochemical differences of the constituents, into the pure or substantially pure geometric or optical isomers, diastereomers, racemates, for example, by chromatography and/or fractional crystallization.

For those compounds containing an asymmetric carbon atom, the compounds exist in individual optically active isomeric forms or as mixtures thereof, e.g. as racemic or diastereomeric mixtures. Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a commercially available chiral HPLC column.

The invention further includes any variant of the present processes, in which the reaction components are used in the form of their salts or optically pure material. Compounds of the invention and intermediates can also be converted into each other according to methods generally known to those skilled in the art.

For illustrative purposes, the reaction schemes depicted below provide potential routes for synthesizing the compounds of the present invention as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### General Methods

The compounds of the Examples were analyzed or purified according to one of the Purification Methods referred to below unless otherwise described.

Where preparative TLC or silica gel chromatography have been used, one skilled in the art may choose any combination of solvents to purify the desired compound. Silica gel column chromatography was performed using 20-40 μM (particle size), 250-400 mesh, or 400-632 mesh silica gel using either a Teledyne ISCO Combiflash RF or a Grace Reveleris X2 with ELSD purification systems or using pressurized nitrogen (˜10-15 psi) to drive solvent through the column (“flash chromatography”).

Wherein an SCX column has been used, the eluant conditions are MeOH followed by methanolic ammonia.

Except where otherwise noted, reactions were run under an atmosphere of nitrogen. Where indicated, solutions and reaction mixtures were concentrated by rotary evaporation under vacuum.

### Analytical Methods

ESI-MS data (also reported herein as simply MS) were recorded using Waters System (Acquity HPLC and a Micromass ZQ mass spectrometer); all masses reported are the m/z of the protonated parent ions unless recorded otherwise.

### LC/MS:

A sample is dissolved in a suitable solvent such as MeCN, dimethyl sulfoxide (DMSO), or MeOH and is injected directly into the column using an automated sample handler. The analysis used one of the following methods: (1) acidic method (1.5, 2, 3.5, 4, or 7 min runs, see Acidic LCMS section for additional details vide infra: conducted on a Shimadzu 2010 Series, Shimadzu 2020 Series, or Waters Acquity UPLC BEH. (MS ionization: ESI) instrument equipped with a C18 column (2.1 mm×30 mm, 3.0 mm or 2.1 mm×50 mm, C18, 1.7 μm), eluting with 1.5 mL/4 L of trifluoroacetic acid (TFA) in water (solvent A) and 0.75 mL/4 L of TFA in MeCN (solvent B) or (2) basic method (3, 3.5, 7 min runs, see Basic LCMS section for additional details vide infra: conducted on a Shimadzu 2020 Series or Waters Acquity UPLC BEH (MS ionization: ESI) instrument equipped with XBridge Shield RP18, 5 um column (2.1 mm×30 mm, 3.0 mm i.d.) or 2.1 mm×50 mm, C18, 1.7 μm column, eluting with 2 mL/4 L NH₃.H₂O in water (solvent A) and MeCN (solvent B).

The invention further includes any variant of the present processes, in which the reaction components are used in the form of their salts or optically pure material. Compounds of the invention and intermediates can also be converted into each other according to methods generally known to those skilled in the art.

### Analytical HPLC

Acidic HPLC: Conducted on a Shimadza 20A instrument with an ultimate C18 3.0×50 mm, 3 μm column eluting with 2.75 mL/4 L TFA in water (solvent A) and 2.5 mL/4 L TFA in acetonitrile (solvent B) by the following methods:

Method A: using the following elution gradient 0%-60% (solvent B) over 6 minutes and holding at 60% for 2 minutes at a flow rate of 1.2 ml/minutes. Wavelength: UV 220 nm, 215 nm and 254 nm.

Method B: using the following elution gradient 10%-80% (solvent B) over 6 minutes and holding at 60% for 2 minutes at a flow rate of 1.2 ml/minutes. Wavelength: UV 220 nm, 215 nm and 254 nm.

Method C: using the following elution gradient 30%-90% (solvent B) over 6 minutes and holding at 60% for 2 minutes at a flow rate of 1.2 ml/minutes. Wavelength: UV 220 nm, 215 nm and 254 nm.

Basic HPLC: Conducted on a Shimadza 20A instrument with Xbrige Shield RP-18, Sum, 2.1×50 mm column eluting with 2 mL/4 L NH3H2O in water (solvent A) and acetonitrile (solvent B), by the following methods:

Method D: using the following elution gradient 0%-60% (solvent B) over 4.0 minutes and holding at 60% for 2 minutes at a flow rate of 1.2 ml/minutes.

Method E: using the following elution gradient 10%-80% (solvent B) over 4.0 minutes and holding at 60% for 2 minutes at a flow rate of 1.2 ml/minutes.

Method F: using the following elution gradient 30%-90% (solvent B) over 4.0 minutes and holding at 60% for 2 minutes at a flow rate of 1.2 ml/minutes.

### Analytical LCMS

Acidic LCMS: Conducted on a Shimadza 2010 Series, Shimadza 2020 Series, or Waters Acquity UPLC BEH. (MS ionization: ESI) instrument equipped with a C18 column (2.1 mm×30 mm, 3.0 mm or 2.1 mm×50 mm, C18, 1.7 μm), eluting with 1.5 mL/4 L TFA in water (solvent A) and 0.75 mL/4 LTFA in acetonitrile (solvent B) using the methods below:

1.5 Minute Methods:

General method: using the following elution gradient 5%-95% (solvent B) over 0.7 minutes and holding at 95% for 0.4 minutes at a flow rate of 1.5 ml/minutes. Wavelength: UV 220 nm and 254 nm.

2 Minute Methods:

Method A: using the following elution gradient 0%-60% (solvent B) over 0.9 minutes and holding at 60% for 0.6 minutes at a flow rate of 1.2 ml/minutes. Wavelength: UV 220 nm and 254 nm.

Method B: using the following elution gradient 10%-80% (solvent B) over 0.9 minutes and holding at 60% for 0.6 minutes at a flow rate of 1.2 ml/minutes. Wavelength: UV 220 nm and 254 nm.

Method C: using the following elution gradient 30%-90% (solvent B) over 0.9 minutes and holding at 60% for 0.6 minutes at a flow rate of 1.2 ml/minutes. Wavelength: UV 220 nm and 254 nm.

3.5 Minute Method:

Initial conditions, solvent A-95%: solvent B-5%; hold at initial from 0.0-0.1 min; Linear Ramp to solvent A-5%: solvent B-95% between 0.1-3.25 min; hold at solvent A-5%: solvent B-95% between 3.25-3.5 min. Diode array/MS detection.

4 Minute Methods:

Method A: using the following elution gradient 0%-60% (solvent B) over 3 minutes and holding at 60% for 0.5 minutes at a flow rate of 0.8 ml/minutes. Wavelength: UV 220 nm and 254 nm.

Method B: using the following elution gradient 10%-80% (solvent B) over 3 minutes and holding at 60% for 0.5 minutes at a flow rate of 0.8 ml/minutes. Wavelength: UV 220 nm and 254 nm.

Method C: using the following elution gradient 30%-90% (solvent B) over 3 minutes and holding at 60% for 0.5 minutes at a flow rate of 0.8 ml/minutes. Wavelength: UV 220 nm and 254 nm.

7 Minute Methods:

Method A: using the following elution gradient 0%-60% (solvent B) over 6 minutes and holding at 60% for 0.5 minutes at a flow rate of 0.8 ml/minutes. Wavelength: UV 220 nm and 254 nm.

Method B: using the following elution gradient 10%-80% (solvent B) over 6 minutes and holding at 60% for 0.5 minutes at a flow rate of 0.8 ml/minutes. Wavelength: UV 220 nm and 254 nm.

Method C: using the following elution gradient 30%-900% (solvent B) over 6 minutes and holding at 60% for 0.5 minutes at a flow rate of 0.8 ml/minutes. Wavelength: UV 220 nm and 254 nm.

Basic LCMS: Conducted on a Shimadza 2020 Series or Waters Acquity UPLC BEH (MS ionization: ESI) instrument equipped with XBridge Shield RP18, 5 um column (2.1 mm×30 mm, 3.0 mm i.d.) or 2.1 mm×50 mm, C18, 1.7 μm column, eluting with 2 mL/4 L NH3.H2O in water (solvent A) and acetonitrile (solvent B) using the methods below:

3 Minute Methods:

Method A: using the following elution gradient 0%-60% (solvent B) over 2 minutes and holding at 60% for 0.48 minutes at a flow rate of 1 ml/minutes. Wavelength: UV 220 nm and 254 nm.

Method B: using the following elution gradient 10%-80% (solvent B) over 2 minutes and holding at 60% for 0.48 minutes at a flow rate of 1 ml/minutes. Wavelength: UV 220 nm and 254 nm.

Method C: using the following elution gradient 30%-90% (solvent B) over 2 minutes and holding at 60% for 0.48 minutes at a flow rate of 1 ml/minutes. Wavelength: UV 220 nm and 254 nm.

3.5 Minute Method:

Initial conditions, solvent A-95%: solvent B-5%; hold at initial from 0.0-0.1 min; Linear Ramp to solvent A-5%: solvent B-95% between 0.1-3.25 min; hold at solvent A-5%: solvent B-95% between 3.25-3.5 min. Diode array/MS detection.

7 Minute Methods:

Method A: using the following elution gradient 0%-60% (solvent B) over 6 minutes and holding at 60% for 0.5 minutes at a flow rate of 0.8 ml/minutes. Wavelength: UV 220 nm and 254 nm.

Method B: using the following elution gradient 10%-80% (solvent B) over 6 minutes and holding at 60% for 0.5 minutes at a flow rate of 0.8 ml/minutes. Wavelength: UV 220 nm and 254 nm.

Method C: using the following elution gradient 30%-90% (solvent B) over 6 minutes and holding at 60% for 0.5 minutes at a flow rate of 0.8 ml/minutes. Wavelength: UV 220 nm and 254 nm.

### SFC Analytical Separation

Instrument: Waters UPC2 analytical SFC (SFC-H). Column: ChiralCel OJ, 150×4.6 mm I.D., 3 μm. Mobile phase: A for CO2 and B for Ethanol (0.05% DEA). Gradient: B 40%. Flow rate: 2.5 mL/min. Back pressure: 100 bar. Column temperature: 35° C. Wavelength: 220 nm

### Preparative HPLC Purification

General Method: Preparative HPLC was performed on a Gilson UV/VIS-156 with UV detection at 220/254 nm Gilson 281 automatic collection.

Acidic condition: Two acid grading systems used: Hydrochloride acid and Formic acid.

Method A: Hydrochloride acid: YMC-Actus Triart C18 150×30 mm×5 um, Gradient used 0-100% acetonitrile with water and corresponding acid (0.05% HCl).

Method B: Formic acid: Phenomenex Synergi C18 150×30 mm×4 um, Gradient used 0-100% acetonitrile with water and corresponding acid (0.225% formic acid), the gradient shape was optimized for individual separations.

Neutral condition: Xtimate C18 150×25 mm×5 um, Gradient used 0-100% (water (10 mM NH₄HCO₃)−ACN), the gradient shape was optimized for individual separations.

Basic condition: Waters Xbridge Prep OBD C18 150×30 10 um, Gradient used 0-100% water (0.04% NH3H₂O+10 mM NH₄HCO₃)-acetonitrile, the gradient shape was optimized for individual separations.

### Preparative HPLC Conditions

### Column: Phenomenex Synergi C18 150×30 mm; 4 μm

Mobile phase A: MeCN
Mobile phase B: H₂O

### Modifier: 0.225% HCO₂H

Gradient (% organic): 0-100% optimised for each example

### Column: Sunfire C18 100×19 mm, 5 μm

Mobile phase A: MeCN
Mobile phase B: H₂O

### Modifier: 0.1% TFA

Gradient (% organic): 5-95% optimised for each example.

### Column: Sunfire C18 100×19 mm, 5 μm

Mobile phase A: MeCN
Mobile phase B: H₂O
Gradient (% organic): 5-95% optimised for each example.

### Column: XBridge C18 100×19 mm; 5 μm

Mobile phase A: MeCN
Mobile phase B: H₂O

### Modifier: 0.1% NH₄OH

Gradient (% organic): 0-100% optimised for each example.

### Column: XSelect C18 50×30 mm; 5 μm

Mobile phase A: MeCN
Mobile phase B: H₂O

### Modifier: 0.1% NH₄OH

Gradient (% organic): 0-100% optimised for each example.

Detectors: Gilson UV/VIS-156 with UV detection at 220/254 nm, Gilson 281 automatic collection, utilizing acidic, basic and neutral methods. For mass-directed peak collection, an ACQUITY QDa Mass Detector (Waters Corporation) was employed.

### Preparative SFC Purification

Instrument: MG III preparative SFC (SFC-1). Column: ChiralCel OJ, 250×30 mm I.D., 5 μm. Mobile phase: A for CO2 and B for Ethanol (0.1% NH₃H2O). Gradient: B 50%. Flow rate: 40 mL/min. Back pressure: 100 bar. Column temperature: 38° C. Wavelength: 220 nm. Cycle time: ˜8 min.

Column: Chiralpak AD-H; 250 mm×30 mm, 5 μm; 40% (EtOH+0.1% DEA)/CO₂
Column: Chiralpak IA; 250 mm×30 mm, 5 μm; 40% (MeOH+0.1% DEA)/CO₂
Column: Chiralpak IB; 250 mm×30 mm, 5 μm; 40% (EtOH+0.1% DEA)/CO₂
Column: Chiralpak AD-H; 250 mm×30 mm, 5 μm; 40% (EtOH+0.1% NH₄OH)/CO₂
Column: Chiralpak OJ-H; 250 mm×30 mm, 5 μm; 30% (EtOH+0.1% NH₄OH)/CO₂
Column: Chiralpak OD; 250 mm×30 mm, 5 μm; 35% (EtOH+0.1% NH₄OH)/CO₂

### 1H-NMR

¹H nuclear magnetic resonance (NMR) spectra were in all cases consistent with the proposed structures. The 1H NMR spectra were recorded on a Bruker Avance III HD 500 MHz, Bruker Avance III 500 MHz, Bruker Avance III 400 MHz, Varian-400 VNMRS, or Varian-400 MR. Characteristic chemical shifts (S) are given in parts-per-million downfield from tetramethylsilane (for ¹H-NMR) using conventional abbreviations for designation of major peaks: e.g. s, singlet; d, doublet; t, triplet; q, quartet; dd, double doublet; dt, double triplet; m, multiplet; br, broad. The following abbreviations have been used for common solvents: CDCl₃, deuterochloroform; DMSO-d₆, hexadeuterodimethyl sulfoxide; and MeOH-d₄, deuteron-methanol. Where appropriate, tautomers may be recorded within the NMR data; and some exchangeable protons may not be visible.

Typically, the compounds of Formula (I) can be prepared according to the schemes provided below. The following examples serve to illustrate the invention without limiting the scope thereof. Methods for preparing such compounds are described hereinafter

### ABBREVIATIONS

Abbreviations used are those conventional in the art or the following:

| | |
|---|---|
| AcOH means Acetic acid; | Min(s): minute(s) |
| Aq. means aqueous; | m/z: mass to charge ratio |
| Ar means argon; | Bn means benzyl; |
| BINAP means (±)-2,2′- | Boc means tert-butoxy carbonyl; |
| Bis(diphenylphosphino)-1,1′- | |
| binaphthalene; | |
| LC and LCMS: liquid chromatography | MeOH: methanol |
| and liquid chromatography-mass | |
| spectrometry | |
| br means broad; | Br₂ means bromine; |
| nBuOH means n-butanol; | |
| tBuOH means tert butanol; | n-BuLi means n-butyl lithium; |
| HRMS: high resolution mass | Pd₂(dba)₃ means |
| spectrometry | Tris(dibenzylideneacetone)dipalladium(0) |
| | CaCl₂ means Calcium chloride; |
| ° C. means degrees Celsius; | CHCl₃ means chloroform; |
| CDCl₃ means deutero-chloroform; | CDI means 1,1′-carbonyldiimidazole; |
| ESI: electrospray ionization | |
| CO means carbon monoxide; | (COCl)₂ means oxalyl chloride; |
| Cs₂CO₃ means cesium carbonate; | δ means chemical shift; |
| d means doublet; | dd means double doublet; |
| DABAL-Me₃ means | DMSO-d₆ means hexadeuterodimethyl sulfoxide; |
| bis(trimethylaluminium)-1,4- | |
| diazabicyclo[2.2.2]octane adduct; | |
| DCM: dichloromethane | DMF: dimethylformamide |
| DBU means 1,8- | DMAP means 4-(dimethylamino)pyridine; |
| Diazabicyclo[5.4.0] undec-7-ene; | |
| DMSO means dimethylsulfoxide | DPPA means Diphenyl phosphoryl azide; |
| Et means ethyl; | |
| Et₂O means ether; | EtOAc means ethyl acetate; |
| EtOH: ethanol | FA means formic acid; |
| Eq. means equivalent; | g means gram; |
| HATU means 1- | HBr means hydrogen bromide; |
| [bis(dimethylamino)methylene]-1H- | |
| 1,2,3-triazolo[4,5-b]pyridinium 3-oxid | |
| hexafluorophosphate; | |
| Na₂SO₄: sodium sulfate | Pd(OAc)₂: Palladium(II) acetate |
| HCl means hydrochloric acid; | HCO₂H means formic acid; |
| ¹HNMR means proton nuclear magnetic | HOAt means 1-hydroxy-7-azabenzotriazole; |
| resonance; | |
| H₂O means water; | |
| DIPEA: diisopropyl ethylamine | SCX: strong cation exchange sorbent, solid phase |
| | purification reagent |
| T3P means 2,4,6-Tripropyl-1,3,5,2,4,6- | N2 or N₂ means nitrogen |
| trioxatriphosphorinane-2,4,6-trioxide | |
| solution | |
| HPLC means high pressure liquid | h means hour; |
| chromatography; | |
| K₂CO₃ means potassium carbonate; | mL means millilitres; |
| KHSO₄ means potassium bisulfate; | mins means minutes; |
| KI means potassium iodide; | mmol means millimole; |
| KOH means potassium hydroxide; | Mukaiyama’s reagent means 2-chloro-1- |
| | methylpyridinium iodide; |
| L means litre; | MTBE means tert-butyl methyl ether; |
| LCMS means liquid chromatography | M/V means Mass volume ratio; |
| mass spectrometry; | |
| LiBr means lithium bromide; | IPA means isopropyl alcohol; |
| LiOH means lithium hydroxide; | Na means sodium; |
| | NaBH₃CN means sodium cyanoborohydride; |
| m means multiplet | MsCl means methanesulfonyl chloride; |
| NaBH₄ means sodium borohydride; | NCS means N-chlorosuccinimide; |
| M means molar; | Na₂CO₃ means sodium carbonate; |
| Me means methyl; | NaH means sodium hydride; |
| MeCN means acetonitrile; | NaHCO₃ means sodium bicarbonate; |
| MeOH means methanol; | NaI means sodium iodide; |
| MeOH-d₄ means deutero-methanol; | NaOH means sodium hydroxide; |
| mg means milligram; | Na₂SO₄ means sodium sulfate; |
| MgSO₄ means magnesium sulfate; | NH₃ means ammonia; |
| MS m/z means mass spectrum peak; | NH₄Cl means ammonium chloride; |
| NH₄HCO₃ means ammonium | NH₄OH is ammonium hydroxide; |
| bicarbonate; | |
| OMs means mesylate; | PE means petroleum ether; |
| P(n-Bu)₃ means Tri-n-butylphosphine; | Psi means pounds per square inch; |
| Pd(OAc)₂ means palladium acetate; | Pd(dppf)Cl₂ means [1,1′- |
| | bis(diphenylphosphino)ferrocene]dichloropalladium(II); |
| Pd(PPh₃)₄ means | PrCN means butyronitrile; |
| tetrakis(triphenylphosphine)palladium(0); | |
| q means quartet; | rt or means room temperature; |
| s means singlet; | sat. means saturated; |
| SFC means supercritical fluid | soln. means solution; |
| chromatography; | |
| SOCl₂ means thionyl chloride; | |
| STAB means sodium | t means triplet; |
| triacetoxyborohydride; | |
| TFA means trifluoroacetic acid; | t-BuONa means sodium tert-butoxide; |
| TEA means triethylamine; | TBDMS means tert-butyldimethylsilyl; |
| TBAF means tetrabutylammonium | T3P ® means propylphosphonic anhydride solution; |
| fluoride; | |
| TLC means thin layer chromatography; | THF means tetrahydrofuran; |
| TMSCHN₂ means | TMS means trimethylsilyl; |
| (trimethylsilyl)diazomethane; | |
| μmol means micromole; | μL means micro litres; |
| Xantphos means 4,5- | XPhos means 2-dicyclohexylphosphino-2′,4′,6′- |
| bis(diphenylphosphino)-9,9- | triisopropylbiphenyl; |
| dimethylxanthene; | |
| BOP: (Benzotriazol-1- | HATU: 1-[Bis(dimethylamino)methylene]-1H-1,2,3- |
| yloxy)tris(dimethylamino)phosphonium | triazolo[4,5-b]pyridinium 3-oxid hexafluorophosphate |
| hexafluorophosphate | |
| Zn(CN)₂ means zinc cyanide; | D₂O means deuterated water; |
| | NBS: N-bromosuccinimide |
| | ABPR: Automated Back Pressure Regulator |
| MBPR: manual back pressure regular | DEA: diethylamine |
| | MHz means mega Hertz; |
| NIS: N-Iodosuccinimide | |
| NaHMDS: Sodium | t-BuOK: Potassium t-butoxide |
| bis(trimethylsilyl)amide | |

For illustrative purposes, the reaction schemes depicted below provide potential routes for synthesizing the compounds of the present invention as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.
SCHEMES: Scheme 1, 2, 3, 4, 5 and 6 provide potential routes for making compounds of Formula (I).

### Scheme 1:

According to a first process, compounds of Formula (I), may be prepared from compounds of Formulae (II), (III), (IV), (V), (VI), (VII) and (VIII) as illustrated by Scheme 1.

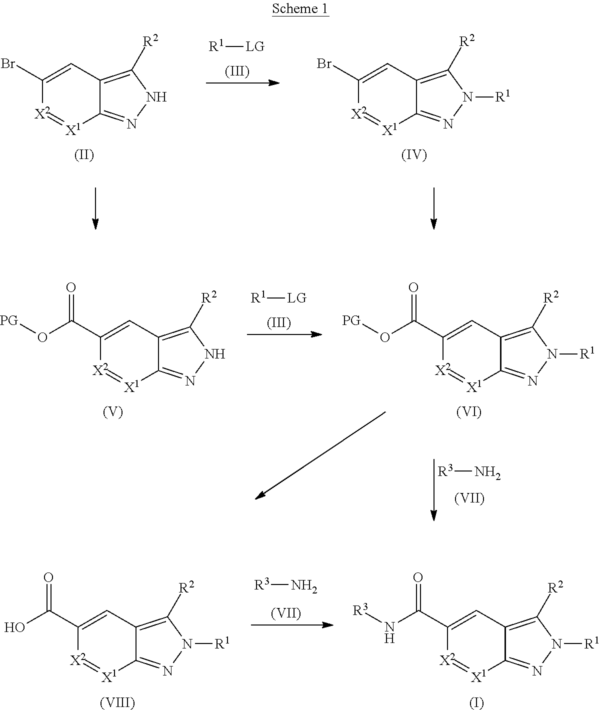

LG is a leaving group, typically mesylate, tosylate, iodo or bromo
PG is a carboxylic acid protecting group, typically C₁-C₄ alkyl or phenyl and preferably Me, Et or phenyl.
Compounds of Formula (IV) may be prepared from the compound of Formula (II) and the compound of Formula (III) by an alkylation reaction in the presence of a suitable inorganic base and a suitable polar aprotic solvent at between 0° C. and elevated temperature. Preferred conditions comprise reaction of the compound of Formula (II) with the compound of Formula (III) in the presence of K₂CO₃ or Cs₂CO₃ in DMF at between 0° C. and 110° C.
Alternatively, compounds of Formula (IV) may be prepared by an addition reaction of the compound of Formula (II) with R^{1′}CH═CH₂, (wherein R^{1′}CH₂—CH₂ is an entity that may be transformed using standard chemical transformations to R¹), in the presence of a non-nucleophilic base, such as DBU in a suitable solvent, such as MeCN at between rt and 50° C., followed by a standard chemical transformation, such as a reduction of an ester, to provide the compound of Formula (IV).
Compounds of Formula (V) may be prepared from the bromide of Formula (II) by a palladium catalysed carbonylation reaction, in the presence of a suitable palladium catalyst, organic base and suitable alcohol at elevated temperature under an atmosphere of CO. When PG is methyl or ethyl, preferred conditions comprise, reaction of the bromide of Formula (II) under an atmosphere of CO in the presence of suitable palladium catalyst such as Pd(dppf)Cl₂, an organic base such as TEA in a solvent such as MeOH or EtOH at between 80 and 100° C.
Alternatively, when PG is phenyl, compounds of Formula (V) may be prepared from the bromide of Formula (II) by a palladium catalyzed reaction with phenyl formate, in the presence of a suitable palladium catalyst such as Pd(OAc)₂ with a phosphine-based ligand such as BINAP or XantPhos, an organic base such as N,N-diethylethanamine, in a solvent such as MeCN at between 80 and 100° C.
Compounds of Formula (VI) may be prepared from the compound of Formula (V) and the compound of Formula (III) by an alkylation reaction as described above, for the preparation of compounds of the Formula (IV).
Alternatively compounds of Formula (VI) may be prepared from the bromide of Formula (IV) via a palladium catalysed carbonylation reaction as previously described above, for the preparation of compounds of the Formula (V).
Compounds of Formula (VIII) may be prepared by the hydrolysis of the ester of Formula (VI) under suitable acidic or basic conditions in a suitable aqueous solvent. Preferred conditions comprise the treatment of the ester of Formula (VI) with an alkali metal base such as LiOH, NaOH or K₂CO₃ in aqueous MeOH and/or THF at between rt and the reflux temperature of the reaction.
The compound of Formula (I) may be prepared by an amide bond formation of the acid of Formula (VIII) and the amine of Formula (VII) in the presence of a suitable coupling agent and organic base, optionally in a suitable polar aprotic solvent. Preferred conditions, comprise the reaction of the acid of Formula (VIII) with the amine of Formula (VII) in the presence of coupling agent preferably, T3P®, CDI, HATU or HOAt, in the presence of a suitable organic base such as TEA, DIPEA or pyridine, optionally in a suitable solvent, such as DMF, DMSO, EtOAc, dioxane or MeCN at between rt and the reflux temperature of the reaction.
Alternatively, compounds of Formula (I) may be prepared directly from compounds of Formula (VI) by reaction with the amine of Formula (VII) in the presence of DABAL-Me₃, according to the method described by Novak et al (Tet. Lett. 2006, 47, 5767). Preferred conditions comprise reaction of the ester of Formula (VI) with the amine of Formula (VII) in the presence of DABAL-Me₃, in a suitable solvent such as THF at rt.
According to a second process, compounds of Formula (I), may be prepared from compounds of Formulae (III), (VII), (IX) and (X) as illustrated by Scheme 2.

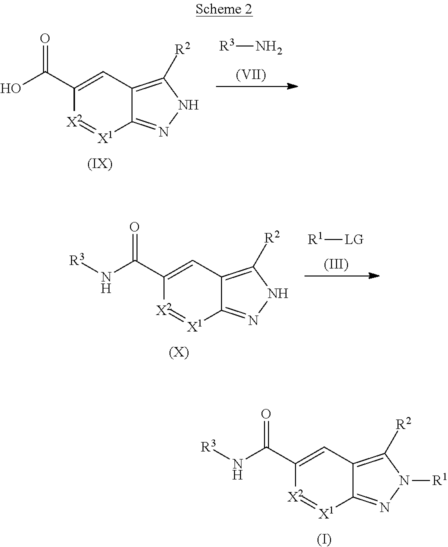

LG is as defined in Scheme 1 The compound of Formula (X) may be prepared by an amide bond formation of the acid of Formula (IX) and the amine of Formula (VII) in the presence of a suitable coupling agent and organic base in a suitable polar aprotic solvent as previously described in Scheme 1.
Compounds of Formula (I) may be prepared from the compound of Formula (X) and the compound of Formula (III) by an alkylation reaction in the presence of a suitable inorganic base and a suitable polar aprotic solvent as previously described in Scheme 1.
According to a third process, compounds of Formula (I), may be prepared from compounds of Formulae (VII), (VIII) and (XI) as illustrated by Scheme 3.

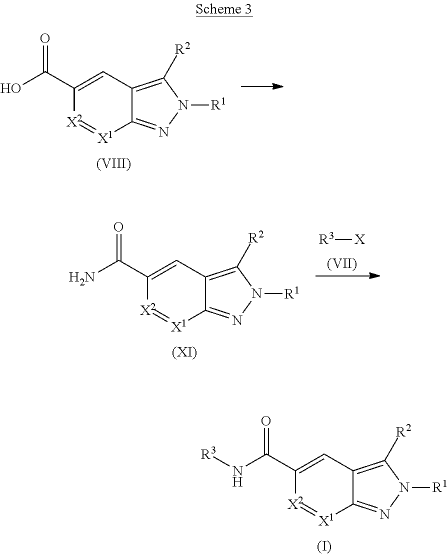

Compounds of Formula (XI) may be prepared by formation of the acid chloride of the acid of Formula (VIII), typically using thionyl chloride and DMF in DCM at rt and the subsequent amide formation, by reaction with NH₄OH, in a suitable solvent such as THF, at rt.
Compounds of Formula (I) may be prepared from compounds of Formula (XI) and the aryl-halide, such as an arylbromide or aryl iodide of Formula (VII) via a suitable palladium catalyzed cross coupling reaction. Typical conditions comprise, reaction in the presence of a suitable palladium catalyst such as Pd₂(dba)₃ with a phosphine-based ligand such as XantPhos, an inorganic base such as Cs₂CO₃, in a solvent such as toluene at about 110° C.

According to a fourth process, compounds of Formula (II)(A), wherein X₂ is C—OR⁶, may be prepared from compounds of Formulae (XII), (XIII) and (XIV) as illustrated by Scheme 4.

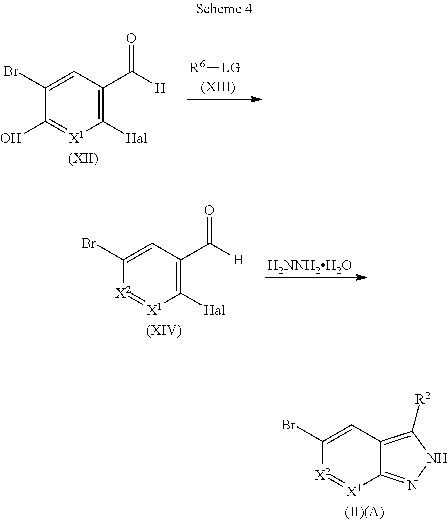

Hal is halogen, preferably fluorine.
LG is as defined in Scheme 1.
Compounds of Formula (XIV) may be prepared from the compound of Formula (XII) and the compound of Formula (XIII) by an alkylation reaction in the presence of a suitable inorganic base and a suitable polar aprotic solvent at between rt and elevated temperature. Preferred conditions comprise reaction of the compound of Formula (XII) with the compound of Formula (XIII) in the presence of K₂CO₃ in DMF at between 50° C. and 100° C.
Compounds of Formula (II)(A) may be prepared by the condensation of the compound of Formula (XIV) with hydrazine hydrate in the presence of a suitable inorganic base such as K₂CO₃ and a suitable polar aprotic solvent, such as DMSO at elevated temperature, such as 100° C.
According to a fifth process, compounds of Formula (IV), may be prepared from compounds of Formulae (III), (XV) and (XVI) as illustrated by Scheme 5.

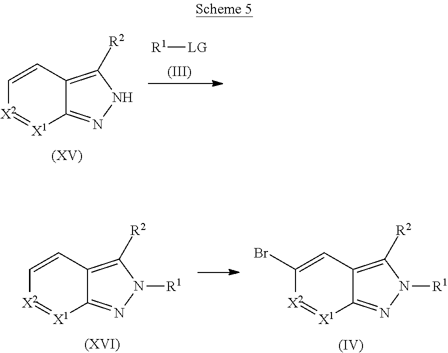

Compounds of Formula (XVI) may be prepared from the compound of Formula (XV) and the compound of Formula (III) by an alkylation reaction, as previously described in Scheme 1.
Compounds of Formula (IV) may be prepared from the compound of Formula (XVI) by a bromination reaction, using Br₂ under acidic conditions, typically in AcOH, at about rt.
According to a sixth process, compounds of Formula (IV), may be prepared from compounds of the Formulae (XVII) and (XVIII) as illustrated in Scheme 6.

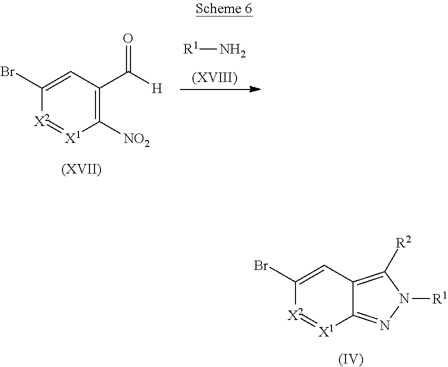

Compounds of Formula (IV) may be prepared from the compound of Formula (XVII) and the amine of Formula (XVIII), by a cyclisation reaction under Cadogan like conditions. Typical conditions comprise reaction of the aldehyde of Formula (XVII) with the amine of Formula (XVIII) in the presence of a suitable organic base, such as TEA in a suitable alcoholic solvent, such as isopropanol, at elevated temperature, followed by treatment with a suitable phosphine ligand, such as P(n-Bu)₃ or PPh₃.
Compounds of Formulae (I), (II), (IV), (V), (VI), (X), (XI), (XVI) may be converted to alternative compounds of Formulae (I), (II), (IV), (V), (VI), (X), (XI), (XVI) by standard chemical transformations such as for example, alkylation of a heteroatom such as N or O, halogenation, or reduction of an ester using methods well known to those skilled in the art.
The compounds of Formulae (II), (III), (V), (VII), (IX), (XII), (XIII), (XV), (XVII) and (XVIII) are commercially available, may be prepared by analogy to methods known in the literature, or the methods described in the Experimental section below.
It will be appreciated by those skilled in the art that it may be necessary to utilise a suitable protecting group strategy for the preparation of compounds of Formula (I). Typical protecting groups may comprise, carbamate and preferably Boc for the protection of amines, a TBDMS, PMB or benzyl group for the protection of a primary or secondary alcohol, a C₁-C₄ alkyl, phenyl or benzyl group for the protection of carboxylic acids or a THP group for the protection of indazole or pyrazolo[1,5-a]pyridine rings.
It will be appreciated by those skilled in the art that the experimental conditions set forth in the schemes that follow are illustrative of suitable conditions for effecting the transformations shown, and that it may be necessary or desirable to vary the precise conditions employed for the preparation of the compound of Formula (I). It will be further appreciated that it may be necessary or desirable to carry out the transformations in a different order from that described in the schemes, or to modify one or more of the transformations, to provide the desired compound of the invention

### PREPARATION OF INTERMEDIATES

### Preparation 1: 5-Bromo-6-methoxy-2H-indazole

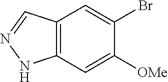 [SMILES: COc1cc2[nH]ncc2cc1Br]

A solution of 5-bromo-2-fluoro-4-methoxy-benzaldehyde (10.0 g, 42.9 mmol) in hydrazine hydrate (52.1 mL, 1.07 mol) was heated at 100° C. for 8 h. The cooled reaction mixture was poured into ice-water, the resulting precipitate filtered off, washed with water and air-dried to afford 5-bromo-6-methoxy-2H-indazole, 6.10 g, 62.6% yield. LCMS m/z=227, 229 [M+H]⁺; ¹H NMR (500 MHz, DMSO-d₆) δ: 3.89 (s, 3H), 7.07 (s, 1H), 7.93 (s, 1H), 7.99 (d, 1H), 12.96 (s, 1H).

### Preparation 2: 5-Bromo-2-fluoro-4-isopropoxybenzaldehyde

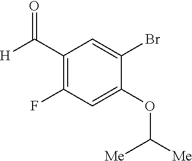 [SMILES: CC(C)Oc1cc(F)c(C=O)cc1Br]

To a solution of 5-bromo-2-fluoro-4-hydroxybenzaldehyde (8.00 g, 36.5 mmol) and 2-iodopropane (9.31 g, 54.8 mmol) in DMF (150 mL) was added K₂CO₃ (10.1 g, 73.1 mmol) and the reaction stirred at 75° C. for 16 h. The cooled mixture was diluted with water (30 mL) and extracted with EtOAc (30 mL×3). The combined organic layers were washed with brine (30 mL), dried over Na₂SO₄ and filtered. The filtrate was concentrated in vacuo to afford 5-bromo-2-fluoro-4-isopropoxybenzaldehyde (8.70 g, 91.2% yield) as a yellow oil. ¹H NMR (500 MHz, CDCl₃) δ: 1.44 (d, 6H), 4.60-4.70 (m, 1H), 6.64 (d, 1H), 8.05 (d, 1H), 10.10 (s, 1H).

### Preparation 3: 5-Bromo-6-isopropoxy-1H-indazole

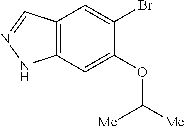 [SMILES: CC(C)Oc1cc2[nH]ncc2cc1Br]

To a solution of 5-bromo-2-fluoro-4-isopropoxybenzaldehyde (Preparation 2, 8.70 g, 33.3 mmol) in DMSO (150 mL) was added K₂CO₃ (4.61 g, 33.3 mmol) and hydrazine hydrate (25.0 g, 500 mmol) and the reaction stirred at 100° C. for 16 h. The cooled mixture was diluted with aq. HCl (15 mL) and extracted with EtOAc (50 mL×3). The combined organic layers were washed with brine (30 mL), dried over Na₂SO₄, filtered and concentrated in vacuo. The crude was purified by silica gel column chromatography using a Combiflash® system, eluting with PE/EtOAc (75/25) to afford 5-bromo-6-isopropoxy-1H-indazole (1.50 g, 17.6% yield) as a yellow oil. ¹H NMR (400 MHz, CDCl₃) δ: 1.44-1.46 (d, 6H), 4.59-4.61 (m, 1H), 6.93 (s, 1H), 7.90-8.00 (m, 2H), 9.93 (br s, 1H).

### Preparation 4: 5-Bromo-7-methoxy-1H-indazole

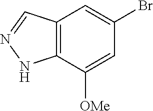 [SMILES: COc1cc(Br)cc2cn[nH]c12]

To a solution of 4-bromo-2-methoxy-6-methylaniline (8.00 g, 37.0 mmol) in AcOH (80 mL) and water (16 mL) was added sodium nitrite (3.83 g, 55.5 mmol) and the reaction stirred at 15° C. for 14 h. The mixture was concentrated in vacuo, the residue was neutralised using saturated aq. NaHCO₃ (100 mL×3) and extracted with EtOAc (250 mL×3). The combined organic layers were washed with brine (80 mL×2), dried over Na₂SO₄, filtered and concentrated in vacuo. The residue was purified by column chromatography on silica gel using a Combiflash® system, eluting with PE/EtOAc (75/25) to afford 5-bromo-7-methoxy-1H-indazole (1.70 g, 17% yield) as a brown solid. ¹HNMR (400 MHz, DMSO-d₆) δ: 3.97 (s, 3H), 6.94 (s, 1H), 7.54 (s, 1H), 8.00 (s, 1H), 13.50 (s, 1H).

### Preparation 5: Methyl 6-methoxy-1H-indazole-5-carboxylate

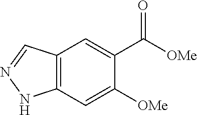 [SMILES: COC(=O)c1cc2cn[nH]c2cc1OC]

5-Bromo-6-methoxy-2H-indazole (Preparation 1, 5.50 g, 24.2 mmol), TEA (4.03 mL, 29.1 mmol) and Pd(dppf)Cl₂ (531 mg, 0.727 mmol) were dissolved in dry MeOH (100 mL) and the reaction heated at 100° C. under 40 atm. CO pressure for 16 h. The cooled mixture was evaporated under reduced pressure and the residue diluted with water (50 mL). The mixture was extracted with EtOAc (2×50 mL), the combined organic phases dried over Na₂SO₄, filtered and evaporated to dryness to afford methyl 6-methoxy-1H-indazole-5-carboxylate, 4.10 g, 79.6% yield, as a yellow solid. LCMS m/z=207.2 [M+H]⁺; ¹H NMR (400 MHz, DMSO-d₆) δ: 3.78 (s, 3H), 3.86 (s, 3H), 7.03 (s, 1H), 8.07 (s, 1H), 8.12 (d, 1H), 13.13 (s, 1H).

### Preparation 6: 5-Bromo-6-methoxy-1-(tetrahydro-2H-pyran-2-yl)-1H-indazole

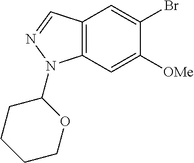 [SMILES: COc1cc2c(cnn2C2CCCCO2)cc1Br]

To a solution of 5-bromo-6-methoxy-1H-indazole (2.00 g, 8.81 mmol) in DCM (50 mL) was added 3,4-dihydro-2H-pyran (1.11 g, 13.2 mmol) and 4-methylbenzenesulfonic acid hydrate (335 mg, 1.76 mmol) and the reaction stirred at 15° C. for 16 h. The reaction was concentrated in vacuo and the residue purified by column chromatography on silica gel using a Combiflash® system, eluting with PE/EA (75/25) to afford 5-bromo-6-methoxy-1-(tetrahydro-2H-pyran-2-yl)-1H-indazole (2.30 g, 82.1% yield) as a white solid. ¹H NMR (400 MHz, CDCl₃) δ: 1.69-1.80 (m, 3H), 2.08-2.10 (m, 1H), 2.16-2.18 (m, 1H), 2.55-2.58 (m, 1H), 3.75-3.79 (m, 1H), 3.99-4.01 (m, 4H), 5.68 (dd, 1H), 6.98 (s, 1H), 7.89 (s, 1H), 7.90 (s, 1H).

### Preparation 7: 5-Bromo-6-isopropoxy-2-(tetrahydro-2H-pyran-2-yl)-2H-indazole

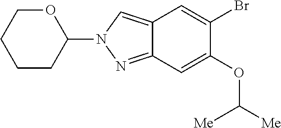 [SMILES: CC(C)Oc1cc2nn(C3CCCCO3)cc2cc1Br]

To a solution of 5-bromo-6-isopropoxy-1H-indazole (Preparation 3, 2.50 g, 9.80 mmol) in DCM (30 mL) was added 3,4-dihydro-2H-pyran (1.24 g, 14.70 mmol) and 4-methylbenzenesulfonic acid hydrate (372 mg, 1.96 mmol) and the reaction stirred at rt for 16 h. The reaction mixture was filtered and concentrated in vacuo. The residue was purified by silica gel column chromatography using a Combiflash® system, eluting with PE/EtOAc (75/25) to afford 5-bromo-6-isopropoxy-2-(tetrahydro-2H-pyran-2-yl)-2H-indazole (2.00 g, 60% yield) as yellow oil. ¹H NMR (400 MHz, CDCl₃) δ: 1.41-1.43 (d, 6H), 1.60-1.80 (m, 4H), 2.10-2.20 (m, 1H), 2.40-2.60 (m, 1H), 3.69-3.74 (m, 1H), 3.90-4.00 (m, 1H), 4.60-4.70 (m, 1H), 5.60-5.70 (m, 1H), 6.98 (s, 1H), 7.77-7.87 (m, 2H).

### Preparation 8: 5-Bromo-7-methoxy-2-(tetrahydro-2H-pyran-2-yl)-2H-indazole

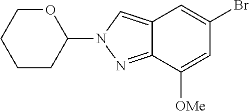 [SMILES: COc1cc(Br)cc2cn(C3CCCCO3)nc12]

To a solution of 5-bromo-7-methoxy-2H-indazole (1.70 g, 7.49 mmol) in DCM (30 mL) was added 4-methylbenzenesulfonic acid hydrate (285 mg, 1.50 mmol) and 3,4-dihydro-2H-pyran (1.26 g, 15.0 mmol) and the reaction stirred at 40° C. for 14 h. The reaction was neutralised using saturated aq. NaHCO₃ (20 mL×2), extracted with DCM (40 mL×3), the combined organic layers dried over Na₂SO₄, filtered and concentrated in vacuo. The residue was purified by column chromatography on silica gel using a Combiflash® system, eluting with PE/EtOAc (75/25) to afford 5-bromo-7-methoxy-2-(tetrahydro-2H-pyran-2-yl)-2H-indazole (1.50 g, 60% yield) as yellow oil. ¹H NMR (400 MHz, CDCl₃) δ: 1.55-1.58 (m, 1H), 1.74-1.77 (m, 2H), 2.05-2.07 (m, 1H), 2.16-2.18 (m, 1H), 2.52-2.55 (m, 1H), 3.75-3.79 (m, 1H), 4.00 (s, 3H), 4.07-4.10 (m, 1H), 6.18 (dd, 1H), 6.86 (s, 1H), 7.44 (s, 1H), 7.94 (s, 1H).

### Preparation 9: Methyl 6-methoxy-1-(tetrahydro-2H-pyran-2-yl)-1H-indazole-5-carboxylate

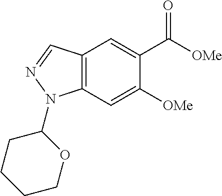 [SMILES: COC(=O)c1cc2cnn(C3CCCCO3)c2cc1OC]

To a solution of 5-bromo-6-methoxy-1-(tetrahydro-2H-pyran-2-yl)-1H-indazole (Preparation 6, 2.30 g, 7.39 mmol) in MeOH (50 mL) was added TEA (3.74 g, 37.0 mmol) and Pd(dppf)Cl₂ (1.08 g, 1.48 mmol) and the reaction stirred at 80° C. under CO (50 psi) for 16 h. The cooled reaction mixture was filtered and concentrated in vacuo. The residue was purified by silica gel column chromatography using a Combiflash® system, eluting with PE/EtOAc (75/25) to afford methyl 6-methoxy-1-(tetrahydro-2H-pyran-2-yl)-1H-indazole-5-carboxylate (1.80 g, 82.3% yield) as a white solid. LCMS m/z=290.9 [M+H]⁺; ¹H NMR (400 MHz, CDCl₃) δ: 1.69-1.78 (m, 3H), 2.10-2.12 (m, 1H), 2.17-2.29 (m, 1H), 2.56-2.58 (m, 1H), 3.74-3.79 (m, 1H), 3.92 (s, 3H), 4.00 (s, 3H), 4.02-4.04 (m, 1H), 5.69 (d, 1H), 6.98 (s, 1H), 7.99 (s, 1H), 8.24 (s, 1H).

### Preparation 10: Methyl 6-isopropoxy-2-(tetrahydro-2H-pyran-2-yl)-2H-indazole-5-carboxylate

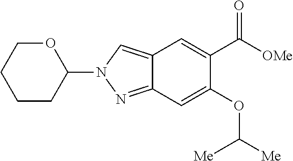 [SMILES: COC(=O)c1cc2cn(C3CCCCO3)nc2cc1OC(C)C]

Methyl 6-isopropoxy-2-(tetrahydro-2H-pyran-2-yl)-2H-indazole-5-carboxylate was obtained as an orange oil, 1.00 g, 53% yield, from 5-bromo-6-isopropoxy-2-(tetrahydro-2H-pyran-2-yl)-2H-indazole (Preparation 7), following a similar procedure to that described in Preparation 9. LCMS m/z=319.0 [M+H]⁺; ¹H NMR (400 MHz, CDCl₃) δ: 1.41 (d, 6H), 1.50-1.75 (m, 3H), 2.00-2.10 (m, 1H), 2.10-2.20 (m, 1H), 3.65-3.75 (m, 1H), 3.90-4.00 (m, 1H), 3.95 (s, 3H), 3.90-4.00 (m, 1H), 4.60-4.70 (m, 1H), 5.62 (d, 1H), 6.98 (s, 1H), 7.95 (s, 1H), 8.17 (s, 1H).

### Preparation 11: Methyl 7-methoxy-2-(tetrahydro-2H-pyran-2-yl)-2H-indazole-5-carboxylate

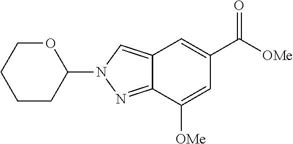 [SMILES: COC(=O)c1cc(OC)c2nn(C3CCCCO3)cc2c1]

Methyl 7-methoxy-2-(tetrahydro-2H-pyran-2-yl)-2H-indazole-5-carboxylate was obtained as a yellow solid, 1.20 g, 85.7% yield, from 5-bromo-7-methoxy-2-(tetrahydro-2H-pyran-2-yl)-2H-indazole (Preparation 8), following the procedure described in Preparation 9. LCMS m/z=291.1 [M+H]⁺; ¹H NMR (400 MHz, CDCl₃) δ: 1.60-1.62 (m, 1H), 1.75-1.80 (m, 2H), 2.05-2.08 (m, 1H), 2.16-2.18 (m, 1H), 2.60-2.64 (m, 1H), 3.72-3.78 (m, 1H), 3.95 (s, 3H), 4.06 (s, 3H), 4.09-4.12 (m, 1H), 6.25 (dd, 1H), 7.45 (s, 1H), 8.10 (s, 1H), 8.11 (s, 1H).

### Preparation 12: 6-Methoxy-1-(tetrahydro-2H-pyran-2-yl)-1H-indazole-5-carboxylic Acid

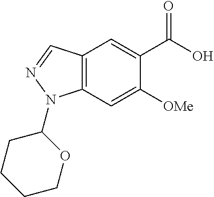 [SMILES: COc1cc2c(cnn2C2CCCCO2)cc1C(=O)O]

LiOH (742 mg, 31.0 mmol) was added to a solution of methyl 6-methoxy-1-(tetrahydro-2H-pyran-2-yl)-1H-indazole-5-carboxylate (Preparation 9, 1.80 g, 6.20 mmol) in THF (8 mL), MeOH (8 mL) and water (8 mL) and the reaction stirred at 20° C. for 16 h. The reaction mixture was concentrated in vacuo, the residue diluted with water (30 mL) and extracted with EtOAc (30 mL). The pH of the aqueous phase was adjusted to 1 using 1 M HCl (5 mL) and the solution extracted with EtOAc (30 mL×3). These combined organic extracts were washed with brine (50 mL), dried over Na₂SO₄, filtered and evaporated under reduced pressure to afford 6-methoxy-1-(tetrahydro-2H-pyran-2-yl)-1H-indazole-5-carboxylic acid (1.70 g, 98.23% yield) as a white solid. LCMS m/z=279.9 [M+H]⁺; ¹H NMR (400 MHz, CDCl₃) δ: 1.72-1.78 (m, 3H), 2.11-2.16 (m, 2H), 2.55-2.58 (m, 1H), 3.75-3.80 (m, 1H), 3.97-4.00 (m, 1H), 4.17 (s, 3H), 5.73 (dd, 1H), 7.10 (s, 1H), 8.08 (s, 1H), 8.68 (s, 1H), 10.53 (br s, 1H).

### Preparation 13: 6-Isopropoxy-2-(tetrahydro-2H-pyran-2-yl)-2H-indazole-5-carboxylic Acid

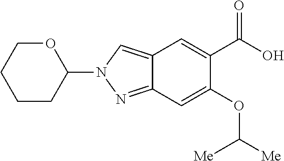 [SMILES: CC(C)Oc1cc2nn(C3CCCCO3)cc2cc1C(=O)O]

NaOH (377 mg, 9.42 mmol) was added to a solution of methyl 6-isopropoxy-2-(tetrahydro-2H-pyran-2-yl)-2H-indazole-5-carboxylate (Preparation 10, 1.00 g, 3.14 mmol) in MeOH (5 mL) and H₂O (5 mL) and the reaction stirred at rt for 16 h. The reaction was diluted with water (10 mL) and neutralised using 1 M HCl. The mixture was extracted with EtOAc (20 mL×3), the combined organic layers washed with brine (20 mL), dried over Na₂SO₄, filtered and evaporated under reduced pressure to afford 6-isopropoxy-2-(tetrahydro-2H-pyran-2-yl)-2H-indazole-5-carboxylic acid (900 mg, 94% yield) as a white solid. LCMS m/z=305.1 [M+H]⁺; ¹H NMR (400 MHz, CDCl₃) δ: 1.53 (d, 6H), 1.60-1.80 (m, 2H), 1.90-2.00 (m, 1H), 2.05-2.20 (m, 2H), 2.45-2.55 (m, 1H), 3.70-3.80 (m, 1H), 3.90-4.00 (m, 1H), 4.90-5.00 (m, 1H), 5.60-5.70 (m, 1H), 7.09 (s, 1H), 8.03 (s, 1H), 8.64 (d, 1H), 11.25 (br s, 1H).

### Preparation 14: 7-Methoxy-2-(tetrahydro-2H-pyran-2-yl)-2H-indazole-5-carboxylic Acid

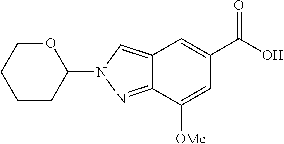 [SMILES: COc1cc(C(=O)O)cc2cn(C3CCCCO3)nc12]

NaOH (55.1 mg, 1.38 mmol) was added to a solution of methyl 7-methoxy-2-tetrahydro-2H-pyran-2-yl)-2H-indazole-5-carboxylate (Preparation 11, 200 mg, 0.689 mmol) in MeOH (1 mL), THF (1 mL) and water (1 mL) and the reaction stirred at 15° C. for 14 h. The mixture was concentrated in vacuo, and then neutralised using aqueous KHSO₄. The mixture was evaporated to afford 7-methoxy-2-(tetrahydro-2H-pyran-2-yl)-2H-indazole-5-carboxylic acid (1.60 g) as a white solid. ¹H NMR (500 MHz, DMSO-d₆) δ: 1.50-1.52 (m, 2H), 1.53-1.56 (m, 1H), 1.95-1.99 (m, 2H), 2.28-2.39 (m, 1H), 3.62-3.64 (m, 1H), 3.90-3.92 (m, 1H), 3.98 (s, 3H), 6.13 (dd, 1H), 7.45 (s, 1H), 7.89 (s, 1H), 8.12 (s, 1H).

### Preparation 15: 6-Methoxy-N-(6-methoxypyridin-2-yl)-1H-indazole-5-carboxamide

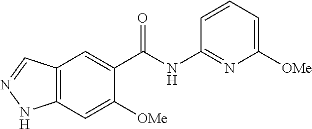 [SMILES: COc1cccc(NC(=O)c2cc3cn[nH]c3cc2OC)n1]

To a mixture of 6-methoxy-1H-indazole-5-carboxylic acid (600 mg, 3.12 mmol), 6-methoxypyridin-2-amine (388 mg, 3.12 mmol) and DIPEA (2.73 mL, 15.6 mmol) in EtOAc (12 mL) was added T3P® (50 wt. % in EtOAc, 5.58 mL, 9.37 mmol) and the reaction stirred at 22° C. for 18 h. The mixture was partitioned between EtOAc and water and the layers separated. The organic phase was washed with brine, dried over anhydrous MgSO₄, filtered and the filtrate evaporated in vacuo. The crude product was purified by column chromatography on silica gel using an ISCO® autopurification system, eluting with EtOAc/heptane (0/100 to 100/0) to afford 6-methoxy-N-(6-methoxypyridin-2-yl)-1H-indazole-5-carboxamide as a white solid (92.0 mg, 9.89%). LCMS m/z=299.1 [M+H]⁺

### Preparation 16: 6-Methoxy-N-(pyridin-2-yl)-1H-indazole-5-carboxamide

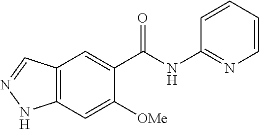 [SMILES: COc1cc2[nH]ncc2cc1C(=O)Nc1ccccn1]

6-Methoxy-N-(pyridin-2-yl)-1H-indazole-5-carboxamide was obtained as a yellow solid, 115 mg, 13.7% yield, from 6-methoxy-1H-indazole-5-carboxylic acid and 2-aminopyridine, following a similar procedure to that described in preparation 15. LCMS m/z=269.1 [M+H]⁺

### Preparation 17: N-(6-(difluoromethyl)pyridin-2-yl)-6-isopropoxy-2-(tetrahydro-2H-pyran-2-yl)-2H-indazole-5-carboxamide

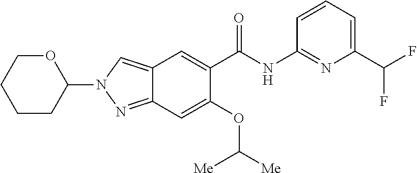 [SMILES: CC(C)Oc1cc2nn(C3CCCCO3)cc2cc1C(=O)Nc1cccc(C(F)F)n1]

T3P® (50 wt. % in EtOAc, 1.25 g, 3.94 mmol) was added to a solution of 6-isopropoxy-2-(tetrahydro-2H-pyran-2-yl)-2H-indazole-5-carboxylic acid (Preparation 13, 1.20 g, 3.94 mmol) and 6-(difluoromethyl)pyridin-2-amine (681 mg, 4.73 mmol) in pyridine (20 mL) and the reaction stirred at 15° C. for 16 h. The reaction mixture was concentrated in vacuo, the residue diluted with water (20 mL) and aqueous NaHCO₃ (20 mL) and extracted with EtOAc (30 mL×3). The combined organic extracts were washed with brine (30 mL), dried over Na₂SO₄, filtered and concentrated in vacuo. The residue was purified by column chromatography on silica gel using a Combiflash® system, eluting with PE/EtOAc (75:25) to afford N-(6-(difluoromethyl)pyridin-2-yl)-6-isopropoxy-2-(tetrahydro-2H-pyran-2-yl)-2H-indazole-5-carboxamide as a white solid (1.30 g, 77% yield). ¹H NMR (400 MHz, CDCl₃) δ: 1.50-1.60 (m, 6H), 1.60-1.80 (m, 3H), 2.00-2.20 (m, 2H), 2.50-2.60 (m, 1H), 3.70-3.80 (m, 1H), 4.00 (m, 1H), 4.90-5.00 (m, 1H), 5.68 (d, 1H), 6.30-6.60 (m, 1H), 7.06 (s, 1H), 7.34 (d, 1H), 7.86 (t, 1H), 8.05 (s, 1H), 8.50-8.60 (m, 1H), 8.72 (s, 1H), 10.93 (s, 1H).

### Preparation 18: N-(6-(difluoromethyl)pyridin-2-yl)-6-methoxy-1-(tetrahydro-2H-pyran-2-yl)-1H-indazole-5-carboxamide

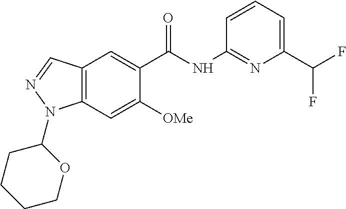 [SMILES: COc1cc2c(cnn2C2CCCCO2)cc1C(=O)Nc1cccc(C(F)F)n1]

N-(6-(difluoromethyl)pyridin-2-yl)-6-methoxy-1-(tetrahydro-2H-pyran-2-yl)-1H-indazole-5-carboxamide (450 mg, 73.3%) was obtained from 6-methoxy-1-(tetrahydro-2H-pyran-2-yl)-1H-indazole-5-carboxylic acid (Preparation 12) and 6-(difluoromethyl)pyridin-2-amine, following the procedure described in preparation 17. ¹H NMR (500 MHz, CDCl₃) δ: 1.72-1.80 (m, 3H), 2.11-2.19 (m, 2H), 2.57-2.60 (m, 1H), 3.77-3.81 (m, 1H), 4.01-4.04 (m, 1H), 4.19 (s, 3H), 5.73 (dd, 1H), 6.56 (dd, 1H), 7.08 (s, 1H), 7.37 (d, 1H), 7.89 (dd, 1H), 8.08 (s, 1H), 8.56 (d, 1H), 8.72 (s, 1H), 10.41 (br s, 1H).

### Preparation 19: 7-Methoxy-N-(6-methoxypyridin-2-yl)-2-(tetrahydro-2H-pyran-2-yl)-2H-indazole-5-carboxamide

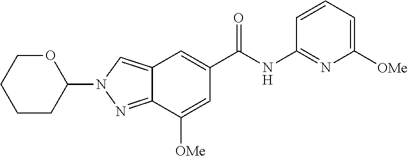 [SMILES: COc1cccc(NC(=O)c2cc(OC)c3nn(C4CCCCO4)cc3c2)n1]

7-Methoxy-N-(6-methoxypyridin-2-yl)-2-(tetrahydro-2H-pyran-2-yl)-2H-indazole-5-carboxamide was obtained, from 7-methoxy-2-(tetrahydro-2H-pyran-2-yl)-2H-indazole-5-carboxylic acid (Preparation 14) and 6-methoxypyridin-2-amine following the procedure described in preparation 17. LCMS m/z=383.1 [M+H]⁺; ¹H NMR (400 MHz, CDCl₃) δ: 1.58-1.64 (m, 2H), 1.78-1.81 (m, 2H), 2.06-2.11 (m, 2H), 2.62-2.64 (m, 1H), 3.74-3.77 (m, 1H), 3.92 (s, 3H), 4.10-4.16 (m, 3H), 6.28 (dd, 1H), 6.53 (dd, 1H), 7.45 (s, 1H), 7.65 (dd, 1H), 7.85 (s, 1H), 7.93 (d, 1H), 8.14 (s, 1H), 8.41 (br s, 1H).

### Preparation 20: N-(6-(difluoromethyl)pyridin-2-yl)-6-isopropoxy-2H-indazole-5-carboxamide

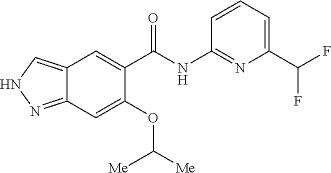 [SMILES: CC(C)Oc1cc2n[nH]cc2cc1C(=O)Nc1cccc(C(F)F)n1]

HCl/EtOAc (4 M, 12 mL) was added to a solution of N-(6-(difluoromethyl)pyridin-2-yl)-6-isopropoxy-2-(tetrahydro-2H-pyran-2-yl)-2H-indazole-5-carboxamide (Preparation 17, 1.30 g, 3.02 mmol) in EtOAc (12 mL) and the reaction stirred at 15° C. for 16 h. The reaction was concentrated in vacuo, the residue diluted with water (10 mL) and the mixture neutralised using NaHCO₃ aq. (20 mL). The mixture was extracted with EtOAc (20 mL×3), the combined organic extracts washed with brine (20 mL), dried over Na₂SO₄, filtered and evaporated under reduced pressure to afford N-(6-(difluoromethyl)pyridin-2-yl)-6-isopropoxy-2H-indazole-5-carboxamide (1.00 g, 96%) as white solid. LCMS m/z=346.9 [M+H]⁺

### Preparation 21: N-(6-(difluoromethyl)pyridin-2-yl)-6-methoxy-2H-indazole-5-carboxamide

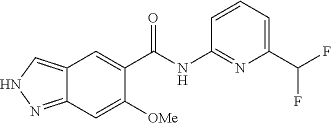 [SMILES: COc1cc2n[nH]cc2cc1C(=O)Nc1cccc(C(F)F)n1]

N-(6-(difluoromethyl)pyridin-2-yl)-6-methoxy-2H-indazole-5-carboxamide was obtained as a white solid (350 mg, 93.3%) from N-(6-(difluoromethyl)pyridin-2-yl)-6-methoxy-1-(tetrahydro-2H-pyran-2-yl)-1H-indazole-5-carboxamide (Preparation 18) following the procedure described in Preparation 20. LCMS/z=318.9 [M+H]⁺; ¹H NMR (400 MHz, CDCl₃) δ: 4.15 (s, 3H), 6.56 (dd, 1H), 7.03 (s, 1H), 7.39 (d, 1H), 7.89 (dd, 1H), 8.14 (s, 1H), 8.57 (dd, 1H), 8.77 (s, 1H), 10.38 (s, 1H).

### Preparation 22: 7-Methoxy-N-(6-methoxypyridin-2-yl)-2H-indazole-5-carboxamide

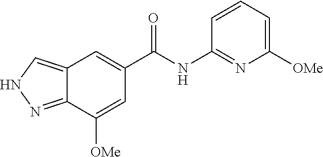 [SMILES: COc1cccc(NC(=O)c2cc(OC)c3n[nH]cc3c2)n1]

TFA (1 mL) was added to a solution of 7-methoxy-N-(6-methoxypyridin-2-yl)-2-(tetrahydro-2H-pyran-2-yl)-2H-indazole-5-carboxamide (Preparation 19, 230 mg, 0.602 mmol) in DCM (1 mL) and the reaction stirred at 15° C. for 2 h. The reaction was neutralised using saturated aq. NaHCO₃ (20 mL), extracted with DCM (40 mL×3), the combined organic layers were washed with brine, dried over Na₂SO₄, filtered and the filtrate concentrated in vacuo. The residue was purified by prep-TLC eluting with DCM/MeOH (20/1) to afford 7-methoxy-N-(6-methoxypyridin-2-yl)-2H-indazole-5-carboxamide as a white solid (25 mg, 10%)._LCMS m/z=298.9 [M+H]⁺

### Preparation 23: tert-Butyl (6-fluoropyrazolo[1,5-a]pyrimidin-3-yl)carbamate

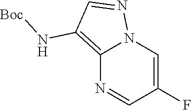 [SMILES: CC(C)(C)OC(=O)Nc1cnn2cc(F)cnc12]

To a solution of 6-fluoropyrazolo[1,5-a]pyrimidine-3-carboxylic acid (100 mg, 0.44 mmol) in t-BuOH (5 mL) was added DPPA (146 mg, 0.53 mmol) and TEA (89.4 mg, 0.88 mmol) and the reaction stirred at 100° C. for 16 h. The reaction mixture was diluted with water (30 mL) and extracted with EtOAc (30 mL×3). The combined organic layers were washed with brine (50 mL), dried over Na₂SO₄ and filtered. The filtrate was concentrated in vacuo and the residue purified by silica gel column chromatography using Combiflash® and eluting with (PE/EtOAc=91/9 to 50/50) to afford tert-butyl (6-fluoropyrazolo[1,5-a]pyrimidin-3-yl)carbamate (30 mg, 26.9% yield) as a yellow solid. LCMS m/z=252.9 [M+H]⁺

### Preparation 24: 6-Fluoropyrazolo[1,5-a]pyrimidin-3-amine Hydrochloride

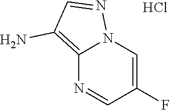 [SMILES: Nc1cnn2cc(F)cnc12 ; Cl]

To a solution of tert-butyl (6-fluoropyrazolo[1,5-a]pyrimidin-3-yl)carbamate (Preparation 23, 30 mg, 0.12 mmol) in EtOAc (2 mL) was added HCl/EtOAc (4 M, 2 mL) and the solution stirred at 15° C. for 1 h. The mixture was evaporated under reduced pressure to afford 6-fluoropyrazolo[1,5-a]pyrimidin-3-amine hydrochloride, as a yellow solid (22.0 mg). LCMS m/z=152.9 [M+H]⁺

### Preparation 25: 3-Methoxy-3-methylbutyl 4-methylbenzenesulfonate

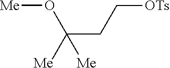 [SMILES: COC(C)(C)CCOS(=O)(=O)c1ccc(C)cc1]

To a solution of 3-methoxy-3-methylbutan-1-ol (1.00 g, 8.46 mmol) and 4-methylbenzenesulfonyl chloride (2.42 g, 12.69 mmol) in DCM (50 mL) was added TEA (2.57 g, 25.38 mmol) and DMAP (103.4 mg, 0.85 mmol) and the reaction stirred at 20° C. for 16 h. The reaction mixture was concentrated in vacuo and the residue purified by Combiflash® (PE/EtOAc=90/10) to afford 3-methoxy-3-methylbutyl 4-methylbenzenesulfonate (2.20 g, 95.5% yield) as yellow oil. ¹H NMR (500 MHz, CDCl₃) δ: 1.01-1.08 (m, 6H), 1.77 (t, 2H), 2.35-2.37 (m, 3H), 3.00-3.05 (m, 3H), 4.03 (t, 2H), 7.25 (d, 2H), 7.70 (d, 2H)

### Preparation 26: (1-Methyl-2-oxabicyclo[2.1.1]hexan-4-yl)methyl 4-methylbenzenesulfonate

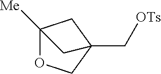 [SMILES: Cc1ccc(S(=O)(=O)OCC23COC(C)(C2)C3)cc1]

(1-Methyl-2-oxabicyclo[2.1.1]hexan-4-yl)methyl 4-methylbenzenesulfonate was obtained as a yellow oil, 600 mg, 90.8% yield, from (1-methyl-2-oxabicyclo[2.1.1]hexan-4-yl)methanol and 4-methylbenzenesulfonyl chloride, following a similar procedure to that described in Preparation 25. ¹HNMR (400 MHz, CDCl₃) δ: 1.40-1.41 (m, 3H), 1.53-1.59 (m, 4H), 2.46 (s, 3H), 3.60-3.61 (m, 2H), 4.25-4.26 (m, 2H), 7.36 (d, 2H), 7.76-7.79 (m, 2H)

### Preparation 27: Tetrahydro-2H-pyran-4-yl 4-methylbenzenesulfonate

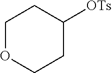 [SMILES: Cc1ccc(S(=O)(=O)OC2CCOCC2)cc1]

To a solution of tetrahydro-2H-pyran-4-ol (5.0 g, 49.0 mmol) in DCM (100 mL) was added pyridine (7.75 g, 97.92 mmol), 4-methylbenzenesulfonyl chloride (9.33 g, 49.0 mmol) and DMAP (598.1 mg, 4.90 mmol) and the reaction stirred at 50° C. for 16 h. The reaction mixture was diluted with water (150 mL), the layers separated and the organic phase washed with water (150 mL×2). The organic layer was concentrated in vacuo and the residue purified by silica gel chromatography with eluent (PE-EtOAc 94/6) to afford tetrahydro-2H-pyran-4-yl 4-methylbenzenesulfonate (6.17 g, 44.2% yield) as a clear oil. LCMS m/z=257.0 [M+H]⁺

### Preparation 28: 3-(Difluoromethyl)cyclobutyl Methanesulfonate

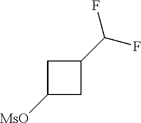 [SMILES: CS(=O)(=O)OC1CC(C(F)F)C1]

To a solution of 3-(difluoromethyl)cyclobutan-1-ol (100 mg, 0.78 mmol) and methanesulfonyl chloride (130 mg, 1.13 mmol) in DCM (5 mL) was added TEA (157 mg, 1.56 mmol) and the reaction stirred at 0° C. for 1 h. The reaction was quenched with water (10 mL) and extracted with DCM (20 mL×3). The combined organic layer was dried over Na₂SO₄, filtered and the filtrate was evaporated under reduced pressure to afford 3-(difluoromethyl)cyclobutyl methanesulfonate (180 mg, 70% purity) as a colorless oil. ¹HNMR (500 MHz, CDCl₃) δ: 2.30-2.40 (m, 3H), 2.50-2.60 (m, 2H), 3.02 (s, 3H), 4.90-5.00 (m, 1H), 5.70-5.90 (m, 1H)

### Preparation 29: 3-Methoxycyclobutyl Methanesulfonate

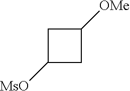 [SMILES: COC1CC(OS(C)(=O)=O)C1]

3-Methoxycyclobutyl methanesulfonate was prepared as a yellow oil, 400 mg, 79.3% yield, from 3-methoxycyclobutan-1-ol and methanesulfonyl chloride, following the procedure described in Preparation 28.

¹H NMR (400 MHz, CDCl₃) δ: 2.20-2.30 (m, 2H), 2.80-2.90 (m, 2H), 3.00 (s, 3H), 3.26 (s, 3H), 3.50-3.60 (m, 1H), 4.60-4.70 (m, 1H)

### Preparation 30: 5-Bromo-4-ethoxy-2-fluorobenzaldehyde

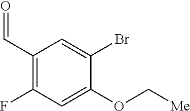 [SMILES: CCOc1cc(F)c(C=O)cc1Br]

To a solution of 5-bromo-2-fluoro-4-hydroxybenzaldehyde (5.0 g, 22.83 mmol) in DMF (20 mL) was added K₂CO₃ (6.31 g, 45.66 mmol) and the solution stirred at 25° C. for 2 h. Iodoethane (5.34 g, 34.24 mmol) was added and the reaction stirred at 50° C. for 16 h. The reaction mixture was concentrated in vacuo and the residue purified by Combiflash® (PE/EtOAc=5/1) to afford 5-bromo-4-ethoxy-2-fluorobenzaldehyde (4.50 g, 79.8% yield) as a white solid.

¹H NMR (500 MHz, CDCl₃) δ: 1.49 (t, 3H), 4.16 (q, 2H), 6.62-6.66 (m, 1H), 8.06 (d, 1H), 10.15 (s, 1H)

### Preparation 31: 5-Bromo-2-fluoro-4-((4-methoxybenzyl)oxy)benzaldehyde

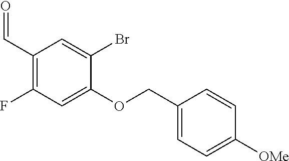 [SMILES: COc1ccc(COc2cc(F)c(C=O)cc2Br)cc1]

5-Bromo-2-fluoro-4-((4-methoxybenzyl)oxy)benzaldehyde was obtained as a white solid, 9.0 g, 27.1% yield, from 5-bromo-2-fluoro-4-hydroxy-benzaldehyde and 1-(chloromethyl)-4-methoxy-benzene, following the procedure described in Preparation 30.

### Preparation 32: 5-Bromo-2-fluoro-4-((tetrahydrofuran-3-yl)oxy)benzaldehyde

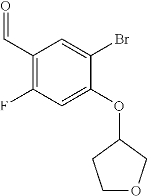 [SMILES: O=Cc1cc(Br)c(OC2CCOC2)cc1F]

To a mixture of tetrahydrofuran-3-yl methanesulfonate (3.80 g, 22.84 mmol) and 5-bromo-2-fluoro-4-hydroxybenzaldehyde (2.50 g, 11.42 mmol) in DMF (30 mL) was added K₂CO₃ (4.74 g, 34.26 mmol) and the reaction stirred at 100° C. for 16 h. The cooled mixture was filtered and concentrated in vacuo. The residue was purified by Combiflash® (PE/EtOAc=from 91/9 to 75/25) to afford 5-bromo-2-fluoro-4-((tetrahydrofuran-3-yl)oxy)benzaldehyde (520 mg, 14.5% yield) as a yellow solid. LCMS m/z=290.9 [M+H]⁺

### Preparation 33: 5-Bromo-6-ethoxy-2H-indazole

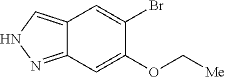 [SMILES: CCOc1cc2n[nH]cc2cc1Br]

To a solution of 5-bromo-4-ethoxy-2-fluorobenzaldehyde (Preparation 30, 4.50 g, 18.21 mmol) in DMSO (60 mL) was added K₂CO₃ (2.52 g, 18.21 mmol) and hydrazine hydrate (13.67 g, 273.2 mmol) and the reaction heated at 100° C. for 16 h. The cooled reaction mixture was diluted with water (50 mL) and extracted with EtOAc (50 mL×3). The combined organic layers were washed with brine (50 mL), dried over Na₂SO₄, filtered and concentrated in vacuo. The residue was purified by Combiflash® (PE/EtOAc=75/25) to afford 5-bromo-6-ethoxy-2H-indazole (2.20 g, 50.1% yield) as a yellow solid.

¹H NMR (500 MHz, CDCl₃) δ: 1.49 (t, 3H), 4.16 (q, 2H), 6.89 (s, 1H), 7.94 (d, 1H), 10.30 (s, 1H)

### Preparation 34: 5-Bromo-6-((4-methoxybenzyl)oxy)-2H-indazole

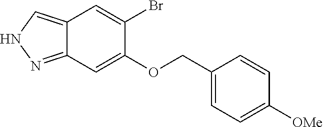 [SMILES: COc1ccc(COc2cc3n[nH]cc3cc2Br)cc1]

5-Bromo-6-((4-methoxybenzyl)oxy)-2H-indazole was obtained, 940 mg, 43.0% yield, from 5-bromo-2-fluoro-4-((4-methoxybenzyl)oxy)benzaldehyde (Preparation 31) and hydrazine hydrate, following a similar procedure to that described in Preparation 33. LCMS m/z=334.2 [M+H]⁺

### Preparation 35: 5-Bromo-6-((tetrahydrofuran-3-yl)oxy)-2H-indazole

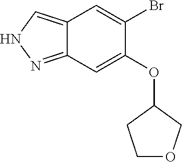 [SMILES: Brc1cc2c[nH]nc2cc1OC1CCOC1]

5-Bromo-6-((tetrahydrofuran-3-yl)oxy)-2H-indazole was obtained as a brown oil, 220 mg, 23.7% yield, from 5-bromo-2-fluoro-4-((tetrahydrofuran-3-yl)oxy)benzaldehyde (Preparation 32) and hydrazine hydrate, following the procedure described in Preparation 33. LCMS m/z=283.0 [M+H]⁺

### Preparation 36: 5-Bromo-6-ethoxy-2-(tetrahydro-2H-pyran-2-yl)-2H-indazole

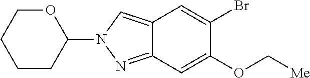 [SMILES: CCOc1cc2nn(C3CCCCO3)cc2cc1Br]

5-Bromo-6-ethoxy-2-(tetrahydro-2H-pyran-2-yl)-2H-indazole was obtained as a white oil, 2.5 g, 97.6% yield, from 5-bromo-6-ethoxy-2H-indazole (Preparation 33) and 3,4-dihydro-2H-pyran, following a similar procedure to that described in Preparation 7. LCMS m/z=327.0 [M+H]⁺

### Preparation 37: 5-Bromo-6-isopropoxy-2-(tetrahydro-2H-pyran-4-yl)-2H-indazole

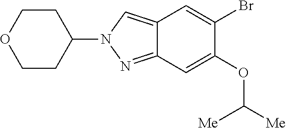 [SMILES: CC(C)Oc1cc2nn(C3CCOCC3)cc2cc1Br]

To a solution of 5-bromo-6-isopropoxy-1H-indazole (Preparation 3, 300 mg, 1.18 mmol) in DMF (20 mL) was tetrahydro-2H-pyran-4-yl 4-methylbenzenesulfonate (Preparation 27, 302.5 mg, 1.18 mmol) and K₂CO₃ (326.2 mg, 2.36 mmol) and the reaction was stirred at 110° C. for 16 h under N₂. The mixture was cooled to rt, the solid was filtered off and the filtrate concentrated in vacuo. The residue was purified by silica gel chromatography eluting with (PE/EtOAc, 82/18) to afford 5-bromo-6-isopropoxy-2-(tetrahydro-2H-pyran-4-yl)-2H-indazole as a clear oil, 50 mg, 12.5% yield. LCMS m/z=341.0 [M+H]⁺

### Preparation 38: 5-Bromo-6-isopropoxy-2-(tetrahydro-2H-pyran-3-yl)-2H-indazole

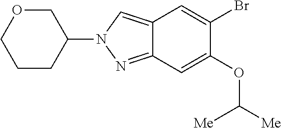 [SMILES: CC(C)Oc1cc2nn(C3CCCOC3)cc2cc1Br]

To a solution of 5-bromo-6-isopropoxy-1H-indazole (Preparation 3, 1.30 g, 5.10 mmol) in DMF (50 mL) was added tetrahydro-2H-pyran-3-yl methanesulfonate (2.76 g, 15.30 mmol) and Cs₂CO₃ (4.99 g, 15.30 mmol) and the reaction stirred at 110° C. for 16 h. The cooled mixture was diluted with water (100 mL) and extracted with EtOAc (100 mL×3). The combined organic layers were washed with brine (50 mL), dried over Na₂SO₄, and filtered. The filtrate was concentrated in vacuo and the residue purified by Combiflash® (PE/EtOAc 5/1) to afford 5-bromo-6-isopropoxy-2-(tetrahydro-2H-pyran-3-yl)-2H-indazole (240 mg, 12.5% yield) as a yellow oil. LCMS m/z=339.1 [M+H]⁺.

### Preparation 39: 5-Bromo-6-((4-methoxybenzyl)oxy)-2-(tetrahydro-2H-pyran-4-yl)-2H-indazole

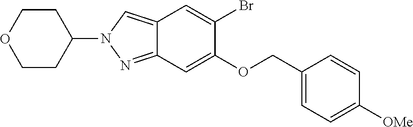 [SMILES: COc1ccc(COc2cc3nn(C4CCOCC4)cc3cc2Br)cc1]

5-Bromo-6-((4-methoxybenzyl)oxy)-2-(tetrahydro-2H-pyran-4-yl)-2H-indazole was obtained, 787 mg, 24.3% yield, from tetrahydro-2H-pyran-4-yl 4-methylbenzenesulfonate (Preparation 27) and 5-bromo-6-((4-methoxybenzyl)oxy)-2H-indazole (Preparation 34), following a similar procedure to that described in Preparation 38. LCMS m/z=419.0 [M+H]⁺

### Preparation 40: 5-Bromo-2-(tetrahydro-2H-pyran-4-yl)-6-((tetrahydrofuran-3-yl)oxy)-2H-indazole

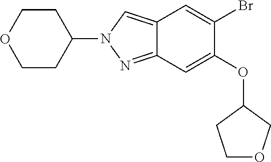 [SMILES: Brc1cc2cn(C3CCOCC3)nc2cc1OC1CCOC1]

5-Bromo-2-(tetrahydro-2H-pyran-4-yl)-6-((tetrahydrofuran-3-yl)oxy)-2H-indazole was obtained as a brown oil, 105 mg, from 5-bromo-6-((tetrahydrofuran-3-yl)oxy)-2H-indazole (Preparation 35) and tetrahydro-2H-pyran-4-yl 4-methylbenzenesulfonate (Preparation 27), following a similar procedure to that described in Preparation 38. LCMS m/z=366.9 [M+H]⁺

### Preparation 41: 2-((1r,3r)-3-(Benzyloxy)cyclobutyl)-5-bromo-6-isopropoxy-2H-indazole

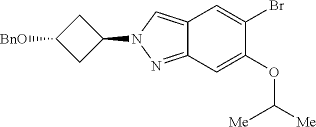 [SMILES: CC(C)Oc1cc2nn([C@H]3C[C@H](OCc4ccccc4)C3)cc2cc1Br]

To a solution of 5-bromo-6-isopropoxy-1H-indazole (Preparation 3, 2.0 g, 7.84 mmol) and (1s,3s)-3-(benzyloxy)cyclobutyl 4-methylbenzenesulfonate (3.21 g, 9.64 mmol) in DMF (50 mL) was added K₂CO₃ (2.17 g, 15.68 mmol) and the reaction stirred at 100° C. for 16 h. The reaction was diluted with water (100 mL) and extracted with EtOAc (100 mL×3). The combined organic layers were washed with brine (200 mL), dried over Na₂SO₄ and filtered. The filtrate was concentrated in vacuo and the residue, purified by Combiflash® (PE/EtOAc=95/5 to 75/25) to afford 2-((1r,3r)-3-(benzyloxy)cyclobutyl)-5-bromo-6-isopropoxy-2H-indazole (700 mg, 22% yield) as colorless oil. ¹H NMR (400 MHz, CDCl₃) δ: 1.44 (d, 6H), 2.60-2.70 (m, 2H), 2.90-3.00 (m, 2H), 3.85-3.90 (m, 1H), 4.53 (s, 2H), 4.60-4.70 (m, 1H), 5.10-5.20 (m, 1H), 6.76 (s, 1H), 7.30-7.40 (m, 5H), 7.89 (s, 2H).

### Preparation 42: Methyl 3-(5-bromo-6-isopropoxy-2H-indazol-2-yl)butanoate

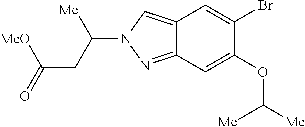 [SMILES: COC(=O)CC(C)n1cc2cc(Br)c(OC(C)C)cc2n1]

To a solution of 5-bromo-6-isopropoxy-1H-indazole (Preparation 3, 500 mg, 1.96 mmol) in MeCN (10 mL) was added methyl (E)-but-2-enoate (294 mg, 2.94 mmol) and DBU (149 mg, 0.98 mmol) and the reaction stirred at 50° C. for 16 h. The mixture was concentrated in vacuo and the residue purified by Combiflash® (PE/EtOAc=85/15 to 50/50) to afford methyl 3-(5-bromo-6-isopropoxy-2H-indazol-2-yl)butanoate (400 mg, 57% yield) as a yellow oil. ¹H NMR (500 MHz, CDCl₃) δ: 1.35-1.45 (m, 6H), 1.60-1.70 (m, 3H), 2.80-2.90 (m, 1H), 3.15-3.25 (m, 1H), 3.63 (s, 3H), 4.60-4.70 (m, 1H), 5.00-5.10 (m, 1H), 7.05 (s, 1H), 7.70-7.80 (m, 2H)

### Preparation 43: 3-(5-Bromo-6-isopropoxy-2H-indazol-2-yl)butan-1-ol

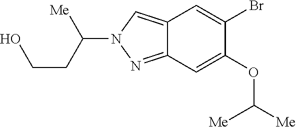 [SMILES: CC(C)Oc1cc2nn(C(C)CCO)cc2cc1Br]

To a solution of methyl 3-(5-bromo-6-isopropoxy-2H-indazol-2-yl)butanoate (Preparation 42, 400 mg, 1.13 mmol) in EtOH (5 mL) was added NaBH₄ (128 mg, 3.39 mmol) and CaCl₂) (124 mg, 1.13 mmol) and the reaction stirred at 20° C. for 1 h. The reaction was diluted with water (20 mL) and extracted with DCM (20 mL×5). The combined organic layers were washed with brine (30 mL), dried over Na₂SO₄ and filtered. The filtrate was evaporated under reduced pressure to afford 3-(5-bromo-6-isopropoxy-2H-indazol-2-yl)butan-1-ol (300 mg, 81% yield) as a colorless oil. LCMS m/z=328.8 [M+H]⁺

### Preparation 44: 5-Bromo-6-isopropoxy-2-(4-methoxybutan-2-yl)-2H-indazole

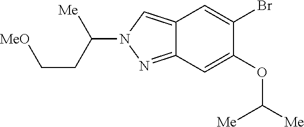 [SMILES: COCCC(C)n1cc2cc(Br)c(OC(C)C)cc2n1]

To a solution of 3-(5-bromo-6-isopropoxy-2H-indazol-2-yl)butan-1-ol (Preparation 43, 300 mg, 0.92 mmol) in THF (5 mL) was added NaH (55 mg, 1.38 mmol, 60% purity) at 0° C. and the solution stirred for 30 min. Iodomethane (1.64 g, 11.5 mmol) was added and the reaction stirred at 25° C. for 1 h. The reaction was quenched with saturated NH₄Cl aq (30 mL) and NH₄OH (28% w/w, 5 mL) and extracted with EtOAc (30 mL×3). The combined organic layers were washed with brine (30 mL), dried over Na₂SO₄, and filtered. The filtrate was concentrated in vacuo and the residue purified by Combiflash® (PE/EtOAc=85/15 to 50/50) to afford 5-bromo-6-isopropoxy-2-(4-methoxybutan-2-yl)-2H-indazole (150 mg, 48% yield) as a yellow oil. LCMS m/z=342.5 [M+H]⁺

### Preparation 45: 5-Bromo-6-isopropoxy-2-(1-methyl-2-oxabicyclo[2.2.2]octan-4-yl)-2H-indazole

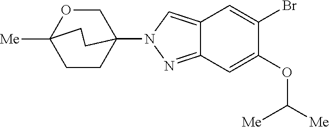 [SMILES: CC(C)Oc1cc2nn(C34CCC(C)(CC3)OC4)cc2cc1Br]

1-Methyl-2-oxabicyclo[2.2.2]octan-4-amine hydrochloride (123 mg, 0.69 mmol) was added in one portion, followed by TEA (70.3 mg, 0.69 mmol) to a solution of 5-bromo-4-isopropoxy-2-nitro-benzaldehyde (200 mg, 0.69 mmol) in isopropanol (4 mL), the vial sealed and the resulting yellow solution heated to 80° C. with stirring overnight. The mixture was cooled to rt and P(n-Bu)₃ (421.4 mg, 2.08 mmol) was added in one portion. The vessel was sealed and the reaction stirred at 80° C. for an additional 16 h. The mixture was cooled to rt, diluted with EtOAc (10 mL), washed with saturated NH₄Cl solution (10 mL), brine (10 mL) and dried over anhydrous MgSO₄. The solution was filtered, and the filtrate concentrated in vacuo. The residue was purified by silica gel chromatography (EtOAc in heptane 0/100 to 50/50) to afford 5-bromo-6-isopropoxy-2-(1-methyl-2-oxabicyclo[2.2.2]octan-4-yl)-2H-indazole (121.6 mg, 46.2% yield) as an orange solid. LCMS m/z=380.3 [M+H]⁺

### Preparation 46: 5-Bromo-6-isopropoxy-2-(1-methyl-2-oxabicyclo[2.2.1]heptan-4-yl)-2H-indazole

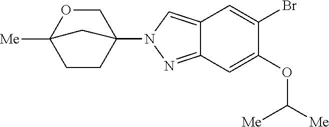 [SMILES: CC(C)Oc1cc2nn(C34CCC(C)(C3)OC4)cc2cc1Br]

1-Methyl-2-oxabicyclo[2.2.1]heptan-4-amine hydrochloride (290 mg, 1.77 mmol) was added in one portion, followed by TEA (179.3 mg, 1.77 mmol) to a solution of 5-bromo-4-isopropoxy-2-nitro-benzaldehyde (510.5 mg, 1.77 mmol) in isopropanol (6 mL), the vial sealed and the resulting yellow solution heated to 80° C. with stirring overnight. The mixture was cooled to rt and P(n-Bu)₃ (1.08 g, 5.32 mmol) was added in one portion. The vessel was sealed and the reaction stirred at 80° C. for an additional 16 h. The mixture was cooled to rt, diluted with EtOAc (15 mL), washed with saturated NH₄Cl solution (10 mL), brine (10 mL) and dried over anhydrous MgSO₄. The solution was filtered, and the filtrate concentrated in vacuo. The residue was purified by silica gel chromatography (EtOAc in heptane 0/100 to 50/50) to afford 5-bromo-6-isopropoxy-2-(1-methyl-2-oxabicyclo[2.2.1]heptan-4-yl)-2H-indazole (308.2 mg, 47.7% yield) as a yellow solid.

### Preparation 47: 5-Bromo-6-isopropoxy-2-(1-methyl-2-oxabicyclo[2.1.1]hexan-4-yl)-2H-indazole

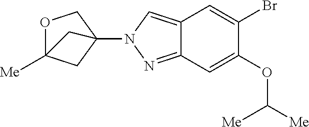 [SMILES: CC(C)Oc1cc2nn(C34COC(C)(C3)C4)cc2cc1Br]

1-Methyl-2-oxabicyclo[2.1.1]hexan-4-amine hydrochloride (1.04 g, 6.94 mmol) was added in one portion, followed by TEA (702.5 mg, 6.94 mmol) to a solution of 5-bromo-4-isopropoxy-2-nitro-benzaldehyde (2.0 g, 6.94 mmol) in isopropanol (15 mL), the vial sealed and the resulting yellow solution heated to 80° C. with stirring overnight. The mixture was cooled to rt and P(n-Bu)₃ (4.21 g, 20.82 mmol) was added in one portion. The vessel was sealed and the reaction stirred at 80° C. for an additional 16 h. The mixture was cooled to rt, diluted with EtOAc (30 mL), washed with saturated NH₄Cl solution (15 mL), brine (15 mL) and dried over anhydrous MgSO₄. The solution was filtered, and the filtrate concentrated in vacuo. The residue was purified by silica gel chromatography (EtOAc in heptane 0/100 to 50/50) to afford 5-bromo-6-isopropoxy-2-(1-methyl-2-oxabicyclo[2.1.1]hexan-4-yl)-2H-indazole (901 mg, 37.0% yield) as an orange yellow solid.

### Preparation 48: 5-Bromo-6-cyclobutoxy-2-(1-methyl-2-oxabicyclo[2.1.1]hexan-4-yl)-2H-indazole

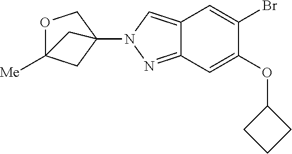 [SMILES: CC12CC(n3cc4cc(Br)c(OC5CCC5)cc4n3)(CO1)C2]

1-Methyl-2-oxabicyclo[2.1.1]hexan-4-amine hydrochloride (99.7 mg, 0.67 mmol) was added in one portion, followed by TEA (67.4 mg, 0.67 mmol) to a solution of 5-bromo-4-(cyclobutoxy)-2-nitro-benzaldehyde (200 mg, 0.67 mmol) in isopropanol (4 mL), the vial sealed and the resulting yellow solution heated to 80° C. with stirring overnight. The mixture was cooled to rt and P(n-Bu)₃ (404.5 mg, 2.0 mmol) was added in one portion. The vessel was sealed and the reaction stirred at 80° C. for an additional 16 h. The mixture was cooled to rt, diluted with EtOAc (10 mL), washed with saturated NH₄Cl solution (10 mL), brine (10 mL) and dried over anhydrous MgSO₄. The solution was filtered, and the filtrate concentrated in vacuo. The residue was purified by silica gel chromatography (EtOAc in heptane 0/100 to 50/50) to afford 5-bromo-6-cyclobutoxy-2-(1-methyl-2-oxabicyclo[2.1.1]hexan-4-yl)-2H-indazole (216 mg, 89.4% yield) as an orange brown solid.

### Preparation 49: 5-Bromo-6-methoxy-2-(1-methyl-2-oxabicyclo[2.1.1]hexan-4-yl)-2H-indazole

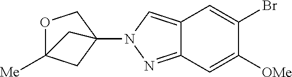 [SMILES: COc1cc2nn(C34COC(C)(C3)C4)cc2cc1Br]

Part A: To an ice cooled solution of 5-bromo-4-fluoro-2-nitro-benzaldehyde (552 mg, 2.23 mmol) in MeOH (6 mL) was added sodium methoxide (180.4 mg, 3.34 mmol) and the solution stirred at rt for 8 h. The reaction was quenched with ice water, the suspension extracted with EtOAc (20 mL×3) and the combined organic layers were dried over anhydrous MgSO₄. The mixture was filtered and the filtrate evaporated under reduced pressure to give 5-bromo-4-methoxy-2-nitro-benzaldehyde (564 mg, 97.3% yield) as a yellow solid.
Part B: 1-Methyl-2-oxabicyclo[2.1.1]hexan-4-amine hydrochloride (324.7 mg, 2.17 mmol) was added in one portion, followed by TEA (219.6 mg, 2.17 mmol) to a solution of 5-bromo-4-methoxy-2-nitro-benzaldehyde (564 mg, 2.17 mmol) in isopropanol (6 mL), the vial sealed and the resulting yellow solution heated to 80° C. with stirring overnight. The mixture was cooled to rt and P(n-Bu)₃ (1.32 g, 6.51 mmol) added in one portion. The vessel was sealed and the orange colored solution stirred at 80° C. for an additional 16 h. The mixture was cooled to rt and diluted with EtOAc (20 mL). The organics were washed with saturated NH₄Cl solution (15 mL), brine (15 mL) and dried over anhydrous MgSO₄. The solution was filtered, and the filtrate was concentrated in vacuo. The residue was purified by silica gel chromatography (EtOAc in heptane 0/100 to 50/50) to afford 5-bromo-6-methoxy-2-(1-methyl-2-oxabicyclo[2.1.1]hexan-4-yl)-2H-indazole (154.4 mg, 22.0% yield) as an orange solid.

### Preparation 50: 5-Bromo-6-ethoxy-2-(1-methyl-2-oxabicyclo[2.1.1]hexan-4-yl)-2H-indazole

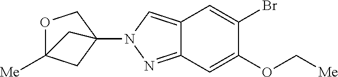 [SMILES: CCOc1cc2nn(C34COC(C)(C3)C4)cc2cc1Br]

Part A: To an ice cooled solution of 5-bromo-4-fluoro-2-nitro-benzaldehyde (300 mg, 1.21 mmol) in EtOH (6 mL) was added sodium ethoxide (123.5 mg, 1.81 mmol) and the solution stirred at rt for 8 h. The reaction was quenched with ice water, the suspension extracted with EtOAc (20 mL×3) and the combined organic layers were dried over anhydrous MgSO₄. The mixture was filtered and the filtrate evaporated under reduced pressure. The residue was purified by silica gel column (0-30% 3:1 EtOAc:EtOH in heptane) to give 5-bromo-4-ethoxy-2-nitro-benzaldehyde (135.6 mg, 40.9% yield) as a yellow solid.
Part B: 5-Bromo-6-ethoxy-2-(1-methyl-2-oxabicyclo[2.1.1]hexan-4-yl)-2H-indazole was obtained, 144.3 mg, 30.4% yield, as an orange solid, from 5-bromo-4-ethoxy-2-nitro-benzaldehyde and 1-methyl-2-oxabicyclo[2.1.1]hexan-4-amine hydrochloride, following a similar procedure to that described in Preparation 49, Part B.

### Preparation 51: 6-Chloro-2-((tetrahydrofuran-3-yl)methyl)-2H-pyrazolo[3,4-b]pyridine Hydrochloride

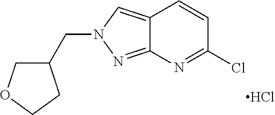 [SMILES: Clc1ccc2cn(CC3CCOC3)nc2n1 ; Cl]

To a solution of 6-chloro-2H-pyrazol[3,4-b]pyridine (2.0 g, 13.02 mmol) in DMF (15 mL) was added Cs₂CO₃ (8.49 g, 26.04 mmol) and (tetrahydrofuran-3-yl)methyl methanesulfonate (3.05 g, 16.93 mmol) and the reaction mixture stirred at 100° C. for 14 h. The reaction was filtered and the filtrate concentrated in vacuo. The residue was purified by prep-HPLC (Phenomenex Synergi C18 150×30 μm, 4 mm; MeCN/H₂O+0.05% HCl; 24-34%) to afford 6-chloro-2-((tetrahydrofuran-3-yl)methyl)-2H-pyrazolo[3,4-b]pyridine (240 mg, 7.8% yield) as a yellow solid.

### Preparation 52: 6-Chloro-2-(3-methoxypropyl)-2H-pyrazolo[3,4-b]pyridine trifluoroacetate

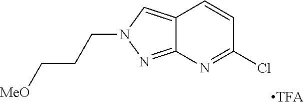 [SMILES: COCCCn1cc2ccc(Cl)nc2n1 ; "·TFA"]

6-Chloro-2-(3-methoxypropyl)-2H-pyrazolo[3,4-b]pyridine was obtained as a brown solid, 1.70 g, 11.4% yield, from 6-chloro-2H-pyrazol[3,4-b]pyridine and 3-methoxypropyl bromide, following a similar procedure to that described in Preparation 51, except the crude product was purified by prep-HPLC (Welch Xtimate C18 250×50 mm, 10 μm, MeCN/H₂O+0.1% TFA; 20-60%).

### Preparation 53: 6-Chloro-2-(tetrahydro-2H-pyran-4-yl)-2H-pyrazolo[3,4-b]pyridine

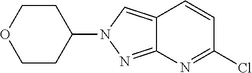 [SMILES: Clc1ccc2cn(C3CCOCC3)nc2n1]

6-Chloro-2-(tetrahydro-2H-pyran-4-yl)-2H-pyrazolo[3,4-b]pyridine was obtained as a yellow solid, 900 mg, 89.2% yield, from 6-chloro-2H-pyrazol[3,4-b]pyridine and tetrahydro-2H-pyran-4-yl 4-methylbenzenesulfonate (Preparation 27), following a similar procedure to that described in Preparation 51, except the crude product was purified by prep-HPLC (Welch Xtimate C18 150×40 mm×10 μm, MeCN/H₂O+0.1% TFA; 24-44%). LCMS m/z=238.0 [M+H]⁺

### Preparation 54: 6-Chloro-2-(tetrahydro-2H-pyran-2-yl)-2H-pyrazolo[3,4-b]pyridine

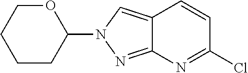 [SMILES: Clc1ccc2cn(C3CCCCO3)nc2n1]

6-Chloro-2-(tetrahydro-2H-pyran-2-yl)-2H-pyrazolo[3,4-b]pyridine was obtained as a yellow oil, 1.40 g, 90.1% yield, from 6-chloro-2H-pyrazol[3,4-b]pyridine and 3,4-dihydro-2H-pyran following the procedure described in Preparation 7. LCMS m/z=237.9 [M+H]⁺

### Preparation 55: 6-Isopropoxy-2-((tetrahydrofuran-3-yl)methyl)-2H-pyrazolo[3,4-b]pyridine

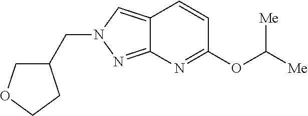 [SMILES: CC(C)Oc1ccc2cn(CC3CCOC3)nc2n1]

To a solution of 6-chloro-2-((tetrahydrofuran-3-yl)methyl)-2H-pyrazolo[3,4-b]pyridine (Preparation 51, 252.4 mg, 1.05 mmol) in THF (5 mL) was added NaH (168 mg, 4.20 mmol, 60% purity) and the mixture stirred at 0° C. for 30 min. Isopropanol (250 mg, 1.05 mmol) was added and the reaction stirred at 60° C. for 3 h. The reaction was quenched with water (one drop), then concentrated in vacuo. The residue was purified by Combiflash® (PE/EtOAc 50/50) to afford 6-isopropoxy-2-((tetrahydrofuran-3-yl)methyl)-2H-pyrazolo[3,4-b]pyridine (130 mg, 47.4% yield) as a yellow oil. LCMS m/z 262.0 [M+H].

### Preparations 56 to 60

The following compounds were prepared from the appropriate 6-chloro pyrazolo[3,4-b]pyridine and alcohol, following a similar procedure to that described in Preparation 55.

| Prep. | |
|---|---|
| No | Structure, Name, Starting Materials (SM), Yield, Data |
| 56 | 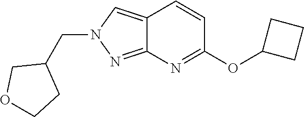 [SMILES: c1cc2cn(CC3CCOC3)nc2nc1OC1CCC1] |
| | 6-Cyclobutoxy-2-((tetrahydrofuran-3-yl)methyl)-2H-pyrazolo |
| | [3,4-b]pyridine |
| | SM: 6-chloro-2-((tetrahydrofuran-3-yl)methyl)-2H-pyrazolo |
| | [3,4-b]pyridine |
| | (Preparation 51) and cyclobutanol |
| | yellow oil, 1.50 g, 65.2% yield. LCMS m/z = 274.4 [M + H]⁺ |
| 57 | 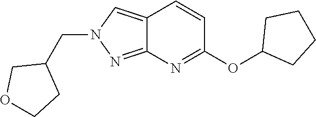 [SMILES: c1cc2cn(CC3CCOC3)nc2nc1OC1CCCC1] |
| | 6-Cyclopentyloxy-2-((tetrahydrofuran-3-yl)methyl)-2H-pyrazolo |
| | [3,4-b]pyridine |
| | SM: 6-chloro-2-((tetrahydrofuran-3-yl)methyl)-2H-pyrazolo |
| | [3,4-b]pyridine |
| | (Preparation 51) and cyclopentanol |
| | yellow oil, 1.30 g, 80.6% yield. LCMS m/z = 288.7 [M + H]⁺ |
| 58 | 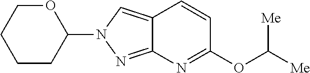 [SMILES: CC(C)Oc1ccc2cn(C3CCCCO3)nc2n1] |
| | 6-Isopropoxy-2-(tetrahydro-2H-pyran-2-yl)-2H-pyrazolo |
| | [3,4-b]pyridine |
| | SM: 6-chloro-2-(tetrahydro-2H-pyran-2-yl)-2H-pyrazolo |
| | [3,4-b]pyridine |
| | (Preparation 54) and isopropanol |
| | yellow oil, 1.1 g, 68.9% yield. LCMS m/z = 262.0 [M + H]⁺ |
| 59 | 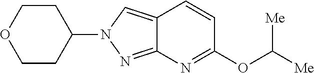 [SMILES: CC(C)Oc1ccc2cn(C3CCOCC3)nc2n1] |
| | 6-Isopropoxy-2-(tetrahydro-2H-pyran-4-yl)-2H-pyrazolo |
| | [3,4-b]pyridine |
| | SM: 6-chloro-2-(tetrahydro-2H-pyran-4-yl)-2H-pyrazolo |
| | [3,4-b]pyridine |
| | (Preparation 53) and isopropanol |
| | yellow solid, 700 mg, 65.6% yield. LCMS m/z = 262.0 [M + H]⁺ |
| 60 | 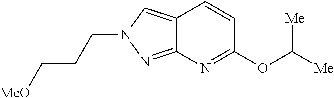 [SMILES: COCCCn1cc2ccc(OC(C)C)nc2n1] |
| | 6-Isopropoxy-2-(3-methoxypropyl)-2H-pyrazolo[3,4-b]pyridine |
| | SM: 6-chloro-2-(3-methoxypropyl)-2H-pyrazolo[3,4-b]pyridine |
| | (Preparation 52) and isopropanol |
| | yellow solid, 1.5 g, 77.7% yield. LCMS m/z = 250.1 [M + H]⁺ |

### Preparation 61: 5-Bromo-6-isopropoxy-2-((tetrahydrofuran-3-yl)methyl)-2H-pyrazolo[3,4-b]pyridine

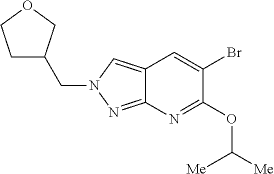 [SMILES: CC(C)Oc1nc2nn(CC3CCOC3)cc2cc1Br]

To a solution of 6-isopropoxy-2-((tetrahydrofuran-3-yl)methyl)-2H-pyrazolo[3,4-b]pyridine (Preparation 55, 1.96 g, 7.5 mmol) in AcOH (40 mL) was added Br₂ (1.2 g, 7.5 mmol) and the reaction stirred at 20° C. for 5 h. The reaction was concentrated in vacuo, the residue was quenched with saturated aq. NaHCO₃ (40 mL) and extracted with EtOAc (80 mL×2). The combined organic layers were dried over Na₂SO₄, filtered and the filtrate was concentrated in vacuo. The residue was purified by Combiflash® (PE/EtOAc=34/66) to afford 5-bromo-6-isopropoxy-2-((tetrahydrofuran-3-yl)methyl)-2H-pyrazolo[3,4-b]pyridine (1.3 g, 46% yield) as a yellow oil. LCMS m/z=339.9 [M+H]⁺

### Preparation 62 to 66

The following compounds were prepared from the appropriate pyrazolo[3,4-b]pyridine, following a similar procedure to that described in Preparation 61.

| Prep | |
|---|---|
| No | Structure, Name, Starting Materials (SM), Yield, Data |
| 62 | 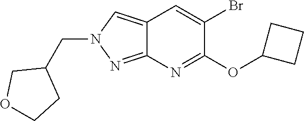 [SMILES: Brc1cc2cn(CC3CCOC3)nc2nc1OC1CCC1] |
| | 5-Bromo-6-cyclobutoxy-2-((tetrahydrofuran-3-yl)methyl)-2H- |
| | pyrazolo[3,4-b]pyridine |
| | SM: 6-cyclobutoxy-2-((tetrahydrofuran-3-yl)methyl)-2H- |
| | pyrazolo[3,4-b]pyridine |
| | (Preparation 56) |
| | yellow solid, 1.40 g, 65.2% yield. LCMS m/z = 353.9 [M + H]⁺ |
| 63 | 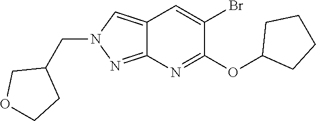 [SMILES: Brc1cc2cn(CC3CCOC3)nc2nc1OC1CCCC1] |
| | 5-Bromo-6-cyclopentyloxy-2-((tetrahydrofuran-3-yl)methyl)- |
| | 2H-pyrazolo[3,4-b]pyridine |
| | SM: 6-cyclopentyloxy-2-((tetrahydrofuran-3-yl)methyl)-2H- |
| | pyrazolo[3,4-b]pyridine |
| | (Preparation 57) yellow solid, 1.15 g, 62.6% yield. |
| | LCMS m/z = 366.5 [M + H]⁺ |
| 64 | 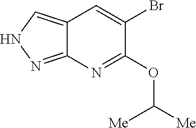 [SMILES: CC(C)Oc1nc2n[nH]cc2cc1Br] |
| | 5-Bromo-6-isopropoxy-2H-pyrazolo[3,4-b]pyridine |
| | SM: 6-isopropoxy-2-(tetrahydro-2H-pyran-2-yl)-2H- |
| | pyrazolo[3,4-b]pyridine |
| | (Preparation 58) white solid, 280 mg, 25.9% yield. |
| | LCMS m/z = 257.9 [M + H]⁺ |
| 65 | 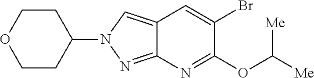 [SMILES: CC(C)Oc1nc2nn(C3CCOCC3)cc2cc1Br] |
| | 5-Bromo-6-isopropoxy-2-(tetrahydro-2H-pyran-4-yl)-2H- |
| | pyrazolo[3,4-b]pyridine |
| | SM: 6-isopropoxy-2-(tetrahydro-2H-pyran-4-yl)-2H- |
| | pyrazolo[3,4-b]pyridine |
| | (Preparation 59) yellow solid. LCMS m/z = 340.0 [M + H]⁺ |
| 66 | 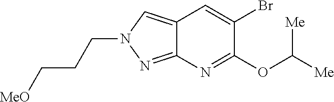 [SMILES: COCCCn1cc2cc(Br)c(OC(C)C)nc2n1] |
| | 5-Bromo-6-isopropoxy-2-(3-methoxypropyl)-2H-pyrazolo |
| | [3,4-b]pyridine |
| | SM: 6-isopropoxy-2-(3-methoxypropyl)-2H-pyrazolo |
| | [3,4-b]pyridine |
| | (Preparation 60) yellow oil, 700 mg, 33.96% yield. |
| | LCMS m/z = 329.9 [M + H]⁺ |

### Preparation 67: 5-Bromo-6-isopropoxy-2-(tetrahydro-2H-pyran-2-yl)-2H-pyrazolo[3,4-b]pyridine

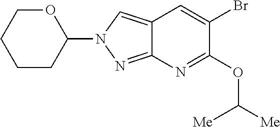 [SMILES: CC(C)Oc1nc2nn(C3CCCCO3)cc2cc1Br]

5-Bromo-6-isopropoxy-2-(tetrahydro-2H-pyran-2-yl)-2H-pyrazolo[3,4-b]pyridine was obtained as a colorless oil, 350 mg, 91.5% yield, from 5-bromo-6-isopropoxy-2H-pyrazolo[3,4-b]pyridine (Preparation 64) and 3,4-dihydro-2H-pyran, following a similar procedure to that described in Preparation 7. LCMS m/z=339.9 [M+H]⁺

### Preparation 68: 5-Bromo-6-isopropoxy-2-(tetrahydro-2H-pyran-3-yl)-2H-pyrazolo[3,4-b]pyridine

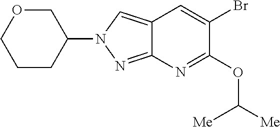 [SMILES: CC(C)Oc1nc2nn(C3CCCOC3)cc2cc1Br]

To a solution of 5-bromo-6-isopropoxy-2H-pyrazolo[3,4-b]pyridine (Preparation 64, 1.20 g, 4.69 mmol) in DMF (30 mL) was added K₂CO₃ (1.30 g, 9.38 mmol) and tetrahydro-2H-pyran-3-yl methanesulfonate (3.38 g, 18.76 mmol) and the reaction stirred at 100° C. for 14 h. The cooled mixture was concentrated in vacuo, the residue was diluted with water (100 mL) and extracted with EtOAc (40 mL×3). The combined organic layers were washed with brine (30 mL×2), dried over Na₂SO₄, filtered and evaporated under reduced pressure. The residue was purified by Combiflash® (PE/EtOAc from 75:25 to 0:100) to give 5-bromo-6-isopropoxy-2-(tetrahydro-2H-pyran-3-yl)-2H-pyrazolo[3,4-b]pyridine (150 mg, 8.5% yield) as a yellow solid. LCMS m/z=340.2 [M+H]⁺

### Preparation 69: Methyl 6-ethoxy-2-(tetrahydro-2H-pyran-2-yl)-2H-indazole-5-carboxylate

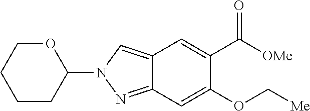 [SMILES: CCOc1cc2nn(C3CCCCO3)cc2cc1C(=O)OC]

To a solution of 5-bromo-6-ethoxy-2-(tetrahydro-2H-pyran-2-yl)-2H-indazole (Preparation 36, 2.20 g, 6.77 mmol) in MeOH (200 mL) was added Pd(dppf)Cl₂ (495.4 mg, 0.68 mmol) and TEA (6.85 g, 67.7 mmol), the reaction charged with CO then stirred at 80° C. under CO (50 psi) for 16 h. The cooled mixture was filtered through Celite®, the filtrate was concentrated in vacuo and the residue purified by Combiflash® (PE/EA=85/15) to afford methyl 6-ethoxy-2-(tetrahydro-2H-pyran-2-yl)-2H-indazole-5-carboxylate (1.90 g, 92.2% yield) as a yellow solid. LCMS m/z=305.1 [M+H]⁺

### Preparation 70 to 80

The compounds in the following table were prepared from the appropriate bromide, following a similar procedure to that described in Preparation 69.

| Prep. | |
|---|---|
| No | Structure/Name/Starting Bromide (SM)/Yield/Data |
| 70 | 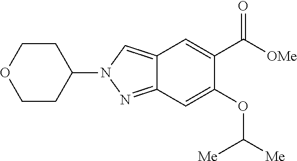 [SMILES: COC(=O)c1cc2cn(C3CCOCC3)nc2cc1OC(C)C] |
| | Methyl 6-isopropoxy-2-(tetrahydro-2H-pyran-4-yl)-2H- |
| | indazole-5-carboxylate |
| | SM: 5-bromo-6-isopropoxy-2-(tetrahydro-2H-pyran-4-yl)- |
| | 2H-indazole (Preparation 37) |
| | 130 mg, 41.6% yield, as a white solid. |
| | LCMS m/z = 319.0 [M + H]⁺ |
| 71 | 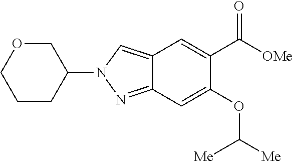 [SMILES: COC(=O)c1cc2cn(C3CCCOC3)nc2cc1OC(C)C] |
| | Methyl 6-isopropoxy-2-(tetrahydro-2H-pyran-3-yl)-2H- |
| | indazole-5-carboxylate |
| | SM: 5-bromo-6-isopropoxy-2-(tetrahydro-2H-pyran-3-yl)- |
| | 2H-indazole (Preparation 38) |
| | 300 mg, 65.2% yield. ¹H NMR (500 MHz, CDCl₃) δ: |
| | 1.41 (d, 6H), 1.80-1.85 (m, 2H), 2.30-2.35 (m, 2H), 3.62- |
| | 3.68 (m, 1H), 3.90 (s, 3H), 3.91-3.97 (m, 2H), 4.20 (dd, |
| | 1H), 4.52-4.57 (m, 1H), 4.59-4.63 (m, 1H), 7.05 (s, 1H), |
| | 8.09 (s, 1H), 8.13 (s, 1H) |
| 72 | 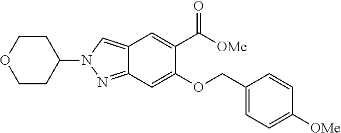 [SMILES: COC(=O)c1cc2cn(C3CCOCC3)nc2cc1OCc1ccc(OC)cc1] |
| | Methyl 6-((4-methoxybenzyl)oxy)-2-(tetrahydro-2H- |
| | pyran-4-yl)-2H-indazole-5-carboxylate |
| | SM: 5-bromo-6-((4-methoxybenzyl)oxy)-2-(tetrahydro- |
| | 2H-pyran-4-yl)-2H-indazole (Preparation 39) |
| | 540 mg, 61.3% yield as a yellow oil. |
| | LCMS m/z = 397.1 [M + H]⁺ |
| 73 | 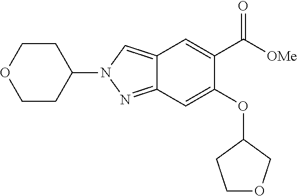 [SMILES: COC(=O)c1cc2cn(C3CCOCC3)nc2cc1OC1CCOC1] |
| | Methyl 2-(tetrahydro-2H-pyran-4-yl)-6-((tetrahydrofuran- |
| | 3-yl)oxy)-2H-indazole-5-carboxylate |
| | SM: 5-bromo-2-(tetrahydro-2H-pyran-4-yl)-6- |
| | ((tetrahydrofuran-3-yl)oxy)-2H-indazole |
| | (Preparation 40) brown oil, 85 mg, 75.5% yield. |
| | LCMS m/z = 347.0 [M + H]⁺ |
| 74 | 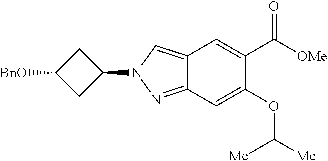 [SMILES: COC(=O)c1cc2cn([C@H]3C[C@H](OCc4ccccc4)C3)nc2cc1OC(C)C] |
| | Methyl 2-((1r,3r)-3-(benzyloxy)cyclobutyl)-6-isopropoxy- |
| | 2H-indazole-5-carboxylate |
| | SM: 2-((1r,3r)-3-(benzyloxy)cyclobutyl)-5-bromo-6- |
| | isopropoxy-2H-indazole (Preparation 41) Colourless oil, |
| | 600 mg, 90.0% yield. LCMS m/z = 395.1 [M + H]⁺ |
| 75 | 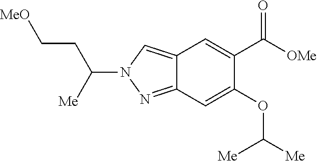 [SMILES: COCCC(C)n1cc2cc(C(=O)OC)c(OC(C)C)cc2n1] |
| | Methyl 6-isopropoxy-2-(4-methoxybutan-2-yl)-2H- |
| | indazole-5-carboxylate |
| | SM: 5-bromo-6-isopropoxy-2-(4-methoxybutan-2-yl)-2H- |
| | indazole (Preparation 44) 170 mg, 86.0% yield, yellow oil. |
| | LCMS m/z = 321.0 [M + H]⁺ |
| 76 | 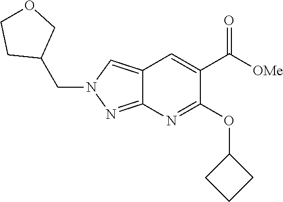 [SMILES: COC(=O)c1cc2cn(CC3CCOC3)nc2nc1OC1CCC1] |
| | Methyl 6-cyclobutoxy-2-((tetrahydrofuran-3-yl)methyl)-2H- |
| | pyrazolo[3,4-b]pyridine-5-carboxylate |
| | SM: 5-bromo-6-cyclobutoxy-2-((tetrahydrofuran-3-yl) |
| | methyl)-2H-pyrazolo[3,4-b]pyridine (Preparation 62) |
| | Orange solid, 1.10 g, 75.3% yield. |
| | LCMS m/z = 332.4 [M + H]⁺ |
| 77 | 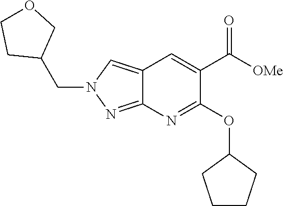 [SMILES: COC(=O)c1cc2cn(CC3CCOC3)nc2nc1OC1CCCC1] |
| | Methyl 6-cyclopentyloxy-2-((tetrahydrofuran-3-yl)methyl)- |
| | 2H-pyrazolo[3,4-b]pyridine-5-carboxylate |
| | SM: 5-bromo-6-cyclopentyloxy-2-((tetrahydrofuran-3-yl) |
| | methyl)-2H-pyrazolo[3,4-b]pyridine (Preparation 63) |
| | Orange solid, 400 mg, 29.5% yield. |
| | LCMS m/z = 346.6 [M + H]⁺ |
| 78 | 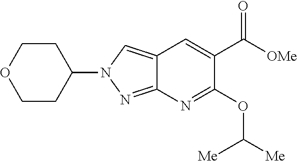 [SMILES: COC(=O)c1cc2cn(C3CCOCC3)nc2nc1OC(C)C] |
| | Methyl 6-isopropoxy-2-(tetrahydro-2H-pyran-4-yl)-2H- |
| | pyrazolo[3,4-b]pyridine-5-carboxylate |
| | SM: 5-bromo-6-isopropoxy-2-(tetrahydro-2H-pyran-4-yl)- |
| | 2H-pyrazolo[3,4-b]pyridine (Preparation 65) |
| | Yellow oil, 80 mg, 98.7% yield. |
| | LCMS m/z = 320.0 [M + H]⁺ |
| 79 | 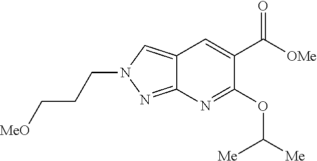 [SMILES: COCCCn1cc2cc(C(=O)OC)c(OC(C)C)nc2n1] |
| | Methyl 6-isopropoxy-2-(3-methoxypropyl)-2H-pyrazolo |
| | [3,4-b]pyridine-5-carboxylate |
| | SM: 5-bromo-6-isopropoxy-2-(3-methoxypropyl)-2H- |
| | pyrazolo[3,4-b]pyridine |
| | (Preparation 66) yellow oil, 600 mg, 91.1% yield. |
| | LCMS m/z = 308.0 [M + H]⁺ |
| 80 | 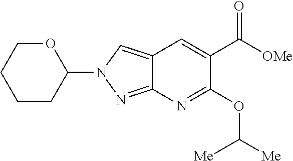 [SMILES: COC(=O)c1cc2cn(C3CCCCO3)nc2nc1OC(C)C] |
| | Methyl 6-isopropoxy-2-(tetrahydro-2H-pyran-2-yl)-2H- |
| | pyrazolo[3,4-b]pyridine-5-carboxylate |
| | SM: 5-bromo-6-isopropoxy-2-(tetrahydro-2H-pyran-2-yl)- |
| | 2H-pyrazolo[3,4-b]pyridine (Preparation 67) |
| | 280 mg, 90.4% yield as a white solid. |
| | LCMS m/z = 320.0 [M + H]⁺ |

### Preparation 81: Methyl 6-isopropoxy-2-(tetrahydro-2H-pyran-3-yl)-2H-pyrazolo[3,4-b]pyridine-5-carboxylate

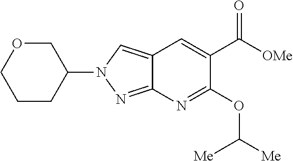 [SMILES: COC(=O)c1cc2cn(C3CCCOC3)nc2nc1OC(C)C]

To a solution of 5-bromo-6-isopropoxy-2-(tetrahydro-2H-pyran-3-yl)-2H-pyrazolo[3,4-b]pyridine (Preparation 68, 150 mg, 0.44 mmol) in MeOH (10 mL) was added TEA (446.2 mg, 4.41 mmol) and Pd(dppf)Cl₂ (32.3 mg, 0.044 mmol) and the reaction stirred at 80° C. under CO (50 psi) for 14 h. The cooled reaction was concentrated in vacuo and the residue was purified by Combiflash® (PE/EtOAc from 75/25 to 0/100) to give methyl 6-isopropoxy-2-(tetrahydro-2H-pyran-3-yl)-2H-pyrazolo[3,4-b]pyridine-5-carboxylate (70 mg, 44.7% yield) as a white solid. LCMS m/z=320.3 [M+H]⁺

### Preparation 82: Methyl 6-isopropoxy-2-((tetrahydrofuran-3-yl)methyl)-2H-pyrazolo[3,4-b]pyridine-5-carboxylate

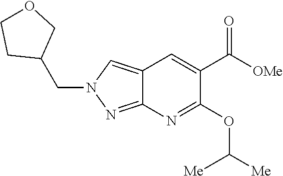 [SMILES: COC(=O)c1cc2cn(CC3CCOC3)nc2nc1OC(C)C]

To a solution of 5-bromo-6-isopropoxy-2-((tetrahydrofuran-3-yl)methyl)-2H-pyrazolo[3,4-b]pyridine (Preparation 61, 90 mg, 0.26 mmol) in MeOH (10 mL) was added TEA (267.7 mg, 2.65 mmol) and Pd(dppf)Cl₂ (38.7 mg, 0.053 mmol) under N₂ and the reaction mixture was stirred at 80° C. under CO (50 psi) for 14 h. The cooled reaction was concentrated in vacuo and the residue was purified by prep-TLC (PE/EtOAc=34/66) to afford methyl 6-isopropoxy-2-((tetrahydrofuran-3-yl)methyl)-2H-pyrazolo[3,4-b]pyridine-5-carboxylate (80 mg, 93.1% yield) as a brown oil. LCMS m/z=320.0 [M+H]⁺

### Preparation 83: Phenyl 6-isopropoxy-2-(1-methyl-2-oxabicyclo[2.2.2]octan-4-yl)-2H-indazole-5-carboxylate

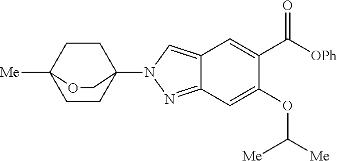 [SMILES: CC(C)Oc1cc2nn(C34CCC(C)(CC3)OC4)cc2cc1C(=O)Oc1ccccc1]

N,N-Diethylethanamine (81.1 mg, 0.80 mmol) was added to a mixture of 5-bromo-6-isopropoxy-2-(1-methyl-2-oxabicyclo[2.2.2]octan-4-yl)-2H-indazole (Preparation 45, 121.6 mg, 0.321 mmol), Pd(OAc)₂ (7.2 mg, 0.032 mmol), Xantphos (37.1 mg, 0.064 mmol) and phenyl formate (97.9 mg, 0.80 mmol) in MeCN (3 mL) at rt. The mixture was sealed and heated at 90° C. overnight. The cooled reaction was filtered through Celite® and the filtrate was concentrated in vacuo. The residue was purified by Isco® automatic purification system (EtOAc in heptane 0/100 to 80/20) to afford phenyl 6-isopropoxy-2-(1-methyl-2-oxabicyclo[2.2.2]octan-4-yl)-2H-indazole-5-carboxylate (98.8 mg, 73.3% yield) as an orange yellow solid. LCMS m/z=421.2 [M+H]⁺

### Preparation 84: Phenyl 6-isopropoxy-2-(1-methyl-2-oxabicyclo[2.2.1]heptan-4-yl)-2H-indazole-5-carboxylate

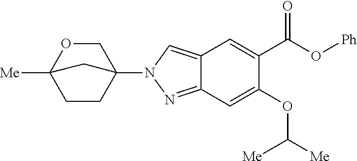 [SMILES: CC(C)Oc1cc2nn(C34CCC(C)(C3)OC4)cc2cc1C(=O)Oc1ccccc1]

N,N-Diethylethanamine (213.5 mg, 2.11 mmol) was added to a mixture of 5-bromo-6-isopropoxy-2-(1-methyl-2-oxabicyclo[2.2.1]heptan-4-yl)-2H-indazole (Preparation 46, 308.2 mg, 0.844 mmol), Pd(OAc)₂ (18.9 mg, 0.084 mmol), Xantphos (97.6 mg, 0.169 mmol) and phenyl formate (257.6 mg, 2.11 mmol) in MeCN (6 mL) at rt. The mixture was sealed and heated at 90° C. overnight. The cooled reaction was filtered through Celite® and the filtrate was concentrated in vacuo. The residue was purified by Isco® automatic purification system (3:1 EtOAc:EtOH in heptanes 0/100 to 50/50) to afford phenyl 6-isopropoxy-2-(1-methyl-2-oxabicyclo[2.2.1]heptan-4-yl)-2H-indazole-5-carboxylate (258.3 mg, 75.3% yield) as a yellow gum. LCMS m/z=407.3 [M+H]⁺

### Preparation 85: Phenyl 6-isopropoxy-2-(1-methyl-2-oxabicyclo[2.1.1]hexan-4-yl)-2H-indazole-5-carboxylate

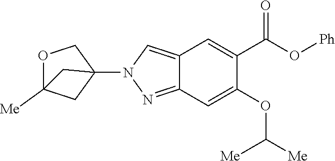 [SMILES: CC(C)Oc1cc2nn(C34COC(C)(C3)C4)cc2cc1C(=O)Oc1ccccc1]

N,N-Diethylethanamine (650.2 mg, 6.42 mmol) was added to a mixture of 5-bromo-6-isopropoxy-2-(1-methyl-2-oxabicyclo[2.1.1]hexan-4-yl)-2H-indazole (Preparation 47, 901 mg, 2.57 mmol), Pd(OAc)₂ (57.7 mg, 0.257 mmol), Xantphos (297.4 mg, 0.514 mmol) and phenyl formate (784.6 mg, 6.42 mmol) in MeCN (9 mL) at rt. The mixture was sealed and heated at 90° C. overnight. The cooled reaction was filtered through Celite® and the filtrate was concentrated in vacuo. The residue was purified by Isco® automatic purification system (3:1 EtOAc:EtOH in heptanes 0/100 to 50/50) to afford phenyl 6-isopropoxy-2-(1-methyl-2-oxabicyclo[2.1.1]hexan-4-yl)-2H-indazole-5-carboxylate (631 mg, 62.6% yield) as an orange solid. LCMS m/z=393.3 [M+H]⁺

### Preparation 86: Phenyl 6-cyclobutoxy-2-(1-methyl-2-oxabicyclo[2.1.1]hexan-4-yl)-2H-indazole-5-carboxylate

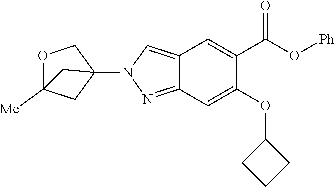 [SMILES: CC12CC(n3cc4cc(C(=O)Oc5ccccc5)c(OC5CCC5)cc4n3)(CO1)C2]

N,N-Diethylethanamine (150.6 mg, 1.49 mmol) was added to a mixture of 5-bromo-6-cyclobutoxy-2-(1-methyl-2-oxabicyclo[2.1.1]hexan-4-yl)-2H-indazole (Preparation 48, 216.3 mg, 0.595 mmol), Pd(OAc)₂ (13.3 mg, 0.06 mmol), Xantphos (68.9 mg, 0.119 mmol) and phenyl formate (181.8 mg, 1.49 mmol) in MeCN (4 mL) at rt. The mixture was sealed and heated at 90° C. overnight. The cooled reaction was filtered through Celite® and the filtrate was concentrated in vacuo. The residue was purified by Isco® automatic purification system (3:1 EtOAc:EtOH in heptanes 0/100 to 50/50) to afford phenyl 6-cyclobutoxy-2-(1-methyl-2-oxabicyclo[2.1.1]hexan-4-yl)-2H-indazole-5-carboxylate (208 mg, 86.4% yield) as an orange yellow solid. LCMS m/z=405.2 [M+H]⁺

### Preparation 87: Phenyl 6-methoxy-2-(1-methyl-2-oxabicyclo[2.1.1]hexan-4-yl)-2H-indazole-5-carboxylate

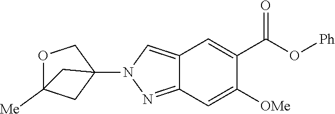 [SMILES: COc1cc2nn(C34COC(C)(C3)C4)cc2cc1C(=O)Oc1ccccc1]

The phenyl 6-methoxy-2-(1-methyl-2-oxabicyclo[2.1.1]hexan-4-yl)-2H-indazole-5-carboxylate was obtained from 5-bromo-6-methoxy-2-(1-methyl-2-oxabicyclo[2.1.1]hexan-4-yl)-2H-indazole (Preparation 49), following the procedure described in Preparation 86.

### Preparation 88: phenyl 6-ethoxy-2-(1-methyl-2-oxabicyclo[2.1.1]hexan-4-yl)-2H-indazole-5-carboxylate

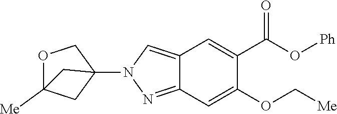 [SMILES: CCOc1cc2nn(C34COC(C)(C3)C4)cc2cc1C(=O)Oc1ccccc1]

Phenyl 6-ethoxy-2-(1-methyl-2-oxabicyclo[2.1.1]hexan-4-yl)-2H-indazole-5-carboxylate was prepared from 5-bromo-6-ethoxy-2-(1-methyl-2-oxabicyclo[2.1.1]hexan-4-yl)-2H-indazole (Preparation 50), following the procedure described in Preparation 86.

### Preparation 89: 6-Ethoxy-2-(tetrahydro-2H-pyran-2-yl)-2H-indazole-5-carboxylic Acid

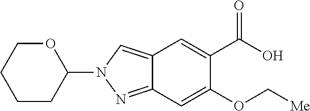 [SMILES: CCOc1cc2nn(C3CCCCO3)cc2cc1C(=O)O]

To a solution of methyl 6-ethoxy-2-(tetrahydro-2H-pyran-2-yl)-2H-indazole-5-carboxylate (Preparation 69, 1.90 g, 6.24 mmol) in H₂O (8 mL), MeOH (8 mL) and THF (8 mL) was added NaOH (748.8 mg, 18.72 mmol) and the reaction stirred at 20° C. for 16 h. The reaction mixture was concentrated in vacuo, the residue diluted with water (30 mL) and extracted with EtOAc (30 mL). The aqueous phase was acidified to pH 3 using 1 M HCl (5 mL) and extracted with EtOAc (30 mL×3). The combined organic layers were washed with brine (50 mL), dried over Na₂SO₄, filtered and evaporated under reduced pressure to afford 6-ethoxy-2-(tetrahydro-2H-pyran-2-yl)-2H-indazole-5-carboxylic acid (1.70 g, 93.8% yield) as a white solid. LCMS m/z=290.9 [M+H]⁺

### Preparation 90: 6-Isopropoxy-2-(4-methoxybutan-2-yl)-2H-indazole-5-carboxylic Acid

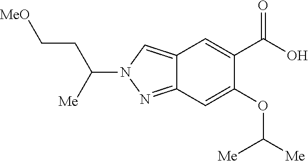 [SMILES: COCCC(C)n1cc2cc(C(=O)O)c(OC(C)C)cc2n1]

To a solution of methyl 6-isopropoxy-2-(4-methoxybutan-2-yl)-2H-indazole-5-carboxylate (Preparation 75, 170 mg, 0.53 mmol) in MeOH (3 mL) and water (1 mL) was added NaOH (64 mg, 1.59 mmol) and the reaction stirred at 20-25° C. for 12 h. The mixture was concentrated in vacuo, the aqueous phase acidified to pH 3 using HCl (1 M) and extracted with EtOAc (20 mL×3). The combined organic layer was washed with brine (20 mL) dried over Na₂SO₄, filtered and the filtrate evaporated under reduced pressure to afford the title compound (150 mg, 92.0% yield) as a yellow oil. LCMS m/z=307.2 [M+H]⁺

### Preparation 91: 6-Isopropoxy-2-(tetrahydro-2H-pyran-4-yl)-2H-indazole-5-carboxylic Acid

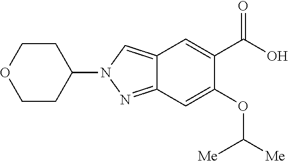 [SMILES: CC(C)Oc1cc2nn(C3CCOCC3)cc2cc1C(=O)O]

6-Isopropoxy-2-(tetrahydro-2H-pyran-4-yl)-2H-indazole-5-carboxylic acid was prepared as a white solid, 290 mg, crude, from methyl 6-isopropoxy-2-(tetrahydro-2H-pyran-4-yl)-2H-indazole-5-carboxylate (Preparation 70), following a similar procedure to that described in Preparation 90. LCMS m/z=305.0 [M+H]⁺

### Preparation 92: 2-(Tetrahydro-2H-pyran-4-yl)-6-((tetrahydrofuran-3-yl)oxy)-2H-indazole-5-carboxylic Acid

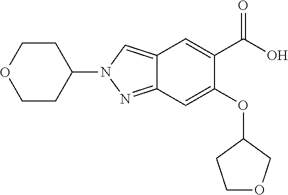 [SMILES: O=C(O)c1cc2cn(C3CCOCC3)nc2cc1OC1CCOC1]

2-(Tetrahydro-2H-pyran-4-yl)-6-((tetrahydrofuran-3-yl)oxy)-2H-indazole-5-carboxylic acid was obtained as a white solid, 70 mg, crude, from methyl 2-(tetrahydro-2H-pyran-4-yl)-6-((tetrahydrofuran-3-yl)oxy)-2H-indazole-5-carboxylate (Preparation 73), following the procedure described in Preparation 90. LCMS m/z=355.0 [M+H]⁺

### Preparation 93: 6-Isopropoxy-2-(tetrahydro-2H-pyran-3-yl)-2H-indazole-5-carboxylic Acid

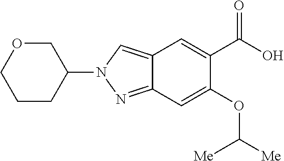 [SMILES: CC(C)Oc1cc2nn(C3CCCOC3)cc2cc1C(=O)O]

To a solution of methyl 6-isopropoxy-2-(tetrahydro-2H-pyran-3-yl)-2H-indazole-5-carboxylate (Preparation 71, 300 mg, 0.94 mmol) in MeOH (2 mL) and water (2 mL) was added LiOH.H₂O (118.6 mg, 2.83 mmol) and the reaction stirred at 25° C. for 3 h. The reaction mixture was neutralized using 1 M HCl aq., concentrated in vacuo and the residue lyophilized to afford 6-isopropoxy-2-(tetrahydro-2H-pyran-3-yl)-2H-indazole-5-carboxylic acid (280 mg, crude) as a white solid. LCMS m/z=304.9 [M+H]⁺

### Preparation 94: 6-((4-Methoxybenzyl)oxy)-2-(tetrahydro-2H-pyran-4-yl)-2H-indazole-5-carboxylic Acid

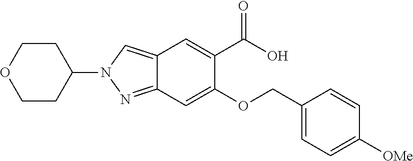 [SMILES: COc1ccc(COc2cc3nn(C4CCOCC4)cc3cc2C(=O)O)cc1]

To a solution of methyl 6-((4-methoxybenzyl)oxy)-2-(tetrahydro-2H-pyran-4-yl)-2H-indazole-5-carboxylate (Preparation 72, 558.2 mg, 1.36 mmol) in MeOH (5 mL) and H₂O (5 mL) was added LiOH.H₂O (32.6 mg, 1.36 mmol) and the reaction stirred at 20° C. for 16 h. The mixture was acidified to pH 3 using 1 M HCl then concentrated in vacuo. The aqueous layer was extracted with EtOAc (20 mL×3), the combined organic layer was washed with brine (30 mL), dried over Na₂SO₄ and filtered. The filtrate was evaporated under reduced pressure to give 6-((4-methoxybenzyl)oxy)-2-(tetrahydro-2H-pyran-4-yl)-2H-indazole-5-carboxylic acid (790 mg, crude) as a white solid. LCMS m/z=383.1 [M+H]⁺

### Preparation 95: 6-Isopropoxy-2-(1-methyl-2-oxabicyclo[2.2.2]octan-4-yl)-2H-indazole-5-carboxylic Acid

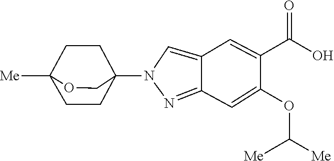 [SMILES: CC(C)Oc1cc2nn(C34CCC(C)(CC3)OC4)cc2cc1C(=O)O]

To a solution of phenyl 6-isopropoxy-2-(1-methyl-2-oxabicyclo[2.2.2]octan-4-yl)-2H-indazole-5-carboxylate (Preparation 83, 98.8 mg, 0.24 mmol) in H₂O (0.5 mL) and THF (1.50 mL) was added LiOH.H₂O (49.3 mg, 1.17 mmol) and the reaction stirred at rt for 16 h. The mixture was neutralized using 1 M HCl, then extracted with EtOAc (8 mL×3). The combined organics were dried over MgSO₄, filtered and the filtrate evaporated under reduced pressure to afford 6-isopropoxy-2-(1-methyl-2-oxabicyclo[2.2.2]octan-4-yl)-2H-indazole-5-carboxylic acid (102 mg, crude), which was used without further purification. LCMS m/z=345.2 [M+H]⁺

### Preparation 96: 6-Isopropoxy-2-(1-methyl-2-oxabicyclo[2.2.1]heptan-4-yl)-2H-indazole-5-carboxylic Acid

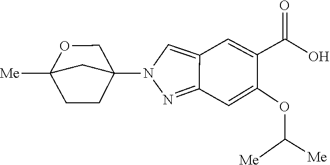 [SMILES: CC(C)Oc1cc2nn(C34CCC(C)(C3)OC4)cc2cc1C(=O)O]

To a solution of phenyl 6-isopropoxy-2-(1-methyl-2-oxabicyclo[2.2.1]heptan-4-yl)-2H-indazole-5-carboxylate (Preparation 84, 258.3 mg, 0.64 mmol) in H₂O (1 mL) and THF (2 mL) was added LiOH.H₂O (53.3 mg, 1.27 mmol) and the reaction stirred at rt for 16 h. The mixture was neutralized using 1 M HCl, then extracted with EtOAc (10 mL×3). The combined organics were dried over MgSO₄, filtered and the filtrate evaporated under reduced pressure to afford 6-isopropoxy-2-(1-methyl-2-oxabicyclo[2.2.1]heptan-4-yl)-2H-indazole-5-carboxylic acid (233 mg, crude) as a yellow gum, which was used without further purification. LCMS m/z=331.1 [M+H]⁺

### Preparation 97: 6-Isopropoxy-2-(1-methyl-2-oxabicyclo[2.1.1]hexan-4-yl)-2H-indazole-5-carboxylic Acid

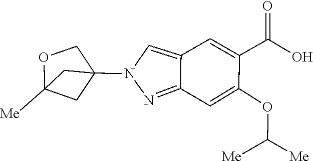 [SMILES: CC(C)Oc1cc2nn(C34COC(C)(C3)C4)cc2cc1C(=O)O]

To a solution of phenyl 6-isopropoxy-2-(1-methyl-2-oxabicyclo[2.1.1]hexan-4-yl)-2H-indazole-5-carboxylate (Preparation 85, 631 mg, 1.61 mmol) in H₂O (2 mL) and THF (6 mL) was added LiOH.H₂O (135.1 mg, 3.22 mmol) and the reaction stirred at rt for 16 h. The mixture was neutralized using 1 M HCl, then extracted with EtOAc (20 mL×3). The combined organics were dried over MgSO₄, filtered and the filtrate evaporated under reduced pressure to afford 6-isopropoxy-2-(1-methyl-2-oxabicyclo[2.1.1]hexan-4-yl)-2H-indazole-5-carboxylic acid (766 mg, crude) as a brown solid, which was used without further purification. LCMS m/z=317.1 [M+H]⁺

### Preparation 98: 6-Cyclobutoxy-2-(1-methyl-2-oxabicyclo[2.1.1]hexan-4-yl)-2H-indazole-5-carboxylic Acid

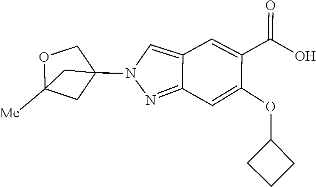 [SMILES: CC12CC(n3cc4cc(C(=O)O)c(OC5CCC5)cc4n3)(CO1)C2]

To a solution of phenyl 6-cyclobutoxy-2-(1-methyl-2-oxabicyclo[2.1.1]hexan-4-yl)-2H-indazole-5-carboxylate (Preparation 86, 208 mg, 0.514 mmol) in H₂O (1 mL) and THF (3 mL) was added LiOH.H₂O (43.2 mg, 1.03 mmol) and the reaction stirred at rt for 16 h. The mixture was neutralized using 1 M HCl, then extracted with EtOAc (10 mL×3). The combined organics were dried over MgSO₄, filtered and the filtrate evaporated under reduced pressure to afford 6-cyclobutoxy-2-(1-methyl-2-oxabicyclo[2.1.1]hexan-4-yl)-2H-indazole-5-carboxylic acid (190 mg, crude), which was used without further purification. LCMS m/z=329.1 [M+H]⁺

### Preparation 99: 6-Methoxy-2-(1-methyl-2-oxabicyclo[2.1.1]hexan-4-yl)-2H-indazole-5-carboxylic Acid

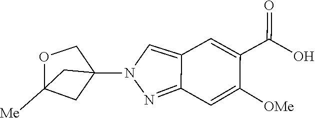 [SMILES: COc1cc2nn(C34COC(C)(C3)C4)cc2cc1C(=O)O]

6-Methoxy-2-(1-methyl-2-oxabicyclo[2.1.1]hexan-4-yl)-2H-indazole-5-carboxylic acid was obtained from phenyl 6-methoxy-2-(1-methyl-2-oxabicyclo[2.1.1]hexan-4-yl)-2H-indazole-5-carboxylate (Preparation 87), following a similar procedure to that described in Preparation 98.

### Preparation 100: 6-Ethoxy-2-(1-methyl-2-oxabicyclo[2.1.1]hexan-4-yl)-2H-indazole-5-carboxylic Acid

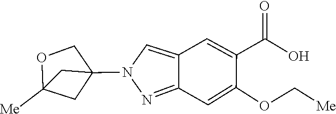 [SMILES: CCOc1cc2nn(C34COC(C)(C3)C4)cc2cc1C(=O)O]

6-Ethoxy-2-(1-methyl-2-oxabicyclo[2.1.1]hexan-4-yl)-2H-indazole-5-carboxylic acid was prepared from phenyl 6-ethoxy-2-(1-methyl-2-oxabicyclo[2.1.1]hexan-4-yl)-2H-indazole-5-carboxylate (Preparation 88), following a similar procedure to that described in Preparation 98.

### Preparation 101: 2-((1r,3r)-3-Hydroxycyclobutyl)-6-isopropoxy-2H-indazole-5-carboxylic Acid

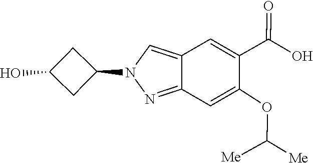 [SMILES: CC(C)Oc1cc2nn([C@H]3C[C@H](O)C3)cc2cc1C(=O)O]

To a solution of methyl 2-((1r,3r)-3-(benzyloxy)cyclobutyl)-6-isopropoxy-2H-indazole-5-carboxylate (Preparation 74, 600 mg, 1.52 mmol) in MeOH (30 mL) was added Pd/C (200 mg, 10% purity, wet) and the reaction stirred at 50° C. under H₂ (50 psi) for 16 h. The mixture was filtered through Celite®, and the filtrate evaporated under reduced pressure to afford 2-((1r,3r)-3-hydroxycyclobutyl)-6-isopropoxy-2H-indazole-5-carboxylic acid (350 mg, 76% yield) as a colorless oil. LCMS m/z=304.9 [M+H]⁺

### Preparation 102: 6-Isopropoxy-2-((1r,3r)-3-methoxycyclobutyl)-2H-indazole-5-carboxylic Acid

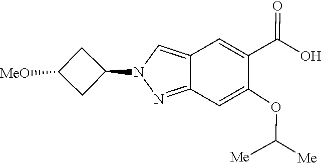 [SMILES: CO[C@H]1C[C@H](n2cc3cc(C(=O)O)c(OC(C)C)cc3n2)C1]

To a solution of methyl 2-((1r,3r)-3-(hydroxy)cyclobutyl)-6-isopropoxy-2H-indazole-5-carboxylate (Preparation 101, 350 mg, 1.15 mmol) in THF (10 mL) was added NaH (92 mg, 2.30 mmol, 60% purity) at 0° C. and the mixture stirred for 30 min. Iodomethane (1.17 g, 8.24 mmol) was added and the reaction stirred at 25° C. for 2 h. The reaction was quenched with water (30 mL) and NH₄OH (28% w/w, 5 mL) then extracted with EtOAc (30 mL). The aqueous layer was acidified to pH 3 using 1 M HCl then extracted with EtOAc (30 mL×3). The combined organic layer was dried over Na₂SO₄, filtered and the filtrate evaporated under reduced pressure to afford 6-isopropoxy-2-((1r,3r)-3-methoxycyclobutyl)-2H-indazole-5-carboxylic acid (300 mg, 73% yield) as yellow oil. LCMS m/z=304.9 [M+H]⁺

### Preparation 103: 6-Isopropoxy-2-(tetrahydro-2H-pyran-3-yl)-2H-pyrazolo[3,4-b]pyridine-5-carboxylic Acid

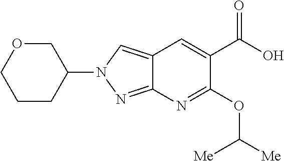 [SMILES: CC(C)Oc1nc2nn(C3CCCOC3)cc2cc1C(=O)O]

To a solution of methyl 6-isopropoxy-2-(tetrahydro-2H-pyran-3-yl)-2H-pyrazolo[3,4-b]pyridine-5-carboxylate (Preparation 81, 70 mg, 0.22 mmol) in MeOH (2 mL) and water (2 mL) was added NaOH (17.5 mg, 0.44 mmol) and the reaction stirred at 20° C. for 14 h. The reaction was concentrated in vacuo and the residue was acidified with aqueous KHSO₄ to pH<7 and evaporated under reduced pressure to afford 6-isopropoxy-2-(tetrahydro-2H-pyran-3-yl)-2H-pyrazolo[3,4-b]pyridine-5-carboxylic acid (65 mg, crude) as a white solid. LCMS m/z=306.3 [M+H]⁺

### Preparation 104: 6-isopropoxy-2-((tetrahydrofuran-3-yl)methyl)-2H-pyrazolo[3,4-b]pyridine-5-carboxylic Acid

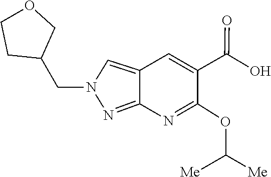

To a solution of methyl 6-isopropoxy-2-((tetrahydrofuran-3-yl)methyl)-2H-pyrazolo[3,4-b]pyridine-5-carboxylate (Preparation 82, 80 mg, 0.25 mmol) in MeOH (1 mL) and water (1 mL) was added NaOH (20 mg, 0.50 mmol) at 20° C. and the reaction stirred at 20° C. for 5 h. The mixture was concentrated in vacuo to remove MeOH, the solution neutralized using aq. KHSO₄ then evaporated under reduced pressure to afford 6-isopropoxy-2-((tetrahydrofuran-3-yl)methyl)-2H-pyrazolo[3,4-b]pyridine-5-carboxylic acid (50 mg, 98.1% yield) as a white solid. ¹H NMR (400 MHz, DMSO-d₆) δ: 1.33 (d, 6H), 1.58-1.67 (m, 1H), 1.88-1.97 (m, 1H), 2.81-2.88 (m, 1H), 3.47-3.53 (m, 1H), 3.61-3.70 (m, 2H), 3.75-3.81 (m, 1H), 4.35 (d, 2H), 5.35-5.42 (m, 1H), 8.45 (s, 1H), 8.51 (s, 1H)

### Preparation 105: 6-Cyclobutoxy-2-((tetrahydrofuran-3-yl)methyl)-2H-pyrazolo[3,4-b]pyridine-5-carboxylic Acid

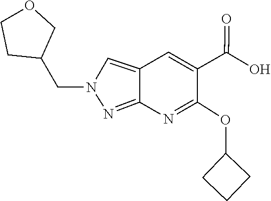

To a solution of methyl 6-cyclobutoxy-2-((tetrahydrofuran-3-yl)methyl)-2H-pyrazolo[3,4-b]pyridine-5-carboxylate (Preparation 76, 600 mg, 1.81 mmol) in MeOH (5 mL) and water (5 mL) was added NaOH (144.8 mg, 3.62 mmol) and the reaction was stirred at 20° C. for 14 h. The mixture was concentrated in vacuo and the aqueous residue acidified with aqueous KHSO₄ to pH<7, then evaporated under reduced pressure to afford 6-cyclobutoxy-2-((tetrahydrofuran-3-yl)methyl)-2H-pyrazolo[3,4-b]pyridine-5-carboxylic acid (550 mg, crude) as a white solid. LCMS m/z=318.4 [M+H]⁺

### Preparation 106: 6-(Cyclopentyloxy)-2-((tetrahydrofuran-3-yl)methyl)-2H-pyrazolo[3,4-b]pyridine-5-carboxylic Acid

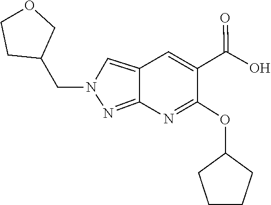

6-(Cyclopentyloxy)-2-((tetrahydrofuran-3-yl)methyl)-2H-pyrazolo[3,4-b]pyridine-5-carboxylic acid was obtained as a white solid, 350 mg, 82.0% yield, from methyl 6-(cyclopentyloxy)-2-((tetrahydrofuran-3-yl)methyl)-2H-pyrazolo[3,4-b]pyridine-5-carboxylate (Preparation 77), following the procedure described in Preparation 105. LCMS m/z=332.3 [M+H]⁺

### Preparation 107: 6-Isopropoxy-2-(3-methoxypropyl)-2H-pyrazolo[3,4-b]pyridine-5-carboxylic Acid

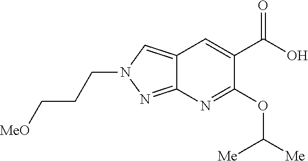 [SMILES: COCCCn1cc2cc(C(=O)O)c(OC(C)C)nc2n1]

6-Isopropoxy-2-(3-methoxypropyl)-2H-pyrazolo[3,4-b]pyridine-5-carboxylic acid was obtained as a white solid from methyl 6-isopropoxy-2-(3-methoxypropyl)-2H-pyrazolo[3,4-b]pyridine-5-carboxylate (Preparation 79) following the procedure described in Preparation 105. ¹H NMR (400 MHz, DMSO-d₆) δ: 1.33 (d, 6H), 2.08-2.15 (m, 2H), 3.23 (s, 3H), 3.28-3.30 (m, 2H), 4.38 (t, 2H), 5.34-5.41 (m, 1H), 8.39 (s, 1H), 8.50 (s, 1H).

### Preparation 108: 6-Isopropoxy-2-(tetrahydro-2H-pyran-4-yl)-2H-pyrazolo[3,4-b]pyridine-5-carboxylic Acid

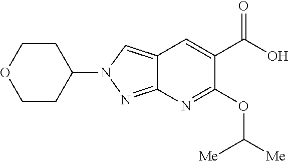 [SMILES: CC(C)Oc1nc2nn(C3CCOCC3)cc2cc1C(=O)O]

6-Isopropoxy-2-(tetrahydro-2H-pyran-4-yl)-2H-pyrazolo[3,4-b]pyridine-5-carboxylic acid was obtained as a white solid, 190 mg, 99.4% yield, from methyl 6-isopropoxy-2-(tetrahydro-2H-pyran-4-yl)-2H-pyrazolo[3,4-b]pyridine-5-carboxylate (Preparation 78) following the procedure described in Preparation 105. ¹H NMR (500 MHz, MeOH-d₄) δ: 1.43 (d, 6H), 2.13-2.16 (m, 2H), 2.19-2.28 (m, 2H), 3.60-3.66 (m, 2H), 4.09-4.13 (m, 2H), 4.63-4.70 (m, 1H), 5.51-5.56 (m, 1H), 8.36 (s, 1H), 8.64 (s, 1H)

### Preparation 109: 6-Isopropoxy-2-(tetrahydro-2H-pyran-2-yl)-2H-pyrazolo[3,4-b]pyridine-5-carboxylic Acid

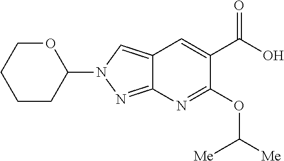 [SMILES: CC(C)Oc1nc2nn(C3CCCCO3)cc2cc1C(=O)O]

6-Isopropoxy-2-(tetrahydro-2H-pyran-2-yl)-2H-pyrazolo[3,4-b]pyridine-5-carboxylic acid was prepared as a white solid, 290 mg, crude, from methyl 6-isopropoxy-2-(tetrahydro-2H-pyran-2-yl)-2H-pyrazolo[3,4-b]pyridine-5-carboxylate (Preparation 80), following the procedure described in Preparation 105. LCMS m/z=306.0 [M+H]⁺

### Preparation 110: Pyrazolo[1,5-a]pyrimidin-3-yl 6-isopropoxy-2-(tetrahydro-2H-pyran-2-yl)-2H-pyrazolo[3,4-b]pyridine-5-carboxylate

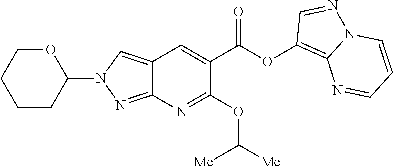 [SMILES: CC(C)Oc1nc2nn(C3CCCCO3)cc2cc1C(=O)Oc1cnn2cccnc12]

To a solution of 6-isopropoxy-2-(tetrahydro-2H-pyran-2-yl)-2H-pyrazolo[3,4-b]pyridine-5-carboxylic acid (Preparation 109, 1.70 g, 5.57 mmol) in pyridine (10 mL) was added pyrazolo[1,5-a]pyrimidin-3-amine (1.49 g, 11.14 mmol) and T3P® (10 mL) and the reaction stirred at 20° C. for 3 h. The reaction was concentrated in vacuo, the residue was diluted with aqueous aq. NaHCO₃ (100 mL) and extracted with EtOAc (60 mL×3). The combined organic layers were dried over Na₂SO₄, filtered and the filtrate evaporated under reduced pressure. The crude product was purified by Combiflash® (PE:EtOAc=75/25 to 0/100) to afford pyrazolo[1,5-a]pyrimidin-3-yl 6-isopropoxy-2-(tetrahydro-2H-pyran-2-yl)-2H-pyrazolo[3,4-b]pyridine-5-carboxylate (1.80 g, 68.9% yield) as a white solid. LCMS m/z=422.3 [M+H]⁺

### Preparations 111 to 115

The following compounds were prepared from the appropriate carboxylic acid and amine, following a similar procedure to that described in Preparation 110.

| Prep. | |
|---|---|
| No | Structure, Name, Starting materials (SM), Yield, Data |
| 111 | 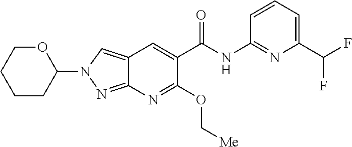 [SMILES: CCOc1nc2nn(C3CCCCO3)cc2cc1C(=O)Nc1cccc(C(F)F)n1] |
| | N-(6-(Difluoromethyl)pyridin-2-yl)-6-ethoxy-2-(tetrahydro- |
| | 2H-pyran-2-yl)-2H-indazole-5-carboxamide |
| | SM: 6-ethoxy-2-(tetrahydro-2H-pyran-2-yl)-2H-indazole-5- |
| | carboxylic acid (Preparation 89) and 6-(difluoromethyl) |
| | pyridine-2-amine white solid, 300 mg, 59.8% yield. |
| | LCMS m/z = 417.0 [M + H]⁺ |
| 112 | 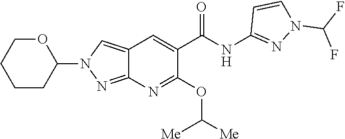 [SMILES: CC(C)Oc1nc2nn(C3CCCCO3)cc2cc1C(=O)Nc1ccn(C(F)F)n1] |
| | N-(1-(Difluoromethyl)-1H-pyrazol-3-yl)-6-isopropoxy-2- |
| | (tetrahydro-2H-pyran-2-yl)-2H-indazole-5-carboxamide |
| | SM: 6-isopropoxy-2-(tetrahydro-2H-pyran-2-yl)-2H-indazole- |
| | 5-carboxylic acid (Preparation 13) and 1-(difluoromethyl)-1H- |
| | pyrazol-3-amine brown solid, 320 mg, 79.0% yield. |
| | LCMS m/z = 411.0 [M + H]⁺ |
| 113 | 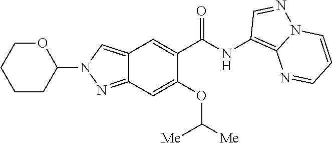 [SMILES: CC(C)Oc1cc2nn(C3CCCCO3)cc2cc1C(=O)Nc1cnn2cccnc12] |
| | 6-Isopropoxy-N-(pyrazol[1,5-a]pyrimidin-3-yl)-2-(tetrahydro- |
| | 2H-pyran-2-yl)-2H-indazole-5-carboxamide |
| | SM: 6-isopropoxy-2-(tetrahydro-2H-pyran-2-yl)-2H-indazole- |
| | 5-carboxylic acid (Preparation 13) and pyrazolo[1,5-a]pyrimidin- |
| | 3-amine white solid, 300 mg, 86.9% yield. |
| | LCMS m/z = 421.1 [M + H]⁺ |
| 114 | 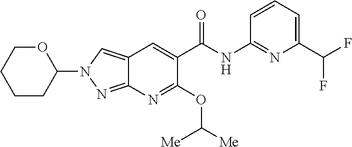 [SMILES: CC(C)Oc1nc2nn(C3CCCCO3)cc2cc1C(=O)Nc1cccc(C(F)F)n1] |
| | N-(6-(Difluoromethyl)pyridin-2-yl)-6-isopropoxy-2- |
| | (tetrahydro-2H-pyran-2-yl)-2H-pyrazolo[3,4-b] |
| | pyridine-5-carboxamide |
| | SM: 6-(difluoromethyl)pyridine-2-amine and 6-isopropoxy- |
| | 2-(tetrahydro-2H-pyran-2-yl)- |
| | 2H-pyrazolo[3,4-b]pyridine-5-carboxylic acid |
| | (Preparation 109) white solid, 330 mg, 80.5% yield. |
| | LCMS m/z = 432.1 [M + H]⁺ |
| 115 | 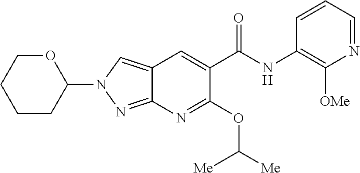 [SMILES: COc1ncccc1NC(=O)c1cc2cn(C3CCCCO3)nc2nc1OC(C)C] |
| | 6-isopropoxy-N-(2-methoxypyridin-3-yl)-2-(tetrahydro- |
| | 2H-pyran-2-yl)-2H-pyrazolo[3,4-b]pyridine-5-carboxamide |
| | SM: 6-isopropoxy-2-(tetrahydro-2H-pyran-2-yl)-2H-pyrazolo |
| | [3,4-b]pyridine-5-carboxylic acid (Preparation 109) and 2- |
| | methoxypyridin-3-amine white solid, 227 mg, 66.0% yield. |
| | LCMS m/z = 412.0 [M + H]⁺ |

### Preparation 116: N-(6-(difluoromethyl)pyridin-2-yl)-6-ethoxy-2H-indazole-5-carboxamide

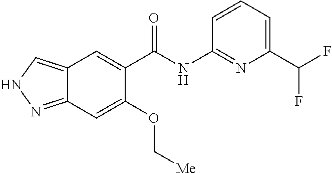 [SMILES: CCOc1cc2n[nH]cc2cc1C(=O)Nc1cccc(C(F)F)n1]

To a solution of N-(6-(difluoromethyl)pyridin-2-yl)-6-ethoxy-2-(tetrahydro-2H-pyran-2-yl)-2H-indazole-5-carboxamide (Preparation 111, 400 mg, 0.96 mmol) in EtOAc (5 mL) was added 4 M HCl/EtOAc (5 mL) and the reaction mixture stirred at 20° C. for 16 h. The mixture was concentrated in vacuo and the residue was neutralized using NaHCO₃ (10 mL) and extracted with EtOAc (20 mL×3). The combined organic layers were washed with brine (20 mL), dried over Na₂SO₄, filtered and evaporated under reduced pressure to afford N-(6-(difluoromethyl)pyridin-2-yl)-6-ethoxy-2H-indazole-5-carboxamide (300 mg, 94.0% yield) as a white solid. LCMS m/z=332.9 [M+H]⁺

### Preparation 117: 6-Isopropoxy-N-(pyrazolo[1,5-a]pyrimidin-3-yl)-2H-indazole-5-carboxamide

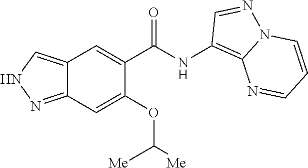 [SMILES: CC(C)Oc1cc2n[nH]cc2cc1C(=O)Nc1cnn2cccnc12]

6-Isopropoxy-N-(pyrazolo[1,5-a]pyrimidin-3-yl)-2H-indazole-5-carboxamide was obtained as a brown solid, 170 mg, 70.8% yield, from 6-isopropoxy-N-(pyrazolo[1,5-a]pyrimidin-3-yl)-2-(tetrahydro-2H-pyran-2-yl)-2H-indazole-5-carboxamide (Preparation 113), following the procedure described in Preparation 116. LCMS m/z=337.0 [M+H]⁺

### Preparation 118: N-(1-(difluoromethyl)-1H-pyrazol-3-yl)-6-isopropoxy-2H-indazole-5-carboxamide

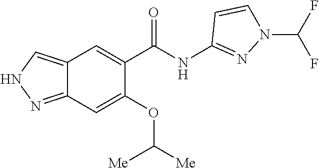 [SMILES: CC(C)Oc1cc2n[nH]cc2cc1C(=O)Nc1ccn(C(F)F)n1]

N-(1-(Difluoromethyl)-1H-pyrazol-3-yl)-6-isopropoxy-2H-indazole-5-carboxamide was obtained as a brown solid, 210 mg, crude, from N-(1-(difluoromethyl)-1H-pyrazol-3-yl)-6-isopropoxy-2-(tetrahydro-2H-pyran-2-yl)-2H-indazole-5-carboxamide (Preparation 112), following the procedure described in Preparation 116.

### Preparation 119: 6-Isopropoxy-N-(pyrazolo[1,5-a]pyrimidin-3-yl)-2H-pyrazolo[3,4-b]pyridine-5-carboxamide

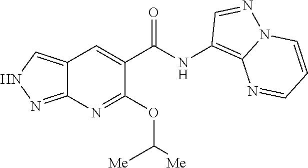 [SMILES: CC(C)Oc1nc2n[nH]cc2cc1C(=O)Nc1cnn2cccnc12]

To a solution of pyrazolo[1,5-a]pyrimidin-3-yl 6-isopropoxy-2-(tetrahydro-2H-pyran-2-yl)-2H-pyrazolo[3,4-b]pyridine-5-carboxylate (Preparation 110, 1.70 g, 4.03 mmol) in DCM (12 mL) was added TFA (4 mL) and the reaction stirred at 20° C. for 14 h. The reaction was neutralized using saturated aq. NaHCO₃ (120 mL) and extracted with DCM (60 mL×3). The combined organic layers were washed with water (40 mL), dried over Na₂SO₄, filtered and the filtrate evaporated under reduced pressure. The residue was purified by Combiflash® (PE:EtOAc=75/25 to 100/0) to afford 6-isopropoxy-N-(pyrazolo[1,5-a]pyrimidin-3-yl)-2H-pyrazolo[3,4-b]pyridine-5-carboxamide (1.10 g, 72.7% yield) as a yellow solid. LCMS m/z=338.2 [M+H]⁺

### Preparation 120: N-(6-(difluoromethyl)pyridin-2-yl)-6-isopropoxy-2H-pyrazolo[3,4-b]pyridine-5-carboxamide

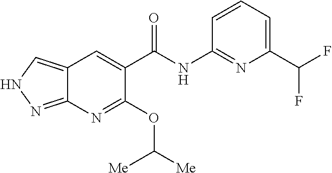 [SMILES: CC(C)Oc1nc2n[nH]cc2cc1C(=O)Nc1cccc(C(F)F)n1]

N-(6-(difluoromethyl)pyridin-2-yl)-6-isopropoxy-2H-pyrazolo[3,4-b]pyridine-5-carboxamide was obtained as a white solid, 130 mg, 59.8% yield from N-(6-(difluoromethyl)pyridin-2-yl)-6-isopropoxy-2-(tetrahydro-2H-pyran-2-yl)-2H-pyrazolo[3,4-b]pyridine-5-carboxamide (Preparation 114) following a similar procedure to that described in Preparation 119, except the compound was purified by prep-TLC (PE/EtOAc=2/1). LCMS m/z=348.0 [M+H]⁺

### Preparation 121: 6-Isopropoxy-N-(2-methoxypyridin-3-yl)-2H-pyrazolo[3,4-b]pyridine-5-carboxamide

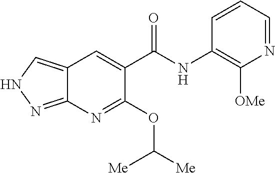 [SMILES: COc1ncccc1NC(=O)c1cc2c[nH]nc2nc1OC(C)C]

6-Isopropoxy-N-(2-methoxypyridin-3-yl)-2H-pyrazolo[3,4-b]pyridine-5-carboxamide was obtained as a white solid, 98 mg, 66.8% yield, from 6-isopropoxy-N-(2-methoxypyridin-3-yl)-2-(tetrahydro-2H-pyran-2-yl)-2H-pyrazolo[3,4-b]pyridine-5-carboxamide (Preparation 115), following the procedure described in Preparation 119. LCMS m/z=327.9 [M+H]⁺

### Preparation 122: N-(6-(difluoromethyl)pyridin-2-yl)-6-((4-methoxybenzyl)oxy)-2-(tetrahydro-2H-pyran-4-yl)-2H-indazole-5-carboxamide

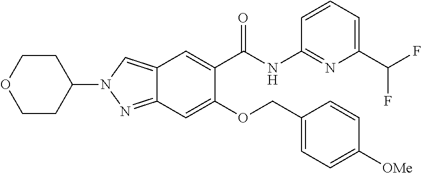 [SMILES: COc1ccc(COc2cc3nn(C4CCOCC4)cc3cc2C(=O)Nc2cccc(C(F)F)n2)cc1]

N-(6-(Difluoromethyl)pyridin-2-yl)-6-((4-methoxybenzyl)oxy)-2-(tetrahydro-2H-pyran-4-yl)-2H-indazole-5-carboxamide was obtained, 250 mg, 43.0% yield, from 6-((4-methoxybenzyl)oxy)-2-(tetrahydro-2H-pyran-4-yl)-2H-indazole-5-carboxylic acid (Preparation 94) and 6-(difluoromethyl)pyridin-2-amine, following a similar procedure to that described in Preparation 110.

### Preparation 123: N-(6-(difluoromethyl)pyridin-2-yl)-6-hydroxy-2-(tetrahydro-2H-pyran-4-yl)-2H-indazole-5-carboxamide

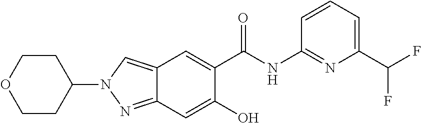 [SMILES: O=C(Nc1cccc(C(F)F)n1)c1cc2cn(C3CCOCC3)nc2cc1O]

A solution of N-(6-(difluoromethyl)pyridin-2-yl)-6-((4-methoxybenzyl)oxy)-2-(tetrahydro-2H-pyran-4-yl)-2H-indazole-5-carboxamide (Preparation 122, 590 mg, 1.16 mmol) in TFA (20 mL) was stirred at 20° C. for 16 h. The mixture was concentrated in vacuo and the residue was neutralized using aq. NaHCO₃ (10 mL). The aqueous solution was extracted with EtOAc (20 mL×3), the combined organic layers were washed with brine (20 mL), dried over Na₂SO₄, filtered and evaporated under reduced pressure to afford N-(6-(difluoromethyl)pyridin-2-yl)-6-hydroxy-2-(tetrahydro-2H-pyran-4-yl)-2H-indazole-5-carboxamide (387 mg, 77.3% yield). LCMS m/z=389.1 [M+H]⁺

### Preparation 124: 6-Isopropoxy-2-((tetrahydrofuran-3-yl)methyl)-2H-pyrazolo[3,4-b]pyridine-5-carboxamide

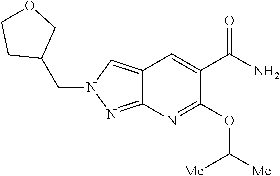 [SMILES: CC(C)Oc1nc2nn(CC3CCOC3)cc2cc1C(N)=O]

To a solution of 6-isopropoxy-2-((tetrahydrofuran-3-yl)methyl)-2H-pyrazolo[3,4-b]pyridine-5-carboxylic acid (Preparation 104, 1.0 g, 0.56 mmol) in DCM (5 mL) was added SOCl₂ (79.5 mg, 0.67 mmol) and a drop of DMF at 0° C. under N₂ and the reaction stirred at 20° C. for 16 h. The mixture was concentrated in vacuo and the residue diluted with THF (5 mL) and NH₄OH (697.0 mg, 5.57 mmol, 28% purity) added. The resulting mixture was stirred at 20° C. for 1 h then diluted with water (30 mL) and extracted with EtOAc (40 mL×3). The combined organic layers were washed with brine (50 mL), dried over Na₂SO₄ and filtered. The filtrate was evaporated under reduced pressure to afford 6-isopropoxy-2-((tetrahydrofuran-3-yl)methyl)-2H-pyrazolo[3,4-b]pyridine-5-carboxamide (150 mg, 85.9% yield) as a white solid. LCMS m/z=305.0 [M+H]⁺

### Preparation 125: 6-Isopropoxy-2-(tetrahydro-2H-pyran-4-yl)-2H-indazole-5-carboxamide

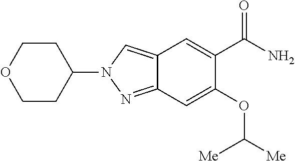 [SMILES: CC(C)Oc1cc2nn(C3CCOCC3)cc2cc1C(N)=O]

6-Isopropoxy-2-(tetrahydro-2H-pyran-4-yl)-2H-indazole-5-carboxamide was obtained as a white solid, 100 mg, crude, from 6-isopropoxy-2-(tetrahydro-2H-pyran-4-yl)-2H-indazole-5-carboxylic acid (Preparation 91), following the procedure described in Preparation 124. LCMS m/z=304.1 [M+H]⁺

### Preparation 126: 6-Cyclobutoxy-2-(tetrahydro-2H-pyran-2-yl)-2H-pyrazolo[3,4-b]pyridine

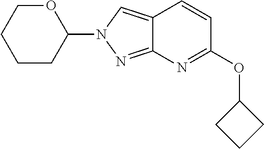 [SMILES: c1cc2cn(C3CCCCO3)nc2nc1OC1CCC1]

To a solution of cyclobutanol (14.56 g, 201.9 mmol, 15.83 mL, 6.0 eq.) in THF (200.00 mL) was added sodium hydride (5.39 g, 134 mmol, 60% purity, 4.0 eq.) at 0° C. under N₂. The mixture was stirred at 0° C. for 30 min, to the reaction mixture was then added 6-chloro-2-(tetrahydro-2H-pyran-2-yl)-2H-pyrazolo[3,4-b]pyridine (preparation 54; 8.00 g, 33.6 mmol, 1.0 eq.). The mixture was stirred at 60° C. for 14 hours. The reaction was quenched with water (20 mL). THF was evaporated under vacuum to give the residue. The residue was diluted with water (80 mL), extracted with EtOAc (50 mL×3). The combined organic layer was washed with brine (80 mL×2), dried over Na₂SO₄, filtered and evaporated under vacuum. The residue was purified by Combi-Flash (PE:EA from 6:1 to 1:1) to give 6-cyclobutoxy-2-(tetrahydro-2H-pyran-2-yl)-2H-pyrazolo[3,4-b]pyridine (8.70 g, 85.1% yield) as a white solid. LCMS: m/z=274.3 [M+H]⁺. ¹H NMR: (400 MHz, CDCl₃) δ: 1.68-1.62 (m, 1H), 1.77-1.68 (m, 1H), 1.85-1.77 (m, 2H), 1.93-1.85 (m, 1H), 2.00-1.95 (m, 1H), 2.27-2.13 (m, 3H), 2.57-2.50 (m, 2H), 2.71-2.62 (m, 1H), 3.83-3.75 (m, 1H), 4.16-4.10 (m, 1H), 5.33-5.25 (m, 1H), 5.94 (dd, J₁=10.4 Hz, J₂=2.0 Hz, 1H), 6.57 (d, J=8.4 Hz, 1H), 7.85 (d, J=8.8 Hz, 1H), 7.91 (s, 1H).

### Preparation 127: 6-Cyclobutoxy-2H-pyrazolo[3,4-b]pyridine

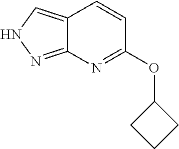 [SMILES: c1cc2c[nH]nc2nc1OC1CCC1]

To a solution of 6-cyclobutoxy-2-(tetrahydro-2H-pyran-2-yl)-2H-pyrazolo[3,4-b]pyridine (8.70 g, 31.8 mmol, 1.0 eq.) in dioxane (80.00 mL) was added HCl/dioxane (4 M, 80.0 mL) at 20° C. The reaction was stirred at 20° C. for 4 hours. TLC (PE:EA=3:1, Rf˜0.4) showed a new main spot was observed. The reaction was slowly poured into saturate aq. NaHCO₃ (500 mL) and extracted with EtOAc (200 mL×3). The combined organic lawyer was washed with brine (100 mL×2), dried over Na₂SO₄, filtered and evaporated under vacuum to give 6-cyclobutoxy-2H-pyrazolo[3,4-b]pyridine (6.00 g, 89.6% yield) as a white solid. LCMS: m/z=190.3 [M+H]⁺. ¹H NMR: (500 MHz, CDCl₃) δ: 1.78-1.71 (m, 1H), 1.92-1.87 (m, 1H), 2.23-2.16 (m, 2H), 2.57-2.54 (m, 2H), 5.38-5.32 (m, 1H), 6.61 (d, J=8.5 Hz, 1H), 7.92 (d, J=8.5 Hz, 1H), 7.95 (s, 1H).

### Preparation 128: 5-Bromo-6-cyclobutoxy-2H-pyrazolo[3,4-b]pyridine

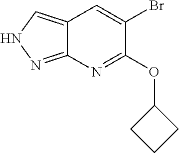 [SMILES: Brc1cc2c[nH]nc2nc1OC1CCC1]

To a solution of -cyclobutoxy-2H-pyrazolo[3,4-b]pyridine (6.00 g, 31.7 mmol, 1.0 eq.) in AcOH (80.00 mL) was added Br₂ (5.07 g, 31.7 mmol, 1.63 mL, 1.0 eq.) at 20° C. The mixture was stirred at 20° C. for 4 hours. LCMS showed 56.7% of the desired product was obtained and 24.9% of the starting material remained. The mixture was slowly poured into aqueous NaHCO₃ (800 mL), extracted with EtOAc (200 mL×3). The combined organic layer was washed with brine (300 mL), dried over Na₂SO₄, filtered; evaporated under vacuum. The residue was purified by Combi-Flash (DCM:EtOAc from 1:0 to 5:1) to give 5-bromo-6-cyclobutoxy-2H-pyrazolo[3,4-b]pyridine (5.40 g, 57.2% yield) as a white solid. LCMS: m/z=268.2 [M+H]⁺. ¹H NMR: (500 MHz, CDCl₃) δ: 1.78-1.73 (m, 1H), 1.93-1.90 (m, 1H), 2.30-2.25 (m, 2H), 2.57-2.54 (m, 2H), 5.34-5.28 (m, 1H), 7.90 (s, 1H), 8.18 (s, 1H).

### Preparation 129: 5-Bromo-6-cyclobutoxy-2-(tetrahydro-2H-pyran-3-yl)-2H-pyrazolo[3,4-b]pyridine

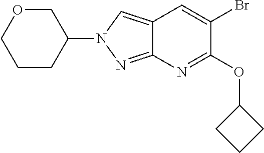 [SMILES: Brc1cc2cn(C3CCCOC3)nc2nc1OC1CCC1]

To a solution of 5-bromo-6-cyclobutoxy-2H-pyrazolo[3,4-b]pyridine (4.60 g, 17.2 mmol, 1.0 eq.) in DMF (200.00 mL) was added K₂CO₃ (14.23 g, 102.9 mmol, 6.0 eq.) and rac-(R)-tetrahydro-2H-pyran-3-yl methanesulfonate (18.55 g, 102.9 mmol, 6.0 eq.) at 20° C. The reaction was stirred at 100° C. for 14 hours. LCMS showed 12.7% of the desired product was obtained and 13.8% of the starting material remained. The reaction was filtered and the filtrate was evaporated under vacuum. The residue was diluted with water (80 mL), extracted with EtOAc (60 mL×3). The combined organic layer was washed with brine (80 mL×2), dried over Na₂SO₄; filtered and evaporated under vacuum. The residue was purified by Combi-Flash (PE:EA from 3:1 to 1:1) to give the crude product (1.3 g). The crude product was purified by Prep-TLC (PE:EA=1:1) to give 5-Bromo-6-cyclobutoxy-2-(tetrahydro-2H-pyran-3-yl)-2H-pyrazolo[3,4-b]pyridine (700 mg, 9.68% yield) as yellow oil. LCMS: m/z=354.2 [M+H]⁺. ¹H NMR: (500 MHz, CDCl₃) δ: 1.73-1.66 (m, 1H), 1.82-1.79 (m, 1H), 1.89-1.83 (m, 1H), 1.96-1.90 (m, 1H), 2.28-2.20 (m, 2H), 2.43-2.37 (m, 1H), 2.62-2.57 (m, 2H), 3.68-3.62 (m, 2H), 3.94-3.89 (m, 1H), 4.00-3.95 (m, 1H), 4.18-4.14 (m, 1H), 4.52-4.46 (m, 1H), 5.46-5.39 (m, 1H), 7.91 (s, 1H), 8.12 (s, 1H).

### Preparation 130: Methyl 6-cyclobutoxy-2-(tetrahydro-2H-pyran-3-yl)-2H-pyrazolo[3,4-b]pyridine-5-carboxylate

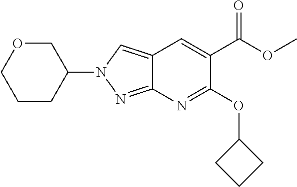 [SMILES: COC(=O)c1cc2cn(C3CCCOC3)nc2nc1OC1CCC1]

To a solution of 5-Bromo-6-cyclobutoxy-2-(tetrahydro-2H-pyran-3-yl)-2H-pyrazolo[3,4-b]pyridine (700 mg, 1.99 mmol, 1.0 eq.) in MeOH (20 mL) was added TEA (2.01 g, 19.9 mmol, 2.77 mL, 10.0 eq.) and Pd(dppf)Cl₂ (145.4 mg, 198.7 μmol, 0.1 eq.) at 20° C. under Argon. The mixture was stirred at 80° C. under carbon monoxide (50 psi) for 14 hours. LCMS showed 37.0% of the desired product was obtained and 41.7% of the starting material remained. The reaction was evaporated under vacuum to give the residue. The residue was purified by Combi-Flash (PE:EtOAc from 3:1 to 1:1) to give methyl 6-cyclobutoxy-2-(tetrahydro-2H-pyran-3-yl)-2H-pyrazolo[3,4-b]pyridine-5-carboxylate (130 mg, 17.8% yield) as a yellow solid. LCMS: m/z=331.9 [M+H]⁺.

### Preparation 131: 6-Cyclobutoxy-2-(tetrahydro-2H-pyran-3-yl)-2H-pyrazolo[3,4-b]pyridine-5-carboxylic Acid

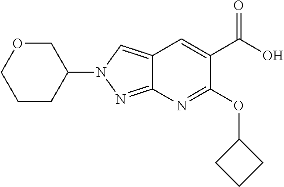 [SMILES: O=C(O)c1cc2cn(C3CCCOC3)nc2nc1OC1CCC1]

To a solution of methyl 6-cyclobutoxy-2-(tetrahydro-2H-pyran-3-yl)-2H-pyrazolo[3,4-b]pyridine-5-carboxylate (130.0 mg, 392.3 μmol, 1.0 eq.) in MeOH (2 mL) and water (2 mL) was added NaOH (31.4 mg, 784.6 μmol, 2.0 eq.) at 20° C. The reaction was stirred at 20° C. for 14 hours. MeOH was evaporated under vacuum. The mixture was acidified with aqueous KHSO₄ to pH<7 and evaporated under vacuum to give 6-cyclobutoxy-2-(tetrahydro-2H-pyran-3-yl)-2H-pyrazolo[3,4-b]pyridine-5-carboxylic acid (110 mg, 79.5% yield) as a white solid. LCMS: m/z=318.3 [M+H]⁺. ¹H NMR: (500 MHz, DMSO-d₆) δ: 1.70-1.61 (m, 2H), 1.81-1.71 (m, 2H), 2.08-1.99 (m, 2H), 2.21-2.16 (m, 2H), 2.44-2.38 (m, 2H), 3.48-3.42 (m, 1H), 3.72 (dd, J₁=10.5 Hz, J₂=9.0 Hz, 1H), 3.85-3.81 (m, 1H), 4.03 (dd, J₁=11.0 Hz, J₂=4.0 Hz, 1H), 4.50-4.43 (m, 1H), 5.17-5.10 (m, 1H), 7.79 (s, 1H), 8.22 (s, 1H).

### Preparation 132: 5-Bromo-6-cyclobutoxy-2-(1-methyl-2-oxabicyclo[2.2.1]heptan-4-yl)-2H-indazole

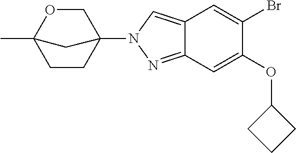 [SMILES: CC12CCC(n3cc4cc(Br)c(OC5CCC5)cc4n3)(CO1)C2]

To a 30 mL vial equipped with a stir bar was added 1-methyl-2-oxabicyclo[2.2.1]heptan-4-amine (203 mg, 1.60 mmol) and iPrOH (8.00 mL). 5-Bromo-4-(cyclobutoxy)-2-nitro-benzaldehyde (400 mg, 1.33 mmol) was added in one portion, followed by TEA (134.6 mg, 1.330 mmol, 185.4 μL). The vial was sealed with a Teflon-lined cap and the resulting yellow solution was heated to 80° C. with stirring for overnight. The mixture was cooled to room temperature and tributylphosphane (807 mg, 3.99 mmol, 996 μL) was added in one portion via a syringe. The vessel was sealed again, and the orange colored solution was stirred at 80° C. for an additional 16 hours. The mixture was cooled to room temperature and diluted with EtOAc (10 mL). The organics were washed with saturated ammonium chloride solution (10 mL), brine (10 ml) and dried over anhydrous Na₂SO₄. The solution was filtered, and the filtrate was concentrated in vacuo. The residue was purified by silica gel (from PE:EA=10:1 to 3:1) to give 5-bromo-6-(cyclobutoxy)-2-(1-methyl-2-oxabicyclo[2.2.1]heptan-4-yl)indazole (240 mg, 40.7% yield) as a yellow solid. LCMS: m/z=379.1 [M+H]⁺.

### Preparation 133: Methyl 6-cyclobutoxy-2-(1-methyl-2-oxabicyclo[2.2.1]heptan-4-yl)-2H-indazole-5-carboxylate

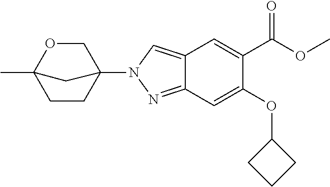

To a solution of 5-bromo-6-(cyclobutoxy)-2-[(1S,4S)-1-methyl-2-oxabicyclo[2.2.1]heptan-4-yl]indazole (165.0 mg, 437.3 μmol) in MeOH (10 mL) was added Pd(dppf)Cl₂ (32.0 mg, 43.7 mol) and TEA (442 mg, 4.37 mmol, 609 μL). The mixture was degassed with CO for 3 times and it was stirred at 80° C. under CO (50 psi) for 16 h. The mixture was concentrated in vacuo to give the residue, which was purified by Combi Flash (PE/EtOAc=1/1) to give methyl 6-(cyclobutoxy)-2-[(1S,4S)-1-methyl-2-oxabicyclo[2.2.1]heptan-4-yl]indazole-5-carboxylate (137 mg, 83.5% yield) as a brown solid. LCMS: m/z=357.5 [M+H]⁺.

### Preparation 134: 6-Cyclobutoxy-2-(1-methyl-2-oxabicyclo[2.2.1]heptan-4-yl)-2H-indazole-5-carboxylic Acid

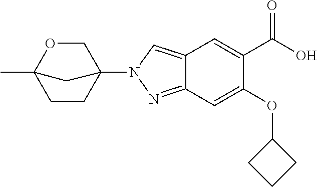 [SMILES: CC12CCC(n3cc4cc(C(=O)O)c(OC5CCC5)cc4n3)(CO1)C2]

To a mixture of methyl 6-(cyclobutoxy)-2-[(1S,4S)-1-methyl-2-oxabicyclo[2.2.1]heptan-4-yl]indazole-5-carboxylate (137 mg, 384 μmol) in MeOH (2 mL) and water (2 mL) was added lithium hydroxide (64.6 mg, 1.54 mmol) in one portion at 15° C. The mixture was stirred at 15° C. for 16 h. The mixture was diluted with saturated HCl aq. till pH=7. The mixture was concentrated in vacuo to give the residue which was re-crystallized from water, dried by lyophilization to afford 6-(cyclobutoxy)-2-[(1S,4S)-1-methyl-2-oxabicyclo[2.2.1]heptan-4-yl]indazole-5-carboxylic acid (130 mg, 353 μmol, 91.9% yield) as a brown solid. LCMS: m/z=343.3 [M+H]⁺.

### Preparation 135: 5-Bromo-6-cyclobutoxy-2-(1-methyl-2-oxabicyclo[2.2.2]octan-4-yl)-2H-indazole

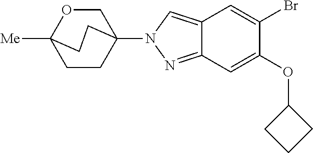 [SMILES: CC12CCC(n3cc4cc(Br)c(OC5CCC5)cc4n3)(CC1)CO2]

To a 100 mL vial equipped with a stir bar was added 5-bromo-4-(cyclobutoxy)-2-nitro-benzaldehyde (3.00 g, 10.0 mmol) and Isopropanol (50 mL). 1-Methyl-2-oxabicyclo[2.2.2]octan-4-amine (1.77 g, 10.0 mmol, hydrochloride) was added in one portion, followed by TEA (1.01 g, 10.0 mmol, 1.39 mL). The vial was sealed with a Teflon-lined cap and the resulting yellow solution was heated to 80° C. with stirring for overnight. The mixture was cooled to room temperature and tributylphosphine (6.27 g, 31.0 mmol, 7.74 mL) was added in one portion via a syringe. The vessel was sealed again, and the orange colored solution was stirred at 80° C. for an additional 16 h. The mixture was cooled to room temperature and diluted with EtOAc (100 mL). The organics were washed with saturated ammonium chloride solution (50 mL), brine (50 ml) and dried over anhydrous Na₂SO₄. The solution was filtered, and the filtrate was concentrated in vacuo to give 5-bromo-6-(cyclobutoxy)-2-(1-methyl-2-oxabicyclo[2.2.2]octan-4-yl)indazole (2.20 g, 5.62 mmol, 56.2% yield) as a white solid. LCMS: m/z=393.0 [M+H]⁺.

### Preparation 136: Methyl 6-cyclobutoxy-2-(1-methyl-2-oxabicyclo[2.2.2]octan-4-yl)-2H-indazole-5-carboxylate

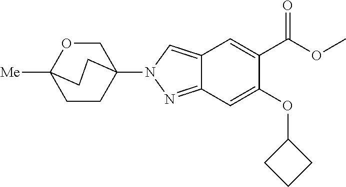 [SMILES: COC(=O)c1cc2cn(C34CCC(C)(CC3)OC4)nc2cc1OC1CCC1]

5-Bromo-6-(cyclobutoxy)-2-(1-methyl-2-oxabicyclo[2.2.2]octan-4-yl)indazole (2.35 g, 6.01 mmol), Pd(dppf)Cl₂ (219.7 mg, 300.3 μmol) and TEA (729 mg, 7.21 mmol, 999 μL) were dissolved in dry MeOH (100.0 mL). The reaction mixture was heated at 150° C. in a pressure vessel at 40 atm. carbon monoxide for 18 hours. The solvent was evaporated and the mixture poured into 50 mL of water. The mixture was extracted with EtOAc (2*50 mL) and the organics were dried over Na₂SO₄ and evaporated to dryness give methyl 6-(cyclobutoxy)-2-(1-methyl-2-oxabicyclo[2.2.2]octan-4-yl)indazole-5-carboxylate (2.15 g, 96.6% yield) as a yellow solid. LCMS: m/z=371.2 [M+H]⁺.

### Preparation 137: 6-Cyclobutoxy-2-(1-methyl-2-oxabicyclo[2.2.2]octan-4-yl)-2H-indazole-5-carboxylic Acid

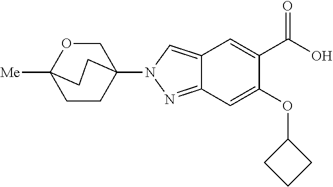 [SMILES: CC12CCC(n3cc4cc(C(=O)O)c(OC5CCC5)cc4n3)(CC1)CO2]

A 250-ml round-bottomed flask, equipped with a magnetic stirrer, was charged with methyl 6-(cyclobutoxy)-2-(1-methyl-2-oxabicyclo[2.2.2]octan-4-yl)indazole-5-carboxylate (2.15 g, 5.80 mmol), lithium hydroxide monohydrate (243.4 mg, 5.80 mmol) in THF (90.00 mL) and water (10.00 mL). The resulting mixture was stirred at r.t. for 48 h. Then, the THF was evaporated in vacuo, H₂O (50 mL) and activated carbon (1 g) were added and the mixture was filtered right away. Then the filtrate was acidified with conc. HCl to pH 3-4 and precipitate was filtered washed with water and air-dried to give compound 6-(cyclobutoxy)-2-(1-methyl-2-oxabicyclo[2.2.2]octan-4-yl)indazole-5-carboxylic acid (2.00 g, 5.50 mmol, 94.8% yield) as a white solid. LCMS: m/z=357.4 [M+H]⁺.

### EXAMPLES

### Example 1: 6-Methoxy-N-(6-methoxypyridin-2-yl)-2-((tetrahydrofuran-3-yl)methyl)-2H-indazole-5-carboxamide Trifluoroacetate

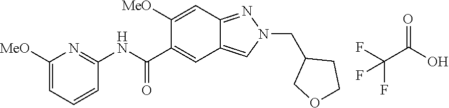

3-(Iodomethyl)tetrahydrofuran (71.1 mg, 0.335 mmol) was added to mixture of 6-methoxy-N-(6-methoxypyridin-2-yl)-1H-indazole-5-carboxamide (Preparation 15, 100 mg, 0.335 mmol) and K₂CO₃ (92.7 mg, 0.670 mmol) in DMF (2 mL) under N₂ at 0° C. The mixture heated at 100° C. overnight. The reaction was cooled, filtered through a pad of Celite® and evaporated to dryness in vacuo and the residue purified by prep HPLC (SunFire C18 column, 60 mL/min flow rate, MeCN/H₂O/0.1% TFA; Gradient (% organic): 10-70) to afford 6-methoxy-N-(6-methoxypyridin-2-yl)-2-((tetrahydrofuran-3-yl)methyl)-2H-indazole-5-carboxamide trifluoroacetate as a white solid (36 mg, 28%). LCMS m/z 383 [M+H]⁺; ¹H NMR (400 MHz, MeOH-d₄) δ: 1.76 (td, 1H), 2.01-2.14 (m, 1H), 2.93-3.06 (m, 1H), 3.65 (dd, 1H), 3.73-3.83 (m, 2H), 3.88-4.00 (m, 4H), 4.12 (s, 3H), 4.46 (d, 2H), 6.58 (d, 1H), 7.16 (s, 1H), 7.72 (t, 1H), 7.87 (br d, 1H), 8.43 (d, 1H), 8.51 (s, 1H)

### Examples 2-6

The title compounds were prepared in an analogous way to that described for Example 1 using the appropriate indazole and alkyl halide as shown in the table below (SunFire C18 column, 60 mL/min flow rate, MeCN/H₂O/0.1% TFA; Gradient (% organic): 10-70):

| Example | Name/Structure/RHal/ | QC Data |
|---|---|---|
| 2 | 6-Methoxy-N-(pyridin-2-yl)-2- | White solid (27 mg, 25%). |
| | ((tetrahydrofuran-3-yl)methyl)-2H- | LCMS m/z = 353 [M + H]⁺ |
| | indazole-5-carboxamide | ¹H NMR (400 MHz, MeOH-d₄) δ: |
| | trifluoroacetate | 1.72-1.83 (m, 1H), 2.04-2.12 (m, |
| | | 1H), 2.95-3.06 (m, 1H), 3.65 (dd, |
| | 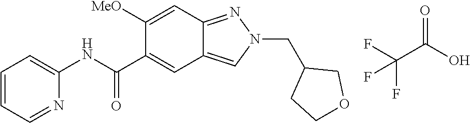 | 1H), 3.76-3.83 (m, 2H), 3.92-3.98 (m, 1H), 4.11-4.15 (m, 3H), 4.47 (d, 2H), 7.20 (s, 1H), 7.53 (td, 1H), 8.01 (d, 1H), 8.31 (ddd, 1H), 8.44 (dd, 1H), 8.47 (s, 1H), 8.52-8.58 (m, 1H). |
| | RHal: 3-(iodomethyl)tetrahydrofuran | |
| | Indazole: 6-methoxy-N-(pyridin-2- | |
| | yl)-1H-indazole-5-carboxamide | |
| | (Preparation 16) | |
| 3 | 6-Methoxy-N-(6-methoxypyridin-2- | White solid (19 mg, 30%). |
| | yl)-2-((1-methyl-2- | LCMS m/z = 409 [M + H]⁺ |
| | oxabicyclo[2.1.1]hexan-4- | ¹H NMR (400 MHz, MeOH-d₄) δ: |
| | yl)methyl)-2H-indazole-5- | 1.29-1.43 (m, 3H), 1.53-1.63 (m, |
| | carboxamide trifluoroacetate | 2H), 1.63-1.74 (m, 2H), 3.32-3.38 |
| | | (m, 1H), 3.66 (d, 2H), 4.01 (d, 3H), |
| | 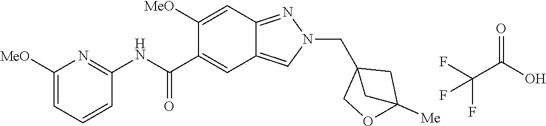 | 4.07-4.19 (m, 3H), 6.76 (dd, 1H), 7.20 (d, 1H), 7.70-7.84 (m, 1H), 7.87-7.97 (m, 1H), 8.52 (d, 1H, 8.58 (d, 1H). |
| | RHal: 4-(bromoethyl)-1-methyl-2- | |
| | oxabicyclo[2.1.1]hexane | |
| | Indazole: 6-methoxy-N-(6- | |
| | methoxypyridin-2-yl)-1H-indazole- | |
| | 5-carboxamide (Preparation 15) | |
| 4 | 6-Methoxy-N-(6-methoxypyridin-2- | White solid (10 mg, 18%). |
| | yl)-2-(tetrahydrofuran-3-yl)-2H- | LCMS m/z = 369 [M + H]⁺ |
| | indazole-5-carboxamide | ¹H NMR (400 MHz, MeOH-d₄) δ: |
| | trifluoroacetate | 2.40-2.52 (m, 1H), 2.56-2.70 (m, |
| | | 1H), 3.91 (s, 3H), 3.97 (td, 1H), 4.10 |
| | 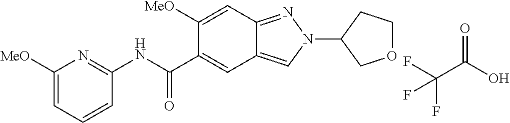 | (s, 3H), 4.13-4.26 (m, 3H), 5.32 (td, 1H), 6.55 (d, 1H), 7.16 (s, 1H), 7.69 (t, 1H), 7.87 (br d, 1H), 8.43 (s, 1H), 8.50 (s, 1H). |
| | RHal: 3-iodotetrahydrofuran | |
| | Indazole: 6-methoxy-N-(6- | |
| | methoxypyridin-2-yl)-1H-indazole- | |
| | 5-carboxamide (Preparation 15) | |
| 5 | 6-Methoxy-N-(6-methoxypyridin-2- | White solid (3 mg, 3.6%). |
| | yl)-2-(tetrahydro-2H-pyran-4-yl)- | LCMS m/z = 383 [M + H]⁺ |
| | 2H-indazole-5-carboxamide | ¹H NMR (400 MHz, MeOH-d₄) δ: |
| | trifluoroacetate | 2.17-2.31 (m, 4H), 3.66 (td, 2H), |
| | | 3.90-3.93 (m, 3H), 4.09-4.18 (m, |
| | 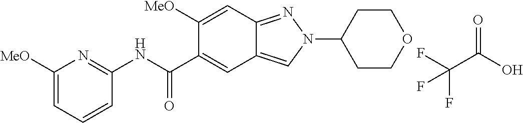 | 6H), 4.69-4.77 (m, 1H), 6.54-6.60 (m, 1H), 7.17 (s, 1H), 7.68-7.75 (m, 1H), 7.88 (br d, 1H), 8.46 (s, 1H), 8.53 (s, 1H). |
| | RHal: 4-iodotetrahydropyran | |
| | Indazole: 6-methoxy-N-(6- | |
| | methoxypyridin-2-yl)-1H-indazole- | |
| | 5-carboxamide (Preparation 15) | |
| 6 | 6-Methoxy-N-(pyridin-2-yl)-2- | White solid (1.4 mg, 2.3%). |
| | (tetrahydro-2H-pyran-4-yl)-2H- | LCMS m/z = 353 [M + H]⁺ |
| | indazole-5-carboxamide | ¹H NMR (400 MHz, MeOH-d₄) δ: |
| | trifluoroacetate | 2.17-2.33 (m, 4H), 3.63-3.70 (m, |
| | | 2H), 4.15 (s, 5H), 4.69-4.77 (m, 1H), |
| | 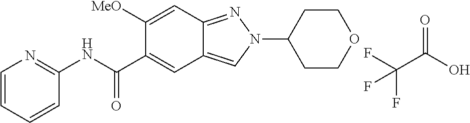 | 7.20 (s, 1H), 7.32-7.38 (m, 1H), 8.05-8.09 (m, 1H), 8.23 (d, 1H), 8.39 (br d, 1H), 8.50 (s, 1H), 8.59 (s, 1H). |
| | RHal: 4-iodotetrahydropyran | |
| | Indazole: 6-methoxy-N-(pyridin-2- | |
| | yl)-1H-indazole-5-carboxamide | |
| | (Preparation 16) | |

### Example 7: N-(6-methoxypyridin-2-yl)-2-((tetrahydrofuran-3-yl)methyl)-2H-pyrazolo[3,4-c]pyridine-5-carboxamide Trifluoroacetate

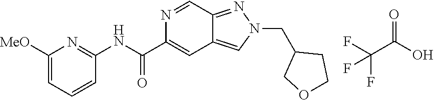

### Part A.

3-(Iodomethyl)tetrahydrofuran (1.20 g, 5.64 mmol) was added to a mixture of methyl 1H-pyrazolo[3,4-c]pyridine-5-carboxylate (500 mg, 2.82 mmol) and K₂CO₃ (780 mg, 5.64 mmol) in DMF (7 mL) under Ar and the reaction mixture heated at 100° C. for 18 h. The cooled reaction was diluted with brine and extracted with EtOAc. The combined organics were washed (brine), dried (Na₂SO₄) and evaporated to dryness in vacuo to afford a mixture of regioisomers (400 mg, 54%) which was used without any further purification in Part B.

### Part B.

DABAL-Me₃ (334 mg, 1.30 mmol) was added to the mixture of Part A (200 mg, 0.765 mmol) and 6-methoxypyridin-2-amine (143 mg, 1.15 mmol) in THF (8 mL) and the mixture stirred at rt overnight. The reaction was quenched with MeOH, followed by addition of EtOAc and Na₂SO₄. The resulting mixture was filtered and evaporated to dryness in vacuo and the residue purified using prep-HPLC (SunFire C18 column, 60 mL/min flow rate, MeCN/H₂O/0.1% TFA; Gradient (% organic): 5-95) to afford N-(6-methoxypyridin-2-yl)-2-((tetrahydrofuran-3-yl)methyl)-2H-pyrazolo[3,4-c]pyridine-5-carboxamide trifluoroacetate (17.3 mg, 4.9%). LCMS m/z=354 [M+H]⁺; ¹H NMR (400 MHz, MeOH-d₄) δ: 1.71-1.85 (m, 1H), 2.03-2.15 (m, 1H), 2.98-3.09 (m, 1H), 3.67 (dd, 1H), 3.74-3.85 (m, 2H), 3.94 (s, 4H), 4.62 (d, 2H), 6.58 (d, 1H), 7.72 (t, 1H), 7.91 (d, 1H), 8.64-8.69 (m, 2H), 9.24 (s, 1H).

### Example 8: N-(6-(difluoromethyl)pyridin-2-yl)-2-((tetrahydrofuran-3-yl)methyl)-2H-pyrazolo[3,4-c]pyridine-5-carboxamide Trifluoroacetate

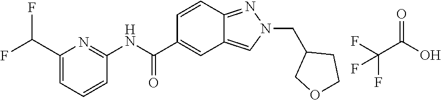

N-(6-(difluoromethyl)pyridin-2-yl)-2-((tetrahydrofuran-3-yl)methyl)-2H-pyrazolo[3,4-c]pyridine-5-carboxamide trifluoroacetate (21 mg, 5.9%) was prepared using an analogous method to that described for Example 7 using 6-(difluoromethyl)pyridin-2-amine in Part B. Purified by prep-HPLC (SunFire C18 column, 60 mL/min flow rate, MeCN/H₂O/0.1% TFA; Gradient (% organic): 10-70). ¹H NMR (500 MHz, MeOH-d₄) δ: 1.78 (td, 1H), 2.00-2.14 (m, 1H), 2.97-3.09 (m, 1H), 3.67 (dd, 2H), 3.73-3.87 (m, 3H), 3.93 (br d, 1H), 4.62 (d, 2H), 6.49-6.83 (m, 1H), 7.45 (d, 1H), 8.02 (t, 1H), 8.53 (d, 1H), 8.66 (s, 2H), 9.23 (s, 1H).

### Example 9: N-(6-methoxypyridin-2-yl)-7-methyl-2-((tetrahydrofuran-3-yl)methyl)-2H-indazole-5-carboxamide Trifluoroacetate

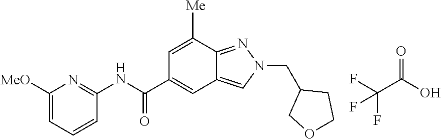

### Part A.

3-(Iodomethyl)tetrahydrofuran (223 mg, 1.05 mmol) was added to a mixture of methyl 7-methyl-1H-indazole-5-carboxylate (200 mg, 1.05 mmol) and K₂CO₃ (290 mg, 2.10 mmol) in DMF (7 mL) under Ar and the reaction mixture heated at 100° C. for 18 h. The cooled reaction was diluted with brine and extracted with EtOAc (4×10 mL). The combined organics were washed (brine), dried (Na₂SO₄) and evaporated to dryness in vacuo to afford a mixture of regioisomers which was used without any further purification in Part B.

### Part B.

DABAL-Me₃ (222 mg, 0.87 mmol) was added to the mixture of Part A and 6-methoxypyridin-2-amine (95 mg, 0.77 mmol) in THF (5 mL) and the mixture stirred at rt overnight. The reaction was quenched with H₂O, followed by addition of NaHCO₃ to basify the mixture and extracted with EtOAc (2×). The combined extracts were dried (Na₂SO₄) and evaporated to dryness in vacuo and the residue purified by prep-HPLC (SunFire C18 column, 60 mL/min flow rate, MeCN/H₂O/0.1% TFA; Gradient (% organic): 5-95) to afford N-(6-methoxypyridin-2-yl)-7-methyl-2-((tetrahydrofuran-3-yl)methyl)-2H-indazole-5-carboxamide trifluoroacetate (1.2 mg, 0.64%). ¹H NMR (500 MHz, MeOH-d₄) δ: 1.73-1.84 (m, 1H), 1.99-2.08 (m, 1H), 2.85-2.92 (m, 1H), 3.61-3.70 (m, 2H), 3.72-3.80 (m, 1H) 3.93 (s, 3H), 3.94-3.99 (m, 1H), 4.60-4.68 (m, 2H), 6.55 (d, 1H), 7.69 (t, 1H), 7.77 (s, 1H), 7.80 (d, 1H), 8.16 (s, 1H), 8.29 (s, 1H).

### Example 10: N-(6-(difluoromethyl)pyridin-2-yl)-6-methoxy-2-(2-methoxyethyl)-2H-indazole-5-carboxamide

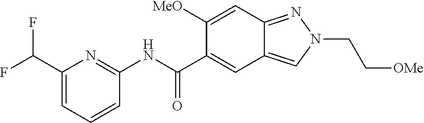 [SMILES: COCCn1cc2cc(C(=O)Nc3cccc(C(F)F)n3)c(OC)cc2n1]

Part 1. 1-Iodo-2-methoxy-ethane (675 mg, 3.63 mmol) was added to a solution of methyl 6-methoxy-1H-indazole-5-carboxylate (Preparation 5, 500 mg, 2.42 mmol) and K₂CO₃ (501.70 mg, 3.63 mmol) in DMF (5.00 mL) at 0° C. under Ar. The resulting mixture was heated at 100° C. for 24 h. The cooled mixture was diluted with H₂O (25 mL) and extracted with EtOAc (4×10 mL). The combined organics were washed with H₂O (25 mL), brine (25 mL), dried (Na₂SO₄) and evaporated to dryness in vacuo to afford a mixture of methyl 6-methoxy-1-(2-methoxyethyl)-1H-indazole-5-carboxylate and methyl 6-methoxy-2-(2-methoxyethyl)-2H-indazole-5-carboxylate as yellow oil which was used without further purification. LCMS m/z=265 [M+H]⁺.
Part 2. A mixture of methyl 6-methoxy-1-(2-methoxyethyl)-1H-indazole-5-carboxylate and methyl 6-methoxy-2-(2-methoxyethyl)-2H-indazole-5-carboxylate (Part 1; 600 mg, 2.46 mmol) and K₂CO₃ (622 mg, 4.50 mmol) in H₂O (5 mL) and MeOH (2 mL) was stirred at rt for 24 h. The reaction mixture was evaporated in vacuo diluted with H₂O and treated with activated carbon. The solids were removed by filtration and the filtrate acidified to pH 4-5 by the addition of c. HCl. The resulting precipitate was removed by filtration, washed with H₂O and air-dried to give a mixture of 6-methoxy-1-(2-methoxyethyl)-1H-indazole-5-carboxylic acid and 6-methoxy-2-(2-methoxyethyl)-2H-indazole-5-carboxylic acid (550 mg, 89%) as a white solid which was used in Part 3 without further purification. LCMS m/z=251 [M+H]⁺.
Part 3. CDI (428 mg, 2.64 mmol) was added to an isomeric mixture of 6-methoxy-1-(2-methoxyethyl)-1H-indazole-5-carboxylic acid and 6-methoxy-2-(2-methoxyethyl)-2H-indazole-5-carboxylic acid (2.40 mmol) in dioxane (10 mL) and stirred at rt for 1 hour. 6-(difluoromethyl)pyridin-2-amine (346 mg, 2.40 mmol) was added to the mixture and the reaction stirred at 80° C. overnight. The reaction mixture was poured into water and extracted with EtOAc. The combined organics were washed with H₂O, NaHCO₃, dried (Na₂SO₄) and evaporated to dryness in vacuo. The residue was purified by preparative-HPLC (XBridge C18 100*19 mm 5 μm; 0.1% NH₄OH-MeOH; % organic: 40-65) to afford N-(6-(difluoromethyl)pyridin-2-yl)-6-methoxy-2-(2-methoxyethyl)-2H-indazole-5-carboxamide (18.0 mg, 1.97%). LCMS m/z=377 [M+H]⁺; ¹H NMR (500 MHz, CDCl₃) δ: 3.35 (s, 3H), 3.87-3.90 (m, 2H), 4.13 (s, 3H), 4.54-4.57 (m, 2H), 6.56 (t, 1H), 7.13 (s, 1H), 7.38 (d, 1H), 7.88 (t, 1H), 8.13 (s, 1H), 8.53-8.59 (m, 1H), 8.73 (s, 1H), 10.48 (s, 1H).

### Example 11: N-(6-(difluoromethyl)pyridin-2-yl)-6-methoxy-2-((tetrahydrofuran-3-yl)methyl)-2H-indazole-5-carboxamide

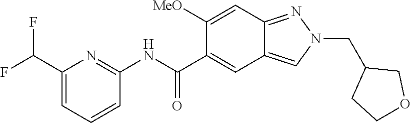 [SMILES: COc1cc2nn(CC3CCOC3)cc2cc1C(=O)Nc1cccc(C(F)F)n1]

Part 1. 3-Bromomethyltetrahydrofuran (600 mg, 3.63 mmol) was added to a solution of methyl 6-methoxy-1H-indazole-5-carboxylate (Preparation 5, 500 mg, 2.42 mmol) and K₂CO₃ (502 mg, 3.63 mmol) in DMF (5.00 mL) at 0° C. under Ar. The resulting mixture was heated at 100° C. for 24 h. The cooled mixture was diluted with
H₂O (25 mL) and extracted with EtOAc (4×10 mL). The combined organics were washed with H₂O (25 mL), brine (25 mL), dried (Na₂SO₄) and evaporated to dryness in vacuo to afford a mixture of 6-methoxy-1-((tetrahydrofuran-3-yl)methyl)-1H-indazole-5-carboxylate and methyl 6-methoxy-2-((tetrahydrofuran-3-yl)methyl)-2H-indazole-5-carboxylate as yellow oil (620 mg) which was used without further purification. LCMS m/z=291 [M+H]⁺.
Part 2. A mixture of methyl 6-methoxy-1-((tetrahydrofuran-3-yl)methyl)-1H-indazole-5-carboxylate and methyl 6-methoxy-2-((tetrahydrofuran-3-yl)methyl)-2H-indazole-5-carboxylate (Part 1; 620 mg, 2.46 mmol) and K₂CO₃ (774 mg, 5.6 mmol) in H₂O (5 mL) and MeOH (2 mL) was stirred at rt for 24 h. The reaction mixture was evaporated to dryness in vacuo, diluted with H₂O and treated with activated carbon. The solids were removed by filtration and the filtrate acidified to pH 4-5 by the addition of c. HCl. The resulting precipitate was removed by filtration, washed (H₂O) and air-dried to give a mixture of 6-methoxy-1-((tetrahydrofuran-3-yl)methyl)-1H-indazole-5-carboxylic acid and 6-methoxy-2-((tetrahydrofuran-3-yl)methyl)-2H-indazole-5-carboxylic acid (580 mg) as a white solid which was used in Part 3 without further purification. LCMS m/z=277 [M+H]⁺.
Part 3. HATU (993 mg, 2.60 mmol) and DIPEA was added to an isomeric mixture of 6-methoxy-1-(2-methoxyethyl)-1H-indazole-5-carboxylic acid and 6-methoxy-2-(2-methoxyethyl)-2H-indazole-5-carboxylic acid (580 mg, 2.17 mmol) in DMF (10 mL) followed by the addition of 6-(difluoromethyl)pyridin-2-amine (313 mg, 2.17 mmol) the reaction stirred at 30° C. for 14 h. The reaction mixture was poured into H₂O (20 mL) and extracted with EtOAc (4×25 mL). The combined organics were washed with H₂O (50 mL), brine (50 mL), dried (Na₂SO₄) and evaporated to dryness in vacuo. The residue was purified by HPLC (Sunfire C18 100*19 mm 5 μm; H₂O-MeOH; % organic: 40-60) to afford N-(6-(difluoromethyl)pyridin-2-yl)-6-methoxy-2-((tetrahydrofuran-3-yl)methyl)-2H-indazole-5-carboxamide (13.5 mg, 1.6%). LCMS m/z=403 [M+H]⁺; ¹H NMR (500 MHz, CDCl₃) δ: 1.61-1.74 (m, 1H), 2.01-2.13 (m, 1H), 2.96-3.08 (m, 1H), 3.54-3.64 (m, 1H), 3.69-3.81 (m, 2H), 3.83-3.94 (m, 1H), 4.10 (s, 3H), 4.30-4.41 (m, 2H), 6.56 (t, 1H), 7.1 (s, 1H), 7.35 (d, 1H), 7.85 (t, 1H), 8.01 (s, 1H), 8.53 (d, 1H), 8.69 (s, 1H), 10.43 (s, 1H).

### Example 12: N-(6-(difluoromethyl)pyridin-2-yl)-6-methoxy-2-(3-methoxy-3-methylbutyl)-2H-indazole-5-carboxamide

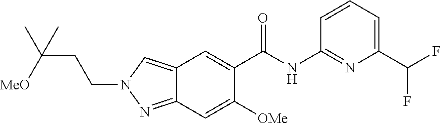 [SMILES: COc1cc2nn(CCC(C)(C)OC)cc2cc1C(=O)Nc1cccc(C(F)F)n1]

N-(6-(difluoromethyl)pyridin-2-yl)-6-methoxy-2-(3-methoxy-3-methylbutyl)-2H-indazole-5-carboxamide was prepared in an analogous way to Example 11 using methyl 6-methoxy-1H-indazole-5-carboxylate (Preparation 5), 1-bromo-3-methoxy-3-methylbutane and 6-(difluoromethyl)pyridin-2-amine. Preparative HPLC: XBridge C18 100*19 mm 5 μm; 0.1% NH₄OH-MeOH; % organic: 50-75) LCMS m/z=419 [M+H]⁺; ¹H NMR (500 MHz, CDCl₃) δ: 1.25 (s, 6H), 2.22-2.25 (m, 2H), 3.25 (s, 3H), 4.12 (s, 3H), 4.47-4.50 (m, 2H), 6.56 (t, 1H), 7.14 (s, 1H), 7.38 (d, 1H), 7.88 (t, 1H), 8.06 (s, 1H), 8.57 (d, 1H), 8.71 (s, 1H), 10.49 (s, 1H).

### Example 13: N-(6-(difluoromethyl)pyridin-2-yl)-2-(3-hydroxy-3-methylbutyl)-6-isopropoxy-2H-indazole-5-carboxamide

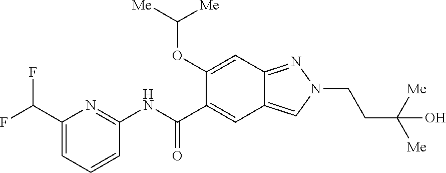 [SMILES: CC(C)Oc1cc2nn(CCC(C)(C)O)cc2cc1C(=O)Nc1cccc(C(F)F)n1]

K₂CO₃ (79.8 mg, 0.577 mmol) was added to a solution of N-(6-(difluoromethyl)pyridin-2-yl)-6-isopropoxy-2H-indazole-5-carboxamide (Preparation 20, 100 mg, 0.289 mmol) and 4-bromo-2-methylbutan-2-ol (57.9 mg, 0.346 mmol) in DMF (2 mL) and the mixture heated at 110° C. for 16 h. The reaction mixture was filtered, and the filtrate purified by prep-HPLC (Column: Welch Xtimate C18 150×30 mm×5 μm; Mobile Phase: 40-70% H₂O (10 mM, NH₄HCO₃)−MeCN) to afford N-(6-(difluoromethyl)pyridin-2-yl)-2-(3-hydroxy-3-methylbutyl)-6-isopropoxy-2H-indazole-5-carboxamides a white solid (23.5 mg, 18.7%). LCMS m/z=433 [M+H]⁺; ¹H NMR (500 MHz, MeOH-d₄) δ: 1.28 (s, 6H), 1.57 (d, 6H), 2.10-2.20 (m, 2H), 4.50-4.63 (m, 2H), 4.96 (dt, 1H), 6.45-6.72 (m, 1H), 7.15 (s, 1H), 7.41 (d, 1H), 7.98 (t, 1H), 8.40 (s, 1H), 8.45 (d, 1H), 8.61 (s, 1H).

### Examples 14-17

The title compounds were prepared in an analogues manner to that described for Example 13 using the appropriate indazole and appropriate alkylating agent and purified by prep-HPLC [Column: Phenomenex Synergi C18 150×30 mm×4 μm; MeCN/H₂O (0.05% HCl)] using the gradient shown in the following table:

| Example | Structure/Name/Reactants/HPLC Conditions | Yield/Data |
|---|---|---|
| 14 | 2-(3-Hydroxy-3-methylbutyl)-7-methoxy-N-(6- | Yellow solid (7.8 mg, |
| | methoxypyridin-2-yl)-2H-indazole-5- | 19.6%) LCMS m/z = 385 |
| | carboxamide hydrochloride | [M + H]⁺ ¹H NMR (500 |
| | | MHz, MeOH-d₄) δ: 1.28 |
| | 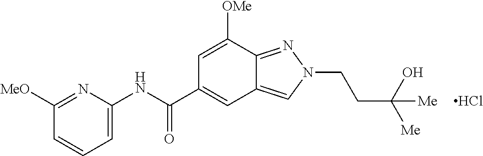 [SMILES: COc1cccc(NC(=O)c2cc(OC)c3nn(CCC(C)(C)O)cc3c2)n1 ; Cl] | (s, 6H), 2.19 (t, 2H), 4.02 (s, 3H), 4.10 (s, 3H), 4.63 (t, 2H), 6.74 (d, 1H), 7.28 (s, 1H), 7.69 (d, 1H), 7.88-7.92 (m, 1H), 8.09 (s, 1H), 8.52 (s, 1H). |
| | Indazole: 7-methoxy-N-(6-methoxypyridin-2- | |
| | yl)-2H-indazole-5-carboxamide (Preparation 22) | |
| | RX: 4-bromo-2-methylbutan-2-ol | |
| | Gradient (% organic): 34-64 | |
| 15 | 7-Methoxy-2-(3-methoxy-3-methylbutyl)-N-(6- | White solid (6.4 mg, |
| | methoxypyridin-2-yl)-2H-indazole-5- | 15.7%) LCMS m/z = 399 |
| | carboxamide hydrochloride | [M + H]⁺ ¹H NMR (500 |
| | | MHz, MeOH-d₄) δ: 1.26 |
| | 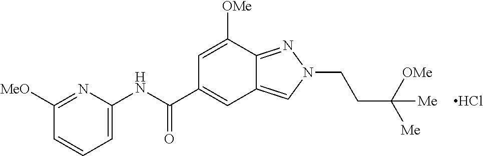 [SMILES: COc1cccc(NC(=O)c2cc(OC)c3nn(CCC(C)(C)OC)cc3c2)n1 ; Cl] | (s, 6H), 2.23 (t, 2H), 3.22 (s, 3H), 4.05 (s, 3H), 4.10 (s, 3H), 4.59 (t, 2H), 7.30 (s, 1H), 6.80 (d, 1H), 7.65 (d, 1H), 7.95-7.99 (m, 1H), 8.11 (s, 1H), 8.59 (s, 1H). |
| | Indazole: 7-methoxy-N-(6-methoxypyridin-2- | |
| | yl)-2H-indazole-5-carboxamide (Preparation 22) | |
| | RX: 3-methoxy-3-methylbutyl 4- | |
| | methylbenzenesulfonate | |
| | Gradient (% organic): 45-75 | |
| 16 | 7-Methoxy-N-(6-methoxypyridin-2-yl)-2- | White solid (14.4 mg, |
| | (tetrahydro-2H-pyran-4-yl)-2H-indazole-5- | 22%) LCMS m/z = 383 |
| | carboxamide hydrochloride | [M + H]⁺ ¹H NMR (500 |
| | | MHz, MeOH-d₄) δ: 2.17- |
| | 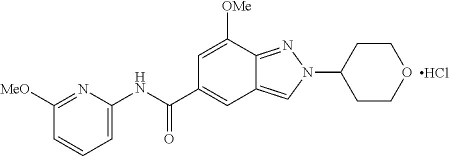 [SMILES: COc1cccc(NC(=O)c2cc(OC)c3nn(C4CCOCC4)cc3c2)n1 ; Cl] | 2.21 (m, 2H), 2.24-2.33 (m, 2H), 3.62-3.67 (m, 2H), 4.03 (s, 3H), 4.10 (s, 3H), 4.11-4.15 (m, 2H), 4.74-4.82 (m, 1H), 6.75 (d, 1H), 7.24 (s, 1H), 7.68 (d, 1H), 7.90-7.94 (m, 1H), 8.09 (s, 1H), 8.56 (s, 1H). |
| | Indazole: 7-methoxy-N-(6-methoxypyridin-2- | |
| | yl)-2H-indazole-5-carboxamide (Preparation 22) | |
| | RX: tetrahydro-2H-pyran-4-yl 4- | |
| | methylbenzenesulfonate | |
| | Gradient (% organic): 34-64 | |
| 17 | N-(6-(difluoromethyl)pyridin-2-yl)-6-methoxy- | White solid (21.8 mg, |
| | 2-(3-methoxypropyl)-2H-indazole-5- | 51%) LCMS m/z = 391 |
| | carboxamide hydrochloride | [M + H]⁺ ¹H NMR (500 |
| | | MHz, MeOH-d₄) δ: 2.25 |
| | 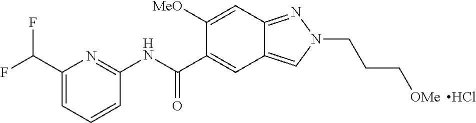 [SMILES: COCCCn1cc2cc(C(=O)Nc3cccc(C(F)F)n3)c(OC)cc2n1 ; Cl] | (q, 2H), 3.32 (s, 3H), 3.39 (t, 2H), 4.12 (s, 3H), 4.58 (t, 2H), 6.53-6.77 (m, 1H), 7.18 (s, 1H), 7.44 (d, 1H), 8.00 (dd, 1H), 8.48 (d, 1H), 8.51 (s, 1H), 8.53 (s, 1H). |
| | Indazole: N-(6-(difluoromethyl)pyridin-2-yl)-6- | |
| | methoxy-2H-indazole-5-carboxamide | |
| | (Preparation 21) | |
| | RX: 1-bromo-3-methoxypropane | |
| | Gradient (% organic): 49-69 | |

### Examples 18 and 19: (R)—N-(6-(difluoromethyl)pyridin-2-yl)-6-methoxy-2-((tetrahydrofuran-3-yl)methyl)-2H-indazole-5-carboxamide and (S)—N-(6-(difluoromethyl)pyridin-2-yl)-6-methoxy-2-((tetrahydrofuran-3-yl)methyl)-2H-indazole-5-carboxamide

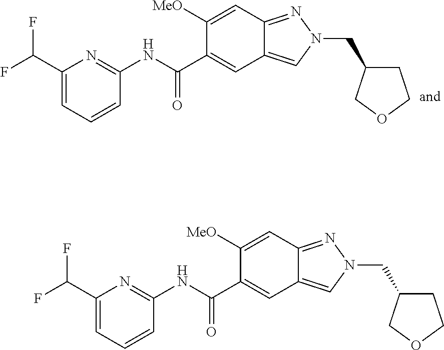 [SMILES: COc1cc2nn(C[C@H]3CCOC3)cc2cc1C(=O)Nc1cccc(C(F)F)n1 ; COc1cc2nn(C[C@@H]3CCOC3)cc2cc1C(=O)Nc1cccc(C(F)F)n1]

### Absolute Stereochemistry Arbitrarily Assigned

(R)—N-(6-(difluoromethyl)pyridin-2-yl)-6-methoxy-2-((tetrahydrofuran-3-yl)methyl)-2H-indazole-5-carboxamide and (S)—N-(6-(difluoromethyl)pyridin-2-yl)-6-methoxy-2-((tetrahydrofuran-3-yl)methyl)-2H-indazole-5-carboxamide were obtained by SFC separation of N-(6-(difluoromethyl)pyridin-2-yl)-6-methoxy-2-((tetrahydrofuran-3-yl)methyl)-2H-indazole-5-carboxamide (Example 11) (Column: Phenomenex-Cellulose-2 (250 mm×30 mm, 5 μm); Mobile Phase: 45% of 0.1% NH₄OH/IPA).
Peak 1: White solid; LCMS m/z=403 [M+H]⁺; ¹H NMR (400 MHz, MeOH-d₄) δ: 1.71-1.80 (m, 1H), 2.03-2.13 (m, 1H), 2.96-3.02 (m, 1H), 3.62-3.66 (m, 1H), 3.75-3.82 (m, 2H), 3.91-3.97 (m, 1H), 4.12 (s, 3H), 4.44 (d, 2H), 6.51-6.80 (m, 1H), 7.16 (s, 1H), 7.43 (d, 1H), 7.99 (t, 1H), 8.41 (s, 1H), 8.49 (d, 1H), 8.57 (s, 1H).
Peak 2: White solid; LCMS m/z=403 [M+H]⁺; ¹H NMR (400 MHz, MeOH-d₄) δ: 1.71-1.80 (m, 1H), 2.03-2.13 (m, 1H), 2.96-3.02 (m, 1H), 3.62-3.66 (m, 1H), 3.75-3.82 (m, 2H), 3.91-3.97 (m, 1H), 4.12 (s, 3H), 4.45 (d, 2H), 6.51-6.80 (m, 1H), 7.16 (s, 1H), 7.44 (d, 1H), 8.00 (t, 1H), 8.42 (s, 1H), 8.49 (d, 1H), 8.54 (s, 1H).

### Example 20 and 21: (R)—N-(6-(difluoromethyl)pyridin-2-yl)-6-isopropoxy-2-((tetrahydrofuran-3-yl)methyl)-2H-indazole-5-carboxamide and (S)—N-(6-(difluoromethyl)pyridin-2-yl)-6-isopropoxy-2-((tetrahydrofuran-3-yl)methyl)-2H-indazole-5-carboxamide

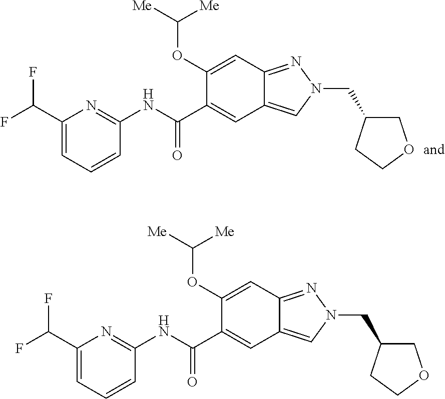 [SMILES: CC(C)Oc1cc2nn(C[C@H]3CCOC3)cc2cc1C(=O)Nc1cccc(C(F)F)n1 ; CC(C)Oc1cc2nn(C[C@@H]3CCOC3)cc2cc1C(=O)Nc1cccc(C(F)F)n1]

### Absolute Stereochemistry Arbitrarily Assigned

K₂CO₃ (160 mg, 1.15 mmol) was added to a solution of N-(6-(difluoromethyl)pyridin-2-yl)-6-isopropoxy-2H-indazole-5-carboxamide (Preparation 20, 200 mg, 0.577 mmol) and (tetrahydrofuran-3-yl)methyl methanesulfonate (J Med Chem, 2018, 145, 770-789, 135.3 mg, 0.751 mol) in DMF (3 mL) and the mixture heated to 95° C. for 16 h. The reaction mixture was filtered and the filtrate purified by prep-HPLC (Column: Phenomenex Synergi C18 150×30 mm×4 μm; 49%-69% of water (0.05% HCl)−MeCN) to give an enantiomeric mixture of Examples 20 and 21 which was separated by SFC (Column: Phenomenex-Cellulose-2 250 mm×30 mm×5 μm; Mobile Phase: 45% of 0.1% NH4OH/IPA) to afford (R)—N-(6-(difluoromethyl)pyridin-2-yl)-6-isopropoxy-2-((tetrahydrofuran-3-yl)methyl)-2H-indazole-5-carboxamide and (S)—N-(6-(difluoromethyl)pyridin-2-yl)-6-isopropoxy-2-((tetrahydrofuran-3-yl)methyl)-2H-indazole-5-carboxamide as white solids.
Peak 1: (23 mg, 9.3%, RT=6.328 min); LCMS m/z=431 [M+H]⁺; ¹H NMR (500 MHz, MeOH-d₄) δ: 8.63 (s, 1H), 8.40-8.50 (m, 2H), 7.99 (t, 1H), 7.42 (d, 1H), 7.17 (s, 1H), 6.40-6.70 (m, 1H), 4.90-5.00 (m, 1H), 4.45 (d, 2H), 3.90-4.00 (m, 1H), 3.70-3.80 (m, 2H), 3.30-3.40 (m, 1H), 2.95-3.05 (m, 1H), 2.00-2.10 (m, 1H), 1.65-1.75 (m, 1H), 1.50-1.60 (m, 6H).
Peak 2: (25 mg, 10%, RT=6.741 min); LCMS m/z=431 [M+H]⁺; ¹H NMR (500 MHz, MeOH-d₄) 8.63 (s, 1H), 8.40-8.50 (m, 2H), 7.99 (t, 1H), 7.42 (d, 1H), 7.17 (s, 1H), 6.40-6.70 (m, 1H), 4.90-5.00 (m, 1H), 4.45 (d, 2H), 3.90-4.00 (m, 1H), 3.70-3.80 (m, 2H), 3.30-3.40 (m, 1H), 2.95-3.05 (m, 1H), 2.00-2.10 (m, 1H), 1.65-1.75 (m, 1H), 1.50-1.60 (m, 6H).

### Example 22 and 23: (S)-6-Methoxy-N-(6-methoxypyridin-2-yl)-2-((tetrahydrofuran-3-yl)methyl)-2H-indazole-5-carboxamide and (R)-6-methoxy-N-(6-methoxypyridin-2-yl)-2-((tetrahydrofuran-3-yl)methyl)-2H-indazole-5-carboxamide

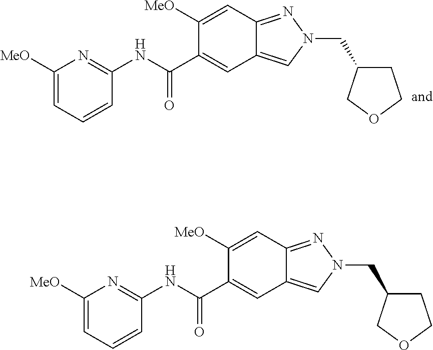 [SMILES: COc1cccc(NC(=O)c2cc3cn(C[C@@H]4CCOC4)nc3cc2OC)n1 ; COc1cccc(NC(=O)c2cc3cn(C[C@H]4CCOC4)nc3cc2OC)n1]

### Absolute Stereochemistry Arbitrarily Assigned

(S)-6-Methoxy-N-(6-methoxypyridin-2-yl)-2-((tetrahydrofuran-3-yl)methyl)-2H-indazole-5-carboxamide and (R)-6-methoxy-N-(6-methoxypyridin-2-yl)-2-((tetrahydrofuran-3-yl)methyl)-2H-indazole-5-carboxamide were obtained by SFC separation (Column: Chiralpak AD-H 250 mm×30 mm, 5 μm; Mobile Phase: 40% EtOH+0.1% DEA in CO₂) of 6-methoxy-N-(6-methoxypyridin-2-yl)-2-((tetrahydrofuran-3-yl)methyl)-2H-indazole-5-carboxamide trifluoroacetate (Example 1).
Peak 1: LCMS m/z=383 [M+H]⁺; ¹H NMR (400 MHz, MeOH-d₄) δ: 1.63-1.67 (m, 1H), 1.92-1.98 (m, 1H), 2.83-2.91 (m, 1H), 3.53-3.55 (m, 1H), 3.66-3.70 (m, 2H), 3.80-3.85 (m, 4H), 4.01 (s, 3H), 4.33 (d, 2H), 6.44 (d, 1H), 7.04 (s, 1H), 7.58 (t, 1H), 7.76 (d, 1H), 8.29 (s, 1H), 8.39 (s, 1H).
Peak 2: LCMS m/z=383 [M+H]⁺; ¹H NMR (400 MHz, MeOH-d₄) δ: 1.63-1.67 (m, 1H), 1.92-1.98 (m, 1H), 2.83-2.91 (m, 1H), 3.53-3.55 (m, 1H), 3.66-3.70 (m, 2H), 3.80-3.85 (m, 4H), 4.01 (s, 3H), 4.33 (d, 2H), 6.44 (d, 1H), 7.04 (s, 1H), 7.58 (t, 1H), 7.76 (d, 1H), 8.29 (s, 1H), 8.39 (s, 1H).

### Example 24: N-(6-(difluoromethyl)pyridin-2-yl)-6-methoxy-2-(tetrahydro-2H-pyran-4-yl)-2H-indazole-5-carboxamide Hydrochloride

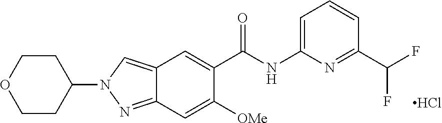 [SMILES: COc1cc2nn(C3CCOCC3)cc2cc1C(=O)Nc1cccc(C(F)F)n1 ; Cl]

To a solution of N-(6-(difluoromethyl)pyridin-2-yl)-6-methoxy-2H-indazole-5-carboxamide (Preparation 21, 80 mg, 0.251 mmol) and tetrahydro-2H-pyran-4-yl 4-methylbenzenesulfonate (77.3 mg, 0.302 mmol) in DMF (2 mL) was added K₂CO₃ (69.5 mg, 0.503 mmol) and the mixture heated at 95° C. for 16 h. The reaction mixture was filtered and the filtrate purified by prep-HPLC (Phenomenex Synergi C18 150×30 mm; 4 μm; 50-70% MeCN/H₂O (0.05% HCl)) to give N-(6-(difluoromethyl)pyridin-2-yl)-6-methoxy-2-(tetrahydro-2H-pyran-4-yl)-2H-indazole-5-carboxamide hydrochloride as a white solid (9.9 mg, 19%). LCMS m/z=403.0 [M+H]⁺; ¹H NMR (400 MHz, MeOH-d₄) δ: 2.05-2.12 (m, 4H), 3.45-3.55 (m, 2H), 3.96 (s, 3H), 4.00 (d, 2H), 4.67-4.76 (m, 1H), 6.74-7.03 (m, 1H), 7.17 (s, 1H), 7.43 (d, 1H), 8.03 (t, 1H), 8.27 (s, 1H), 8.41 (d, 1H), 8.54 (s, 1H), 10.69 (s, 1H).

### Example 25-41

The title compounds were prepared from the appropriate indazole (Indzole-1 to 7) and an appropriate alkylating agent (R—X) using a similar method to that described for Example 24. The table contains the following codes for the indazoles used:
Indazole-1: N-(6-(difluoromethyl)pyridin-2-yl)-6-isopropoxy-2H-indazole-5-carboxamide (Preparation 20); Indazole-2: N-(6-(difluoromethyl)pyridin-2-yl)-6-ethoxy-2H-indazole-5-carboxamide (Preparation 116); Indazole-3: N-(6-(difluoromethyl)pyridin-2-yl)-6-isopropoxy-2H-pyrazolo[3,4-b]pyridine-5-carboxamide (Preparation 120); Indazole-4: N-(1-(difluoromethyl)-1H-pyrazol-3-yl)-6-isopropoxy-2H-indazole-5-carboxamide (Preparation 118); Indazole-5:6-isopropoxy-N-(pyrazolo[1,5-a]pyrimidin-3-yl)-2H-indazole-5-carboxamide (Preparation 117); Indazole-6:6-isopropoxy-N-(pyrazolo[1,5-a]pyrimidin-3-yl)-2H-pyrazolo[3,4-b]pyridine-5-carboxamide (Preparation 119); Indazole-7:6-isopropoxy-N-(2-methoxypyridin-3-yl)-2H-pyrazolo[3,4-b]pyridine-5-carboxamide (Preparation 121).

| Example | |
|---|---|
| No. | Name/Structure/Indazole/R-X)/Data |
| 25 | N-(6-(difluoromethyl)pyridin-2-yl)-6-isopropoxy-2-(tetrahydro-2H-pyran-4-yl)- |
| | 2H-indazole-5-carboxamide hydrochloride |
| | 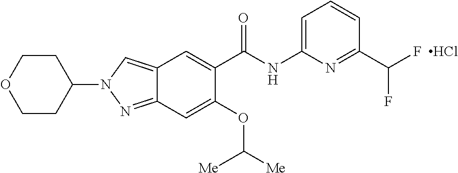 [SMILES: CC(C)Oc1cc2nn(C3CCOCC3)cc2cc1C(=O)Nc1cccc(C(F)F)n1 ; Cl] |
| | Indazole-1; R-X: tetrahydro-2H-pyran-4-yl 4-methylbenzenesulfonate |
| | Gradient: 49-69%; Yield: 10 mg, 11.4%; LCMS m/z = 431.1 [M + H]⁺; ¹H NMR |
| | (500 MHz, MeOH-d₄) δ: 1.59 (d, 6H), 2.22-2.32 (m, 4H), 3.67 (d, 2H), 4.14- |
| | 4.17 (m, 2H), 4.84-4.90 (m, 1H), 4.90-5.00 (m, 1H), 6.52-6.75 (m, 1H), 7.22 (s, |
| | 1H), 7.45 (d, 1H), 8.02 (t, 1H), 8.48 (d, 1H), 8.66 (s, 2H). |
| 26 | N-(6-(difluoromethyl)pyridin-2-yl)-6-isopropoxy-2-(3-methoxypropyl)-2H- |
| | indazole-5-carboxamide hydrochloride |
| | 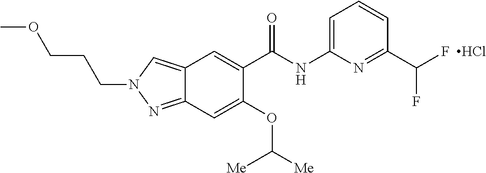 [SMILES: COCCCn1cc2cc(C(=O)Nc3cccc(C(F)F)n3)c(OC(C)C)cc2n1 ; Cl] |
| | Indazole-1; R-X: 1-bromo-3-methoxypropane |
| | Gradient: 49-69%; Yield: 13 mg, 15.4%; LCMS m/z = 419.1 [M + H]⁺; ¹H NMR |
| | (400 MHz, MeOH-d₄) δ: 1.56 (d, 6H), 2.22-2.32 (m, 2H), 3.67 (d, 3H), 3.39 (t, |
| | 2H), 4.57-4.62 (m, 2H), 4.90-5.00 (m, 1H), 6.52-6.74 (m, 1H), 7.18 (s, 1H), |
| | 7.42 (d, 1H), 7.99 (t, 1H), 8.46-8.52 (m, 2H), 8.62 (s, 1H). |
| 27 | N-(6-(difluoromethyl)pyridin-2-yl)-6-isopropoxy-2-((1-methyl-2- |
| | oxabicyclo[2.1.1]hexan-4-yl)methyl)-2H-indazole-5-carboxamide |
| | hydrochloride |
| | 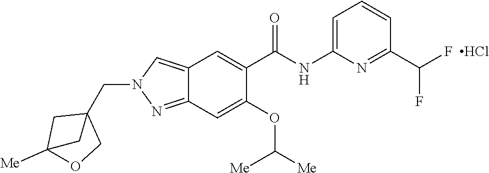 [SMILES: CC(C)Oc1cc2nn(CC34COC(C)(C3)C4)cc2cc1C(=O)Nc1cccc(C(F)F)n1 ; Cl] |
| | Indazole-1; R-X: (1-methyl-2-oxabicyclo[2.1.1]hexan-4-yl)methyl 4- |
| | methylbenzenesulfonate (Preparation 26) |
| | Gradient: 57-77%; Yield: 47 mg, 18%; LCMS m/z = 457.1 [M + H]⁺; ¹H NMR |
| | (400 MHz, MeOH-d₄) δ: 1.37 (s, 3H), 1.55-1.60 (m, 8H), 1.66 (d, 2H), 3.65 (s, |
| | 2H), 4.79 (s, 2H), 4.97-5.00 (m, 1H), 6.47-6.75 (m, 1H), 7.18 (s, 1H), 7.41 (d, |
| | 1H), 7.99 (t, 1H), 8.41 (s, 1H), 8.45 (d, 1H), 8.63 (s, 1H). |
| 28 | N-(6-(difluoromethyl)pyridin-2-yl)-6-isopropoxy-2-(2-methoxyethyl)-2H- |
| | indazole-5-carboxamide hydrochloride |
| | 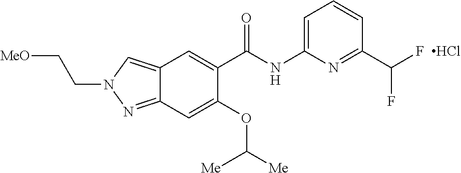 [SMILES: COCCn1cc2cc(C(=O)Nc3cccc(C(F)F)n3)c(OC(C)C)cc2n1 ; Cl] |
| | Indazole-1; R-X: 1-bromo-2-methoxyethane |
| | Gradient: 49-69%; Yield: 57 mg, 49%; LCMS m/z = 405.0 [M + H]⁺; ¹H NMR |
| | (500 MHz, MeOH-d₄) δ: 1.59 (d, 6H), 3.22-3.42 (m, 3H), 3.75 (t, 2H), 4.60 (t, |
| | 2H), 4.90-5.00 (m, 1H), 6.52-6.75 (m, 1H), 7.20 (s, 1H), 7.45 (d, 1H), 8.01 (t, |
| | 1H), 8.46-8.52 (m, 2H), 8.64 (s, 1H). |
| 29 | N-(6-(difluoromethyl)pyridin-2-yl)-6-isopropoxy-2-(3-methoxy-3- |
| | methylbutyl)-2H-indazole-5-carboxamide hydrochloride |
| | 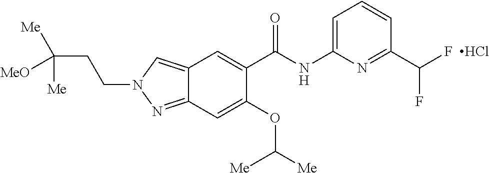 [SMILES: COC(C)(C)CCn1cc2cc(C(=O)Nc3cccc(C(F)F)n3)c(OC(C)C)cc2n1 ; Cl] |
| | Indazole-1; R-X: 3-methoxy-3-methylbutyl 4-methylbenzenesulfonate |
| | (Preparation 25) |
| | Gradient: 49-69%; Yield: 35 mg, 27%; LCMS m/z = 447.1 [M + H]⁺; ¹H NMR |
| | (500 MHz, MeOH-d₄) δ: 1.23 (s, 6H), 1.59 (d, 6H), 2.22 (t, 2H), 3.12-3.22 (m, |
| | 3H), 4.50 (t, 2H), 4.90-5.00 (m, 1H), 6.42-6.75 (m, 1H), 7.2 (s, 1H), 7.35 (d, |
| | 1H), 7.95 (t, 1H), 8.38-8.48 (m, 2H), 8.64 (s, 1H). |
| 30 | N-(6-(difluoromethyl)pyridin-2-yl)-6-isopropoxy-2-(oxetan-3-yl)-2H-indazole- |
| | 5-carboxamide |
| | 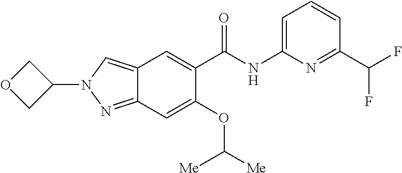 [SMILES: CC(C)Oc1cc2nn(C3COC3)cc2cc1C(=O)Nc1cccc(C(F)F)n1] |
| | Indazole-1; R-X: oxetan-3-yl 4-methylbenzenesulfonate |
| | Gradient: 57-77%; Yield: 15 mg, 8%; LCMS m/z = 403.0 [M + H]⁺; ¹H NMR |
| | (500 MHz, MeOH-d₄) δ: 1.58 (d, 6H), 4.88 (d, 1H), 5.16-5.19 (m, 4H), 5.83- |
| | 5.87 (m, 1H), 6.50-6.72 (m, 1H), 7.23 (s, 1H), 7.41-7.43 (m, 1H), 7.98 (t, 1H), |
| | 8.46 (d, 1H), 8.50 (s, 1H), 8.63 (s, 1H). |
| 31 | N-(6-(difluoromethyl)pyridin-2-yl)-6-ethoxy-2-(3-hydroxy-3-methylbutyl)-2H- |
| | indazole-5-carboxamide |
| | 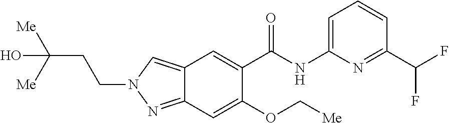 [SMILES: CCOc1cc2nn(CCC(C)(C)O)cc2cc1C(=O)Nc1cccc(C(F)F)n1] |
| | Indazole-2; R-X: 4-bromo-2-methylbutan-2-ol |
| | Prep-HPLC-Xtimate; 45-75%. |
| | Gradient: 45-75%; Yield: 20 mg, 10%; LCMS m/z = 419.1 [M + H]⁺; ¹H NMR |
| | (500 MHz, MeOH-d₄) δ: 1.27 (s, 6H), 1.63 (t, 3H), 2.14-2.18 (m, 2H), 4.31 (q, |
| | 2H), 4.51-4.55 (m, 2H), 6.67-6.70 (m, 1H), 7.08 (s, 1H), 7.40 (d, 1H), 7.96 (t, |
| | 1H), 8.36 (s, 1H), 8.43 (d, 1H), 8.54 (s, 1H). |
| 32 | N-(6-(difluoromethyl)pyridin-2-yl)-2-(3-hydroxy-3-methylbutyl)-6-isopropoxy- |
| | 2H-pyrazolo[3,4-b]pyridine-5-carboxamide |
| | 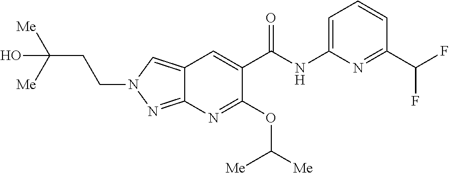 [SMILES: CC(C)Oc1nc2nn(CCC(C)(C)O)cc2cc1C(=O)Nc1cccc(C(F)F)n1] |
| | Indazole-3; R-X: 4-bromo-2-methylbutan-2-ol |
| | Prep-HPLC-Xtimate; 45-75%. |
| | Gradient: 45-75%; Yield: 62.2 mg, 49.6%; LCMS m/z = 434.0 [M + H]⁺; ¹H |
| | NMR (500 MHz, MeOH-d₄) δ: 1.28 (s, 6H), 1.58 (d, 6H), 2.14-2.19 (m, 2H), |
| | 4.51-4.54 (m, 2H), 5.64-5.71 (m, 1H), 6.49-6.72 (m, 1H), 7.42 (d, 1H), 7.95- |
| | 7.99 (m, 1H), 8.38 (s, 1H), 8.43 (d, 1H), 8.93 (s, 1H). |
| 33 | N-(1-(difluoromethyl)-1H-pyrazol-3-yl)-2-(3-hydroxy-3-methylbutyl)-6- |
| | isopropoxy-2H-indazole-5-carboxamide |
| | 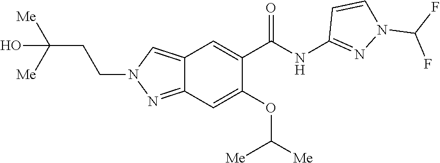 [SMILES: CC(C)Oc1cc2nn(CCC(C)(C)O)cc2cc1C(=O)Nc1ccn(C(F)F)n1] |
| | Indazole-4; R-X: 4-bromo-2-methylbutan-2-ol |
| | Prep-HPLC-Xtimate; 33-63%. |
| | Gradient: 33-63%; Yield: 37.9 mg, 14.7%; LCMS m/z = 422.3 [M + H]⁺; ¹H |
| | NMR (500 MHz, MeOH-d₄) δ: 1.27 (s, 6H), 1.53 (d, 6H), 2.14-2.18 (m, 2H), |
| | 4.53-4.57 (m, 2H), 4.89-4.95 (m, 1H), 7.01 (d, 1H), 7.13 (s, 1H), 7.27-7.51 (m, |
| | 1H), 7.99 (d, 1H), 8.38 (s, 1H), 8.54 (s, 1H). |
| 34 | 2-(3-Hydroxy-3-methylbutyl)-6-isopropoxy-N-(pyrazolo[1,5-a]pyrimidin-3-yl)- |
| | 2H-indazole-5-carboxamide |
| | 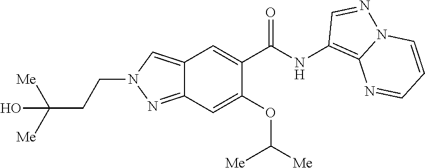 [SMILES: CC(C)Oc1cc2nn(CCC(C)(C)O)cc2cc1C(=O)Nc1cnn2cccnc12] |
| | Indazole-5; R-X: 4-bromo-2-methylbutan-2-ol |
| | Prep-HPLC-Xtimate; 29-49%. |
| | Yield: 22 mg, 10%; LCMS m/z = 423.1 [M + H]⁺; ¹H NMR (500 MHz, MeOH- |
| | d₄) δ: 1.31 (m, 6H), 1.29 (d, 6H), 2.18-2.22 (m, 2H), 4.57-4.60 (m, 2H), 5.02- |
| | 5.07 (m, 1H), 7.01-7.04 (m, 1H), 7.21 (s, 1H), 8.43 (s, 1H), 8.52-8.54 (m, 2H), |
| | 8.70 (s, 1H), 8.84-8.82 (m, 1H). |
| 35 | 6-Isopropoxy-2-(3-methoxypropyl)-N-(pyrazolo[1,5-a]pyrimidin-3-yl)-2H- |
| | indazole-5-carboxamide |
| | 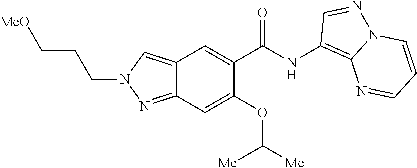 [SMILES: COCCCn1cc2cc(C(=O)Nc3cnn4cccnc34)c(OC(C)C)cc2n1] |
| | Indazole-5; R-X: 1-bromo-3-methoxypropane |
| | Prep-HPLC-Xtimate; 31-61%. |
| | Yield: 10.1 mg, 10.4%; LCMS m/z = 409.1 [M + H]⁺; ¹H NMR (400 MHz, |
| | MeOH-d₄) δ: 1.64 (d, 6H), 2.21-2.28 (m, 2H), 3.33 (s, 3H), 3.37 (t, 2H), 4.53 (t, |
| | 2H), 4.95-5.05 (m, 1H), 7.00 (dd, 1H), 7.18 (s, 1H), 8.37 (s, 1H), 8.50 (dd, 1H), |
| | 8.68 (s, 1H), 8.82 (s, 1H), 8.83 (d, 1H). |
| 36 | 6-Isopropoxy-N-(pyrazolo[1,5-a]pyrimidin-3-yl)-2-(tetrahydro-2H-pyran-3-yl)- |
| | 2H-pyrazolo[3,4-b]pyridine-5-carboxamide |
| | 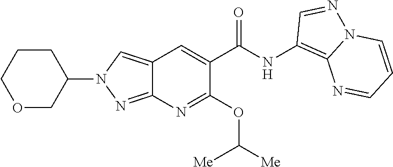 [SMILES: CC(C)Oc1nc2nn(C3CCCOC3)cc2cc1C(=O)Nc1cnn2cccnc12] |
| | Indazole-6; R-X: tetrahydro-2H-pyran-3-yl methanesulfonate |
| | Prep-HPLC-Xtimate; 35-65% |
| | Yield: 16.6 mg, 13%; LCMS m/z = 422.2 [M + H]⁺; ¹H NMR (400 MHz, CDCl₃) δ: |
| | 1.66 (d, 6H), 1.81-1.86 (m, 2H), 2.33-2.35 (m, 1H), 2.42-2.45 (m, 1H), 3.65- |
| | 3.66 (m, 1H), 3.90-4.00 (m, 1H), 4.00-4.06 (m, 1H), 4.20-4.24 (m, 1H), 4.55- |
| | 4.57 (m, 1H), 5.87-5.94 (m, 1H), 6.81 (dd, 1H), 8.17 (s, 1H), 8.42 (dd, 1H), |
| | 8.61 (dd, 1H), 9.01 (s, 1H), 9.11 (s, 1H), 10.80 (s, 1H). |
| 37 | 6-Isopropoxy-N-(pyrazolo[1,5-a]pyrimidin-3-yl)-2-(tetrahydrofuran-3-yl)-2H- |
| | pyrazolo[3,4-b]pyridine-5-carboxamide |
| | 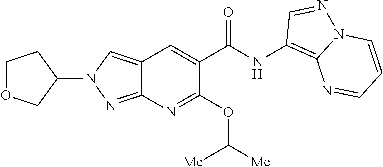 [SMILES: CC(C)Oc1nc2nn(C3CCOC3)cc2cc1C(=O)Nc1cnn2cccnc12] |
| | Indazole-6; R-X: tetrahydrofuran-3-yl methanesulfonate |
| | Prep-HPLC-Xtimate; 30-60% |
| | Yield: 29.4 mg, 24%; LCMS m/z = 430.1 [M + Na]⁺; ¹H NMR (500 MHz, |
| | CDCl₃) δ: 1.66 (d, 6H), 2.55-2.63 (m, 2H), 4.01-4.06 (m, 1H), 4.18-4.22 (m, |
| | 1H), 4.22-4.27 (m, 2H), 5.21-5.23 (m, 1H), 5.89-5.92 (m, 1H), 6.81 (dd, 1H), |
| | 8.10 (s, 1H), 8.42 (d, 1H), 8.62 (d, 1H), 9.00 (s, 1H), 9.10 (s, 1H), 10.80 (s, 1H). |
| 38 | 6-Isopropoxy-2-(oxetan-3-yl)-N-(pyrazolo[1,5-a]pyrimidin-3-yl)-2H-indazole- |
| | 5-carboxamide |
| | 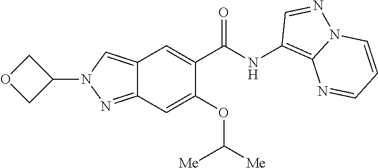 [SMILES: CC(C)Oc1cc2nn(C3COC3)cc2cc1C(=O)Nc1cnn2cccnc12] |
| | Indazole-5; R-X: oxetan-3-yl methanesulfonate |
| | Prep-HPLC-Xtimate; 30-60%. |
| | Yield: 29.4 mg, 24%; LCMS m/z = 393.1 [M + H]⁺; ¹H NMR (500 MHz, |
| | MeOH-d₄) δ: 1.64 (d, 6H), 5.03-5.07 (m, 1H), 5.14-5.20 (m, 4H), 5.82-5.88 (m, |
| | 1H), 7.00 (dd, 1H), 7.26 (s, 1H), 8.49-8.51 (m, 2H), 8.69 (s, 1H), 8.81-8.85 (m, |
| | 2H). |
| 39 | 6-Isopropoxy-N-(pyrazolo[1,5-a]pyrimidin-3-yl)-2-((tetrahydrofuran-3- |
| | yl)methyl)-2H-indazole-5-carboxamide |
| | 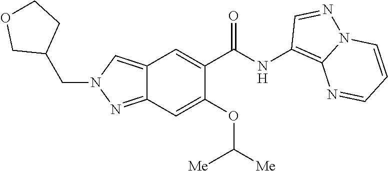 [SMILES: CC(C)Oc1cc2nn(CC3CCOC3)cc2cc1C(=O)Nc1cnn2cccnc12] |
| | Indazole-5; R-X: (tetrahydrofuran-3-yl)methyl methanesulfonate |
| | Prep-HPLC-Xtimate; 30-60%. |
| | Yield: 22.7 mg, 22.5%; LCMS m/z = 421.2 [M + H]⁺; ¹H NMR (500 MHz, |
| | MeOH-d₄) δ: 1.63 (d, 6H), 1.70-1.79 (m, 1H), 2.00-2.10 (m, 1H), 2.94-3.04 (m, |
| | 1H), 3.64 (dd, 1H), 3.75-3.81 (m, 2H), 3.90-3.96 (m, 1H), 4.44 (d, 2H), 4.98- |
| | 5.04 (m, 1H), 6.99 (dd, 1H), 7.18 (s, 1H), 8.42 (s, 1H), 8.49 (dd, 1H), 8.67 (s, |
| | 1H), 8.81-8.84 (m, 2H). |
| 40 | 2-(3-Hydroxy-3-methylbutyl)-6-isopropoxy-N-(2-methoxypyridin-3-yl)-2H- |
| | pyrazolo[3,4-b]pyridine-5-carboxamide |
| | 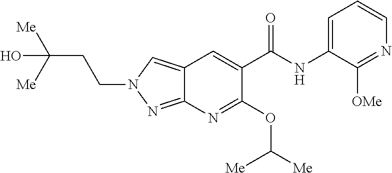 [SMILES: COc1ncccc1NC(=O)c1cc2cn(CCC(C)(C)O)nc2nc1OC(C)C] |
| | Indazole-7; R-X: 4-bromo-2-methylbutan-2-ol |
| | Prep-HPLC-Xtimate; 45-75%. |
| | Yield: 10.1 mg, 11.8%; LCMS m/z = 414.1 [M + H]⁺; ¹H NMR (500 MHz, |
| | MeOH-d₄) δ: 1.28 (s, 6H), 1.59 (d, 6H), 2.15-2.19 (m, 2H), 4.10 (s, 3H), 4.50- |
| | 4.60 (m, 2H), 5.81-5.87 (m, 1H), 7.00 (d, 1H), 7.88 (d, 1H), 8.41 (s, 1H), 8.81 |
| | (dd, 1H), 9.01 (s, 1H). |
| 41 | N-(6-(difluoromethyl)pyridin-2-yl)-6-isopropoxy-2-methyl-2H-indazole-5- |
| | carboxamide |
| | 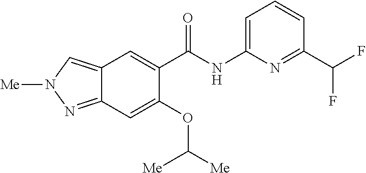 [SMILES: CC(C)Oc1cc2nn(C)cc2cc1C(=O)Nc1cccc(C(F)F)n1] |
| | Indazole-1; R-X: methyl methanesulfonate |
| | Prep-HPLC-YMC; 55-85%. |
| | Gradient: 55-85%; Yield: 28.4 mg, 22.8%; LCMS m/z = 361.1 [M + H]⁺; ¹H |
| | NMR (500 MHz, MeOH-d₄) δ: 1.54 (d, 6H), 4.18 (s, 3H), 4.90-4.97 (m, 1H), |
| | 6.44-6.73 (m, 1H), 7.13 (s, 1H), 7.40 (d, 1H), 7.96 (t, 1H), 8.32 (s, 1H), 8.44 (d, |
| | 1H), 8.59 (s, 1H). |

### Example 42 and 43: (S)—N-(6-(difluoromethyl)pyridin-2-yl)-6-isopropoxy-2-(tetrahydro-2H-pyran-3-yl)-2H-indazole-5-carboxamide and (R)—N-(6-(difluoromethyl)pyridin-2-yl)-6-isopropoxy-2-(tetrahydro-2H-pyran-3-yl)-2H-indazole-5-carboxamide

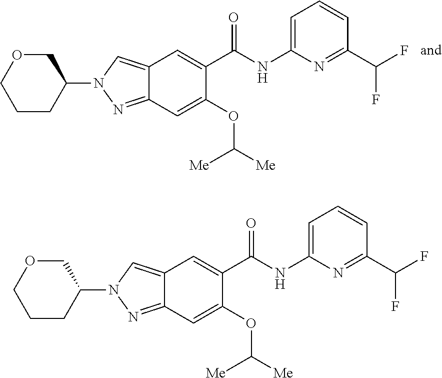 [SMILES: CC(C)Oc1cc2nn([C@H]3CCCOC3)cc2cc1C(=O)Nc1cccc(C(F)F)n1 ; "and" ; CC(C)Oc1cc2nn([C@@H]3CCCOC3)cc2cc1C(=O)Nc1cccc(C(F)F)n1]

### *Stereochemistry Arbitrarily Assigned

To a solution of N-(6-(difluoromethyl)pyridin-2-yl)-6-isopropoxy-2H-indazole-5-carboxamide (Preparation 20, 500 mg, 1.44 mmol) and tetrahydro-2H-pyran-3-yl 4-methylbenzenesulfonate (442 mg, 1.73 mmol) in DMF (8 mL) was added K₂CO₃ (398 mg, 2.88 mmol) and the mixture was heated at 100° C. for 16 h. The reaction mixture was filtered and the filtrate purified by prep-HPLC (Phenomenex Synergi C18 150×30 mm, 4 μm; 58-78% MeCN/H₂O (0.05% HCl)) to N-(6-(difluoromethyl)pyridin-2-yl)-6-isopropoxy-2-(tetrahydro-2H-pyran-3-yl)-2H-indazole-5-carboxamide as a white solid (50 mg, 8%). Further purification by prep-SFC (Daicel Chiralcel OD-H; 250×30 mm, 5 μm; 30% IPA+0.1% NH₄OH in CO₂) afforded (S)—N-(6-(difluoromethyl)pyridin-2-yl)-6-isopropoxy-2-(tetrahydro-2H-pyran-3-yl)-2H-indazole-5-carboxamide and (R)—N-(6-(difluoromethyl)pyridin-2-yl)-6-isopropoxy-2-(tetrahydro-2H-pyran-3-yl)-2H-indazole-5-carboxamide.
*Peak 1, Example 42; Yield: 22 mg, 44%; LCMS m/z=431.1 [M+H]⁺; ¹H NMR (400 MHz, MeOH-d₄) δ: 1.57 (d, 6H), 1.80-1.82 (m, 2H), 2.31-2.33 (m, 2H), 3.61-3.64 (m, 1H), 3.90-3.92 (m, 2H), 4.15 (d, 1H), 4.58-4.60 (m, 1H), 4.93-4.95 (m, 1H), 6.58-6.72 (m, 1H), 7.14 (s, 1H), 7.40 (d, 1H), 7.98 (t, 1H), 8.44-8.50 (m, 2H), 8.61 (s, 1H).
*Peak 2, Example 43; Yield: 18 mg, 36%; LCMS m/z=431.1 [M+H]⁺; ¹H NMR (400 MHz, MeOH-d₄) δ: 1.57 (d, 6H), 1.80-1.82 (m, 2H), 2.31-2.33 (m, 2H), 3.61-3.64 (m, 1H), 3.90-3.92 (m, 2H), 4.15 (d, 1H), 4.58-4.60 (m, 1H), 4.93-4.95 (m, 1H), 6.58-6.72 (m, 1H), 7.14 (s, 1H), 7.40 (d, 1H), 7.98 (t, 1H), 8.44-8.50 (m, 2H), 8.61 (s, 1H).

### Example 44 and 45: (S)—N-(6-(difluoromethyl)pyridin-2-yl)-6-isopropoxy-2-(tetrahydrofuran-3-yl)-2H-indazole-5-carboxamide and (R)—N-(6-(difluoromethyl)pyridin-2-yl)-6-isopropoxy-2-(tetrahydrofuran-3-yl)-2H-indazole-5-carboxamide

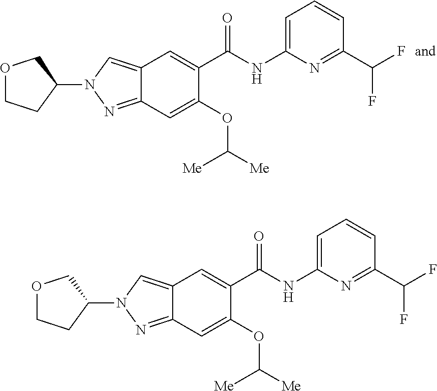 [SMILES: CC(C)Oc1cc2nn([C@H]3CCOC3)cc2cc1C(=O)Nc1cccc(C(F)F)n1 ; "and" ; CC(C)Oc1cc2nn([C@@H]3CCOC3)cc2cc1C(=O)Nc1cccc(C(F)F)n1]

### *Stereochemistry Arbitrarily Assigned

The title compounds were prepared from N-(6-(difluoromethyl)pyridin-2-yl)-6-isopropoxy-2H-indazole-5-carboxamide (Preparation 20) and tetrahydrofuran-3-yl 4-methylbenzenesulfonate using an analogous method to that described for Examples 42 and 43. Prep-SFC (Daicel Chiralcel OD-H; 250×30 mm, 5 μm; 45% IPA+0.1% NH₄OH in CO₂) afforded (S)—N-(6-(difluoromethyl)pyridin-2-yl)-6-isopropoxy-2-(tetrahydrofuran-3-yl)-2H-indazole-5-carboxamide and (R)—N-(6-(difluoromethyl)pyridin-2-yl)-6-isopropoxy-2-(tetrahydrofuran-3-yl)-2H-indazole-5-carboxamide.
*Peak 1, Example 44; Yield: 27 mg, 38.6%; LCMS m/z=417.1 [M+H]⁺; ¹H NMR (500 MHz, MeOH-d₄) δ: 1.46 (d, 6H), 2.35-2.37 (m, 1H), 2.51-2.53 (m, 1H), 3.86-3.89 (m, 1H), 4.06-4.09 (m, 2H), 4.12 (d, 1H), 4.82-4.84 (m, 1H), 5.22-5.34 (m, 1H), 6.50-6.61 (m, 1H), 7.06 (s, 1H), 7.31 (d, 1H), 7.87 (t, 1H), 8.33-8.35 (m, 2H), 8.51 (s, 1H).
*Peak 1, Example 45; Yield: 25 mg, 35.7%; LCMS m/z=417.1 [M+H]⁺; ¹H NMR (500 MHz, MeOH-d₄) δ: 1.46 (d, 6H), 2.35-2.37 (m, 1H), 2.51-2.53 (m, 1H), 3.86-3.89 (m, 1H), 4.06-4.09 (m, 2H), 4.12 (d, 1H), 4.82-4.84 (m, 1H), 5.22-5.34 (m, 1H), 6.50-6.61 (m, 1H), 7.06 (s, 1H), 7.31 (d, 1H), 7.87 (t, 1H), 8.33-8.35 (m, 2H), 8.51 (s, 1H).

### Example 46 and 47: (R)—N-(6-(difluoromethyl)pyridin-2-yl)-2-(2,2-dimethyltetrahydro-2H-pyran-4-yl)-6-isopropoxy-2H-indazole-5-carboxamide and (S)—N-(6-(difluoromethyl)pyridin-2-yl)-2-(2,2-dimethyltetrahydro-2H-pyran-4-yl)-6-isopropoxy-2H-indazole-5-carboxamide

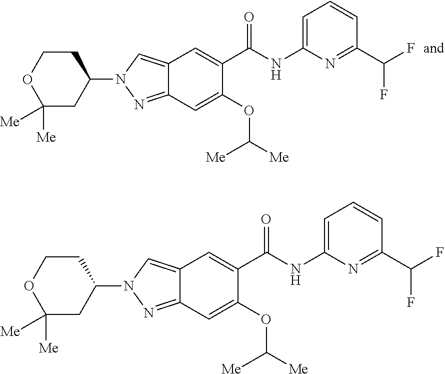 [SMILES: CC(C)Oc1cc2nn([C@@H]3CCOC(C)(C)C3)cc2cc1C(=O)Nc1cccc(C(F)F)n1 ; "and" ; CC(C)Oc1cc2nn([C@H]3CCOC(C)(C)C3)cc2cc1C(=O)Nc1cccc(C(F)F)n1]

### *Stereochemistry Arbitrarily Assigned

The title compounds were prepared from N-(6-(difluoromethyl)pyridin-2-yl)-6-isopropoxy-2H-indazole-5-carboxamide (Preparation 20) and 2,2-dimethyltetrahydro-2H-pyran-4-yl methanesulfonate using an analogous method to that described for Examples 42 and 43. Prep-SFC (Daicel Chiralcel OD-H; 250×30 mm, 10 μm; 55% EtOH+0.1% NH₄OH in CO₂) afforded (R)—N-(6-(difluoromethyl)pyridin-2-yl)-2-(2,2-dimethyltetrahydro-2H-pyran-4-yl)-6-isopropoxy-2H-indazole-5-carboxamide and (S)—N-(6-(difluoromethyl)pyridin-2-yl)-2-(2,2-dimethyltetrahydro-2H-pyran-4-yl)-6-isopropoxy-2H-indazole-5-carboxamide.
*Peak 1, Example 46; Yield: 16 mg, 34.9%; LCMS m/z=459.1 [M+H]⁺; ¹H NMR (500 MHz, MeOH-d₄) δ: 1.35 (s, 3H), 1.42 (s, 3H), 1.59 (d, 6H), 2.08-2.10 (m, 1H), 2.17-2.21 (m, 3H), 3.94-3.96 (m, 2H), 4.98-5.01 (m, 2H), 6.52-6.74 (m, 1H), 7.19 (s, 1H), 7.43 (d, 1H), 8.01 (t, 1H), 8.19-8.47 (m, 2H), 8.65 (s, 1H).
*Peak 2, Example 47; Yield: 15 mg, 37.5%; LCMS m/z=459.1 [M+H]⁺; ¹H NMR (500 MHz, MeOH-d₄) δ: 1.35 (s, 3H), 1.42 (s, 3H), 1.59 (d, 6H), 2.08-2.10 (m, 1H), 2.17-2.21 (m, 3H), 3.94-3.96 (m, 2H), 4.98-5.01 (m, 2H), 6.52-6.74 (m, 1H), 7.19 (s, 1H), 7.43 (d, 1H), 8.01 (t, 1H), 8.19-8.47 (m, 2H), 8.65 (s, 1H).

### Example 48: 6-((1R,3R)-3-(difluoromethyl)cyclobutoxy)-N-(6-(difluoromethyl)pyridin-2-yl)-2-(tetrahydro-2H-pyran-4-yl)-2H-indazole-5-carboxamide Hydrochloride

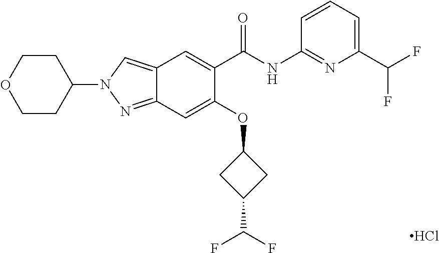 [SMILES: O=C(Nc1cccc(C(F)F)n1)c1cc2cn(C3CCOCC3)nc2cc1O[C@H]1C[C@H](C(F)F)C1 ; Cl]

To a solution of N-(6-(difluoromethyl)pyridin-2-yl)-6-hydroxy-2-(tetrahydro-2H-pyran-4-yl)-2H-indazole-5-carboxamide (Preparation 123, 50 mg, 0.129 mmol) and 3-(difluoromethyl)cyclobutyl methanesulfonate (Preparation 28, 55 mg, 70% purity) in DMF (3 mL) was added K₂CO₃ (53 mg, 0.39 mmol) and the mixture was stirred at 100° C. for 14 h. The mixture was filtered through a pad of Celite® and the filtrate purified by prep-HPLC (Phenomenex Synergi C18 150×30 mm 4 μm; 20-40% MeCN/H₂O (0.05% HCl) to give 6-((1r,3r)-3-(difluoromethyl)cyclobutoxy)-N-(6-(difluoromethyl)pyridin-2-yl)-2-(tetrahydro-2H-pyran-4-yl)-2H-indazole-5-carboxamide hydrochloride as a yellow solid (8.2 mg, 12.9%). LCMS m/z=493.1 [M+H]⁺; ¹H NMR (500 MHz, MeOH-d₄) δ: 2.10-2.20 (m, 2H), 2.25-2.35 (m, 2H), 2.50-2.60 (m, 2H), 2.75-2.85 (m, 2H), 2.90-3.00 (m, 1H), 3.60-3.70 (m, 2H), 4.10-4.20 (m, 2H), 4.70-4.80 (m, 1H), 5.10-5.20 (m, 1H), 6.00-6.25 (m, 1H), 6.50-6.80 (m, 1H), 6.90 (s, 1H), 7.43 (d, 1H), 8.00 (t, 1H), 8.40-8.50 (m, 2H), 8.58 (s, 1H).

### Example 49: 6-((3,3-Difluorocyclobutyl)methoxy)-N-(6-(difluoromethyl)pyridin-2-yl)-2-(tetrahydro-2H-pyran-4-yl)-2H-indazole-5-carboxamide Hydrochloride

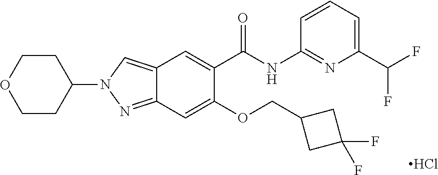 [SMILES: O=C(Nc1cccc(C(F)F)n1)c1cc2cn(C3CCOCC3)nc2cc1OCC1CC(F)(F)C1 ; Cl]

6-((3,3-difluorocyclobutyl)methoxy)-N-(6-(difluoromethyl)pyridin-2-yl)-2-(tetrahydro-2H-pyran-4-yl)-2H-indazole-5-carboxamide hydrochloride was prepared from N-(6-(difluoromethyl)pyridin-2-yl)-6-hydroxy-2-(tetrahydro-2H-pyran-4-yl)-2H-indazole-5-carboxamide (Preparation 123) and (3,3-difluorocyclobutyl)methyl methanesulfonate using an analogous method to that described for Example 48. LCMS m/z=493.1 [M+H]⁺; ¹H NMR (500 MHz, MeOH-d₄) δ: 2.17-2.30 (m, 4H), 2.55-2.67 (m, 2H), 2.85-2.94 (m, 3H), 3.64-3.69 (m, 2H), 4.13-4.17 (m, 2H), 4.34-4.36 (m, 2H), 4.77-4.80 (m, 1H), 6.52-6.75 (m, 1H), 7.19 (s, 1H), 7.44 (d, 1H), 8.01 (t, 1H), 8.48 (d, 2H), 8.56 (d, 1H).

### Example 50: N-(6-(difluoromethyl)pyridin-2-yl)-6-((1R,3R)-3-methoxycyclobutoxy)-2-(tetrahydro-2H-pyran-4-yl)-2H-indazole-5-carboxamide Hydrochloride

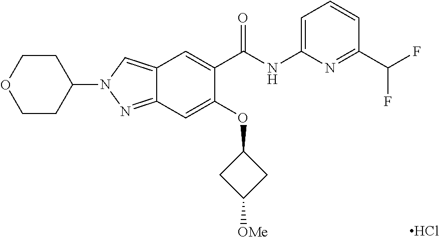 [SMILES: CO[C@H]1C[C@H](Oc2cc3nn(C4CCOCC4)cc3cc2C(=O)Nc2cccc(C(F)F)n2)C1 ; Cl]

N-(6-(difluoromethyl)pyridin-2-yl)-6-((1r,3r)-3-methoxycyclobutoxy)-2-(tetrahydro-2H-pyran-4-yl)-2H-indazole-5-carboxamide hydrochloride was prepared from N-(6-(difluoromethyl)pyridin-2-yl)-6-hydroxy-2-(tetrahydro-2H-pyran-4-yl)-2H-indazole-5-carboxamide (Preparation 123) and 3-methoxycyclobutyl methanesulfonate (Preparation 29) using an analogous method to that described for Example 48. LCMS m/z=473.1 [M+H]⁺; ¹H NMR (400 MHz, MeOH-d₄) δ: 2.20-2.30 (m, 4H), 2.60-2.70 (m, 4H), 3.29 (s, 3H), 3.60-3.70 (m, 2H), 4.10-4.20 (m, 2H), 4.30-4.35 (m, 1H), 4.70-4.80 (m, 1H), 5.10-5.20 (m, 1H), 6.50-6.75 (m, 1H), 6.92 (s, 1H), 7.43 (d, 1H), 7.99 (t, 1H), 8.45-8.46 (m, 2H), 8.59 (s, 1H).

### Example 51: 6-Isopropoxy-N-(pyrazolo[1,5-a]pyridin-7-yl)-2-(tetrahydro-2H-pyran-4-yl)-2H-indazole-5-carboxamide

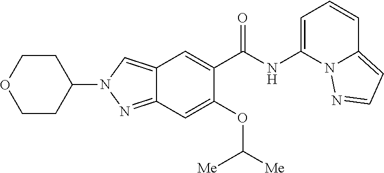 [SMILES: CC(C)Oc1cc2nn(C3CCOCC3)cc2cc1C(=O)Nc1cccc2ccnn12]

To a solution of 7-iodopyrazolo[1,5-a]pyridine (30 mg, 0.123 mmol) in toluene (3 mL) was added 6-isopropoxy-2-(tetrahydro-2H-pyran-4-yl)-2H-indazole-5-carboxamide (Preparation 125, 44.8 mg, 0.148 mmol), Pd₂(dba)₃ (11.3 mg, 12.3 μmol), Xantphos (14.2 mg, 24.6 μmol) and Cs₂CO₃ (80.1 mg, 0.246 mmol) and the mixture was stirred at 110° C. for 16 h under N₂. The reaction mixture was filtered and the filtrate evaporated to dryness in vacuo. The residue was purified by prep-HPLC (YMC-Actus Triart C18 150×30 mm×5 μm; 58-85% MeCN/H₂O (0.225% FA)) to afford 6-isopropoxy-N-(pyrazolo[1,5-a]pyridin-7-yl)-2-(tetrahydro-2H-pyran-4-yl)-2H-indazole-5-carboxamide as a white solid (21.8 mg, 42.3%). LCMS m/z=420.3 [M+H]⁺; ¹H NMR (500 MHz, CDCl₃) δ: 1.68 (d, 6H), 2.16-2.36 (m, 4H), 3.55-3.69 (m, 2H), 4.19 (d, 2H), 4.62-4.65 (m, 1H), 4.94-5.01 (m, 1H), 6.57 (s, 1H), 7.17-7.26 (m, 2H), 7.31-7.33 (m, 1H), 7.94-8.12 (m, 3H), 8.80 (s, 1H), 11.99 (s, 1H).

### Example 52 and 53: (S)-6-Isopropoxy-N-(pyrazolo[1,5-a]pyridin-7-yl)-2-((tetrahydrofuran-3-yl)methyl)-2H-pyrazolo[3,4-b]pyridine-5-carboxamide and (R)-6-isopropoxy-N-(pyrazolo[1,5-a]pyridin-7-yl)-2-((tetrahydrofuran-3-yl)methyl)-2H-pyrazolo[3,4-b]pyridine-5-carboxamide

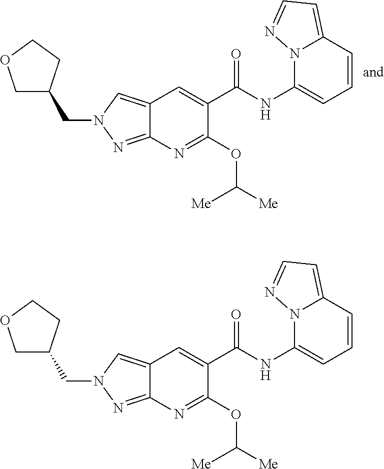 [SMILES: CC(C)Oc1nc2nn(C[C@@H]3CCOC3)cc2cc1C(=O)Nc1cccc2ccnn12 ; CC(C)Oc1nc2nn(C[C@H]3CCOC3)cc2cc1C(=O)Nc1cccc2ccnn12]

### *Stereochemistry Arbitrarily Assigned

To a solution of 6-isopropoxy-2-((tetrahydrofuran-3-yl)methyl)-2H-pyrazolo[3,4-b]pyridine-5-carboxamide (Preparation 124, 123 mg, 0.406 mmol) in toluene (2 mL) was added 7-bromopyrazolo[1,5-a]pyridine (40 mg, 0.203 mmol), Cs₂CO₃ (132 mg, 0.406 mmol), Xantphos (23.5 mg, 0.041 mmol) and Pd₂(dba)₃ (18.6 mg, 0.020 mmol) and the mixture was stirred at 20° C. under N₂. The reaction was evaporated in vacuo and the residue purified by prep-HPLC (Boston Prime C18 150×30 mm×5 μm, 60-90% MeCN/H₂O (0.04% NH₄OH+10 mM NH₄HCO₃) to afford 6-isopropoxy-N-(pyrazolo[1,5-a]pyridin-7-yl)-2-((tetrahydrofuran-3-yl)methyl)-2H-pyrazolo[3,4-b]pyridine-5-carboxamide as a white solid (50 mg, 58%). Further purification by prep-SFC (Daicel Chiralcel OJ-H; 250×30 mm, 5 μm; 25-30% EtOH+0.1% NH₄OH in CO₂) afforded (S)-6-isopropoxy-N-(pyrazolo[1,5-a]pyridin-7-yl)-2-((tetrahydrofuran-3-yl)methyl)-2H-pyrazolo[3,4-b]pyridine-5-carboxamide and (R)-6-isopropoxy-N-(pyrazolo[1,5-a]pyridin-7-yl)-2-((tetrahydrofuran-3-yl)methyl)-2H-pyrazolo[3,4-b]pyridine-5-carboxamide.
*Peak 1, Example 52 (12.5 mg, 24.4%); LCMS m/z=421.1 [M+H]⁺; ¹H NMR (500 MHz, MeOH-d₄) δ: 1.69 (d, 6H), 1.72-1.80 (m, 1H), 2.03-2.12 (m, 1H), 2.97-3.03 (m, 1H), 3.62-3.66 (m, 1H), 3.75-3.83 (m, 2H), 3.91-3.96 (m, 1H), 4.44 (d, 2H), 5.92-5.98 (m, 1H), 6.65 (d, 1H), 7.25-7.29 (m, 1H), 7.42 (d, 1H), 7.92 (d, 1H), 8.04 (d, 1H), 8.45 (s, 1H), 9.11 (s, 1H).
*Peak 2, Example 53 (11.1 mg, 21.9%); LCMS m/z=421.1 [M+H]⁺; ¹H NMR (500 MHz, MeOH-d₄) δ: 1.70 (d, 6H), 1.73-1.80 (m, 1H), 2.04-2.12 (m, 1H), 2.98-3.04 (m, 1H), 3.63-3.67 (m, 1H), 3.76-3.83 (m, 2H), 3.92-3.97 (m, 1H), 4.44 (d, 2H), 5.92-5.98 (m, 1H), 6.66 (d, 1H), 7.25-7.29 (m, 1H), 7.43 (d, 1H), 7.92 (d, 1H), 8.04 (d, 1H), 8.46 (s, 1H), 9.11 (s, 1H).

### Example 54: 6-Isopropoxy-N-(pyrazolo[1,5-a]pyrimidin-3-yl)-2-((tetrahydrofuran-3-yl)methyl)-2H-pyrazolo[3,4-b]pyridine-5-carboxamide

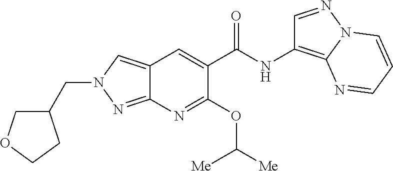 [SMILES: CC(C)Oc1nc2nn(CC3CCOC3)cc2cc1C(=O)Nc1cnn2cccnc12]

To a solution of 6-isopropoxy-2-((tetrahydrofuran-3-yl)methyl)-2H-pyrazolo[3,4-b]pyridine-5-carboxylic acid (Preparation 104, 80 mg, 0.262 mmol) in pyridine (2 mL) was added pyrazolo[1,5-a]pyrimidin-3-amine (70.3 mg, 0.524 mmol) and T3P® (50 wt. % in EtOAc, 2 mL) under N₂ and the mixture was stirred at rt for 14 h. The reaction was evaporated to dryness in vacuo and the residue diluted with aqueous NaHCO₃ (40 mL) and extracted with EtOAc (2×30 mL). The combined organics were dried (Na₂SO₄) and evaporated to dryness. The residue was purified by Combi-Flash (PE/EtOAc; 3/1-0/1) to afford 6-isopropoxy-N-(pyrazolo[1,5-a]pyrimidin-3-yl)-2-((tetrahydrofuran-3-yl)methyl)-2H-pyrazolo[3,4-b]pyridine-5-carboxamide as a yellow solid (70 mg, 60%). LCMS m/z=422.0 [M+H]⁺; ¹H NMR (400 MHz, MeOH-d₄) δ: 1.65 (d, 6H), 1.71-1.80 (m, 1H), 2.03-2.12 (m, 1H), 2.96-3.04 (m, 1H), 3.62-3.66 (m, 1H), 3.75-3.83 (m, 2H), 3.90-3.97 (m, 1H), 4.43 (d, 2H), 5.74-5.81 (m, 1H), 6.99-7.03 (m, 1H), 8.43 (s, 1H), 8.52 (s, 1H), 8.81 (s, 1H), 8.85 (d, 1H), 9.01 (s, 1H).

### Example 55: 6-Isopropoxy-N-(1-methyl-2-oxo-1,2-dihydropyridin-3-yl)-2-(1-methyl-2-oxabicyclo[2.1.1]hexan-4-yl)-2H-indazole-5-carboxamide Trifluoroacetate

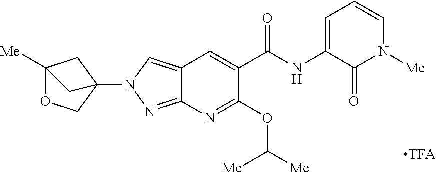 [SMILES: CC(C)Oc1nc2nn(C34COC(C)(C3)C4)cc2cc1C(=O)Nc1cccn(C)c1=O]

To a solution of 6-isopropoxy-2-(1-methyl-2-oxabicyclo[2.1.1]hexan-4-yl)-2H-indazole-5-carboxylic acid (Preparation 97, 38.3 mg, 0.121 mmol) in pyridine (1 mL) was added 3-amino-1-methylpyridin-2 (1H)-one (29.2 mg, 0.182 mmol) and T3P (50 wt. % in EtOAc, 0.36 mL) under N₂ and the mixture was stirred at rt for 16 h. The reaction was evaporated to dryness in vacuo and the residue diluted with aqueous NaHCO₃ (40 mL) and extracted with EtOAc (2×30 mL). The combined organics were dried (Na₂SO₄) and evaporated to dryness. The residue was purified by prep-HPLC-Sunfire (gradient, 5-55%) to afford 6-isopropoxy-N-(1-methyl-2-oxo-1,2-dihydropyridin-3-yl)-2-(1-methyl-2-oxabicyclo[2.1.1]hexan-4-yl)-2H-indazole-5-carboxamide trifluoroacetate as a white solid (37.1 mg, 57%). LCMS m/z=423.2 [M+H]⁺; ¹H NMR (500 MHz, DMSO-d₆) δ: 1.41-1.57 (m, 9H), 2.18 (dd, 2H), 2.33-2.44 (m, 2H), 3.56 (s, 3H), 4.10 (s, 2H), 5.00 (spt, 1H), 6.31 (t, 1H), 7.29 (s, 1H), 7.44 (dd, 1H), 8.51 (dd, 1H), 8.60 (s, 1H), 8.68 (s, 1H), 10.89 (s, 1H).

### Example 56: 6-Cyclobutoxy-2-(1-methyl-2-oxabicyclo[2.1.1]hexan-4-yl)-N-(6-methylpyrazolo[1,5-a]pyrimidin-3-yl)-2H-indazole-5-carboxamide Trifluoroacetate

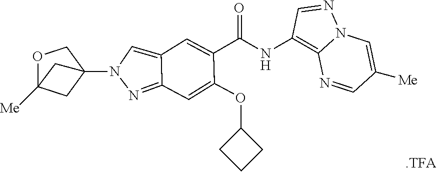 [SMILES: Cc1cnc2c(NC(=O)c3cc4cn(C56COC(C)(C5)C6)nc4cc3OC3CCC3)cnn2c1]

To a mixture of 6-cyclobutoxy-2-(1-methyl-2-oxabicyclo[2.1.1]hexan-4-yl)-2H-indazole-5-carboxylic acid (Preparation 98, 40 mg, 0.122 mmol) and 6-methylpyrazolo[1,5-a]pyrimidin-3-amine hydrochloride (33.7 mg, 0.183 mmol) in pyridine (1 mL) was added T3P® (50 wt. % in EtOAc, 388 mg, 0.609 mmol) and the mixture stirred at rt for 18 h. The mixture was diluted with EtOAc and H₂O and the aqueous phase extracted with further EtOAc (3×5 mL). The combined organics were dried (MgSO₄) and evaporated to dryness in vacuo. The residue was dissolved in DMSO (3 mL) and purified by prep-HPLC-Sunfire (gradient, 5-65%) to afford 6-cyclobutoxy-2-(1-methyl-2-oxabicyclo[2.1.1]hexan-4-yl)-N-(6-methylpyrazolo[1,5-a]pyrimidin-3-yl)-2H-indazole-5-carboxamide as a yellow solid (13.8 mg, 24.7%). LCMS m/z=459.1 [M+H]⁺. ¹H NMR (500 MHz, DMSO-d₆) δ: 1.50 (s, 3H), 1.72-1.85 (m, 1H), 1.89-2.00 (m, 1H), 2.18 (dd, 2H), 2.34 (d, 3H) 2.39-2.43 (m, 2H), 2.44-2.49 (m, 2H), 2.60-2.70 (m, 2H), 4.10 (s, 2H), 5.06 (quin, 1H), 7.05 (s, 1H), 8.46 (d, 1H), 8.61 (s, 1H), 8.70 (d, 2H), 8.93 (d, 1H), 10.65 (s, 1H).

### Example 57: 6-Isopropoxy-N-(1-methyl-2-oxo-1,2-dihydropyridin-3-yl)-2-(1-methyl-2-oxabicyclo[2.2.1]heptan-4-yl)-2H-indazole-5-carboxamide

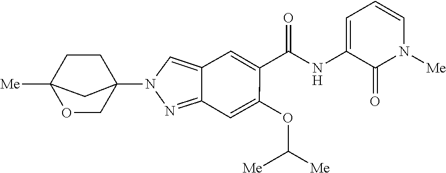 [SMILES: CC(C)Oc1cc2nn(C34CCC(C)(C3)OC4)cc2cc1C(=O)Nc1cccn(C)c1=O]

To a mixture of 6-isopropoxy-2-(1-methyl-2-oxabicyclo[2.2.1]heptan-4-yl)-2H-indazole-5-carboxylic acid (Preparation 96, 38.7 mg, 0.117 mmol) and 3-amino-1-methylpyridin-2 (1H)-one (28.2 mg, 0.176 mmol) in pyridine (1 mL) was added T3P® (50 wt. % in EtOAc, 373 mg, 0.586 mmol) and mixture stirred at rt for 18 h. The mixture was diluted with EtOAc and H₂O and the aqueous phase extracted with further EtOAc (3×5 mL). The combined organics were dried (MgSO₄) and evaporated to dryness in vacuo. The residue was dissolved in DMSO (3 mL) and purified by prep-HPLC-XSelect (gradient, 5-65%) to afford 6-isopropoxy-N-(1-methyl-2-oxo-1,2-dihydropyridin-3-yl)-2-(1-methyl-2-oxabicyclo[2.2.1]heptan-4-yl)-2H-indazole-5-carboxamide as a yellow solid (22.3 mg, 34.6%). LCMS m/z=437.2 [M+H]⁺. ¹H NMR (500 MHz, DMSO-d₆) δ: 1.36-1.47 (m, 3H), 1.51 (d, 6H), 1.78-1.89 (m, 1H), 1.97 (td, 1H), 2.20-2.31 (m, 2H), 2.31-2.40 (m, 2H), 3.52-3.63 (m, 3H), 4.01 (dd, 1H), 4.08 (d, 1H), 5.00 (spt, 1H), 6.31 (t, 1H), 7.28 (s, 1H), 7.44 (dd, 1H), 8.51 (dd, 1H), 8.59 (s, 1H), 8.64 (s, 1H), 10.90 (s, 1H).

### Example 58: 6-Isopropoxy-N-(1-methyl-2-oxo-1,2-dihydropyridin-3-yl)-2-(1-methyl-2-oxabicyclo[2.2.2]octan-4-yl)-2H-indazole-5-carboxamide Trifluoroacetate

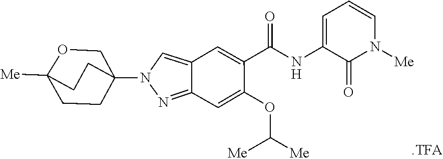 [SMILES: CC(C)Oc1cc2nn(C34CCC(C)(CC3)OC4)cc2cc1C(=O)Nc1cccn(C)c1=O]

To a mixture of 6-isopropoxy-2-(1-methyl-2-oxabicyclo[2.2.2]octan-4-yl)-2H-indazole-5-carboxylic acid (Preparation 95, 40 mg, 0.116 mmol) and 3-amino-1-methylpyridin-2 (1H)-one (28.0 mg, 0.174 mmol) in pyridine (1 mL) was added T3P® (50 wt. % in EtOAc, 370 mg, 0.581 mmol) and mixture stirred at rt for 18 h. The mixture was diluted with EtOAc and H₂O and the aqueous phase extracted with further EtOAc (3×5 mL). The combined organics were dried (MgSO₄) and evaporated to dryness in vacuo. The residue was dissolved in DMSO (3 mL) and purified by prep-HPLC-Sunfire (gradient, 5-60%) to afford 6-isopropoxy-N-(1-methyl-2-oxo-1,2-dihydropyridin-3-yl)-2-(1-methyl-2-oxabicyclo[2.2.2]octan-4-yl)-2H-indazole-5-carboxamide as a yellow solid (34.4 mg, 65.7%). LCMS m/z=451.2 [M+H]⁺. ¹H NMR (500 MHz, DMSO-d₆) δ: 1.13 (s, 3H), 1.51 (d, 6H), 1.81-2.01 (m, 4H), 2.25 (td, 2H), 2.38 (td, 2H), 3.45-3.63 (m, 3H), 4.02-4.22 (m, 2H), 5.00 (spt, 1H), 6.30 (t, 1H), 7.27 (s, 1H), 7.44 (dd, 1H), 8.51 (dd, 1H), 8.58 (s, 1H), 8.62 (s, 1H), 10.90 (s, 1H).

### Example 59-103

The title compounds were prepared from the appropriate carboxylic acid (Acid-1 to Acid-13, below) and the appropriate amine (R—NH₂) using an analogous method to that described for Example 58. Purification as outlined in the table.
Acid-1: 6-isopropoxy-2-(4-methoxybutan-2-yl)-2H-indazole-5-carboxylic acid (Preparation 90); Acid-2: 6-isopropoxy-2-(3-methoxypropyl)-2H-pyrazolo[3,4-b]pyridine-5-carboxylic acid (Preparation 107); Acid-3: 6-isopropoxy-2-(tetrahydro-2H-pyran-4-yl)-2H-pyrazolo[3,4-b]pyridine-5-carboxylic acid (Preparation 108); Acid-4: 6-isopropoxy-2-(tetrahydro-2H-pyran-4-yl)-2H-indazole-5-carboxylic acid (Preparation 91); Acid-5: 6-isopropoxy-2-((1r,3r)-3-methoxycyclobutyl)-2H-indazole-5-carboxylic acid (Preparation 102); Acid-6: 2-(tetrahydro-2H-pyran-4-yl)-6-((tetrahydrofuran-3-yl)oxy)-2H-indazole-5-carboxylic acid (Preparation 92); Acid-7: 6-isopropoxy-2-((tetrahydrofuran-3-yl)methyl)-2H-pyrazolo[3,4-b]pyridine-5-carboxylic acid (Preparation 104); Acid-8: 6-cyclobutoxy-2-(1-methyl-2-oxabicyclo[2.1.1]hexan-4-yl)-2H-indazole-5-carboxylic acid (Preparation 98); Acid-9: 6-ethoxy-2-(1-methyl-2-oxabicyclo[2.1.1]hexan-4-yl)-2H-indazole-5-carboxylic acid (Preparation 100); Acid-10: 6-methoxy-2-(1-methyl-2-oxabicyclo[2.1.1]hexan-4-yl)-2H-indazole-5-carboxylic acid (Preparation 99); Acid-11: 6-isopropoxy-2-(1-methyl-2-oxabicyclo[2.1.1]hexan-4-yl)-2H-indazole-5-carboxylic acid (Preparation 97); Acid-12: 6-isopropoxy-2-(1-methyl-2-oxabicyclo[2.2.1]heptan-4-yl)-2H-indazole-5-carboxylic acid (Preparation 96); Acid-13: 6-isopropoxy-2-(1-methyl-2-oxabicyclo[2.2.2]octan-4-yl)-2H-indazole-5-carboxylic acid (Preparation 95); Acid-17: 6-cyclobutoxy-2-(1-methyl-2-oxabicyclo[2.2.2]octan-4-yl)-2H-indazole-5-carboxylic acid (Preparation 137).
HPLC conditions in the following table refer to the following columns. Prep-HPLC-Synergi=Phenomenex Synergi C18 150×30 mm, 4 mm; MeCN/H₂O+0.05% HCl; Prep-HPLC-YMC=YMC-Actus Triart C18 150×30 mm, 5 μm; MeCN/H₂O (0.225% HCO₂H); Prep-HPLC-DuraShell=Agela DuraShell C18 150×25 mm, 5 μm; MeCN/H₂O (0.04% NH₄OH+10 mM NH₄HCO₃)); Prep-HPLC-Xtimate=Welch Xtimate C18 150×25 mm, 5 μm; MeCN/H₂O (10 mM NH₄HCO₃); Prep-HPLC-Gemini=Phenomenex Gemini NX-C1 75×30 mm, 3 μm; MeCN/H₂O (0.04% NH₄OH+10 mM NH₄HCO₃); Prep-HPLC-Sunfire=Waters Sunfire C18 100×19 mm, 5 μm; MeOH/H₂O+0.1% TFA; Prep-HPLC-XSelect=Waters XSelect CSH Prep C18 100×19 mm, 5 μm; MeOH/H₂O+0.1% NH₄OH

| Example | |
|---|---|
| No. | Name/Structure/HPLC/Reactants/Data |
| 59 | N-(6-(difluoromethyl)pyridin-2-yl)-6-isopropoxy-2-(4-methoxybutan-2-yl)-2H- |
| | indazole-5-carboxamide hydrochloride |
| | 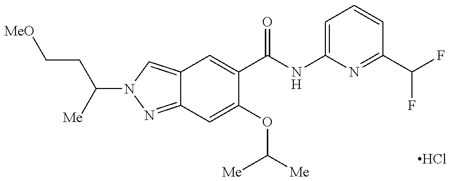 [SMILES: COCCC(C)n1cc2cc(C(=O)Nc3cccc(C(F)F)n3)c(OC(C)C)cc2n1 ; Cl] |
| | Pre-HPLC-Synergi; 49-69% |
| | Reactants: Acid-1; RNH₂: 6-(difluoromethyl)pyridin-2-amine |
| | 51 mg, 22%. LCMS m/z = 433.1 [M + H]⁺; ¹H NMR (500 MHz, MeOH-d₄) δ: |
| | 1.57 (d, 6H), 1.66 (d, 3H), 2.15-2.20 (m, 1H), 2.25-2.30 (m, 1H), 3.10-3.15 (m, |
| | 1H), 3.25 (s, 3H), 3.70-3.80 (m, 2H), 4.90-5.00 (m, 1H), 6.50-6.75 (m, 1H), |
| | 7.18 (s, 1H), 7.40-7.50 (m, 1H), 7.90-8.00 (m, 1H), 8.40-8.50 (m, 2H), 8.63 (s, |
| | 1H). |
| 60 | 6-Isopropxy-2-(3-methoxypropyl)-N-(6-(trifluoromethyl)pyridin-2-yl)-2H- |
| | pyrazolo[3,4-b]pyridine-5-carboxamide hydrochloride |
| | 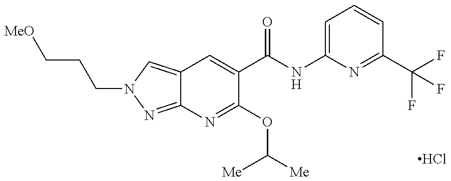 [SMILES: COCCCn1cc2cc(C(=O)Nc3cccc(C(F)(F)F)n3)c(OC(C)C)nc2n1 ; Cl] |
| | 66-86% Prep-HPLC-Synergi |
| | Reactants: Acid-2; RNH₂: 6-(trifluoromethyl)pyridin-2-amine |
| | 10.5 mg, 8.7%. LCMS m/z = 438.0 [M + H]⁺; ¹H NMR (500 MHz, MeOH-d₄) δ: |
| | 1.58 (d, 6H), 2.21-2.27 (m, 2H), 3.33 (s, 3H), 3.37 (t, 2H), 4.51 (t, 2H), 5.67- |
| | 5.72 (m, 1H), 7.54 (d, 1H), 8.03-8.07 (m, 1H), 8.38 (s, 1H), 8.56-8.58 (m, 1H), |
| | 8.97 (s, 1H). |
| 61 | 6-Isopropoxy-2-(3-methoxypropyl)-N-(6-methoxypyridin-2-yl)-2H- |
| | pyrazolo[3,4-b]pyridine-5-carboxamide hydrochloride |
| | 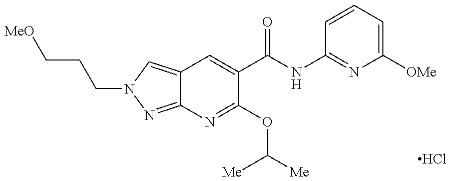 [SMILES: COCCCn1cc2cc(C(=O)Nc3cccc(OC)n3)c(OC(C)C)nc2n1 ; Cl] |
| | 58-78% Pre-HPLC-Synergi |
| | Reactants: Acid-2; RNH₂: 6-methoxypyridin-2-amine |
| | 67 mg, 61%. LCMS m/z = 400.1 [M + H]⁺; ¹H NMR (500 MHz, MeOH-d₄) δ: |
| | 1.60 (d, 6H), 2.21-2.27 (m, 2H), 3.33 (s, 3H), 3.37 (t, 2H), 3.90 (s, 3H), 4.50 (t, |
| | 2H), 5.67-5.72 (m, 1H), 6.55 (d, 1H), 7.66-7.70 (m, 1H), 7.83 (d, 1H), 8.37 (s, |
| | 1H), 8.96 (s, 1H). |
| 62 | N-(6-(difluoromethyl)pyridin-2-yl)-6-isopropoxy-2-(3-methoxsypropyl)-2H- |
| | pyrazolo[3,4-b]pyridine-5-carboxamide hydrochloride |
| | 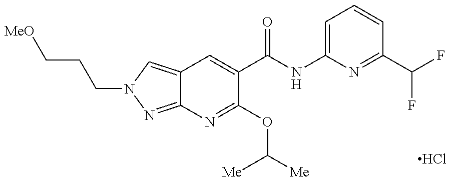 [SMILES: COCCCn1cc2cc(C(=O)Nc3cccc(C(F)F)n3)c(OC(C)C)nc2n1 ; Cl] |
| | 56-66% Prep-HPLC-Synergi |
| | Reactants: Acid-2; RNH₂: 6-(difluoromethyl)pyridin-2-amine |
| | 45.7 mg, 39.8%. LCMS m/z = 420.0 [M + H]⁺; ¹H NMR (500 MHz, MeOH-d₄) |
| | δ: 1.58 (d, 6H), 2.21-2.27 (m, 2H), 3.33 (s, 3H), 3.37 (t, 2H), 4.50 (t, 2H), 5.67- |
| | 5.73 (m, 1H), 6.50-6.73 (m, 1H), 7.43 (d, 1H), 7.97-8.01 (m, 1H), 8.37 (s, 1H), |
| | 8.45 (d, 1H), 8.96 (s, 1H). |
| 63 | N-(6-(difluoromethyl)pyridin-2-yl)-6-isopropoxy-2-(tetrahydro-2H-pyran-4-yl)- |
| | 2H-pyrazolo[3,4-b]pyridine-5-carboxamide |
| | 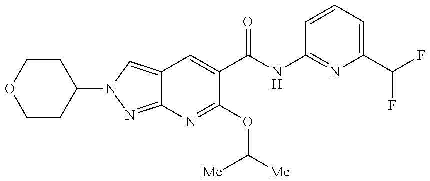 [SMILES: CC(C)Oc1nc2nn(C3CCOCC3)cc2cc1C(=O)Nc1cccc(C(F)F)n1] |
| | 55-75% Prep-HPLC-Synergi |
| | Reactants: Acid-3; RNH₂: 6-(difluoromethyl)pyridin-2-amine |
| | 14.7 mg, 13.5%. LCMS m/z = 432.0 [M + H]⁺; ¹H NMR (400 MHz, MeOH-d₄) |
| | δ: 1.59 (d, 6H), 2.15-2.20 (m, 2H), 2.21-2.32 (m, 2H), 3.61-3.68 (m, 2H), 4.10- |
| | 4.15 (m, 2H), 4.68-4.76 (m, 1H), 5.67-5.74 (m, 1H), 6.48-6.76 (m, 1H), 7.44 (d, |
| | 1H), 7.98-8.02 (m , 1H), 8.45-8.48 (m, 2H), 8.97 (s, 1H). |
| 64 | 6-Isopropoxy-N-(pyridin-2-yl)-2-(tetrahydro-2H-pyran-4-yl)-2H-indazole-5- |
| | carboxamide formate |
| | 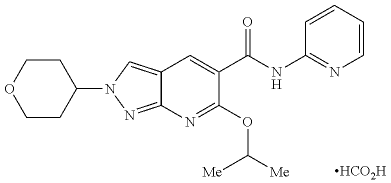 [SMILES: CC(C)Oc1nc2nn(C3CCOCC3)cc2cc1C(=O)Nc1ccccn1 ; "·HCO2H"] |
| | Prep-HPLC-YMC; 44-68% |
| | Reacetants: Acid-4; RNH₂: pyridin-2-amine |
| | 45 mg, 34.5%. LCMS m/z = 381.1 [M + H]⁺; ¹H NMR (400 MHz, DMSO-d₆) δ: |
| | 1.45-1.47 (d, 6H), 2.07-2.13 (m, 4H), 3.49-3.54 (m, 2H), 3.99-4.03 (m, 2H), |
| | 4.76-4.77 (m, 1H), 4.96-4.97 (m, 1H), 7.13-7.16 (m, 1H), 7.26 (s, 1H), 7.83- |
| | 7.85 (d, 1H), 8.28-8.30 (d, 1H), 8.36-8.37 (m, 1H), 8.54 (s, 1H), 8.60 (s, 1H), |
| | 10.84 (s, 1H). |
| 65 | N-(4-(difluoromethyl)pyrimidin-2-yl)-6-isopropoxy-2-(tetrahydro-2H-pyran-4- |
| | yl)-2H-indazole-5-carboxamide hydrochloride |
| | 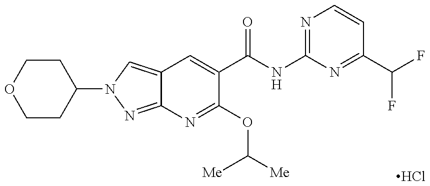 [SMILES: CC(C)Oc1nc2nn(C3CCOCC3)cc2cc1C(=O)Nc1nccc(C(F)F)n1 ; Cl] |
| | 41-61% Prep-HPLC-Synergi |
| | Reactants: Acid-4; RNH₂: 4-(difluoromethyl)pyrimidin-2-amine |
| | 33 mg, 46%. LCMS m/z = 432.3 [M + H]⁺; ¹H NMR (500 MHz, CDCl₃) δ: 1.61 |
| | (d, 6H), 2.16-2.22 (m, 2H), 2.52-2.55 (m, 2H), 3.70 (t, 2H), 4.18-4.22 (m, 2H), |
| | 4.98-5.01 (m, 1H), 5.30 (brs, 1H), 6.41-6.64 (m, 1H), 7.33 (s, 1H), 7.37-7.38 |
| | (m, 1H), 8.33 (s, 1H), 8.88-8.91 (m, 2H), 10.88 (s, 1H). |
| 66 | 6-Isopropoxy-N-(6-methoxypyridin-2-yl)-2-(tetrahydro-2H-pyran-4-yl)-2H- |
| | indazole-5-carboxamide |
| | 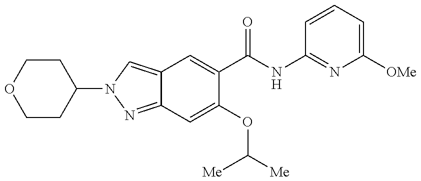 [SMILES: COc1cccc(NC(=O)c2cc3cn(C4CCOCC4)nc3cc2OC(C)C)n1] |
| | Prep-HPLC-DuraShell; 45-75%. |
| | Reactants: Acid-4; RNH₂: 6-methoxypyridin-2-amine |
| | 31.5 mg, 46.7%. LCMS m/z = 411.2 [M + H]⁺; ¹H NMR (500 MHz, CDCl₃) δ: |
| | 1.61 (d, 6H), 2.18-2.33 (m, 4H), 3.58-3.65 (m, 2H), 3.90 (s, 3H), 4.15-4.23 (m, |
| | 2H), 4.58-4.65 (m, 1H), 4.84-4.93 (m, 1H), 6.50 (d, 1H), 7.13 (s, 1H), 7.63 (t, |
| | 1H), 7.97 (d, 1H), 8.08 (s, 1H), 8.78 (s, 1H), 10.85 (s, 1H). |
| 67 | 6-Isopropoxy-N-(1-methyl-1H-pyrazol-3-yl)-2-(tetrahydro-2H-pyran-4-yl)-2H- |
| | indazole-5-carboxamide |
| | 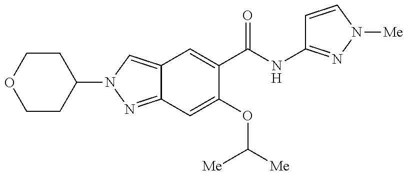 [SMILES: CC(C)Oc1cc2nn(C3CCOCC3)cc2cc1C(=O)Nc1ccn(C)n1] |
| | Prep-HPLC-DuraShell; 26-56%. |
| | Reactants: Acid-4; RNH₂: 1-methyl-1H-pyrazol-3-amine |
| | 35.2 mg, 55.9%. LCMS m/z = 384.1 [M + H]⁺; ¹H NMR (500 MHz, CDCl₃) δ: |
| | 1.57 (d, 6H), 2.19-2.32 (m, 4H), 3.58-3.65 (m, 2H), 3.84 (s, 3H), 4.15-4.20 (m, |
| | 2H), 4.55-4.65 (m, 1H), 4.78-4.88 (m, 1H), 6.83 (d, 1H), 7.11 (s, 1H), 7.29 (d, |
| | 1H), 8.06 (s, 1H), 8.76 (s, 1H), 10.56 (s, 1H). |
| 68 | N-(2-fluoro-3-methylphenyl)-6-isopropoxy-2-(tetrahydro-2H-pyran-4-yl)-2H- |
| | indazole-5-carboxamide |
| | 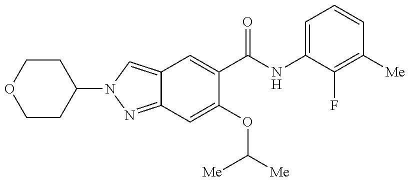 [SMILES: Cc1cccc(NC(=O)c2cc3cn(C4CCOCC4)nc3cc2OC(C)C)c1F] |
| | Prep-HPLC-YMC; 65-90%. |
| | Reactants: Acid-4; RNH₂: 2-fluoro-3-methylaniline |
| | 45.7 mg, 67.9%. LCMS m/z = 412.1 [M + H]⁺; ¹H NMR (500 MHz, CDCl₃) δ: |
| | 1.56 (d, 6H), 2.22-2.30 (m, 4H), 2.33 (s, 3H), 3.58-3.85 (m, 2H), 4.19 (d, 2H), |
| | 4.56-4.65 (m, 1H), 4.85-4.93 (m, 1H), 6.92 (t, 1H), 7.07 (t, 1H), 7.14 (s, 1H), |
| | 8.07 (s, 1H), 8.48 (t, 1H), 8.79 (s, 1H), 10.53 (s, 1H). |
| 69 | 6-Isopropoxy-N-(2-methoxypyridin-3-yl)-2-(tetrahydro-2H-pyran-4-yl)-2H- |
| | indazole-5-carboxamide |
| | 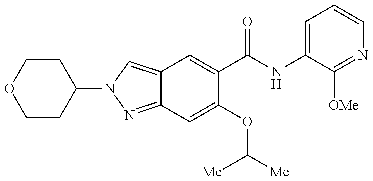 [SMILES: COc1ncccc1NC(=O)c1cc2cn(C3CCOCC3)nc2cc1OC(C)C] |
| | Prep-HPLC-Xtimate; 39-69%. |
| | Reactants: Acid-4; RNH₂: 2-methoxypyridin-3-amine |
| | 45.9 mg, 67.9%. LCMS m/z = 411.1 [M + H]⁺; ¹H NMR (500 MHz, CDCl₃) δ: |
| | 1.56 (d, 6H), 2.20-2.32 (m, 4H), 3.56-3.65 (m, 2H), 4.08 (s, 3H), 4.15-4.22 (m, |
| | 2H), 4.56-4.65 (m, 1H), 4.86-4.93 (m, 1H), 6.93-6.97 (m, 1H), 7.14 (s, 1H), |
| | 7.88 (dd, 1H), 8.07 (s, 1H), 8.75 (s, 1H), 8.91 (dd, 1H), 10.49 (s, 1H). |
| 70 | N-(2-(difluoromethoxy)pyridin-3-yl)-6-isopropoxy-2-(tetrahydro-2H-pyran-4- |
| | yl)-2H-indazole-5-carboxamide hydrochloride |
| | 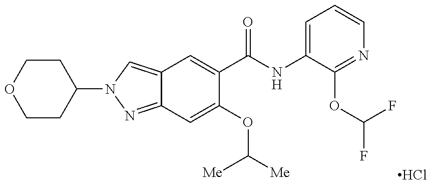 [SMILES: CC(C)Oc1cc2nn(C3CCOCC3)cc2cc1C(=O)Nc1cccnc1OC(F)F ; Cl] |
| | 52-72% Prep-HPLC-Synergi |
| | Reactants: Acid-4; RNH₂: 2-(difluoromethoxy)pyridin-3-amine |
| | 50 mg, 40%. LCMS m/z = 447.1 [M + H]⁺; ¹H NMR (400 MHz, MeOH-d₄) δ: |
| | 1.58 (d, 6H), 2.20-2.28 (m, 4H), 3.62-3.67 (m, 2H), 4.11-4.15 (m, 2H), 4.77- |
| | 4.79 (m, 1H), 4.99-5.04 (m, 1H), 7.21 (s, 1H), 7.22-7.26 (m, 1H), 7.60-7.90 (m, |
| | 1H), 7.94 (q, 1H), 8.59 (s, 1H), 8.70 (s, 1H), 8.90-8.92 (m, 1H). |
| 71 | 6-Isopropoxy-N-(pyrazolo[1,5-a]pyridin-4-yl)-2-(tetrahydro-2H-pyran-4-yl)- |
| | 2H-indazole-5-carboxamide hydrochloride |
| | 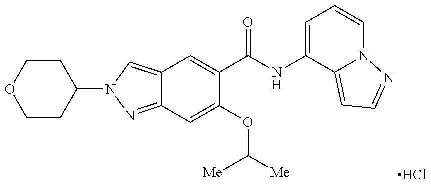 [SMILES: CC(C)Oc1cc2nn(C3CCOCC3)cc2cc1C(=O)Nc1cccn2nccc12 ; Cl] |
| | 41-61% Prep-HPLC-Synergi |
| | Reactants: Acid-4; RNH₂: pyrazolo[1,5-a]pyridin-4-amine |
| | 60 mg, 87%. LCMS m/z = 420.0 [M + H]⁺; ¹H NMR (500 MHz, CDCl₃) δ: 1.62 |
| | (d, 6H), 2.19-2.25 (m, 2H), 2.41-2.44 (m, 2H), 3.67 (t, 2H), 4.19-4.23 (m, 2H), |
| | 5.01-5.05 (m, 2H), 6.64 (s, 1H), 6.90-6.94 (m, 1H), 7.33 (s, 1H), 8.02 (s, 1H), |
| | 8.21-8.25 (m, 2H), 8.45-8.47 (m, 1H), 8.86 (s, 1H), 10.15 (s, 1H). |
| 72 | 6-Isopropoxy-N-(pyrazolo[1,5-a]pyrimidin-3-yl)-2-(tetrahydro-2H-pyran-4-yl)- |
| | 2H-indazole-5-carboxamide |
| | 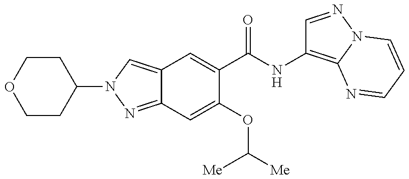 [SMILES: CC(C)Oc1cc2nn(C3CCOCC3)cc2cc1C(=O)Nc1cnn2cccnc12] |
| | Prep-HPLC-YMC; 40-65%. |
| | Reactants: Acid-4; RNH₂: pyrazolo[1,5-a]pyrimidin-3-amine |
| | 55.7 mg, 57.5%. LCMS m/z = 421.1 [M + H]⁺; ¹H NMR (400 MHz, MeOH-d₄) |
| | δ: 1.64 (d, 6H), 2.16-2.29 (m, 4H), 3.62-3.68 (m, 2H), 4.11-4.15 (m, 2H), 4.73- |
| | 4.76 (m, 1H), 5.01-5.04 (m, 1H), 6.99-7.02 (m, 1H), 7.20 (s, 1H), 8.47-8.51 (m, |
| | 2H), 8.69 (s, 1H), 8.82-8.86 (m, 2H). |
| 73 | N-(2,3-dihydrobenzofuran-7-yl)-6-isopropoxy-2-(tetrahydro-2H-pyran-4-yl)- |
| | 2H-indazole-5-carboxamide hydrochloride |
| | 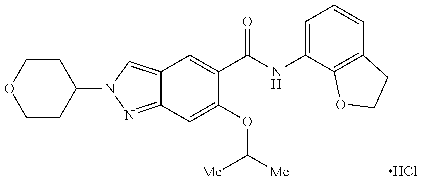 [SMILES: CC(C)Oc1cc2nn(C3CCOCC3)cc2cc1C(=O)Nc1cccc2c1OCC2 ; Cl] |
| | 56-76% Prep-HPLC-Synergi |
| | Reactants: Acid-4; RNH₂: 2,3-dihydrobenzofuran-7-amine |
| | 56 mg, 80%. LCMS m/z = 422.1 [M + H]⁺; ¹H NMR (500 MHz, CDCl₃) δ: |
| | 1.56 (d, 6H), 2.24-2.32 (m, 4H), 3.31 (t, 2H), 3.64 (t, 2H), 4.17-4.21 (m, 2H), |
| | 4.64-4.67 (m, 2H), 4.68 (brs, 1H), 4.89-4.92 (m, 1H), 6.89-6.92 (m, 1H), 6.96- |
| | 6.99 (m, 1H), 7.17 (s, 1H), 8.10 (s, 1H), 8.36-8.38 (m, 1H), 8.80 (s, 1H), 10.31 |
| | (s, 1H). |
| 74 | N-(6-(difluoromethyl)pyridin-2-yl)-6-isopropoxy-2-((1R,3R)-3- |
| | methoxycyclobutyl)-2H-indazole-5-carboxamide |
| | 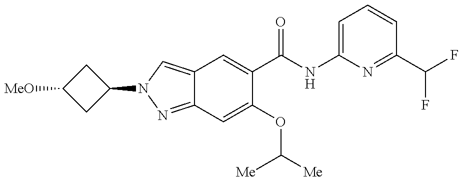 [SMILES: CO[C@H]1C[C@H](n2cc3cc(C(=O)Nc4cccc(C(F)F)n4)c(OC(C)C)cc3n2)C1] |
| | Reactants: Acid-5; RNH₂: 6-(difluoromethyl)pyridin-2-amine |
| | CombiFlash: EtOAc/PE; 0.1-1:1 |
| | 175 mg, 49%. LCMS m/z = 431.1 [M + H]⁺; ¹H NMR (400 MHz, MeOH-d₄) δ: |
| | 1.57 (d, 6H), 2.60-2.70 (m, 2H), 2.80-2.90 (m, 2H), 3.33 (s, 3H), 4.30-4.40 (m, |
| | 1H), 4.90-5.00 (m, 1H), 5.20-5.30 (m, 1H), 6.50-6.75 (m, 1H), 7.18 (s, 1H), |
| | 7.40-7.45 (m, 1H), 7.90-8.00 (m, 1H), 8.40-8.50 (m, 2H), 8.61 (s, 1H). |
| 75 | N-(6-(difluoromethyl)pyridin-2-yl)-2-(tetrahydro-2H-pyran-4-yl)-6- |
| | ((tetrahydrofuran-3-yl)oxy)-2H-indazole-5-carboxamide |
| | 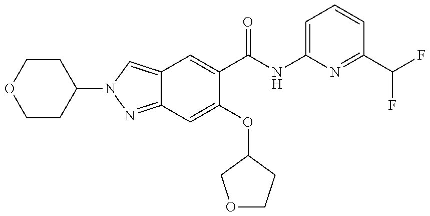 [SMILES: O=C(Nc1cccc(C(F)F)n1)c1cc2cn(C3CCOCC3)nc2cc1OC1CCOC1] |
| | Reactants: Acid-6; RNH₂: 6-(difluoromethyl)pyridin-2-amine |
| | Prep-HPLC-Xtimate; 38-65%. |
| | 35 mg, 36%. LCMS m/z = 459.0 [M + H]⁺; ¹H NMR (500 MHz, CDCl₃) δ: |
| | 2.23-2.33 (m, 4H), 2.38-2.54 (m, 2H), 3.62 (td, 2H), 4.00-4.05 (m, 1H), 4.12- |
| | 4.28 (m, 5H), 4.58-4.66 (m, 1H), 5.22-5.26 (m, 1H), 6.38-6.62 (m, 1H), 7.02 (s, |
| | 1H), 7.36 (d, 1H), 7.88 (t, 1H), 7.10 (s, 1H), 8.52 (d, 1H), 8.75 (s, 1H), 10.69 (s, |
| | 1H). |
| 76 | N-(2-fluoro-3-methylphenyl)-6-isopropoxy-2-((tetrahydrofuran-3-yl)methyl)- |
| | 2H-pyrazolo[3,4-b]pyridine-5-carboxamide |
| | 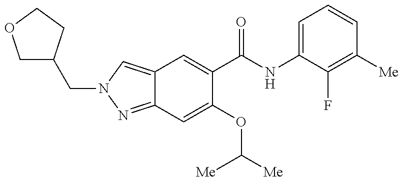 [SMILES: Cc1cccc(NC(=O)c2cc3cn(CC4CCOC4)nc3cc2OC(C)C)c1F] |
| | Reactants: Acid-7; RNH₂: 2-fluoro-3-methylaniline |
| | 60-80% Prep-HPLC-Synergi |
| | 15.1 mg, 18.6%. LCMS m/z = 413.1 [M + H]⁺; ¹H NMR (400 MHz, MeOH-d₄) |
| | δ: 1.57 (d, 6H), 1.71-1.80 (m, 1H), 2.04-2.12 (m, 1H), 2.34 (s, 3H), 2.97-3.05 |
| | (m, 1H), 3.61-3.66 (m, 1H), 3.74-3.82 (m, 2H), 3.90-3.96 (m, 1H), 4.43 (d, 2H), |
| | 5.76-5.83 (m, 1H), 7.00-7.11 (m, 2H), 8.28-8.30 (m, 1H), 8.43 (s, 1H), 9.01 (s, |
| | 1H), 10.49 (s, 1H). |
| 77 | 6-Isopropoxy-N-(pyridin-2-yl)-2-((tetrahydrofuran-3-yl)methyl)-2H- |
| | pyrazolo[3,4-b]pyridine-5-carboxamide |
| | 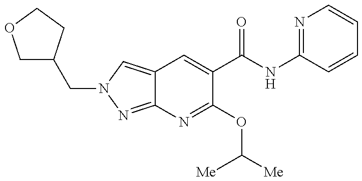 [SMILES: CC(C)Oc1nc2nn(CC3CCOC3)cc2cc1C(=O)Nc1ccccn1] |
| | Reactants: Acid-7; RNH₂: pyridin-2-amine |
| | 36-56% Prep-HPLC-Synergi |
| | 32.7 mg, 41.5%. LCMS m/z = 382.0 [M + H]⁺; ¹H NMR (400 MHz, MeOH-d₄) |
| | δ: 1.58 (d, 6H), 1.71-1.80 (m, 1H), 2.02-2.12 (m, 1H), 2.96-3.03 (m, 1H), 3.61- |
| | 3.66 (m, 1H), 3.74-3.82 (m, 2H), 3.90-3.97 (m, 1H), 4.43 (d, 2H), 5.68-5.75 (m, |
| | 1H), 7.14-7.18 (m, 1H), 7.81-7.87 (m, 1H), 8.32-8.36 (m, 2H), 8.42 (s, 1H), |
| | 8.96 (s, 1H). |
| 78 | 6-Isopropoxy-N-(2-methoxypyridin-3-yl)-2-((tetrahydrofuran-3-yl)methyl)-2H- |
| | pyrazolo[3,4-b]pyridine-5-carboxamide |
| | 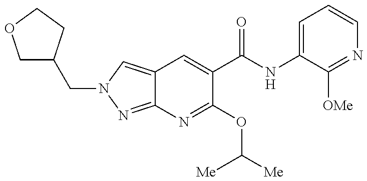 [SMILES: COc1ncccc1NC(=O)c1cc2cn(CC3CCOC3)nc2nc1OC(C)C] |
| | Reactants: Acid-7; RNH₂: 2-methoxypyridin-3-amine |
| | 51-71% Prep-HPLC-Synergi |
| | 30.6 mg, 44.4%. LCMS m/z = 412.0 [M + H]⁺; ¹H NMR (400 MHz, MeOH-d₄) |
| | δ: 1.59 (d, 6H), 1.71-1.80 (m, 1H), 2.02-2.12 (m, 1H), 2.06-3.03 (m, 1H), 3.61- |
| | 3.66 (m, 1H), 3.74-3.82 (m, 2H), 3.90-3.96 (m, 1H), 4.10 (s, 3H), 4.26 (d, 2H), |
| | 5.80-5.87 (m, 1H), 6.98-7.02 (m, 1H), 7.87-7.90 (m, 1H), 8.42 (m, 1H), 8.79- |
| | 8.82 (m, 1H), 9.02 (s, 1H). |
| 79 | 6-Isopropoxy-N-(1-methyl-1H-pyrazol-3-yl)-2-((tetrahydrofuran-3-yl)methyl)- |
| | 2H-pyrazolo[3,4-b]pyridine-5-carboxamide |
| | 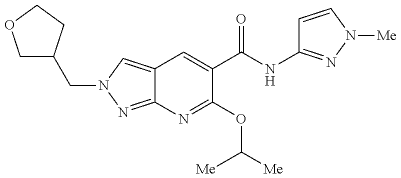 [SMILES: CC(C)Oc1nc2nn(CC3CCOC3)cc2cc1C(=O)Nc1ccn(C)n1] |
| | Reactants: Acid-7; RNH₂: 1-methyl-1H-pyrazol-3-amine |
| | 32-52% Prep-HPLC-Synergi |
| | 26.2 mg, 40.8%. LCMS m/z = 385.0 [M + H]⁺; ¹H NMR (400 MHz, MeOH-d₄) |
| | δ: 1.56 (d, 6H), 1.70-1.79 (m, 1H), 2.95-3.03 (m, 2H), 3.61-3.65 (m, 1H), 3.74- |
| | 3.82 (m, 2H), 3.85 (s, 3H), 3.90-3.96 (m, 1H), 4.42 (d, 2H), 5.67-5.74 (m, 1H), |
| | 6.69 (d, 1H), 7.52 (d, 1H), 8.40 (s, 1H), 8.89 (s, 1H). |
| 80 | N-(1-(difluoromethyl)-1H-pyrazol-3-yl)-6-isopropoxy-2-((tetrahydrofuran-3- |
| | yl)methyl)-2H-pyrazolo[3,4-b]pyridine-5-carboxamide |
| | 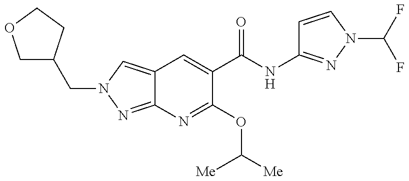 [SMILES: CC(C)Oc1nc2nn(CC3CCOC3)cc2cc1C(=O)Nc1ccn(C(F)F)n1] |
| | Reactants: Acid-7; RNH₂: 1-(difluoromethyl)-1H-pyrazol-3-amine |
| | Prep-HPLC-Gemini; 33-63%. |
| | 28.6 mg, 48.6%. LCMS m/z = 443.0 [M + Na]⁺; ¹H NMR (500 MHz, MeOH-d₄) |
| | δ: 1.55 (d, 6H), 1.71-1.78 (m, 1H), 2.03-2.10 (m, 1H), 2.96-3.02 (m, 1H), 3.61- |
| | 3.65 (m, 1H), 3.75-3.81 (m, 2H), 3.90-3.95 (m, 1H), 4.42 (d, 2H), 5.66-5.72 (m, |
| | 1H), 7.01 (d, 1H), 7.28-7.53 (m, 1H), 8.00 (d, 1H), 8.41 (s, 1H), 8.88 (s, 1H). |
| 81 | N-(5-fluoro-1-methyl-2-oxo-1,2-dihydroxypyridin-3-yl)-6-isopropxy-2- |
| | ((tetrahydrofuran-3-yl)methyl)-2H-pyrazolo[3,4-b]pyridine-5-carboxamide |
| | 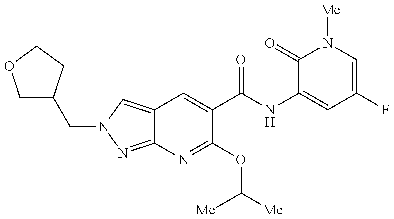 [SMILES: CC(C)Oc1nc2nn(CC3CCOC3)cc2cc1C(=O)Nc1cc(F)cn(C)c1=O] |
| | Reactants: Acid-7; RNH₂: 3-amino-5-fluoro-1-methylpyridin-2(1H)-one |
| | Prep-HPLC-Xtimate; 32-62%. |
| | 47.8 mg, 68%. LCMS m/z = 452.0 [M + Na]⁺; ¹H NMR (400 MHz, CDCl₃) δ: |
| | 1.63 (d, 6H), 1.67-1.80 (m, 1H), 2.04-2.16 (m, 1H), 3.06-3.17 (m, 1H), 3.61- |
| | 3.67 (m, 4H), 3.75-3.85 (m, 2H), 3.93-4.00 (m, 1H), 4.34 (d, 2H), 5.85-5.98 (m, |
| | 1H), 6.96 (t, 1H), 7.98 (s, 1H), 8.60-8.70 (m, 1H), 9.00 (s, 1H), 11.11 (s, 1H). |
| 82 | 6-Isopropoxy-N-(pyrazolo[1,5-a]pyrimidin-3-yl)-2-(tetrahydro-2H-pyran-4-yl)- |
| | 2H-pyrazolo[3,4- b]pyridine-5-carboxamide |
| | 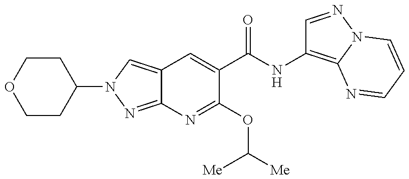 [SMILES: CC(C)Oc1nc2nn(C3CCOCC3)cc2cc1C(=O)Nc1cnn2cccnc12] |
| | Reactants: Acid-3; RNH₂: pyrazolo[1,5-a]pyrimdiin-3-amine |
| | Prep-HPLC-Xtimate; 28-58%. |
| | 10.7 mg, 19.2%. LCMS m/z = 444.1 [M + Na]⁺; ¹H NMR (400 MHz, CDCl₃) δ: |
| | 1.66 (d, 6H), 2.23-2.27 (m, 2H), 2.28-2.34 (m, 2H), 3.58-3.64 (m, 2H), 4.17- |
| | 4.21 (m, 2H), 4.56-4.63 (m, 1H), 5.88-5.94 (m, 1H), 6.80-6.83 (m, 1H), 8.06 (s, |
| | 1H), 8.41-8.43 (m, 1H), 8.60-8.63 (m, 1H), 9.00 (s, 1H), 9.11 (s, 1H), 10.81 (s, |
| | 1H). |
| 83 | 6-Cyclobutoxy-N-(1-methyl-2-oxo-1,2-dihydropyridin-3-yl)-2-(1-methyl-2- |
| | oxabicyclo[2.1.1]hexan-4-yl)-2H-indazole-5-carboxamide trifluoroacetate |
| | 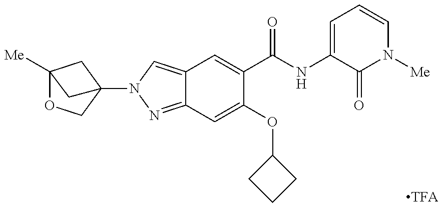 [SMILES: Cn1cccc(NC(=O)c2cc3cn(C45COC(C)(C4)C5)nc3cc2OC2CCC2)c1=O] |
| | Prep-HPLC-Sunfire; 5-60% |
| | Reactants: Acid-8; RNH₂: 3-amino-1-methylpyridin-2(1H)-one |
| | 34 mg, 36.7%. LCMS m/z = 435.2 [M + H]⁺; ¹H NMR (500 MHz, DMSO-d₆) δ: |
| | 1.37-1.57 (m, 3H), 1.66-1.85 (m, 1H), 1.91 (qt, 1H), 2.17 (dd, 2H), 2.34-2.43 |
| | (m, 2H), 2.55-2.64 (m, 4H), 3.51-3.62 (m, 3H), 4.05-4.18 (m, 2H), 5.00 (quin, |
| | 1H), 6.27-6.37 (m, 1H), 7.02 (s, 1H), 7.45 (dd, 1H), 8.52 (dd, 1H), 8.62 (s, 1H), |
| | 8.69 (s, 1H), 10.91 (s, 1H). |
| 84 | 6-Cyclobutoxy-N-(1-(difluoromethyl)-2-oxo-1,2-dihydropyridin-3-yl)-2-(1- |
| | methyl-2-oxabicyclo[2.1.1]hexan-4-yl)-2H-indazole-5-carboxamide |
| | trifluoroacetate |
| | 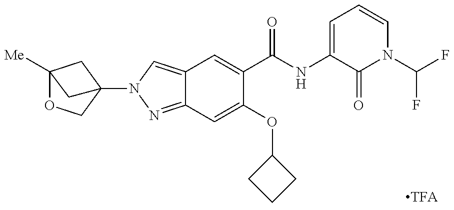 [SMILES: CC12CC(n3cc4cc(C(=O)Nc5cccn(C(F)F)c5=O)c(OC5CCC5)cc4n3)(CO1)C2] |
| | Prep-HPLC-Sunfire; 10-70% |
| | Reactants: Acid-8; RNH₂: 3-amino-1-(difluoromethyl)pyridin-2(1H)-one |
| | 9.1 mg, 15.9%. LCMS m/z = 471.1 [M + H]⁺; ¹H NMR (500 MHz, DMSO-d₆) |
| | δ: 1.46-1.54 (m, 3H), 1.69-1.80 (m, 1H), 1.85-1.95 (m, 1H), 2.18 (dd, 2H), |
| | 2.36-2.42 (m, 2H), 2.45-2.49 (m, 2H), 2.57-2.68 (m, 2H), 4.10 (s, 2H), 5.02 |
| | (quin, 1H), 6.55 (t, 1H), 7.04 (s, 1H), 7.49-7.63 (m, 1H), 7.87-8.16 (m, 1H), |
| | 8.60 (dd, 1H), 8.64 (s, 1H), 8.71 (s, 1H), 10.87 (s, 1H). |
| 85 | 6-Ethoxy-N-(2-methoxypyridin-3-yl)-2-(1-methyl-2-oxabicyclo[2.1.1]hexan-4- |
| | yl)-2H-indazole-5-carboxamide tfifluoroacetate |
| | 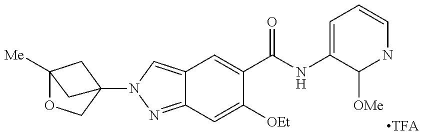 [SMILES: CCOc1cc2nn(C34COC(C)(C3)C4)cc2cc1C(=O)NC1=CC=CNC1OC] |
| | Prep-HPLC-Sunfire; 5-65% |
| | Reactants: Acid-9; RNH₂: 2-methoxypyridin-3-amine |
| | 34.1 mg, 60.8%. LCMS m/z = 409.2 [M H]⁺; ¹H NMR (500 MHz, DMSO-d₆) |
| | δ: 1.50 (s, 3H), 1.59 (t, 3H), 2.18 (dd, 2H), 2.37-2.42 (m, 2H), 4.01 (s, 3H), 4.11 |
| | (s, 2H), 4.34 (q, 2H), 7.05 (dd, 1H), 7.27 (s, 1H), 7.91 (dd, 1H), 8.64 (s, 1H), |
| | 8.70 (s, 1H), 8.78 (dd, 1H), 10.48 (s, 1H). |
| 86 | 6-Ethoxy-N-(1-methyl-2-oxo-1,2-dihydropyridin-3-yl)-2-(1-methyl-2- |
| | oxabicyclo[2.1.1]hexan-4-yl)-2H-indazole-5-carboxamide tfifluoroacetate |
| | 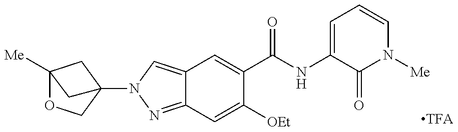 [SMILES: CCOc1cc2nn(C34COC(C)(C3)C4)cc2cc1C(=O)Nc1cccn(C)c1=O] |
| | Prep-HPLC-Sunfire; 5-50% |
| | Reactants: Acid-9; RNH₂: 3-amino-1-methylpyridin-2(1H)-one |
| | 33.7 mg, 60.1%. LCMS m/z = 409.2 [M + H]⁺; ¹H NMR (500 MHz, DMSO-d₆) |
| | δ: 1.50 (s, 3H), 1.62 (t, 3H), 2.18 (dd, 2H), 2.36-2.44 (m, 2H), 3.55 (s, 2H), 4.10 |
| | (s, 2H), 4.31 (q, 2H), 6.27-6.38 (m, 1H), 7.23 (s, 1H), 7.44 (dd, 1H), 8.50 (dd, |
| | 1H), 8.59 (s, 1H), 8.68 (s, 1H), 10.87 (s, 1H). |
| 87 | N-(6-(difluoromethyl)pyridin-2-yl)-6-methoxy-2-(1-methyl-2- |
| | oxabicyclo[2.1.1]hexan-4-yl)-2H-indazole-5-carboxamide trifluoroacetate |
| | 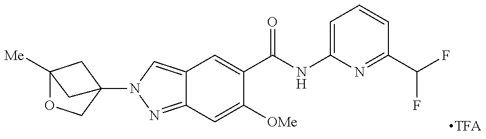 [SMILES: COc1cc2nn(C34COC(C)(C3)C4)cc2cc1C(=O)Nc1cccc(C(F)F)n1] |
| | Prep-HPLC-Sunfire; 5-60% |
| | Reactants: Acid-10; RNH₂: 6-(difluoromethyl)pyridin-2-amine |
| | 21.7 mg, 38.7%. LCMS m/z = 415.2 [M + H]⁺; ¹H NMR (500 MHz, DMSO-d₆) |
| | δ: 1.50 (s, 3H), 2.18 (dd, 2H), 2.35-2.44 (m, 2H), 3.96 (s, 3H), 4.11 (s, 2H), |
| | 6.78-7.04 (m, 1H), 7.20 (s, 1H), 7.46 (d, 1H), 8.06 (t, 1H), 8.26 (s, 1H), 8.42 (d, |
| | 1H), 8.63 (s, 1H), 10.75 (s, 1H). |
| 88 | N-(6-(difluoromethyl)pyridin-2-yl)-6-isopropoxy-2-(1-methyl-2- |
| | oxabicyclo[2.1.1]hexan-4-yl)-2H-indazole-5-carboxamide trifluoroacetate |
| | 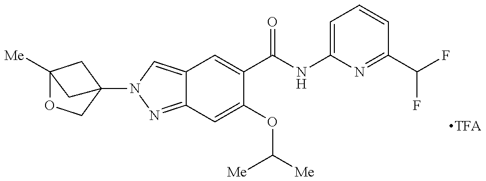 [SMILES: CC(C)Oc1cc2nn(C34COC(C)(C3)C4)cc2cc1C(=O)Nc1cccc(C(F)F)n1] |
| | Prep-HPLC-Sunfire; 10-90% |
| | Reactants: Acid-11; RNH₂: 6-(difluoromethyl)pyridin-2-amine |
| | 57.7 mg, 64.3%. LCMS m/z = 443.1 [M + H]⁺; ¹H NMR (400 MHz, CDCl₃) δ: |
| | 1.62 (d, 6H), 1.67 (s, 3H), 2.39-2.54 (m, 4H), 4.27 (s, 2H), 4.97 (spt, 1H), 6.59 |
| | (t, 1H), 7.41 (s, 1H), 7.48 (d, 1H), 7.99 (t, 1H), 8.20 (s, 1H), 8.43 (d, 1H), 8.78 |
| | (s, 1H), 10.92 (br s, 1H). |
| 89 | 6-Isopropoxy-N-(6-methoxypyridin-2-yl)-2-(1-methyl-2- |
| | oxabicyclo[2.1.1]hexan-4-yl)-2H-indazole-5-carboxamide trifluoroacetae |
| | 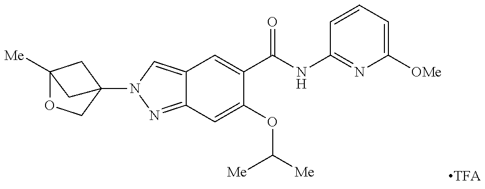 [SMILES: COc1cccc(NC(=O)c2cc3cn(C45COC(C)(C4)C5)nc3cc2OC(C)C)n1] |
| | Prep-HPLC-Sunfire; 10-70% |
| | Reactants: Acid-11; RNH₂: 6-methoxypyridin-2-amine |
| | 32.4 mg, 63.3%. LCMS m/z = 423.2 [M + H]⁺; ¹H NMR (500 MHz, DMSO-d₆) |
| | δ: 1.42-1.62 (m, 9H), 2.18 (dd, 2H), 2.34-2.44 (m, 2H), 3.85 (s, 3H), 4.11 (s, |
| | 2H), 5.00 (dt, 1H), 6.59 (d, 1H), 7.31 (s, 1H), 7.70-7.81 (m, 1H), 7.85 (br d, |
| | 1H), 8.60 (s, 1H), 8.70 (s, 1H), 10.87 (s, 1H). |
| 90 | 6-Isopropoxy-N-(2-methoxypyridin-3-yl)-2-(1-methyl-2- |
| | oxabicyclo[2.1.1]hexan-4-yl)-2H-indazole-5-carboxamide trifluoroacetate |
| | 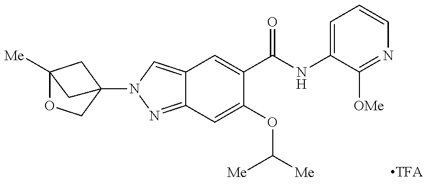 [SMILES: COc1ncccc1NC(=O)c1cc2cn(C34COC(C)(C3)C4)nc2cc1OC(C)C] |
| | Prep-HPLC-Sunfire; 5-65% |
| | Reactants: Acid-11; RNH₂: 2-methoxypyridin-3-amine |
| | 40.9 mg, 63%. LCMS m/z = 423.2 [M + H]⁺; ¹H NMR (500 MHz, DMSO-d₆) δ: |
| | 1.44-1.57 (m, 9H), 2.18 (dd, 2H), 2.35-2.44 (m, 2H), 3.95-4.06 (m, 3H), 4.11 (s, |
| | 2H), 5.04 (spt, 1H), 7.02-7.06 (m, 1H), 7.33 (s, 1H), 7.91 (dd, 1H), 8.63 (s, 1H), |
| | 8.70 (s, 1H), 8.78 (dd, 1H), 10.46 (s, 1H). |
| 91 | N-(1-(difluoromethyl)-2-oxo-1,2-dihydropyridin-3-yl)-6-isopropoxy-2-(1- |
| | methyl-2-oxabicyclo[2.1.]hexan-4-yl)-2H-indazole-5-carboxamide |
| | tfifluoroacetate |
| | 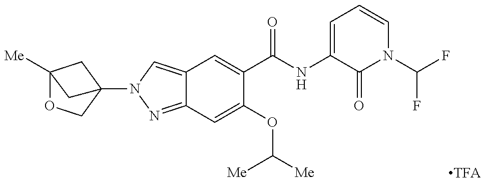 [SMILES: CC(C)Oc1cc2nn(C34COC(C)(C3)C4)cc2cc1C(=O)Nc1cccn(C(F)F)c1=O] |
| | Prep-HPLC-Sunfire; 5-70% |
| | Reactants: Acid-11; RNH₂: 3-amino-1-(difluoromethyl)pyridin-2(1H)-one |
| | 36.9 mg, 66.5%. LCMS m/z = 459.1 [M + H]⁺; ¹H NMR (500 MHz, DMSO-d₆) |
| | δ: 1.45-1.56 (m, 9H), 2.18 (dd, 2H), 2.36-2.43 (m, 2H), 4.10 (s, 2H), 4.95-5.10 |
| | (m, 1H), 6.54 (t, 1H), 7.31 (s, 1H), 7.56 (dd, 1H), 7.88-8.20 (m, 1H), 8.59 (dd, |
| | 1H), 8.62 (s, 1H), 8.70 (s, 1H), 10.87 (s, 1H). |
| 92 | 6-Isopropoxy-N-(1-methyl-1H-pyrazol-3-yl)-2-(1-methyl-2- |
| | oxabicyclo[2.1.1]hexan-4-yl)-2H-indazole-5-carboxamide |
| | 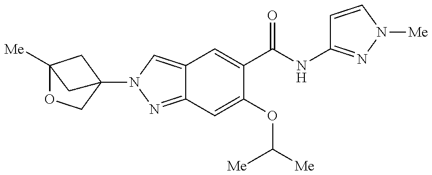 [SMILES: CC(C)Oc1cc2nn(C34COC(C)(C3)C4)cc2cc1C(=O)Nc1ccn(C)n1] |
| | Prep-HPLC-XSelect; 5-55% |
| | Reactants: Acid-11; RNH₂: 1-methyl-1H-pyrazol-3-amine |
| | 30.2 mg, 63.1%. LCMS m/z = 396.2 [M + H]⁺; ¹H NMR (500 MHz, DMSO-d₆) |
| | δ: 1.43 (d, 6H), 1.50 (s, 3H), 2.17 (dd, 2H), 2.33-2.45 (m, 2H), 3.78 (s, 3H), |
| | 4.03-4.15 (m, 2H), 4.90 (spt, 1H), 6.61 (d, 1H), 7.23 (s, 1H), 7.61 (d, 1H), 8.41 |
| | (s, 1H), 8.61-8.70 (m, 1H), 8.65 (s, 1H), 10.54 (s, 1H). |
| 93 | 6-Isopropoxy-2-(1-methyl-2-oxabicyclo[2.1.1]hexan-4-yl)-N-(pyrazolo[1,5- |
| | a]pyrimidin-3-yl)-2H-indazole-5-carboxamide |
| | 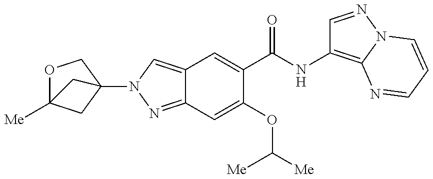 [SMILES: CC(C)Oc1cc2nn(C34COC(C)(C3)C4)cc2cc1C(=O)Nc1cnn2cccnc12] |
| | Prep-HPLC-XSelect; 5-55% |
| | Reactants: Acid-11; RNH₂: pyrazolo[1,5-a]pyrimidin-3-amine |
| | 33.1 mg, 63.2%. LCMS m/z = 433.2 [M + H]⁺; ¹H NMR (500 MHz, DMSO-d₆) |
| | δ: 1.50 (s, 3H), 1.56 (d, 6H), 2.15-2.25 (m, 2H), 2.38-2.44 (m, 2H), 4.11 (s, |
| | 2H), 5.04 (quin, 1H), 6.98-7.12 (m, 1H), 7.33 (s, 1H), 8.54 (dd, 1H), 8.63 (s, |
| | 1H), 8.71 (d, 1H), 8.80 (s, 1H), 9.03-9.15 (m, 1H), 10.74 (s, 1H). |
| 94 | 6-Isopropoxy-2-(1-methyl-2-oxabicyclo[2.1.1]hexan-4-yl)-N-(6- |
| | methylpyrazolo[1,5-a]pyrimidin-3-yl)-2H-indazole-5-carboxamide |
| | 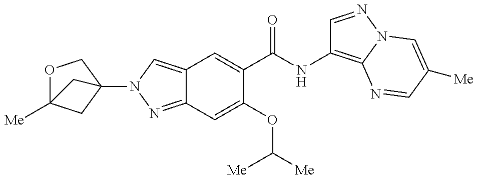 [SMILES: Cc1cnc2c(NC(=O)c3cc4cn(C56COC(C)(C5)C6)nc4cc3OC(C)C)cnn2c1] |
| | Prep-HPLC-XSelect; 5-65% |
| | Reactants: Acid-11; RNH₂: 6-methylpyrazolo[1,5-a]pyrimidin-3-amine |
| | 9.8 mg, 18.1%. LCMS m/z = 447.1 [M + H]⁺; ¹H NMR (500 MHz, DMSO-d₆) |
| | δ: 1.50 (s, 3H), 1.54 (d, 6H), 2.14-2.21 (m, 2H), 2.30-2.36 (m, 3H), 2.40 (dd, |
| | 2H), 4.11 (s, 2H), 5.03 (spt, 1H), 7.26-7.41 (m, 1H), 8.45 (d, 1H), 8.62 (s, 1H), |
| | 8.66-8.75 (m, 2H), 8.92 (d, 1H), 10.71 (s, 1H). |
| 95 | N-(6-chloropyrazolo[1,5-a]pyrimidin-3-yl)-6-isopropoxy-2-(1-methyl-2- |
| | oxabicyclo[2.1.1]hexan-4-yl)-2H-indazole-5-carboxamide |
| | 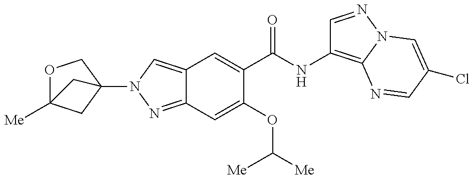 [SMILES: CC(C)Oc1cc2nn(C34COC(C)(C3)C4)cc2cc1C(=O)Nc1cnn2cc(Cl)cnc12] |
| | Prep-HPLC-XSelect; 5-70% |
| | Reactants: Acid-11; RNH₂: 6-chloropyrazolo[1,5-a]pyrimidin-3-amine |
| | 4.8 mg, 8.5%. LCMS m/z = 467.1 [M + H]⁺; ¹H NMR (500 MHz, DMSO-d₆) δ: |
| | 1.50 (s, 3H), 1.53 (d, 6H), 2.14-2.21 (m, 2H), 2.36-2.42 (m, 2H), 4.11 (s, 2H), |
| | 5.02 (spt, 1H), 7.32 (s, 1H), 8.58-8.59 (m, 1H), 8.61 (s, 1H), 8.70 (s, 1H), 8.76- |
| | 8.85 (m, 1H), 9.47-9.58 (m, 1H), 10.74 (s, 1H). |
| 96 | N-(6-fluoropyrazolo[1,5-a]pyrimidine-3-yl)-6-isopropoxy-2-(1-methyl-2- |
| | oxabicyclo[2.1.1]hexan-4-yl)-2H-indazole-5-carboxamide trifluoroacetate |
| | 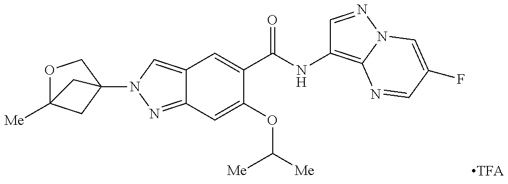 [SMILES: CC(C)Oc1cc2nn(C34COC(C)(C3)C4)cc2cc1C(=O)Nc1cnn2cc(F)cnc12] |
| | Prep-HPLC-Sunfire; 5-70% |
| | Reactants: Acid-11; RNH₂: 6-fluoropyrazolo[1,5-a]pyrimidin-3-amine |
| | 6.7 mg, 6.7%. LCMS m/z = 451.2 [M + H]⁺; ¹H NMR (400 MHz, CDCl₃) δ: |
| | 1.63 (s, 3H), 1.67 (d, 7H), 2.33-2.49 (m, 4H), 4.25 (s, 2H), 4.96 (quin, 1H). |
| | 7.29-7.33 (m, 1H), 8.10-8.18 (m, 1H), 8.47 (d, 1H), 8.64 (dd, 1H), 8.86 (s, 1H), |
| | 9.03 (s, 1H), 10.91 (s, 1H). |
| 97 | N-(6-(difluoromethyl)pyridin-2-yl)-6-isopropoxy-2-(1-methyl-2- |
| | oxabicyclo[2.2.1]heptan-4-yl)-2H-indazole-5-carboxamide |
| | 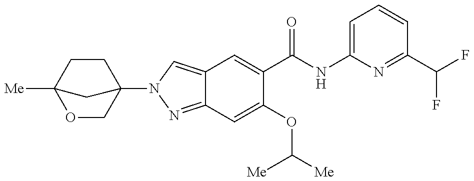 [SMILES: CC(C)Oc1cc2nn(C34CCC(C)(C3)OC4)cc2cc1C(=O)Nc1cccc(C(F)F)n1] |
| | Prep-HPLC-Sunfire; 20-75% |
| | Reactant: Acid-12; RNH₂: 6-(difluoromethyl)pyridin-2-amine |
| | 22.3 mg, 34.6%; LCMS m/z = 457.2 [M + H]⁺. ¹H NMR (400 MHz, DMSO-d₆) |
| | δ: 1.50 (s, 3H), 1.58 (d, 6H), 1.90-2.11 (m, 2H), 2.30-2.39 (m, 1H), 2.41 (s, |
| | 2H), 2.44-2.55 (m, 1H), 4.12 (dd, 1H), 4.21 (s, 1H), 4.90-5.04 (m, 1H), 6.44- |
| | 6.79 (m, 1H), 7.16 (s, 1H), 7.43 (d, 1H), 7.99 (t, 1H), 8.43-8.51 (m, 2H), 8.64 |
| | (s, 1H), 11.05 (s, 1H). |
| 98 | 6-Isopropoxy-2-(1-methyl-2-oxabicyclo[2.2.1]heptan-4-yl)-N-(pyrazolo[1,5- |
| | a]pyrimidin-3-yl)-2H-indazole-5-carboxamide |
| | 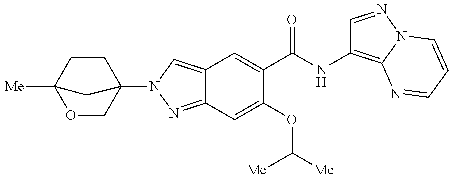 [SMILES: CC(C)Oc1cc2nn(C34CCC(C)(C3)OC4)cc2cc1C(=O)Nc1cnn2cccnc12] |
| | Prep-HPLC-XSelect; 5-60% |
| | Reactants: Acid-12; RNH₂: pyrazolo[1,5-a]pyrimidin-3-amine |
| | 26.7 mg, 19.9%; LCMS m/z = 447.2 [M + H]⁺. ¹H NMR (500 MHz, DMSO-d₆) |
| | δ: 1.42 (s, 3H), 1.55 (d, 6H), 1.80-1.90 (m, 1H), 1.97 (dt, 1H), 2.20-2.40 (m, |
| | 4H), 4.02 (dd, 1H), 4.09 (d, 1H), 5.05 (spt, 1H), 7.04 (dd, 1H), 7.32 (s, 1H), |
| | 8.54 (d, 1H), 8.62 (s, 1H), 8.66 (s, 1H), 8.80 (s, 1H), 9.05-9.10 (m, 1H), 10.75 |
| | (s, 1H). |
| 99 | 6-Isopropoxy-2-(1-methyl-2-oxabicyclo[2.2.1]heptan-4-yl)-N-(6- |
| | methylpyrazolo[1,5-a]pyrimidin-3-yl)-2H-indazole-5-carboxamide |
| | trifluoroacetate |
| | 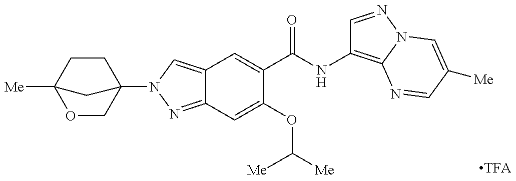 [SMILES: Cc1cnc2c(NC(=O)c3cc4cn(C56CCC(C)(C5)OC6)nc4cc3OC(C)C)cnn2c1] |
| | Prep-HPLC-Sunfire; 5-65% |
| | Reactants: Acid-12; RNH₂: 6-methylpyrazolo[1,5-a]pyrimidin-3-amine |
| | 12.9 mg, 19.9%; LCMS m/z = 461.2 [M + H]⁺. ¹H NMR (500 MHz, DMSO-d₆) |
| | δ: 1.42 (s, 3H), 1.55 (d, 6H), 1.84 (ddt, 1H), 1.91-2.02 (m, 1H), 2.21-2.32 (m, |
| | 2H), 2.32-2.41 (m, 5H), 4.02 (dd, 1H), 4.09 (d, 1H), 4.99-5.10 (m, 1H), 7.32 (s, |
| | 1H), 8.45 (d, 1H), 8.62 (s, 1H), 8.66 (s, 1H), 8.70 (s, 1H), 8.86-8.99 (m, 1H), |
| | 10.73 (s, 1H). |
| 100 | N-(6-fluoropyrazolo[1,5-a]pyrimidin-3-yl)-6-isopropoxy-2-(1-methyl-2- |
| | oxabicyclo[2.2.1]heptan-4-yl)-2H-indazole-5-carboxamide |
| | 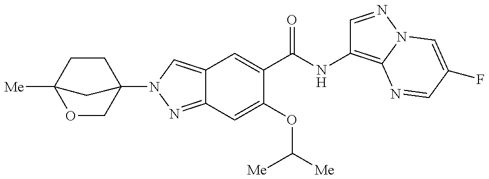 [SMILES: CC(C)Oc1cc2nn(C34CCC(C)(C3)OC4)cc2cc1C(=O)Nc1cnn2cc(F)cnc12] |
| | Prep-HPLC-XSelect; 5-65% |
| | Reactants: Acid-12; RNH₂: 6-fluoropyrazolo[1,5-a]pyrimidin-3-amine |
| | 0.5mg, 0.8%; LCMS m/z = 465.2 [M + H]⁺. |
| 101 | 6-Isopropoxy-N-(1-methyl-1H-pyrazol-3-yl)-2-(1-methyl-2- |
| | oxabicyclo[2.2.1]heptan-4-yl)-2H-indazole-5-carboxamide |
| | 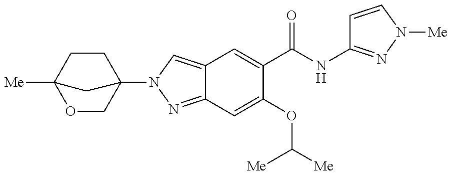 [SMILES: CC(C)Oc1cc2nn(C34CCC(C)(C3)OC4)cc2cc1C(=O)Nc1ccn(C)n1] |
| | Prep-HPLC-XSelect; 5-55% |
| | Reactants: Acid-12; RNH₂: 1-methyl-1H-pyrazol-3-amine |
| | 28.8 mg, 49.9%; LCMS m/z = 410.2 [M + H]⁺. ¹H NMR (500 MHz, DMSO-d₆) |
| | δ: 1.33-1.49 (m, 9H), 1.77-1.90 (m, 1H), 1.96 (td, 1H), 2.17-2.29 (m, 2H), 2.29- |
| | 2.39 (m, 2H), 3.65-3.81 (m, 3H), 4.00 (dd, 1H), 4.07 (d, 1H), 4.90 (spt, 1H), |
| | 6.60 (d, 1H), 7.23 (s, 1H), 7.61 (d, 1H), 8.41 (s, 1H), 8.60 (d, 1H), 10.53 (s, |
| | 1H). |
| 102 | 6-Isopropoxy-2-(1-methyl-2-oxabicyclo[2.2.2]octan-4-yl)-N-(6- |
| | methylpyrazolo[1,5-a]pyrimidin-3-yl)-2H-indazole-5-carboxamide |
| | 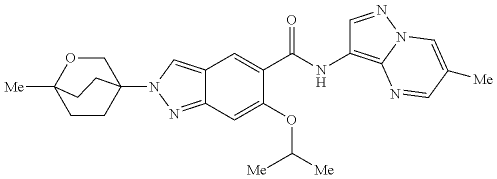 [SMILES: Cc1cnc2c(NC(=O)c3cc4cn(C56CCC(C)(CC5)OC6)nc4cc3OC(C)C)cnn2c1] |
| | Prep-HPLC-Sunfire; 5-70% |
| | Reactants: Acid-13; RNH₂: 6-methylpyrazolo[1,5-a]pyrimidin-3-amine |
| | 1.3 mg, 3%. LCMS m/z = 475.3 [M + H]⁺ |
| 103 | 6-Isopropoxy-N-(1-methyl-1H-pyrazol-3-yl)-2-(1-methyl-2- |
| | oxabicyclo[2.2.2]octan-4-yl)-2H-indazole-5-carboxamide |
| | 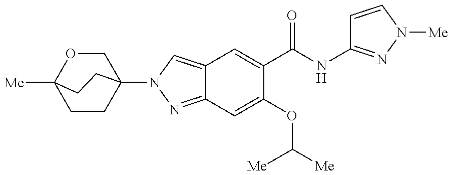 [SMILES: CC(C)Oc1cc2nn(C34CCC(C)(CC3)OC4)cc2cc1C(=O)Nc1ccn(C)n1] |
| | Prep-HPLC-Sunfire; 5-70% |
| | Reactants: Acid-13; RNH₂: 1-methyl-1H-pyrazol-3-amine |
| | 21.9 mg, 57.5%. LCMS m/z = 424.3 [M + H]⁺; ¹H NMR (500 MHz, DMSO-d₆) |
| | δ: 1.13 (s, 3H), 1.42 (d, 6H), 1.80-2.00 (m, 4H), 2.25 (td, 2H), 2.31-2.43 (m, |
| | 2H), 3.77 (s, 3H), 4.07-4.20 (m, 2H), 4.84-4.98 (m, 1H), 6.61 (d, 1H), 7.22 (s, |
| | 1H), 7.61 (d, 1H), 8.41 (s, 1H), 8.59 (s, 1H), 10.54 (s, 1H). |
| 139 | 6-cyclobutoxy-N-(1-(difluoromethyl)-1H-pyrazol-3-yl)-2-(1-methyl-2- |
| | oxabicyclo[2.2.2]octan-4-yl)-2H-indazole-5-carboxamide |
| | 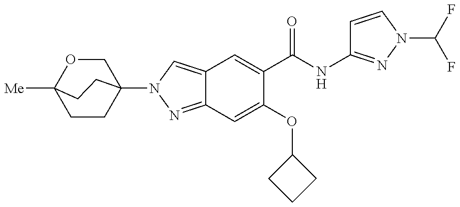 [SMILES: CC12CCC(n3cc4cc(C(=O)Nc5ccn(C(F)F)n5)c(OC5CCC5)cc4n3)(CC1)CO2] |
| | Reactants: Acid-17; RNH₂: 1-(difluoromethyl)-1H-pyrazol-3-amine |
| | 7.7 mg, 11% yield. LCMS m/z = 472.2 [M + H]+ |
| 140 | 6-cyclobutoxy-N-(1-methyl-1H-pyrazol-3-yl)-2-(1-methyl-2- |
| | oxabicyclo[2.2.2]octan-4-yl)-2H-indazole-5-carboxamide |
| | 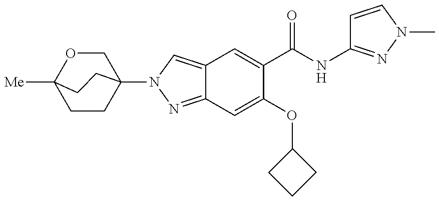 [SMILES: Cn1ccc(NC(=O)c2cc3cn(C45CCC(C)(CC4)OC5)nc3cc2OC2CCC2)n1] |
| | Reactants: Acid-17; R—NH₂;: 1-methyl-1H-pyrazol-3-amine |
| | 8.5 mg, 14% yield. LCMS m/z = 436.2 [M + H]⁺ |
| | ¹H NMR: (500 MHz, DMSO-d₆) δ: 1.10 (s, 3H), 1.73-1.68 (m, 1H), 1.93-1.83 |
| | (m, 3H), 2.24-2.13 (m, 4H), 2.32-2.29 (m, 2H), 2.56-2.54 (m, 2H), 3.74 (s, 3H), |
| | 4.08 (s, 2H), 4.93-4.89 (m, 1H), 6.57 (s, 1H), 6.91 (s, 1H), 7.58 (s, 1H), 8.31 (s, |
| | 1H), 8.53 (s, 1H), 10.41 (s, 1H). |

### Example 104 and 105: (R)-6-isopropoxy-N-(6-methylpyrazolo[1,5-a]pyrimidin-3-yl)-2-((tetrahydrofuran-3-yl)methyl)-2H-pyrazolo[3,4-b]pyridine-5-carboxamide and (S)-6-isopropoxy-N-(6-methylpyrazolo[1,5-a]pyrimidin-3-yl)-2-((tetrahydrofuran-3-yl)methyl)-2H-pyrazolo[3,4-b]pyridine-5-carboxamide

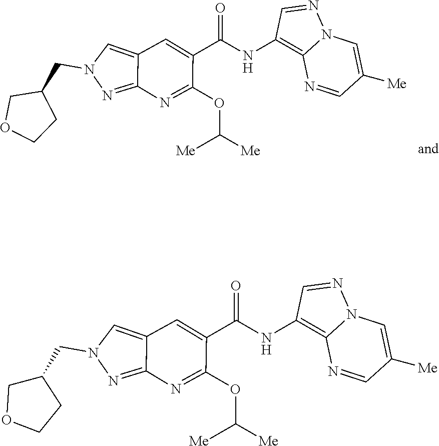 [SMILES: Cc1cnc2c(NC(=O)c3cc4cn(C[C@H]5CCOC5)nc4nc3OC(C)C)cnn2c1 ; Cc1cnc2c(NC(=O)c3cc4cn(C[C@@H]5CCOC5)nc4nc3OC(C)C)cnn2c1]

### *Stereochemistry Arbitrarily Assigned

To a solution of 6-isopropoxy-2-((tetrahydrofuran-3-yl)methyl)-2H-pyrazolo[3,4-b]pyridine-5-carboxylic acid (Preparation 104, 130 mg, 0.426 mmol) and 6-methylpyrazolo[1,5-a]pyrimidin-3-amine (126 mg, 0.852 mmol) in pyridine (4 mL) was added T3P® (50 wt. % in EtOAc, 135 mg, 0.426 mmol) and the mixture stirred at 20° C. for 16 h. The reaction mixture was evaporated to dryness in vacuo and the residue diluted with saturated aq. NaHCO₃ (pH 7) and extracted with EtOAc (3×50 mL). The combined organics were washed with brine (50 mL), dried (Na₂SO₄) and evaporated to dryness and the residue was purified by Combi-Flash (3:1 PE/EtOAc) to give 6-isopropoxy-N-(6-methylpyrazolo[1,5-a]pyrimidin-3-yl)-2-((tetrahydrofuran-3-yl)methyl)-2H-pyrazolo[3,4-b]pyridine-5-carboxamide as a yellow solid (185 mg, 97.8%). LCMS m/z=436.0 [M+H]⁺. Further purification by prep-SFC (Chiralpak AY-3; 100×4.6 mm, 3 μm; 40% EtOH+0.05% DEA in CO₂ afforded (R)-6-isopropoxy-N-(6-methylpyrazolo[1,5-a]pyrimidin-3-yl)-2-((tetrahydrofuran-3-yl)methyl)-2H-pyrazolo[3,4-b]pyridine-5-carboxamide and (S)-6-isopropoxy-N-(6-methylpyrazolo[1,5-a]pyrimidin-3-yl)-2-((tetrahydrofuran-3-yl)methyl)-2H-pyrazolo[3,4-b]pyridine-5-carboxamide.
*Peak 1, Example 104; 60 mg, 31.1%; LCMS m/z=436.1 [M+H]⁺; ¹H NMR (400 MHz, MeOH-d₄) δ: 1.65 (d, 6H), 1.74-1.79 (m, 1H), 2.12-2.22 (m, 1H), 2.41 (s, 3H), 2.93-3.06 (m, 1H), 3.64-3.66 (m, 1H), 3.74-3.83 (m, 2H), 3.78-3.95 (m, 1H), 4.43 (d, 2H), 5.77 (m, 1H), 8.42-8.46 (m, 2H), 8.62-8.67 (m, 1H), 8.71 (s, 1H), 9.01 (s, 1H).
*Peak 2, Example 105; 80 mg, 43.2%; LCMS m/z=436.0 [M+H]⁺; ¹H NMR (400 MHz, MeOH-d₄) δ: 1.65 (d, 6H), 1.71-1.79 (m, 1H), 2.05-2.09 (m, 1H), 2.41 (s, 3H), 2.94-3.05 (m, 1H), 3.63-3.66 (m, 1H), 3.75-3.84 (m, 2H), 3.90-3.98 (m, 1H), 4.43 (d, 2H), 5.78 (m, 1H), 8.42-8.46 (m, 2H), 8.66 (s, 1H), 8.71 (s, 1H), 9.01 (s, 1H).

### Examples 106-117

The following pairs of enantiomers (*Stereochemistry arbitrarily assigned) were obtained from the appropriate carboxylic acid (Acid-7, 14, 15 or 16) and amine (RNH₂) using an analogous method to that described for Examples 104 and 105. The following codes describe the prep-SFC conditions used in the table below: SFC-A: CHIRALPAK IC; 250×30 mm, 5 μm; 45% MeOH+0.1% NH₄OH in CO₂; SFC-B: CHIRALPAK AD-3; 150×4.6 mm, 3 μm; 40% EtOH+0.05% DEA in CO₂; SFC-C: REGIS (S,S) WHELK-Ol; 250×30 mm, 5 μm); 50% MeOH+0.1% NH₄OH in CO₂; SFC-D: CHIRALPAK AY-H; 250×30 mm, 5 μm; 40% EtOH+0.1% NH₄OH in CO₂; SFC-E: Phenomenex Cellulose 2 100×4.6 mm, 3 um; 20% MeCN in MeOH+0.05% DEA in CO₂; SFC-F: Phenomenex Cellulose-2 250×30 mm, 10 μm; 50% EtOH+0.1% NH₄OH in CO₂.
Acid-7: 6-isopropoxy-2-((tetrahydrofuran-3-yl)methyl)-2H-pyrazolo[3,4-b]pyridine-5-carboxylic acid (Preparation 104); Acid-14: 6-isopropoxy-2-(tetrahydro-2H-pyran-3-yl)-2H-pyrazolo[3,4-b]pyridine-5-carboxylic acid (Preparation 103); Acid-15: 6-cyclobutoxy-2-((tetrahydrofuran-3-yl)methyl)-2H-pyrazolo[3,4-b]pyridine-5-carboxylic acid (Preparation 105); Acid-16: 6-(cyclopentyloxy)-2-((tetrahydrofuran-3-yl)methyl)-2H-pyrazolo[3,4-b]pyridine-5-carboxylic acid (Preparation 106).

| Ex- | | |
|---|---|---|
| ample | *Peak 1 | *Peak 2 |
| 106 | (R)-N-(6-(difluoromethyl)pyridin-2- | (S)-N-(6-(difluoromethyl)pyridin-2- |
| and | yl)-6-isopropoxy-2-((tetrahydrofuran- | yl)-6-isopropoxy-2-((tetrahydrofuran- |
| 107 | 3-yl)methyl)-2H-pyrazolo[3,4- | 3-yl)methyl)-2H-pyraozlo[3,4- |
| | b]pyridine-5-carboxamide | b]pyridine-5-carboxamide |
| | 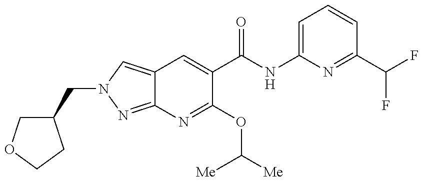 [SMILES: CC(C)Oc1nc2nn(C[C@H]3CCOC3)cc2cc1C(=O)Nc1cccc(C(F)F)n1] | 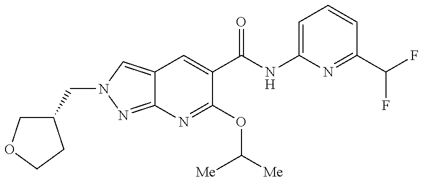 [SMILES: CC(C)Oc1nc2nn(C[C@@H]3CCOC3)cc2cc1C(=O)Nc1cccc(C(F)F)n1] |
| | Acid-7; RNH₂: 6- | Acid 7; RNH₂; 6- |
| | (difluoromethyl)pyridin-2-amine; | (difluoromethyl)pyridin-2-amine; |
| | SFC-A | SFC-A |
| | 14.1 mg, 27.3%; LCMS m/z = 432.0 | 10.9 mg, 21.6%; LCMS m/z = 432.0 |
| | [M + H]⁺; ¹H NMR (400 MHz, MeOH- | [M + H]⁺; ¹H NMR (400 MHz, MeOH- |
| | d₄) δ: 1.59 (d, 6H), 1.71-1.80 (m, 1H), | d₄) δ: 1.58 (d, 6H), 1.70-1.80 (m, 1H), |
| | 2.03-2.12 (m, 1H), 2.96-3.04 (m, 1H), | 2.02-2.12 (m, 1H), 2.96-3.03 (m, 1H), |
| | 3.62-3.66 (m, 1H), 3.75-3.83 (m, 2H), | 3.61-3.66 (m, 1H), 3.74-3.82 (m, 2H), |
| | 3.91-3.97 (m, 1H), 4.43 (d, 2H), 5.69- | 3.90-3.95 (m, 1H), 4.43 (d, 2H), 5.66- |
| | 5.74 (m, 1H), 6.48-6.76 (m, 1H), 7.44 | 5.73 (m, 1H), 6.47-6.76 (m, 1H), 7.43 |
| | (d, 1H), 7.98-8.02 (m, 1H), 8.43 (s, | (d, 1H), 7.96-8.01 (m, 1H), 8.42 (s, |
| | 1H), 8.46 (d, 1H), 8.97 (s, 1H). | 1H), 8.45 (d, 1H), 8.96 (s, 1H). |
| 108 | (S)-6-isopropoxy-N-(pyrazolo[1,5- | (R)-6-isopropoxy-N-(pyrazolo[1,5- |
| and | a]pyrimidin-3-yl)-2-(tetrahydro-2H- | a]pyrimidin-3-yl)-2-(tetrahydro-2H- |
| 109 | pyran-3-yl)-2H-indazole-5- | pyran-3-yl)-2H-indazole-5- |
| | carboxamide | carboxamide |
| | 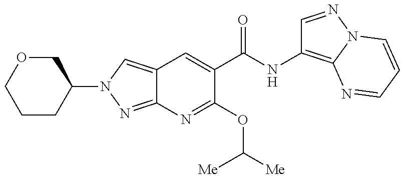 [SMILES: CC(C)Oc1nc2nn([C@H]3CCCOC3)cc2cc1C(=O)Nc1cnn2cccnc12] | 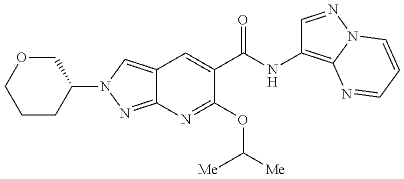 [SMILES: CC(C)Oc1nc2nn([C@@H]3CCCOC3)cc2cc1C(=O)Nc1cnn2cccnc12] |
| | Acid-14; 6-isopropoxy-2-(tetrahydro- | Acid-14; RNH₂: pyrazolo[1,5- |
| | 2H-pyran-3-yl)-2H-pyrazolo[3,4- | a]pyrimidin-3-amine; SFC-B |
| | b]pyridine-5-carboxylic acid | 70.9 mg, 35.5%; LCMS m/z = 421.0 |
| | (Preparation 103); RNH₂: | [M + H]⁺; ¹H NMR (500 MHz, MeOH- |
| | pyrazolo[1,5-a]primidin-3-amine; | d₄) δ: 1.64 (d, 6H), 1.81-1.86 (m, 2H), |
| | SFC-B | 2.31-2.36 (m, 2H), 3.61-3.67 (m, 1H), |
| | 72.4 mg, 36.2%; LCMS m/z = 421.0 | 3.90-3.95 (m, 2H), 4.14 (dd, 1H), |
| | [M + H]⁺; ¹H NMR (500 MHz, MeOH- | 4.60-4.64 (m, 1H), 5.00-5.03 (m, 1H), |
| | d₄) δ: 1.64 (d, 6H), 1.81-1.86 (m, 2H), | 7.00 (dd, 1H), 7.18 (s, 1H), 8.49-8.51 |
| | 2.30-2.36 (m, 2H), 3.61-3.67 (m, 1H), | (m, 1H), 8.53 (s, 1H), 8.69 (s, 1H), |
| | 3.90-3.95 (m, 2H), 4.18 (dd, 1H), | 8.81-8.85 (m, 2H). |
| | 4.62-4.64 (m, 1H), 5.00-5.03 (m, 1H), | |
| | 6.99 (dd, 1H), 7.18 (s, 1H), 8.49-8.51 | |
| | (m, 1H), 8.52 (s, 1H), 8.69 (s, 1H), | |
| | 8.81-8.85 (m, 2H). | |
| 110 | (R)-N-(1-(difluoromethyl)-1H- | (S)-N-(1-(difluoromethyl)-1H- |
| and | pyrazol-3-yl)-6-isopropoxy-2- | pyrazol-3-yl)-6-isopropoxy-2- |
| 111 | ((tetrahydrofuran-3-yl)methyl)-2H- | ((tetrahydrofuran-3-yl)methyl)-2H- |
| | pyrazolo[3,4-b]pyridine-5- | pyrazolo[3,4-b]pyridine-5- |
| | carboxamide | carboxamide |
| | 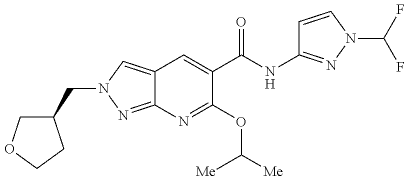 [SMILES: CC(C)Oc1nc2nn(C[C@H]3CCOC3)cc2cc1C(=O)Nc1ccn(C(F)F)n1] | 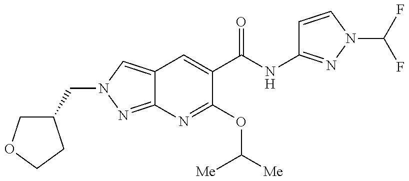 [SMILES: CC(C)Oc1nc2nn(C[C@@H]3CCOC3)cc2cc1C(=O)Nc1ccn(C(F)F)n1] |
| | Acid-7; RNH₂: 1-(difluoromethyl)- | Acid-7; RNH₂: 1-(difluoromethyl)- |
| | 1H-pyrazol-3-amine; SFC-C | 1H-pyrazol-3-amine; SFC-C |
| | 37.4 mg, 41.6%; LCMS m/z = 421.0 | 29.9 mg, 33.2%; LCMS m/z = 421.0 |
| | [M + H]⁺; ¹H NMR (500 MHz, MeOH- | [M + H]⁺; ¹H NMR (500 MHz, MeOH- |
| | d₄) δ: 1.55 (d, 6H), 1.72-1.79 (m, 1H), | d₄) δ: 1.55 (d, 6H), 1.71-1.79 (m, 1H), |
| | 2.03-2.09 (m, 1H), 2.96-3.02 (m, 1H), | 2.03-2.09 (m, 1H), 2.96-3.02 (m, 1H), |
| | 3.61-3.65 (m, 1H), 3.74-3.81 (m, 2H), | 3.61-3.65 (m, 1H), 3.76-3.82 (m, 2H), |
| | 3.92-3.95 (m, 1H), 4.42 (d, 2H), 5.66- | 3.90-3.94 (m, 1H), 4.42 (d, 2H), 5.66- |
| | 5.72 (m, 1H), 7.01 (d, 1H), 7.28-7.52 | 5.72 (m, 1H), 7.01 (d, 1H), 7.28-7.52 |
| | (m, 1H), 8.00 (d, 1H), 8.40 (s, 1H), | (m, 1H), 8.00 (d, 1H), 8.41 (s, 1H), |
| | 8.88 (s, 1H). | 8.88 (s, 1H). |
| 112 | (R)-6-cyclobutoxy-N-(1-methyl-2- | (S)-6-cyclobutoxy-N-(1-methyl-2- |
| and | oxo-1,2-dihydropyridin-3-yl)-2- | oxo-1,2-dihydropyridin-3-yl)-2- |
| 113 | ((tetrahydrofuran-3-yl)methyl)-2H- | ((tetrahydrofuran-3-yl)methyl)-2H- |
| | pyrazolo[3,4-b]pyridine-5- | pyrazolo[3,4-b]pyridine-5- |
| | carboxamide | carboxamide |
| | 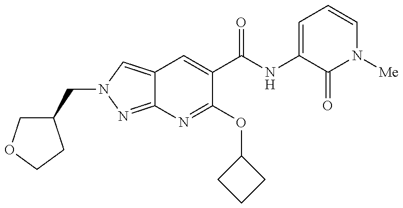 [SMILES: Cn1cccc(NC(=O)c2cc3cn(C[C@H]4CCOC4)nc3nc2OC2CCC2)c1=O] | 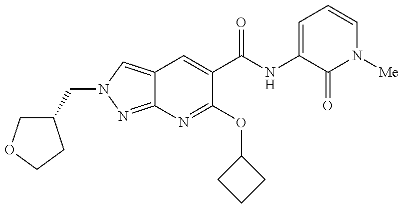 [SMILES: Cn1cccc(NC(=O)c2cc3cn(C[C@@H]4CCOC4)nc3nc2OC2CCC2)c1=O] |
| | RCO₂H-15; RNH₂; 3-amino-1- | RCO₂H-15; RNH₂: 3-amino-1- |
| | methylpyridin-2(1H)-one; SFC-D | methylpyridin-2(1H)-one; SFC-D |
| | 21.3 mg, 26.6%; LCMS m/z = 424.1 | 23 mg, 28.2%; LCMS m/z = 424.1 |
| | [M + H]⁺; ¹H NMR (400 MHz, DMSO- | [M + H]⁺; ¹H NMR (400 MHz, DMSO- |
| | d₆) δ: 1.59-1.67 (m, 1H), 1.67-1.78 (m, | d₆) δ: 1.60-1.78 (m, 2H), 1.88-1.98 (m, |
| | 1H), 1.86-1.98 (m, 2H), 2.53-2.58 (m, | 2H), 2.54-2.57 (m, 2H), 2.83-2.90 (m, |
| | 2H), 2.83-2.91 (m, 1H), 3.30 (s, 2H), | 1H), 3.30 (s, 2H), 3.50-3.54 (m, 1H), |
| | 3.49-3.54 (m, 1H), 3.57 (s, 3H), 3.63- | 3.57 (s, 3H), 3.61-3.71 (m, 2H), 3.76- |
| | 3.71 (m, 2H), 3.76-3.82 (m, 1H), 4.39 | 3.82 (m, 1H), 4.39 (d, 2H), 5.45-5.23 |
| | (d, 2H), 5.45-5.53 (m, 1H), 6.32 (t, | (m, 1H), 6.32 (t, 1H), 7.47 (dd, 1H), |
| | 1H), 7.45-7.48 (m, 1H), 8.49-8.52 (m, | 8.51 (dd, 1H), 8.59 (s, 1H), 8.98 (s, |
| | 1H), 8.59 (s, 1H), 8.98 (s, 1H), 10.93 | 1H), 10.93 (s, 1H). |
| | (s, 1H). | |
| 114 | (R)-6-(cyclopentyloxy)-N-(1-methyl- | (S)-6-(cyclopentyloxy)-N-(1-methyl- |
| and | 2-oxo-1,2-dihydropyridin-3-yl)-2- | 2-oxo-1,2-dihydropyridin-3-yl)-2- |
| 115 | ((tetrahydrofuran-3-yl)methyl)-2H- | ((tetrahydrofuran-3-yl)methyl)-2H- |
| | pyrazolo[3,4-b]pyridine-5- | pyrazolo[3,4-b]pyridine-5- |
| | carboxamide | carboxamide |
| | 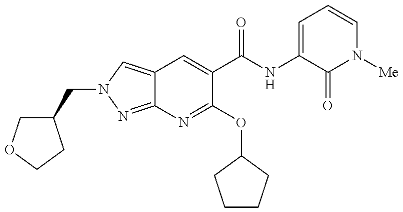 [SMILES: Cn1cccc(NC(=O)c2cc3cn(C[C@H]4CCOC4)nc3nc2OC2CCCC2)c1=O] | 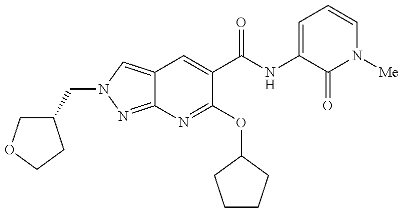 [SMILES: Cn1cccc(NC(=O)c2cc3cn(C[C@@H]4CCOC4)nc3nc2OC2CCCC2)c1=O] |
| | RCO₂H-16; RNH₂: 3-amino-1- | RCO₂H-16; RNH₂: 3-amino-1- |
| | methylpyridin-2(1H)-one; SFC-E | methylpyridin-2(1H)-one; SFC-E |
| | 12.3 mg, 13.2%; LCMS m/z = 438.1 | 17.6 mg, 18.9%; LCMS m/z = 438.0 |
| | [M + H]⁺; ¹H NMR (400 MHz, CDCl₃) | [M + H]⁺; ¹H NMR (400 MHz, CDCl₃) |
| | δ: 1.68-1.77 (m, 3H), 1.94-1.95 (m, | δ: 1.66-1.72 (m, 3H), 1.94-1.95 (m, |
| | 2H), 2.05-2.13 (m, 1H), 2.22 (d, 4H), | 2H), 2.10 (br s, 1H), 2.22 (br s, 4H), |
| | 3.10-3.14 (m, 1H), 3.62-3.66 (m, 4H), | 3.13 (br s, 1H), 3.62-3.65 (m, 4H), |
| | 3.76-3.83 (m, 2H), 3.94-4.00 (m, 1H), | 3.77-3.82 (m, 2H), 3.94-4.00 (m, 1H), |
| | 4.34 (d, 2H), 5.86-5.93 (m, 1H), 6.22- | 4.34 (br s, 2H), 5.90 (br s, 1H), 6.23- |
| | 6.27 (m, 1H), 7.02 (d, 1H), 7.96 (s, | 6.27 (m, 1H), 7.02 (d, 1H), 7.97 (s, |
| | 1H), 8.61-8.64 (m, 1H), 8.98 (s, 1H), | 1H), 8.63 (d, 1H), 8.98 (s, 1H), 10.74 |
| | 10.82 (s, 1H). | (s, 1H). |
| 116 | (R)-6-(cyclopentyloxy)-N-(1- | (S)-6-(cyclopentyloxy)-N-(1- |
| and | (difluoromethyl)-1H-pyrazol-3-yl)-2- | (difluoromethyl)-1H-pyrazol-3-yl)-2- |
| 117 | ((tetrahydrofuran-3-yl)methyl)-2H- | ((tetrahydrofuran-3-yl)methyl)-2H- |
| | pyrazolo[3,4-b]pyridine-5- | pyrazolo[3,4-b]pyridine-5- |
| | carboxamide | carboxamide |
| | 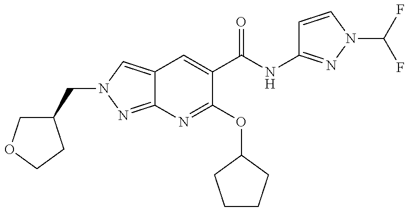 [SMILES: O=C(Nc1ccn(C(F)F)n1)c1cc2cn(C[C@H]3CCOC3)nc2nc1OC1CCCC1] | 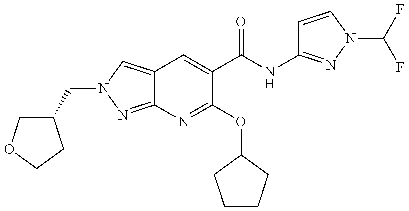 [SMILES: O=C(Nc1ccn(C(F)F)n1)c1cc2cn(C[C@@H]3CCOC3)nc2nc1OC1CCCC1] |
| | RCO₂₁H-16; RNH₂: 1- | RCO₂H-16; RNH₂: 1- |
| | (difluoromethyl)-1H-pyrazol-3-amine; | (difluoromethyl)-1H-pyrazol-3-amine; |
| | SFC-F | SFC-F |
| | 31.6 mg, 31%; LCMS m/z = 447.3 | 34.9 mg, 34.9%; LCMS m/z = 447.3 |
| | [M + H]⁺; ¹H NMR (400 MHz, CDCl₃) | [M + H]⁺; ¹H NMR (400 MHz, CDCl₃) |
| | δ: 1.68-1.74 (m, 1H), 1.74-1.83 (m, | δ: 1.68-1.74 (m, 1H), 1.74-1.83 (m, |
| | 2H), 1.94-2.00 (m, 2H), 2.00-2.05 (m, | 2H), 1.91-2.00 (m, 2H), 2.00-2.05 (m, |
| | 1H), 2.05-2.10 (m, 2H), 2.11-2.20 (m, | 1H), 2.05-2.10 (m, 2H), 2.11-2.20 (m, |
| | 2H), 3.08-3.16 (m, 1H), 3.65 (dd, 1H), | 2H), 3.09-3.16 (m, 1H), 3.64 (dd, 1H), |
| | 3.76-3.84 (m, 2H), 3.94-4.00 (m, 1H), | 3.76-3.84 (m, 2H), 3.94-4.00 (m, 1H), |
| | 4.35 (d, 2H), 5.93-5.98 (m, 1H), 6.94- | 4.35 (d, 2H), 5.94-5.97 (m, 1H), 6.94- |
| | 7.25 (m, 2H), 7.76 (d, 1H), 8.00 (s, | 7.25 (m, 2H), 7.76 (d, 1H), 8.00 (s, |
| | 1H), 9.04 (s, 1H), 10.58 (br s, 1H). | 1H), 9.04 (s, 1H), 10.58 (br s, 1H). |

### Example 118 and 119: (R)-6-Isopropoxy-N-(pyrazolo[1,5-a]pyrimidin-3-yl)-2-((tetrahydrofuran-3-yl)methyl)-2H-pyrazolo[3,4-b]pyridine-5-carboxamide and (S)-6-isopropoxy-N-(pyrazolo[1,5-a]pyrimidin-3-yl)-2-((tetrahydrofuran-3-yl)methyl)-2H-pyrazolo[3,4-b]pyridine-5-carboxamide

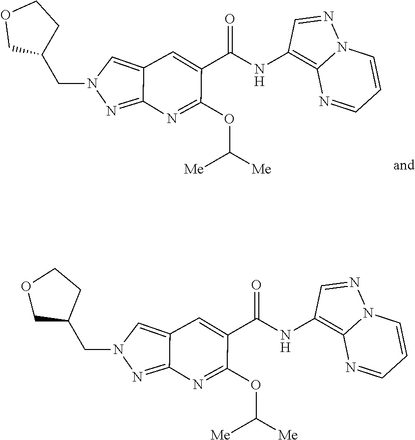 [SMILES: CC(C)Oc1nc2nn(C[C@H]3CCOC3)cc2cc1C(=O)Nc1cnn2cccnc12 ; CC(C)Oc1nc2nn(C[C@@H]3CCOC3)cc2cc1C(=O)Nc1cnn2cccnc12]

### *Stereochemistry Arbitrarily Assigned

6-Isopropoxy-N-(pyrazolo[1,5-a]pyrimidin-3-yl)-2-((tetrahydrofuran-3-yl)methyl)-2H-pyrazolo[3,4-b]pyridine-5-carboxamide (Example 54) was purified by prep-SFC (DAICEL CHIRALPAK AY-H; 250×30 mm, 5 μm); 50% IPA+0.1% NH₄OH in CO₂) to afford (R)-6-isopropoxy-N-(pyrazolo[1,5-a]pyrimidin-3-yl)-2-((tetrahydrofuran-3-yl)methyl)-2H-pyrazolo[3,4-b]pyridine-5-carboxamide and (S)-6-isopropoxy-N-(pyrazolo[1,5-a]pyrimidin-3-yl)-2-((tetrahydrofuran-3-yl)methyl)-2H-pyrazolo[3,4-b]pyridine-5-carboxamide.
*Peak 1, Example 118. 26.1 mg, 37.3%; LCMS m/z=444.0 [M+Na]+. ¹H NMR (400 MHz, CDCl₃) δ: 1.66 (d, 6H). 1.69-1.78 (m, 1H), 2.06-2.15 (m, 1H), 3.09-3.16 (m, 1H), 3.66 (dd, 1H), 3.76-3.85 (m, 2H), 3.94-4.01 (m, 1H), 4.36 (d, 2H), 5.86-5.93 (m, 1H), 6.81 (dd, 1H), 8.01 (s, 1H), 8.42 (dd, 1H), 8.62 (dd, 1H), 9.00 (s, 1H), 9.10 (s, 1H), 10.80 (brs, 1H).
*Peak 2, Example 119. 30.5 mg, 43.6%; LCMS m/z=444.0 [M+Na]+. ¹H NMR (400 MHz, CDCl₃) δ: 1.66 (d, 6H), 1.69-1.78 (m, 1H), 2.06-2.15 (m, 1H), 3.09-3.16 (m, 1H), 3.66 (dd, 1H), 3.76-3.85 (m, 2H), 3.94-4.01 (m, 1H), 4.36 (d, 2H), 5.86-5.93 (m, 1H), 6.81 (dd, 1H), 8.01 (s, 1H), 8.42 (dd, 1H), 8.62 (dd, 1H), 9.00 (s, 1H), 9.10 (s, 1H), 10.80 (brs, 1H).

### Example 120 and 121: (R)-6-Isopropoxy-N-(pyrazolo[1,5-a]pyrimidin-3-yl)-2-(tetrahydro-2H-pyran-3-yl)-2H-pyrazolo[3,4-b]pyridine-5-carboxamide and (S)-6-isopropoxy-N-(pyrazolo[1,5-a]pyrimidin-3-yl)-2-(tetrahydro-2H-pyran-3-yl)-2H-pyrazolo[3,4-b]pyridine-5-carboxamide

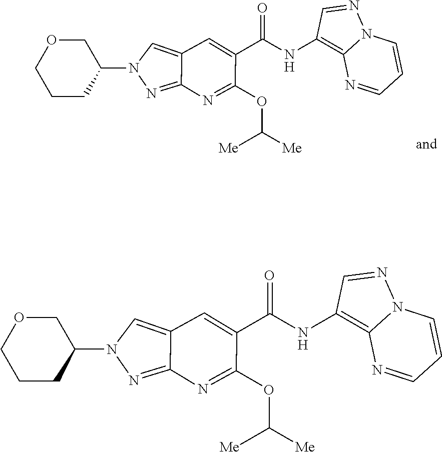 [SMILES: CC(C)Oc1nc2nn([C@@H]3CCCOC3)cc2cc1C(=O)Nc1cnn2cccnc12 ; CC(C)Oc1nc2nn([C@H]3CCCOC3)cc2cc1C(=O)Nc1cnn2cccnc12]

### *Stereochemistry Arbitrarily Assigned

To a solution of 6-isopropoxy-2-(tetrahydro-2H-pyran-3-yl)-2H-pyrazolo[3,4-b]pyridine-5-carboxylic acid (Preparation 103, 55 mg, 0.180 mmol) in pyridine (3 mL) was added pyrazolo[1,5-a]pyrimidin-3-amine (48.3 mg, 0.360 mmol) and T3P® (50 wt. % in EtOAc, 3 mL) and the reaction mixture stirred at 20° C. for 14 h. The reaction was evaporated to dryness in vacuo and the residue diluted with aqueous NaHCO₃ (30 mL) and extracted with EtOAc (3×30 mL). The combined organics were dried (Na₂SO₄) and evaporated to dryness in vacuo and the residue purified by Combi-Flash (PE/EA; 1:1 to 0:1) to afford 6-isopropoxy-N-(pyrazolo[1,5-a]pyrimidin-3-yl)-2-(tetrahydro-2H-pyran-3-yl)-2H-pyrazolo[3,4-b]pyridine-5-carboxamide as a yellow solid (80 mg, 94.8%). LCMS m/z=422.3 [M+H]⁺. 6-Isopropoxy-N-(pyrazolo[1,5-a]pyrimidin-3-yl)-2-(tetrahydro-2H-pyran-3-yl)-2H-pyrazolo[3,4-b]pyridine-5-carboxamide was purified by prep-SFC (DAICEL CHIRALPAK AY-H; 250×30 mm, 5 μm); 50% IPA+0.1% NH₄OH in CO₂) to give (R)-6-isopropoxy-N-(pyrazolo[1,5-a]pyrimidin-3-yl)-2-(tetrahydro-2H-pyran-3-yl)-2H-pyrazolo[3,4-b]pyridine-5-carboxamide and (S)-6-isopropoxy-N-(pyrazolo[1,5-a]pyrimidin-3-yl)-2-(tetrahydro-2H-pyran-3-yl)-2H-pyrazolo[3,4-b]pyridine-5-carboxamide.
*Peak 1, Example 120; 23 mg, 28%; LCMS m/z=422.0 [M+H]⁺. ¹H NMR (400 MHz, CDCl₃) δ: 1.66 (d, 6H), 1.75-1.90 (m, 2H), 2.29-2.35 (m, 1H), 2.39-2.49 (m, 1H), 3.62-3.69 (m, 1H), 3.91-3.97 (m, 1H), 4.00-4.06 (m, 1H), 4.22 (dd, 1H), 4.52-4.59 (m, 1H), 5.87-5.94 (m, 1H), 6.81 (dd, 1H), 8.17 (s, 1H), 8.42 (dd, 1H), 8.62 (dd, 1H), 9.01 (s, 1H), 9.11 (s, 1H), 10.80 (brs, 1H).
*Peak 2, Example 121; 23.9 mg, 29%; LCMS m/z=444.2 [M+H]⁺. ¹H NMR (400 MHz, CDCl₃) δ: 1.66 (d, 6H), 1.79-1.89 (m, 2H), 2.29-2.35 (m, 1H), 2.39-2.49 (m, 1H), 3.62-3.69 (m, 1H), 3.91-3.97 (m, 1H), 4.00-4.06 (m, 1H), 4.22 (dd, 1H), 4.52-4.59 (m, 1H), 5.54-5.87 (m, 1H), 6.81 (dd, 1H), 8.17 (s, 1H), 8.42 (dd, 1H), 8.62 (dd, 1H), 9.01 (s, 1H), 9.11 (s, 1H), 10.80 (brs, 1H).

### Example 122 and 123: (R)-6-Cyclobutoxy-N-(6-methylpyrazolo[1,5-a]pyrimidin-3-yl)-2-((tetrahydrofuran-3-yl)methyl)-2H-pyrazolo[3,4-b]pyridine-5-carboxamide and (S)-6-cyclobutoxy-N-(6-methylpyrazolo[1,5-a]pyrimidin-3-yl)-2-((tetrahydrofuran-3-yl)methyl)-2H-pyrazolo[3,4-b]pyridine-5-carboxamide

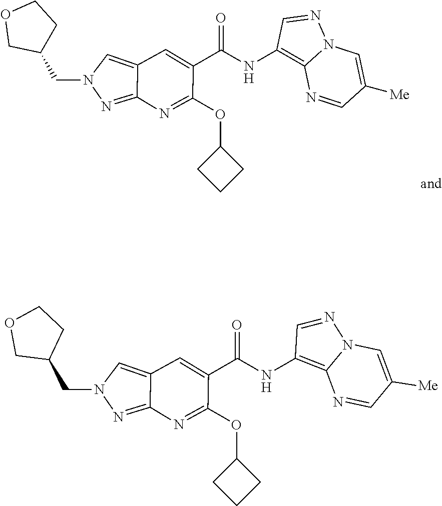 [SMILES: Cc1cnc2c(NC(=O)c3cc4cn(C[C@H]5CCOC5)nc4nc3OC3CCC3)cnn2c1 ; Cc1cnc2c(NC(=O)c3cc4cn(C[C@@H]5CCOC5)nc4nc3OC3CCC3)cnn2c1]

### *Stereochemistry Arbitrarily Assigned.

To a solution 6-cyclobutoxy-2-((tetrahydrofuran-3-yl)methyl)-2H-pyrazolo[3,4-b]pyridine-5-carboxylic acid (Preparation 105, 100 mg, 0.315 mmol) in pyridine (3 mL) was added 6-methylpyrazolo[1,5-a]pyrimidin-3-amine (93.4 mg, 0.630 mmol) and T3P® (50 wt. % in EtOAc, 4 mL) and the reaction stirred at 20° C. for 14 h. The reaction was evaporated to dryness in vacuo and the residue diluted with aqueous NaHCO₃ (30 mL) and extracted with EtOAc (3×30 mL). The combined organics were dried (Na₂SO₄) and evaporated to dryness in vacuo and the residue purified by Combi-Flash (PE/EA; 1:1 to 0:1) to give 6-cyclobutoxy-N-(6-methylpyrazolo[1,5-a]pyrimidin-3-yl)-2-((tetrahydrofuran-3-yl)methyl)-2H-pyrazolo[3,4-b]pyridine-5-carboxamide as a yellow solid (100 mg, 64%) which was purified by prep-SFC purification (DAICEL CHIRALPAK AD; 250×30 mm, 10 μm); 40% EtOH+0.1% NH₄OH in CO₂) to afford (R)-6-cyclobutoxy-N-(6-methylpyrazolo[1,5-a]pyrimidin-3-yl)-2-((tetrahydrofuran-3-yl)methyl)-2H-pyrazolo[3,4-b]pyridine-5-carboxamide and (S)-6-cyclobutoxy-N-(6-methylpyrazolo[1,5-a]pyrimidin-3-yl)-2-((tetrahydrofuran-3-yl)methyl)-2H-pyrazolo[3,4-b]pyridine-5-carboxamide.
*Peak 1, Example 122; 47.8 mg, 47.8%; LCMS m/z=448.2 [M+H]⁺. ¹H NMR (500 MHz, CDCl₃) δ: 1.69-1.76 (m, 1H), 1.76-1.86 (m, 1H), 1.96-2.04 (m, 1H), 2.06-2.14 (m, 1H), 2.40 (s, 3H), 2.51-2.59 (m, 2H), 2.69-2.76 (m, 2H), 3.09-3.16 (m, 1H), 3.65 (dd, 1H), 3.77-3.84 (m, 2H), 3.95-4.00 (m, 1H), 4.35 (d, 2H), 5.66-5.73 (m, 1H), 8.00 (s, 1H), 8.32 (d, 1H), 8.41 (dd, 1H), 8.90 (s, 1H), 9.09 (s, 1H), 10.75 (s, 1H).
*Peak 2, Example 123; 45.7 mg, 45.7%; LCMS m/z=448.2 [M+H]⁺. ¹H NMR (500 MHz, CDCl₃) δ: 1.69-1.77 (m, 1H), 1.78-1.86 (m, 1H), 1.97-2.04 (m, 1H), 2.06-2.14 (m, 1H), 2.40 (s, 3H), 2.51-2.61 (m, 2H), 2.69-2.76 (m, 2H), 3.09-3.15 (m, 1H), 3.65 (dd, 1H), 3.77-3.84 (m, 2H), 3.95-4.00 (m, 1H), 4.35 (d, 2H), 5.66-5.73 (m, 1H), 8.00 (s, 1H), 8.32 (d, 1H), 8.41 (dd, 1H), 8.90 (s, 1H), 9.09 (s, 1H), 10.75 (brs, 1H).

### Example 124: 7-Chloro-N-(6-(difluoromethyl)pyridin-2-yl)-6-isopropoxy-2-(tetrahydro-2H-pyran-4-yl)-2H-indazole-5-carboxamide Trifluoroacetate

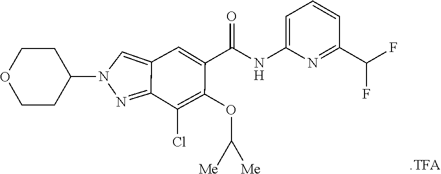 [SMILES: CC(C)Oc1c(C(=O)Nc2cccc(C(F)F)n2)cc2cn(C3CCOCC3)nc2c1Cl]

A solution of N-(6-(difluoromethyl)pyridin-2-yl)-6-isopropoxy-2-(tetrahydro-2H-pyran-4-yl)-2H-indazole-5-carboxamide (Example 25, 20 mg, 46.46 μmol) and NCS (6.2 mg, 46.46 mol) in MeCN (3 mL) was heated overnight at 50° C. The reaction mixture was purified by prep-HPLC (5-70% MeCN/H₂O+TFA) to afford 7-chloro-N-(6-(difluoromethyl)pyridin-2-yl)-6-isopropoxy-2-(tetrahydro-2H-pyran-4-yl)-2H-indazole-5-carboxamide trifluoroacetate (9.1 mg, 33.9%). LCMS m/z=465.0 [M+H]⁺; ¹H NMR (500 MHz, MeOH-d₄) δ: 1.38 (d, 6H), 2.16-2.23 (m, 2H), 2.23-2.34 (m, 2H), 3.60-3.69 (m, 2H), 4.13 (dd, 2H), 4.66-4.74 (m, 1H), 4.76-4.84 (m, 1H), 6.63 (t, 1H), 7.44 (d, 1H), 8.01 (t, 1H), 8.41 (d, 1H), 8.44 (d, 1H), 8.61 (d, 1H).

### Example 125 and 126: N-(6-(difluoromethyl)pyridin-2-yl)-6-isopropoxy-2-((1R,4R)-1-methyl-2-oxabicyclo[2.2.1]heptan-4-yl)-2H-indazole-5-carboxamide and N-(6-(difluoromethyl)pyridin-2-yl)-6-isopropoxy-2-((1S,4S)-1-methyl-2-oxabicyclo[2.2.1]heptan-4-yl)-2H-indazole-5-carboxamide

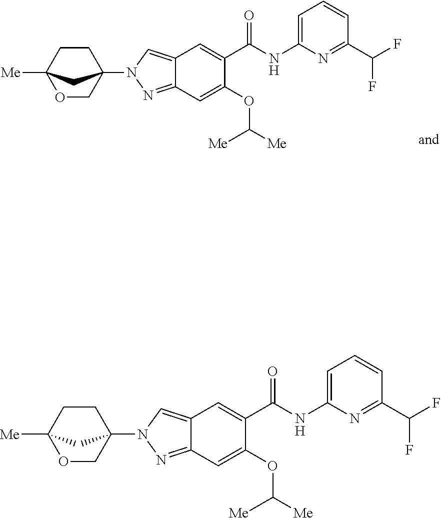 [SMILES: CC(C)Oc1cc2nn([C@]34CC[C@](C)(C3)OC4)cc2cc1C(=O)Nc1cccc(C(F)F)n1 ; CC(C)Oc1cc2nn([C@@]34CC[C@@](C)(C3)OC4)cc2cc1C(=O)Nc1cccc(C(F)F)n1]

### *Stereochemistry Arbitrarily Assigned

Example 97 was purified by prep-SFC (CHIRALPAK AD-H; 250×30 mm, 5 μm; 40% EtOH+0.1% DEA in CO₂) to afford N-(6-(difluoromethyl)pyridin-2-yl)-6-isopropoxy-2-((1R,4R)-1-methyl-2-oxabicyclo[2.2.1]heptan-4-yl)-2H-indazole-5-carboxamide and N-(6-(difluoromethyl)pyridin-2-yl)-6-isopropoxy-2-((1S,4S)-1-methyl-2-oxabicyclo[2.2.1]heptan-4-yl)-2H-indazole-5-carboxamide.
*Peak 1, Example 125, LCMS m/z=457.1 [M+H]⁺. ¹H NMR (400 MHz, MeOH-d₄) δ: 1.50 (s, 3H), 1.59 (d, 6H), 1.90-2.10 (m, 2H), 2.30-2.55 (m, 4H), 4.13 (dd, 1H), 4.22 (d, 1H), 4.97 (spt, 1H), 6.62 (t, 1H), 7.16 (s, 1H), 7.43 (d, 1H), 7.99 (t, 1H), 8.46 (d, 1H), 8.49 (s, 1H), 8.64 (s, 1H).
*Peak 2, Example 126, LCMS m/z=457.1 [M+H]⁺. ¹H NMR (400 MHz, MeOH-d₄) δ: 1.50 (s, 3H), 1.59 (d, 6H), 1.90-2.10 (m, 2H), 2.30-2.55 (m, 4H), 4.13 (dd, 1H), 4.22 (d, 1H), 4.97 (spt, 1H), 6.62 (t, 1H), 7.16 (s, 1H), 7.43 (d, 1H), 7.99 (t, 1H), 8.46 (d, 1H), 8.49 (s, 1H), 8.64 (s, 1H).

### Example 127 and 128: 6-Isopropoxy-2-((1R,4R)-1-methyl-2-oxabicyclo[2.2.1]heptan-4-yl)-N-(pyrazolo[1,5-a]pyrimidin-3-yl)-2H-indazole-5-carboxamide and 6-isopropoxy-2-((1S,4S)-1-methyl-2-oxabicyclo[2.2.1]heptan-4-yl)-N-(pyrazolo[1,5-a]pyrimidin-3-yl)-2H-indazole-5-carboxamide

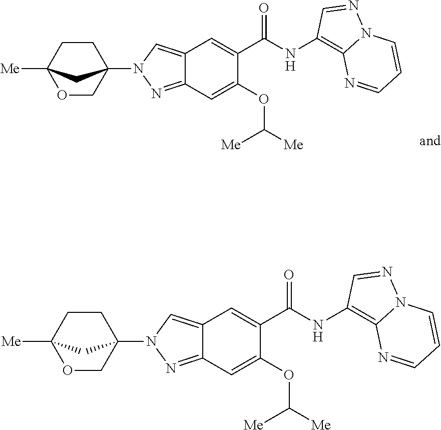 [SMILES: CC(C)Oc1cc2nn([C@]34CC[C@](C)(C3)OC4)cc2cc1C(=O)Nc1cnn2cccnc12 ; CC(C)Oc1cc2nn([C@@]34CC[C@@](C)(C3)OC4)cc2cc1C(=O)Nc1cnn2cccnc12]

### *Stereochemistry Arbitrarily Assigned

Example 98 was purified by prep-SFC (CHIRALPAK AD-H; 250×30 mm, 5 μm; 40% IPA+0.1% DEA in CO₂) to afford 6-isopropoxy-2-((1R,4R)-1-methyl-2-oxabicyclo[2.2.1]heptan-4-yl)-N-(pyrazolo[1,5-a]pyrimidin-3-yl)-2H-indazole-5-carboxamide and 6-isopropoxy-2-((1S,4S)-1-methyl-2-oxabicyclo[2.2.1]heptan-4-yl)-N-(pyrazolo[1,5-a]pyrimidin-3-yl)-2H-indazole-5-carboxamide.
*Peak 1, Example 127, LCMS m/z=447.2 [M+H]⁺. ¹H NMR (400 MHz, MeOH-d₄) δ: 1.50 (s, 3H), 1.65 (d, 6H), 2.00-2.15 (m, 2H), 2.30-2.55 (m, 4H), 4.13 (dd, 1H), 4.22 (d, 1H), 5.02 (spt, 1H), 7.01 (dd, 1H), 7.18 (s, 1H), 8.45-8.55 (m, 2H), 8.70 (s, 1H), 8.80-8.90 (m, 2H).
*Peak 2, Example 128, LCMS m/z=447.2 [M+H]⁺. ¹H NMR (400 MHz, MeOH-d₄) δ: 1.50 (s, 3H), 1.65 (d, 6H), 2.00-2.15 (m, 2H), 2.30-2.55 (m, 4H), 4.13 (dd, 1H), 4.22 (d, 1H), 5.02 (spt, 1H), 7.01 (dd, 1H), 7.18 (s, 1H), 8.45-8.55 (m, 2H), 8.70 (s, 1H), 8.80-8.90 (m, 2H).

### Examples 129 and 130: re1-(S)-6-cyclobutoxy-N-(1-methyl-1H-pyrazol-3-yl)-2-(tetrahydro-2H-pyran-3-yl)-2H-pyrazolo[3,4-b]pyridine-5-carboxamide and re1-(R)-6-cyclobutoxy-N-(1-methyl-1H-pyrazol-3-yl)-2-(tetrahydro-2H-pyran-3-yl)-2H-pyrazolo[3,4-b]pyridine-5-carboxamide

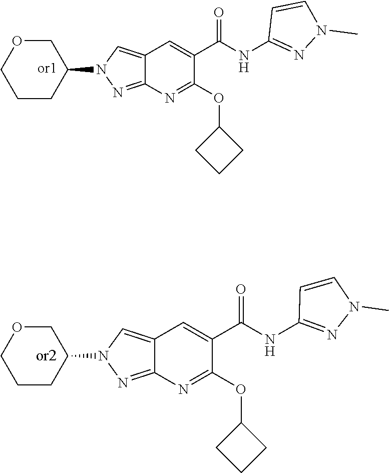 [SMILES: Cn1ccc(NC(=O)c2cc3cn([C@H]4CCCOC4)nc3nc2OC2CCC2)n1 ; Cn1ccc(NC(=O)c2cc3cn([C@@H]4CCCOC4)nc3nc2OC2CCC2)n1]

### *Stereochemistry Arbitrarily Assigned

To a solution of 1-methyl-1H-pyrazol-3-amine (48.9 mg, 504 μmol, 2.0 eq.) in pyridine (3 mL) was added 6-cyclobutoxy-2-(tetrahydro-2H-pyran-3-yl)-2H-pyrazolo[3,4-b]pyridine-5-carboxylic acid (preparation 131, 80.0 mg, 252 μmol, 1.0 eq.) and T3P (3 mL) at 20° C. The reaction was stirred at 20° C. for 14 hours. The reaction was evaporated under vacuum. The residue was diluted with aqueous NaHCO₃ (30 mL), extracted with EtOAc (30 mL×3). The organic layer was dried over Na₂SO₄; filtered and evaporated under vacuum. The residue was purified by Combi-Flash (PE:EA from 1:1 to 0:1) to give racemic title compound (95.0 mg, 85.5% yield) as a white solid, which was purified by prep-SFC (Column: DAICEL CHIRALPAK AD (250 mm×30 mm, 10 um); Mobile Phase: from 35% to 35% of 0.1% NH₃H2O ETOH; Flow Rate (ml/min): 80; Column temp: 35° C.) to give two enantiomers as yellow solids.
*Peak 1, Example 129; 40.8 mg, 42.9% yield; LCMS: m/z=397.0 [M+H]⁺. ¹H NMR: (500 MHz, CDCl₃) δ: 1.78-1.73 (m, 1H), 1.87-1.79 (m, 2H), 1.98-1.91 (m, 1H), 2.45-2.29 (m, 4H), 2.72-2.66 (m, 2H), 3.68-3.62 (m, 1H), 3.86 (s, 3H), 3.95-3.91 (m, 1H), 4.01 (dd, J₁=11.5 Hz, J₂=8.5 Hz, 1H), 4.20 (dd, J₁=11.5 Hz, J₂=3.5 Hz, 1H), 4.57-4.51 (m, 1H), 5.65-5.60 (m, 1H), 6.83 (s, 1H), 7.31 (s, 1H), 8.15 (s, 1H), 9.04 (s, 1H), 10.40 (brs, 1H).
*Peak 2, Example 130; 42.8 mg, 45.0% yield; LCMS: m/z=397.0 [M+H]⁺. ¹H NMR: (500 MHz, CDCl₃) δ: 1.77-1.73 (m, 1H), 1.86-1.77 (m, 2H), 1.98-1.91 (m, 1H), 2.45-2.29 (m, 4H), 2.73-2.66 (m, 2H), 3.67-3.62 (m, 1H), 3.86 (s, 3H), 3.95-3.90 (m, 1H), 4.01 (dd, J₁=11.5 Hz, J₂=8.5 Hz, 1H), 4.20 (dd, J₁=11.5 Hz, J₂=3.0 Hz, 1H), 4.55-4.52 (m, 1H), 5.65-5.58 (m, 1H), 6.83 (d, J=2.0 Hz, 1H), 7.31 (s, 1H), 8.15 (s, 1H), 9.04 (s, 1H), 10.40 (brs, 1H).

### Examples 131 and 132: re1-(S)-6-cyclobutoxy-N-(6-methylpyrazolo[1,5-a]pyrimidin-3-yl)-2-(tetrahydro-2H-pyran-3-yl)-2H-pyrazolo[3,4-b]pyridine-5-carboxamide and re1-(R)-6-cyclobutoxy-N-(6-methylpyrazolo[1,5-a]pyrimidin-3-yl)-2-(tetrahydro-2H-pyran-3-yl)-2H-pyrazolo[3,4-b]pyridine-5-carboxamide

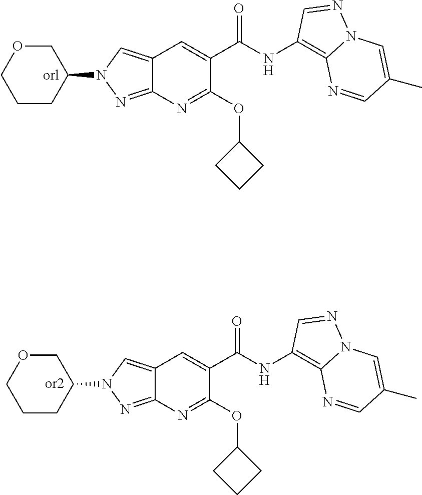 [SMILES: Cc1cnc2c(NC(=O)c3cc4cn([C@H]5CCCOC5)nc4nc3OC3CCC3)cnn2c1 ; Cc1cnc2c(NC(=O)c3cc4cn([C@@H]5CCCOC5)nc4nc3OC3CCC3)cnn2c1]

### *Stereochemistry Arbitrarily Assigned

To a solution of 6-methylpyrazolo[1,5-a]pyrimidin-3-amine (74.7 mg, 504 μmol, 2.0 eq.) in pyridine (3 mL) was added 6-cyclobutoxy-2-(tetrahydro-2H-pyran-3-yl)-2H-pyrazolo[3,4-b]pyridine-5-carboxylic acid (preparation 131, 80.0 mg, 252 μmol, 1.0 eq.) and T3P (3 mL) at 20° C. The reaction was stirred at 20° C. for 14 hours. Solvent was evaporated under vacuum. The residue was diluted with aqueous NaHCO₃ (30 mL), extracted with EtOAc (30 mL×3). The organic layer was dried over Na₂SO₄; filtered and evaporated under vacuum. The residue was purified by Combi-Flash (PE:EA from 1:1 to 0:1) to give racemic title compound (100 mg, 79.8% yield) as a yellow solid, which was purified by prep-SFC (Column: DAICEL CHIRALPAK AD (250 mm×30 mm, 10 um); Mobile Phase: from 50% to 50% of 0.1% NH₃H2O ETOH; Flow Rate (ml/min): 80; Column temp: 40° C.) to give two enantiomers as yellow solids.
*Peak 1, Example 131; 21.4 mg, 21.4% yield; LCMS: m/z=448.2 [M+H]⁺. ¹H NMR: (500 MHz, CDCl₃) δ: 1.81-1.75 (m, 1H), 1.88-1.81 (m, 2H), 2.05-1.96 (m, 1H), 2.34-2.28 (m, 1H), 2.40 (s, 3H), 2.47-2.41 (m, 1H), 2.61-2.53 (m, 2H), 2.76-2.69 (m, 2H), 3.68-3.62 (m, 1H), 3.96-3.91 (m, 1H), 4.02 (dd, J₁=11.5 Hz, J₂=8.0 Hz, 1H), 4.23-4.19 (m, 1H), 4.58-4.52 (m, 1H), 5.74-5.67 (m, 1H), 8.17 (s, 1H), 8.32 (d, J=2.0 Hz, 1H), 8.41 (d, J=1.0 Hz, 1H), 8.90 (s, 1H), 9.10 (s, 1H), 10.76 (brs, 1H).
*Peak 2, Example 132; 29.8 mg, 29.8% yield; LCMS: m/z=448.1 [M+H]⁺. ¹H NMR: (500 MHz, CDCl₃) δ: 1.81-1.75 (m, 1H), 1.88-1.81 (m, 2H), 2.05-1.97 (m, 1H), 2.34-2.29 (m, 1H), 2.40 (s, 3H), 2.44-2.41 (m, 1H), 2.59-2.51 (m, 2H), 2.75-2.71 (m, 2H), 3.68-3.62 (m, 1H), 3.96-3.91 (m, 1H), 4.02 (dd, J₁=11.0 Hz, J₂=8.0 Hz, 1H), 4.23-4.20 (m, 1H), 4.58-4.52 (m, 1H), 5.74-5.67 (m, 1H), 8.17 (s, 1H), 8.32 (d, J=2.0 Hz, 1H), 8.41 (d, J=1.5 Hz, 1H), 8.90 (s, 1H), 9.10 (s, 1H), 10.76 (brs, 1H).

### Examples 133 and 134: re1-(S)-6-cyclobutoxy-N-(5-methylpyrazolo[1,5-a]pyrimidin-3-yl)-2-(tetrahydro-2H-pyran-3-yl)-2H-pyrazolo[3,4-b]pyridine-5-carboxamide and re1-(R)-6-cyclobutoxy-N-(5-methylpyrazolo[1,5-a]pyrimidin-3-yl)-2-(tetrahydro-2H-pyran-3-yl)-2H-pyrazolo[3,4-b]pyridine-5-carboxamide

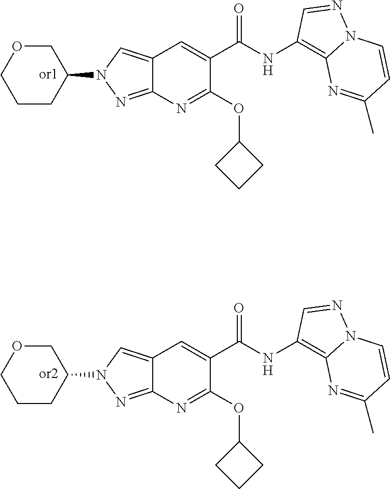 [SMILES: Cc1ccn2ncc(NC(=O)c3cc4cn([C@H]5CCCOC5)nc4nc3OC3CCC3)c2n1 ; Cc1ccn2ncc(NC(=O)c3cc4cn([C@@H]5CCCOC5)nc4nc3OC3CCC3)c2n1]

To a solution of 5-methylpyrazolo[1,5-a]pyrimidin-3-amine (56.0 mg, 378 μmol, 2.0 eq.) in pyridine (2 mL) was added 6-cyclobutoxy-2-(tetrahydro-2H-pyran-3-yl)-2H-pyrazolo[3,4-b]pyridine-5-carboxylic acid (preparation 131, 60.0 mg, 189 μmol, 1.0 eq.) and T3P (2 mL) at 20° C. The reaction was stirred at 20° C. for 14 hours. The reaction was evaporated under vacuum. The residue was diluted with aqueous NaHCO₃ (30 mL), extracted with EtOAc (30 mL×3). The organic layer was dried over Na₂SO₄; filtered and evaporated under vacuum. The residue was purified by Combi-Flash (PE:EA from 1:1 to 0:1) to give racemic title compound (80.0 mg, 85.1% yield) as a yellow solid, which was purified by prep-SFC (Column: DAICEL CHIRALPAK IC (250 mm×30 mm, 5 um); Mobile Phase: from 50% to 50% of MeOH-ACN; Flow Rate (ml/min): 25; Gradient Time (min): 60; Column temp: 25° C.) to give two enantiomers as yellow solids.
*Peak 1, Example 133; 33.1 mg, 33.1% yield; LCMS: m/z=448.0 [M+H]⁺. ¹H NMR: (500 MHz, CDCl₃) δ: 1.81-1.76 (m, 1H), 1.88-1.81 (m, 2H), 2.06-1.98 (m, 1H), 2.33-2.29 (m, 1H), 2.46-2.38 (m, 1H), 2.62 (s, 3H), 2.68-2.62 (m, 2H), 2.78-2.71 (m, 2H), 3.68-3.62 (m, 1H), 3.96-3.91 (m, 1H), 4.02 (dd, J₁=11.5 Hz, J₂=8.0 Hz, 1H), 4.23-4.19 (m, 1H), 4.58-4.52 (m, 1H), 5.77-5.70 (m, 1H), 6.67 (d, J=7.5 Hz, 1H), 8.17 (s, 1H), 8.47 (d, J=7.5 Hz, 1H), 8.93 (s, 1H), 9.10 (s, 1H), 10.80 (brs, 1H).
*Peak 2, Example 134; 35.6 mg, 35.6% yield; LCMS: m/z=448.1 [M+H]⁺. ¹H NMR: (500 MHz, CDCl₃) δ: 1.81-1.77 (m, 1H), 1.88-1.81 (m, 2H), 2.06-1.98 (m, 1H), 2.34-2.29 (m, 1H), 2.47-2.38 (m, 1H), 2.63 (s, 3H), 2.69-2.63 (m, 2H), 2.78-2.71 (m, 2H), 3.68-3.62 (m, 1H), 3.96-3.91 (m, 1H), 4.02 (dd, J₁=11.5 Hz, J₂=8.0 Hz, 1H), 4.23-4.20 (m, 1H), 4.58-4.52 (m, 1H), 5.77-5.71 (m, 1H), 6.67 (d, J=7.5 Hz, 1H), 8.17 (s, 1H), 8.47 (d, J=7.5 Hz, 1H), 8.93 (s, 1H), 9.10 (s, 1H), 10.80 (brs, 1H).

### Examples 135 and 136: re1-6-isopropoxy-N-(6-methoxypyrazolo[1,5-a]pyrimidin-3-yl)-2-((1S,4S)-1-methyl-2-oxabicyclo[2.2.1]heptan-4-yl)-2H-indazole-5-carboxamide and re1-6-isopropoxy-N-(6-methoxypyrazolo[1,5-a]pyrimidin-3-yl)-2-((1R,4R)-1-methyl-2-oxabicyclo[2.2.1]heptan-4-yl)-2H-indazole-5-carboxamide

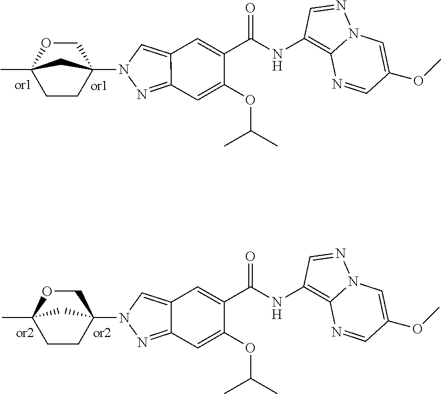

To a solution of rac-6-isopropoxy-2-((1R,4R)-1-methyl-2-oxabicyclo[2.2.1]heptan-4-yl)-2H-indazole-5-carboxylic acid (56.0 mg, 169 μmol, 1.0 eq.) and 6-methoxypyrazolo[1,5-a]pyrimidin-3-amine (41.7 mg, 254 μmol, 1.5 eq.) in Pyridine (2 mL) was added T₃P (2 mL). The mixture was stirred at 20° C. for 16 hours. The mixture was concentrated in vacuo to give the residue, which was diluted with saturated NaHCO₃ aq. till pH=7. And this mixture was extracted with EtOAc (50 mL×3). The combined organic layer was washed with brine (50 mL) and dried over Na₂SO₄, filtered. The filtrate was concentrated in vacuo to give the residue, which was purified by Combi-Flash (PE/EtOAc=0/1) to give racemic title compound (73.0 mg, 84.9% yield) as a yellow solid, which was purified by SFC (Column: Chiralcel OJ-3 100×4.6 mm×3 um; Mobile phase: A: CO₂ B:ethanol (0.05% DEA); Gradient: from 5% to 40% of B in 4 min and hold 40% for 2.5 min, then 5% of B for 1.5 min; Flow rate: 2.8 mL/min; Column temp.: 35° C.) to give two enantiomers as yellow solids.
*Peak 1, Example 135; 15.6 mg, 19.8% yield; LCMS: m/z=447.1 [M+H]⁺. ¹H NMR: (500 MHz, CDCl₃) δ: 1.53 (s, 3H), 1.65 (d, J=6.0 Hz, 6H), 2.09-1.97 (m, 2H), 2.37-2.30 (m, 2H), 2.44-2.41 (m, 1H), 2.52-2.45 (m, 1H), 3.90 (s, 3H), 4.20-4.18 (m, 1H), 4.24 (d, J=7.0 Hz, 1H), 4.93-4.88 (m, 1H), 7.16 (s, 1H), 8.07 (d, J=0.5 Hz, 1H), 8.14 (d, J=2.5 Hz, 1H), 8.27 (d, J=2.5 Hz, 1H), 8.82 (s, 1H), 8.87 (s, 1H), 10.83 (s, 1H).
*Peak 2, Example 136; 25.4 mg, 34.2% yield; LCMS: m/z=447.1 [M+H]⁺. ¹H NMR: (500 MHz, CDCl₃) δ: 1.53 (s, 3H), 1.65 (d, J=6.0 Hz, 6H), 2.09-1.97 (m, 2H), 2.37-2.30 (m, 2H), 2.44-2.41 (m, 1H), 2.52-2.45 (m, 1H), 3.90 (s, 3H), 4.20-4.17 (m, 1H), 4.24 (d, J=6.5 Hz, 1H), 4.93-4.88 (m, 1H), 7.16 (s, 1H), 8.08 (s, 1H), 8.14 (d, J=2.5 Hz, 1H), 8.27 (d, J=2.5 Hz, 1H), 8.82 (s, 1H), 8.87 (s, 1H), 10.83 (s, 1H).

### Examples 137 and 138: re1-6-cyclobutoxy-2-((1S,4S)-1-methyl-2-oxabicyclo[2.2.1]heptan-4-yl)-N-(pyrazolo[1,5-a]pyrimidin-3-yl)-2H-indazole-5-carboxamide and re1-6-cyclobutoxy-2-((1R,4R)-1-methyl-2-oxabicyclo[2.2.1]heptan-4-yl)-N-(pyrazolo[1,5-a]pyrimidin-3-yl)-2H-indazole-5-carboxamide

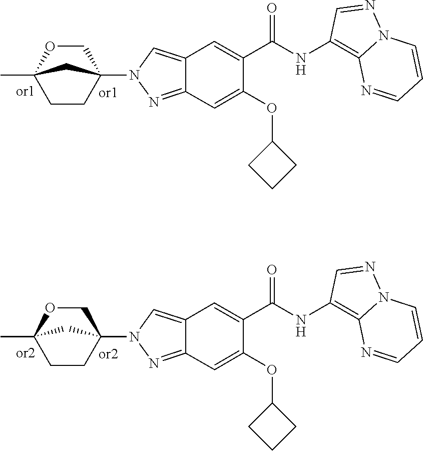 [SMILES: C[C@@]12CC[C@@](n3cc4cc(C(=O)Nc5cnn6cccnc56)c(OC5CCC5)cc4n3)(CO1)C2 ; C[C@]12CC[C@](n3cc4cc(C(=O)Nc5cnn6cccnc56)c(OC5CCC5)cc4n3)(CO1)C2]

To a solution of 6-(cyclobutoxy)-2-[(1S,4S)-1-methyl-2-oxabicyclo[2.2.1]heptan-4-yl]indazole-5-carboxylic acid (70.0 mg, 204 μmol, 1.0 eq.) and pyrazolo[1,5-a]pyrimidin-3-amine (41.1 mg, 306 μmol, 1.5 eq.) in Pyridine (2 mL) was added T₃P (2 mL). The mixture was stirred at 20° C. for 16 hours. The mixture was concentrated in vacuo to give the residue, which was diluted with saturated NaHCO₃ aq. till pH=7. And this mixture was extracted with EtOAc (50 mL×3). The combined organic layer was washed with brine (50 mL) and dried over Na₂SO₄, filtered. The filtrate was concentrated in vacuo to give the residue, which was purified by Combi-Flash (PE/EtOAc=0/1) to give racemic title compound (60.0 mg, 62.1% yield) as a yellow solid, which was purified by SFC(Column: Chiralpak AD-3 50; Á4.6 mm I.D., 3 um; Mobile phase: A: CO2 B:ethanol (0.05% DEA); Isocratic: 40% B; Flow rate: 4 mL/min; Column temp.: 35° C.; ABPR: 1500 psi) to give two enantiomers as yellow solids.
*Peak 1, Example 137; 25.4 mg, 25.2% yield; LCMS: m/z=459.0 [M+H]⁺. ¹H NMR: (500 MHz, CDCl₃) δ: 1.53 (s, 3H), 1.91-1.84 (m, 1H), 2.09-1.96 (m, 3H), 2.37-2.30 (m, 2H), 2.44-2.41 (m, 1H), 2.52-2.45 (m, 1H), 2.65-2.59 (m, 2H), 2.74-2.66 (m, 2H), 4.20-4.17 (m, 1H), 4.24 (d, J=6.5 Hz, 1H), 5.00-4.93 (m, 1H), 6.81-6.78 (s, 1H), 6.98 (s, 1H), 8.08 (d, J=1.0 Hz, 1H), 8.40-8.38 (m, 1H), 8.63-8.60 (m, 1H), 8.83 (s, 1H), 9.03 (s, 1H), 10.83 (s, 1H).
*Peak 2, Example 138; 35.4 mg, 35.0% yield; LCMS: m/z=459.0 [M+H]⁺. ¹H NMR: (500 MHz, CDCl₃) δ: 1.53 (s, 3H), 1.91-1.82 (m, 1H), 2.10-1.96 (m, 3H), 2.37-2.30 (m, 2H), 2.52-2.41 (m, 2H), 2.65-2.59 (m, 2H), 2.73-2.66 (m, 2H), 4.20-4.17 (m, 1H), 4.23 (d, J=6.5 Hz, 1H), 5.00-4.93 (m, 1H), 6.81-6.78 (s, 1H), 6.98 (s, 1H), 8.08 (d, J=0.5 Hz, 1H), 8.40-8.38 (m, 1H), 8.63-8.60 (m, 1H), 8.83 (s, 1H), 9.03 (s, 1H), 10.83 (s, 1H).

### Assays

Compounds of the invention were assessed for their ability to inhibit IRAK4 activity. The inhibitory properties of the compounds of the invention described herein can be evidenced by testing in any one of the following assays.

### Biochemical Assay

The 2-hour 10 μM ATP Biochemical Assay employs a MesoScale Detection (MSD) format. The kinase reaction is based on the IRAK4 phosphorylation of a biotin labeled peptide (IRAK1 activation loop sequence 360-389).

The kinase reaction in 30 μl is carried out in wells of a 384 well polypropylene assay plate, with 0.1 nM IRAK4, 1.6 μM of biotinylated peptide substrate and 10 μM ATP in 50 mM Hepes, pH 7.5, 60 mM NaCl, 5 mM MgCl₂, 0.25 mM MnCl₂, 2 mM DTT, 0.01% BSA, 0.01% BSA, and 1% DMSO (from compound DMSO stocks), for 2 hour at room temperature. The activity is quenched with 11 μl of 70 mM EDTA, pH 8.

To detect the phosphorylated biotinylated peptide substrate, 30 μl of the quenched reaction mixture is added to equivalent wells of a 384 well streptavidin coated MesoScale plate (Meso Scale Discovery #L21SA-1). After a 1 hour incubation of the plate for 1 hour at room temperature with gentle mixing, the plate wells are washed 3 times with 50 mM Tris, pH 7.5, 150 mM NaCl, 0.02% Tween-20.

A 25 μl volume of 1:500 anti-P-Threonine Rabbit polyclonal Antibody plus 1:500 Goat-anti-Rabbit Sulfo Tag Antibody (Meso Scale Discovery R32AB-1) in 50 mM Tris, pH 7.5, 150 mM NaCl, 0.02% Tween-20 plus 2% BSA is then added to each well. After a 1-hour incubation of the plate for 1 hour at room temperature with gentle mixing, the plate wells are washed, 3 times with 50 mM Tris, pH 7.5, 150 mM NaCl, 0.02% Tween-20. A 40 μl volume of 2×MSD Read Buffer (Meso Scale Discovery R92TC-1) is added to each well, and the plate is read immediately in an MSD Plate Reader (Meso Scale Discovery).

The 2-hour 1 mM ATP IRAK4 Biochemical assay was performed as described above, but with 100 μM IRAK4 and 1 mM ATP.

### MDR1-MDCK Assay Procedure

The assay makes use of human MDR1 transfected MDCK cells (NIH cell line in-licensed from Absorption Systems)
The compounds are tested at 1 μM concentration prepared in transport buffer (Hank's balanced salt solution with HEPES)
MDR1-MDCK cell are cultured for 7 days in 96 well transwell insert plates (Corning). Insert plates are washed before the assay and TEER (Trans epithelial electric resistance) is measured.
These plates are loaded with test compound solution 85 μL for A-B transport and 260 μL for B-A transport in the respective donor compartment. The volume of receiver buffer (Transport buffer supplemented with 1% BSA) in the respective receiver compartment is 250 and 75 μL.
10 μL samples is taken from donor compartment (T=0 timepoint)
Assay plates are incubated for 120 minutes.
At 120 minutes (T=120 timepoint) samples from respective donor (10 uL) and receiver (50 μL) compartments is taken.
After addition of 40 μL transport buffer with BSA to donor samples, crash solution (Acetonitrile with internal standard, 110 μL) is added to all samples.
After centrifugation 50 μL supernatant is transferred to separate plate and mixed with 50 μL water.
Samples are analyzed using LC-MS/MS coupled with high throughput injection system.
Analyte/internal standard area ratios are used for apparent permeability (Pₐₚₚ), efflux ratio and mass recovery estimation based on equations below.

Pₐₚₚ=(dCᵣ/dt)×Vᵣ/(A×C_{E})

Mass balance=100×((Vᵣ×Cᵣ^{final})+(V_{d}×C_{d}^{final}))/(V_{d}×C_{E})
Where:
dCᵣ/dt is the cumulative concentration in the receiver compartment versus time in μM s⁻¹
Vᵣ is the volume of the receiver compartment in cm³
V_{d} is the volume of the donor compartment in cm³
A is the area of the insert (0.143 cm² for 96-well insert)
C_{E} is the estimated experimental concentration (Time=0) of the dosing solution
Cᵣ^{final} is the concentration of the receiver at the end of the incubation period
C_{d}^{final} is the concentration of the donor at the end of the incubation period.

### Potency Data Table:

| | IRAK4 MSD | IRAK4 MSD | |
|---|---|---|---|
| | Biochemical Assay | Biochemical Assay | MDR1-MDCK |
| Example | (1 mM ATP, 2 h) | (10 μM ATP, 2 h) | Efflux Ratio; |
| Number | IC₅₀ (nM) | IC₅₀ (nM) | (B-A/A-B) |
| 1 | 32.7 | | 1.1 |
| 2 | 373.0 | | 1.7 |
| 3 | 10000.0 | | 0.9 |
| 4 | 3380.2 | | 0.4 |
| 5 | 21.0 | | 0.6 |
| 6 | 186.5 | | 1.1 |
| 7 | 1388.3 | | 0.6 |
| 8 | 275.4 | | 0.6 |
| 9 | 2739.3 | | 0.5 |
| 10 | | 26 | 0.9 |
| 11 | 6.8 | 2 | 1.1 |
| 12 | 5.5 | 2 | 1.4 |
| 13 | 0.4 | | 1.7 |
| 14 | 246.8 | | 24.4 |
| 15 | 123.5 | | 2.5 |
| 16 | 210.9 | | 3.0 |
| 17 | 11.3 | | 0.4 |
| 18 | 7.1 | | 0.9 |
| 19 | 5.4 | | 1.0 |
| 20 | 0.5 | | 0.7 |
| 21 | 0.5 | | 0.7 |
| 22 | 9.2 | | 0.6 |
| 23 | 51.3 | | 0.8 |
| 24 | 6.3 | | 0.7 |
| 25 | 0.3 | | 0.6 |
| 26 | 0.9 | | 0.7 |
| 27 | 5.4 | | 1.3 |
| 28 | 4.4 | | 0.4 |
| 29 | 0.6 | | 0.8 |
| 30 | 6.2 | | 0.6 |
| 31 | 0.7 | | 1.9 |
| 32 | 0.4 | | 3.4 |
| 33 | 4.3 | | 8.6 |
| 34 | 1.0 | | 14.2 |
| 35 | 5.0 | | 1.1 |
| 36 | 0.5 | | 2.2 |
| 37 | 6.4 | | 2.2 |
| 38 | 11.2 | | 2.3 |
| 39 | 1.8 | | 3.1 |
| 40 | 0.7 | | 5.7 |
| 41 | 3.2 | | 0.3 |
| 42 | 0.4 | | 0.9 |
| 43 | 0.2 | | 0.6 |
| 44 | 2.2 | | 0.8 |
| 45 | 2.1 | | 1.0 |
| 46 | 0.3 | | 1.2 |
| 47 | 0.2 | | 1.5 |
| 48 | 0.5 | | 1.3 |
| 49 | 2.0 | | 1.4 |
| 50 | 0.2 | | 1.4 |
| 51 | 1.3 | | 0.9 |
| 52 | 1.0 | | 1.0 |
| 53 | 1.5 | | 1.0 |
| 54 | 1.3 | | 3.6 |
| 55 | 1.2 | | 1.2 |
| 56 | 0.5 | | 2.0 |
| 57 | 0.8 | | 1.1 |
| 58 | 0.4 | | 0.9 |
| 59 | 1.0 | | 0.4 |
| 60 | 2.2 | | 1.5 |
| 61 | 3.7 | | 1.1 |
| 62 | 0.6 | | 1.7 |
| 63 | 0.3 | | 1.0 |
| 64 | 9.9 | | 1.6 |
| 65 | 15.4 | | 5.4 |
| 66 | 1.4 | | 0.9 |
| 67 | 9.5 | | 4.6 |
| 68 | 2.0 | | 1.4 |
| 69 | 1.0 | | 1.0 |
| 70 | 8.7 | | 1.1 |
| 71 | 6.6 | | 1.6 |
| 72 | 0.8 | | 2.1 |
| 73 | 1.8 | | 0.7 |
| 74 | 0.4 | | 0.5 |
| 75 | 1.1 | | 2.9 |
| 76 | 1.3 | | 1.0 |
| 77 | 13.6 | | 1.9 |
| 78 | 1.2 | | 1.4 |
| 79 | 10.6 | | 9.4 |
| 80 | 6.4 | | 3.1 |
| 81 | 9.9 | | 5.9 |
| 82 | 1.2 | | 2.6 |
| 83 | 1.4 | | 1.2 |
| 84 | 0.5 | | 0.9 |
| 85 | 0.9 | | 0.8 |
| 86 | 3.7 | | 1.8 |
| 87 | 2.4 | | 0.7 |
| 88 | 0.1 | | 0.5 |
| 89 | 1.0 | | 0.6 |
| 90 | 0.6 | | 0.7 |
| 91 | 0.3 | | 0.8 |
| 92 | 4.8 | | 1.6 |
| 93 | 0.4 | | 1.4 |
| 94 | 1.3 | | 1.4 |
| 95 | 0.3 | | 0.9 |
| 96 | 1.4 | | 1.6 |
| 97 | 0.2 | | 1.1 |
| 98 | 0.5 | | 1.0 |
| 99 | 0.3 | | 1.2 |
| 100 | 1.2 | | |
| 101 | 4.1 | | 1.5 |
| 103 | 1.9 | | 0.9 |
| 104 | 0.9 | | 4.8 |
| 105 | 1.5 | | 4.8 |
| 106 | 0.4 | | 0.8 |
| 107 | 0.3 | | 1.0 |
| 108 | 0.6 | | 1.8 |
| 109 | 1.0 | | 1.6 |
| 110 | 2.8 | | 2.2 |
| 111 | 2.7 | | 2.1 |
| 112 | 3.4 | | 8.3 |
| 113 | 2.1 | | 5.3 |
| 114 | 1.8 | | 1.5 |
| 115 | 1.0 | | 2.3 |
| 116 | 3.8 | | 2.5 |
| 117 | 2.0 | | 2.6 |
| 118 | 0.8 | | 3.1 |
| 119 | 1.0 | | 3.0 |
| 120 | 0.5 | | 0.9 |
| 121 | 0.7 | | 1.1 |
| 122 | 0.4 | | 4.2 |
| 123 | 0.5 | | 5.9 |
| 124 | 20.0 | | 1.1 |
| 125 | 0.4 | | 0.5 |
| 126 | 0.2 | | 0.5 |
| 127 | 0.4 | | 1.0 |
| 128 | 0.5 | | 1.4 |
| 129 | 5.7 | | 3.4 |
| 130 | 1.7 | | |
| 131 | 0.2 | | 2.6 |
| 132 | 0.1 | | 2.0 |
| 133 | 1.7 | | 2.6 |
| 134 | 1.0 | | 2.1 |
| 135 | 0.6 | | 2.3 |
| 136 | 1.4 | | 2.3 |
| 137 | 0.2 | | 2.1 |
| 138 | 0.2 | | 1.7 |
| 139 | 0.9 | | 0.8 |
| 140 | 0.9 | | 0.9 |

1. A compound of formula (I′): 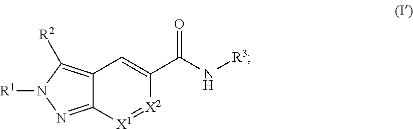
or a pharmaceutically acceptable salt thereof, wherein:
R¹ is selected from the group consisting of C₁₋₅ alkyl, C₃₋₆ cycloalkyl, —C₁₋₂ alkyl-C₃₋₆ cycloalkyl, a fully saturated 4 to 7 membered heterocycle containing 1 to 2 heteroatoms independently selected from nitrogen, sulfur and oxygen, —C₁₋₂ alkyl-C₄₋₇ heterocycle, wherein the C₄₋₇ heterocycle may be fully or partially saturated and contains 1 to 2 heteroatoms independently selected from nitrogen, sulfur and oxygen, —C₁₋₄ alkyl-O—C₁₋₂ alkyl, a fully saturated 5 to 8 membered bridged-carbocyclic ring, a fully saturated 5 to 8 membered bridged-heterocyclic ring system having 1 to 2 heteroatoms independently selected from nitrogen and oxygen, a 5 to 10 membered fused heterobicyclic ring system having 1 to 2 heteroatoms independently selected from nitrogen and oxygen and a 5 to 10 membered spiro heterobicyclic ring system having 1 to 2 heteroatoms independently selected from nitrogen and oxygen, wherein R¹ may be optionally substituted with 1, 2 or 3 substituents which are independently selected from halo, nitrile, oxo, halo-substitutedC₁₋₄ alkyl, hydroxy-substitutedC₁₋₄ alkyl, C₁₋₄ alkyl, C₄₋₇ heterocycle containing 1 to 2 heteroatoms independently selected from nitrogen and oxygen, a fully saturated 5 to 8 membered bridged-heterocyclic ring system having 1 to 2 heteroatoms independently selected from nitrogen and oxygen, C₁₋₄ alkyl-O—C₁₋₂ alkyl, hydroxyl and C₁₋₄ alkoxy;
R² is hydrogen, C₁₋₄ alkyl or halogen;
R³ is selected from the group consisting of
i. a 5 or 6 membered heteroaryl having 1 to 3 heteroatoms independently selected from nitrogen, oxygen and sulfur, said heteroaryl is optionally substituted with 1 to 3 R⁴;
ii. Phenyl optionally substituted with 1 to 3 R⁴,
iii. a 5-6 membered partially or fully saturated heterocycle having 1 to 2 heteroatoms independently selected from oxygen and nitrogen, said heterocycle may be optionally substituted with 1 to 3 R⁴;
iv. a partially or fully saturated C₃₋₆ cycloalkyl which may be optionally substituted with 1 to 3 R⁴;
v. a 7 to 10 membered fused heterobicyclic ring system having 1, 2 or 3 heteroatoms independently selected from nitrogen and oxygen, said ring system is optionally substituted with 1 to 3 R⁴; and
vi. a 7 to 10 membered fused bicyclic ring system optionally having 1, 2 or 3 heteroatoms independently selected from nitrogen and oxygen, said ring system is optionally substituted with 1 to 3 R⁴;
X₁ and X₂ are independently selected from N, CH and CR⁵, wherein only one of X₁ or X₂ may be N;
R⁵ is selected from halogen, C₁₋₄ alkyl, nitrile and —OR⁶;
R⁶ is hydrogen, a C₁₋₅ alkyl, a C₃₋₆ cycloalkyl or a fully saturated 4 to 7 membered heterocycle containing 1 or 2 heteroatoms selected from nitrogen and oxygen, wherein the C₁₋₅ alkyl represented by R⁶ is optionally substituted with 1 to 3 substituents R^{6a} independently selected from halogen, hydroxyl, C₁₋₄ alkoxy, C₃₋₆ cycloalkyl, phenyl and a 4 to 7 membered partially or fully saturated heterocycle containing 1 or 2 heteroatoms selected from nitrogen and oxygen, the C₃₋₆ cycloalkyl represented by R⁶ is optionally substituted with 1-3 substituent R^{6b} independently selected from halogen, C₁₋₄ alkyl, halo-substitutedC₁₋₄ alkyl and C₁₋₄ alkoxy; wherein said C₃₋₆ cycloalkyl and phenyl represented by R^{6a} may be optionally substituted with 1 to 3 R⁷;
each R⁷ is independently selected from oxo, halo, halo-substitutedC₁₋₄ alkyl and C₁₋₄ alkyl;
R⁴ for each occurrence, is independently selected from CN, hydroxyl, C₁₋₄ alkyl, CN-substitutedC₁₋₄ alkyl, oxo, halo, halo-substitutedC₁₋₄ alkyl, —NR⁸R⁹, C₁₋₄ alkoxy, C₁₋₄ alkoxy-C₁₋₄ alkoxy, hydroxy-substituted C₁₋₄ alkyl, halo-substitutedC₁₋₄ alkoxy, C₃₋₆ cycloalkyl, C(O)NR¹⁰R¹¹ and a 5 or 6 membered heteroaryl having 1 to 2 heteroatoms independently selected from nitrogen, oxygen and sulfur, said C₃₋₆ cycloalkyl and heteroaryl may be optionally substituted with 1 to 2 substituents independently selected from the group consisting of C₁₋₄ alkyl, hydroxyl and halogen; or two R⁴ groups on the same atom may form a C₃₋₆ cycloalkyl, or two R⁴ groups on adjacent ring atoms may form phenyl, C₄₋₆ carbocycle, C₄₋₆ heterocycle, or a 7 membered bridged ring system optionally having 1 heteroatom selected from nitrogen and oxygen, wherein said phenyl, C₃₋₆ cycloalkyl C₄₋₆ carbocycle and C₄₋₆ heterocycle may be optionally substituted with 1 to 2 C₁₋₄ alkyl, halo or halo-substitutedC₁₋₄ alkyl;
R⁸ and R⁹ are each independently selected from hydrogen, —C(O)C₁₋₄ alkyl and C₁₋₄ alkyl; or R⁸ and R⁹ may combine to form a 4 to 6 membered saturated ring optionally containing one additional heteroatom selected from nitrogen or oxygen wherein said additional nitrogen may be optionally substituted with C₁₋₄ alkyl; and
R¹⁰ and R¹¹ are each independently selected from hydrogen and C₁₋₄ alkyl.

2. The compound of claim 1, wherein the compound is of formula (I): 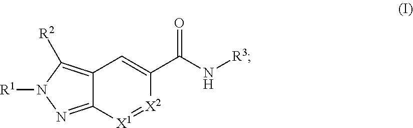
or a pharmaceutically acceptable salt thereof, wherein:
R¹ is selected from the group consisting of C₁₋₅ alkyl, C₃₋₆ cycloalkyl, —C₁₋₂ alkyl-C₃₋₆ cycloalkyl, a fully saturated 4 to 7 membered heterocycle containing 1 to 2 heteroatoms independently selected from nitrogen, sulfur and oxygen, —C₁₋₂ alkyl-C₄₋₇ heterocycle, wherein the C₄₋₇ heterocycle may be fully or partially saturated and contains 1 to 2 heteroatoms independently selected from nitrogen, sulfur and oxygen, —C₁₋₄ alkyl-O—C₁₋₂ alkyl, a fully saturated 5 to 8 membered bridged-carbocyclic ring, a fully saturated 5 to 8 membered bridged-heterocyclic ring system having 1 to 2 heteroatoms independently selected from nitrogen and oxygen, a 5 to 10 membered fused heterobicyclic ring system having 1 to 2 heteroatoms independently selected from nitrogen and oxygen and a 5 to 10 membered spiro heterobicyclic ring system having 1 to 2 heteroatoms independently selected from nitrogen and oxygen, wherein R¹ may be optionally substituted with 1, 2 or 3 substituents which are independently selected from halo, nitrile, oxo, halo-substitutedC₁₋₄ alkyl, hydroxy-substitutedC₁₋₄ alkyl, C₁₋₄ alkyl, C₄₋₇ heterocycle containing 1 to 2 heteroatoms independently selected from nitrogen and oxygen, C₁₋₄ alkyl-O—C₁₋₂ alkyl, hydroxyl and C₁₋₄ alkoxy;
R² is hydrogen, C₁₋₄ alkyl or halogen;
R³ is selected from the group consisting of
i. a 5 or 6 membered heteroaryl having 1 to 2 heteroatoms independently selected from nitrogen, oxygen and sulfur, said heteroaryl is optionally substituted with 1 to 3 R⁴;
ii. Phenyl optionally substituted with 1 to 3 R⁴,
iii. a 5-6 membered partially or fully saturated heterocycle having 1 to 2 heteroatoms independently selected from oxygen and nitrogen, said heterocycle may be optionally substituted with 1 to 3 R⁴;
iv. a partially or fully saturated C₃₋₆ cycloalkyl which may be optionally substituted with 1 to 3 R⁴;
v. a 7 to 10 membered fused heterobicyclic ring system having 1, 2 or 3 heteroatoms independently selected from nitrogen and oxygen, said ring system is optionally substituted with 1 to 3 R⁴; and
vi. a 7 to 10 membered fused bicyclic ring system optionally having 1, 2 or 3 heteroatoms independently selected from nitrogen and oxygen, said ring system is optionally substituted with 1 to 3 R⁴;
X₁ and X₂ are independently selected from N, CH and CR⁵, wherein only one of X₁ or X₂ may be N;
R⁵ is selected from halogen, C₁₋₄ alkyl, nitrile and —OR⁶;
R⁶ is hydrogen or an optionally substituted C₁₋₅ alkyl having 1 to 3 substituents independently selected from halogen, hydroxyl, C₁₋₄ alkoxy, C₃₋₆ cycloalkyl, phenyl and a 4 to 7 membered partially or fully saturated heterocycle containing 1 or 2 heteroatoms selected from nitrogen and oxygen, wherein said C₃₋₆ cycloalkyl and phenyl may be optionally substituted with 1 to 3 R⁷;
each R⁷ is independently selected from oxo, halo, halo-substitutedC₁₋₄ alkyl and C₁₋₄ alkyl;
R⁴ for each occurrence, is independently selected from CN, hydroxyl, C₁₋₄ alkyl, CN-substitutedC₁₋₄ alkyl, oxo, halo, halo-substitutedC₁₋₄ alkyl, —NR⁸R⁹, C₁₋₄ alkoxy, C₁₋₄ alkoxy-C₁₋₄ alkoxy, hydroxy-substituted C₁₋₄ alkyl, halo-substitutedC₁₋₄ alkoxy, C₃₋₆ cycloalkyl, C(O)NR¹⁰R¹¹ and a 5 or 6 membered heteroaryl having 1 to 2 heteroatoms independently selected from nitrogen, oxygen and sulfur, said C₃₋₆ cycloalkyl and heteroaryl may be optionally substituted with 1 to 2 substituents independently selected from the group consisting of C₁₋₄ alkyl, hydroxyl and halogen; or two R⁴ groups on the same atom may form a C₃₋₆ cycloalkyl, or two R⁴ groups on adjacent ring atoms may form phenyl, C₄₋₆ carbocycle, C₄₋₆ heterocycle, or a 7 membered bridged ring system optionally having 1 heteroatom selected from nitrogen and oxygen, wherein said phenyl, C₃₋₆ cycloalkyl C₄₋₆ carbocycle and C₄₋₆ heterocycle may be optionally substituted with 1 to 2 C₁₋₄ alkyl, halo or halo-substitutedC₁₋₄ alkyl;
R⁸ and R⁹ are each independently selected from hydrogen, —C(O)C₁₋₄ alkyl and C₁₋₄ alkyl; or R⁸ and R⁹ may combine to form a 4 to 6 membered saturated ring optionally containing one additional heteroatom selected from nitrogen or oxygen wherein said additional nitrogen may be optionally substituted with C₁₋₄ alkyl; and
R¹⁰ and R¹¹ are each independently selected from hydrogen and C₁₋₄ alkyl.

3. The compound of claim 1 or 2 of formula (I): 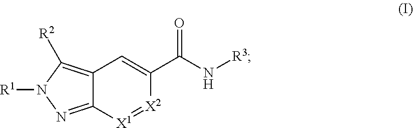
or a pharmaceutically acceptable salt thereof, wherein:
R² is H; and
X₁ is N or CH; and X₂ is CR⁵.

4. The compound of claim 1 or 2 of formula (I): 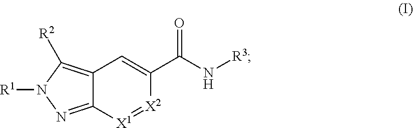
or a pharmaceutically acceptable salt thereof, wherein:
R² is H; and
X₁ is CR⁵ and X₂ is N or CH.

5. The compound of claim 1 or 2 of formula (Ia): 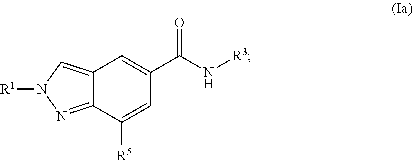
or a pharmaceutically acceptable salt thereof.

6. The compound of claim 1 or 2 of formula (Ib): 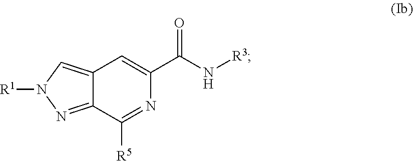
or a pharmaceutically acceptable salt thereof.

7. The compound of claim 1 or 2 of formula (Ic): 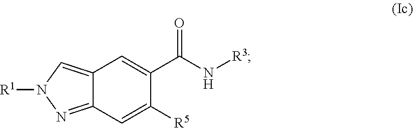
or a pharmaceutically acceptable salt thereof.

8. The compound of claim 1 or 2 of formula (Id): 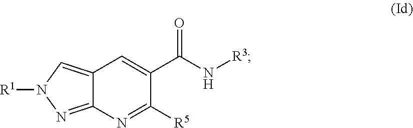
or a pharmaceutically acceptable salt thereof.

9. The compound of any one of the preceding claims, or a pharmaceutically acceptable salt thereof, wherein:
R³ is selected from the group consisting of
i. a 5 or 6 membered heteroaryl having 1 to 2 heteroatoms independently selected from nitrogen, oxygen and sulfur, said heteroaryl is optionally substituted with 1 to 3 R⁴;
ii. Phenyl optionally substituted with 1 to 3 R⁴,
iii. a 5-6 membered partially or fully saturated heterocycle having 1 to 2 heteroatoms independently selected from oxygen and nitrogen, said heterocycle may be optionally substituted with 1 to 3 R⁴;
iv. a partially or fully saturated C₃₋₆ cycloalkyl which may be optionally substituted with 1 to 3 R⁴;
v. a 7 to 10 membered fused heterobicyclic ring system having 1, 2 or 3 heteroatoms independently selected from nitrogen and oxygen, said ring system is optionally substituted with 1 to 3 R⁴; and
vi. a 7 to 10 membered fused bicyclic ring system optionally having 1, 2 or 3 heteroatoms independently selected from nitrogen and oxygen, said ring system is optionally substituted with 1 to 3 R⁴;

10. The compound of claim 9, or a pharmaceutically acceptable salt thereof, wherein:
R³ is phenyl, a 5 or 6 membered monocyclic heteroaryl having 1 to 2 heteroatoms independently selected from nitrogen and oxygen, pyridinyl-2 (1H)-one or a 9 to 10 membered bicyclic heteroaryl having 1 to 3 heteroatoms independently selected from nitrogen and oxygen, wherein the monocyclic heteroaryl, pyridinyl-2 (1H)-one or the bicyclic heteroaryl are each optionally substituted with 1 or 2 R⁴.

11. The compound of claim 10, or a pharmaceutically acceptable salt thereof, wherein:
R³ is phenyl, a 5 or 6 membered monocyclic heteroaryl having 1 to 2 nitrogen atoms, pyridinyl-2 (1H)-one or a 9 to 10 membered bicyclic heteroaryl having 2 to 3 nitrogen atoms, wherein the monocyclic heteroaryl, pyridinyl-2 (1H)-one or the bicyclic heteroaryl are each optionally substituted with 1 or 2 R⁴.

12. The compound of any one of claims 1 to 11, or a pharmaceutically acceptable salt thereof, wherein R⁴, for each occurrence, is independently selected from hydroxyl, halo, halo-substitutedC₁₋₄ alkyl, —NR⁸R⁹, and C₁₋₄ alkyl.

13. The compound of any one of claims 1 to 8, or a pharmaceutically acceptable salt thereof, wherein:
R³ is selected from pyridyl, oxazolyl, pyrazinyl, oxadiazoyl, thiophenyl, thiazolyl, isothiazolyl, pyrazolyl, imidazolyl, said R³ is optionally substituted with 1 to 2 substituents independently selected from the group consisting of halo, halo-substitutedC₁₋₄ alkyl, —NR⁸R⁹, and C₁₋₄ alkyl.

14. The compound of any one of claims 1 to 8, or a pharmaceutically acceptable salt thereof wherein:
R³ is pyridinyl-2 (1H)-one optionally substituted with 1 to 2 substituents independently selected from the group consisting of halo, halo-substitutedC₁₋₄ alkyl, —NR⁸R⁹, and C₁₋₄ alkyl.

15. The compound of any one of claims 1 to 8, or a pharmaceutically acceptable salt thereof, wherein:
R³ is phenyl, said phenyl is optionally substituted with 1 to 2 substituents independently selected from the group consisting of halo, halo-substitutedC₁₋₄ alkyl, —NR⁸R⁹, and C₁₋₄ alkyl.

16. The compound of any one of claims 1 to 8, or a pharmaceutically acceptable salt thereof, wherein:
R³ is selected from the group consisting of 1,3-dihydroisobenzofuran, 2,3-dihydrobenzofuran, 4-oxaspiro[bicyclo[3.2.0]heptane-6,1′-cyclobutane], oxaspiro[bicyclo[3.2.0]heptane-6,1′-cyclobutane], bicyclo[3.1.0]hexane, cyclohexyl, spiro[2.5]octane, 1S,5R)-1-methylbicyclo[3.1.0]hexane, 2,3-dihydro-1H-indene, spiro[2.5]octane, 1,2,3,4-tetrahydronaphthalen, tetrahydrofuran, 2,3-dihydrobenzofuran, 2,3-dihydro-1H-indene, 4-methyl-3,4-dihydro-2H-benzo[b][1,4]oxazine, pyrido[3,2-d]pyrimidinyl, 1,2,3,4-tetrahydro-1,4-epoxynaphthalene, 5,6-dihydro-4H-pyrrolo[1,2-b]pyrazole, 6,7-dihydro-5H-cyclopenta[b]pyridine, 1,2,3,4-tetrahydronaphthalene, indolin-2-one, 2,3-dihydrobenzofuran, pyrazolo[1,5-a]pyrimidine, 1-methyl-2-oxo-1,2,3,4-tetrahydroquinoline, 3,4-dihydroquinolin-2 (1H)-one, chromane, and isochromane, wherein said R³ is optionally substituted with 1 to 2 substituents independently selected from the group consisting halo, halo-substitutedC₁₋₄ alkyl, —NR⁸R⁹, and C₁₋₄ alkyl.

17. The compound of any one of claims 1 to 4 of formula (II): 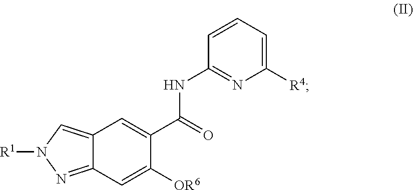
or a pharmaceutically acceptable salt thereof, wherein:
R⁶ is an optionally substituted C₁₋₅ alkyl having 1 to 3 substituents independently selected from halogen, hydroxyl, C₁₋₄ alkoxy, C₃₋₆ cycloalkyl, phenyl and a 4 to 7 membered partially or fully saturated heterocycle containing 1 or 2 heteroatoms selected from nitrogen and oxygen, wherein said C₃₋₆ cycloalkyl and phenyl may be optionally substituted with 1 to 3 R⁷.

18. The compound of any one of claims 1 to 4 of formula (III): 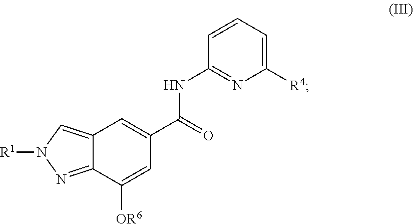
or a pharmaceutically acceptable salt thereof, wherein:
R⁶ is an optionally substituted C₁₋₅ alkyl having 1 to 3 substituents independently selected from halogen, hydroxyl, C₁₋₄ alkoxy, C₃₋₆ cycloalkyl, phenyl and a 4 to 7 membered partially or fully saturated heterocycle containing 1 or 2 heteroatoms selected from nitrogen and oxygen, wherein said C₃₋₆ cycloalkyl and phenyl may be optionally substituted with 1 to 3 R⁷.

19. The compound of any one of claims 1 to 4 of formula (IV): 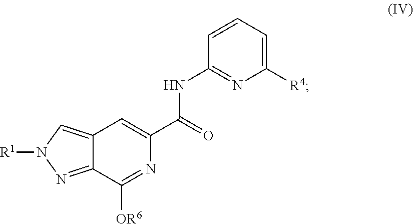
or a pharmaceutically acceptable salt thereof, wherein:
R⁶ is an optionally substituted C₁₋₅ alkyl having 1 to 3 substituents independently selected from halogen, hydroxyl, C₁₋₄ alkoxy, C₃₋₆ cycloalkyl, phenyl and a 4 to 7 membered partially or fully saturated heterocycle containing 1 or 2 heteroatoms selected from nitrogen and oxygen, wherein said C₃₋₆ cycloalkyl and phenyl may be optionally substituted with 1 to 3 R⁷.

20. The compound of any one of the preceding claims, or a pharmaceutically acceptable salt thereof, wherein:
R¹ is a fully saturated C₄₋₇ heterocycle or a 5 to 8 membered bridged-heterocyclic ring system which contain 1 to 2 heteroatoms independently selected from nitrogen and oxygen, said C₄₋₇ heterocycle or a 5 to 8 membered bridged-heterocyclic ring system may be optionally substituted with 1 or 2 substituents independently selected from the group consisting of C₁₋₄ alkyl, halogen, halo-substitutedC₁₋₄ alkyl, hydroxyl and C₁₋₄ alkoxy; or R¹ is a C₁₋₅ alkyl which is optionally substituted with 1 or 3 substituents independently selected from the group consisting of halogen, halo-substitutedC₁₋₄ alkyl, hydroxy-substitutedC₁₋₄ alkyl, hydroxyl, C₁₋₄ alkoxy and C₃₋₆ cycloalkyl, wherein said C₃₋₆ cycloalkyl is optionally substituted with 1 or 2 substituents independently selected from the group consisting of halogen, halo-substitutedC₁₋₄ alkyl, hydroxyl and C₁₋₄ alkoxy.

21. The compound of any one of the preceding claims, or a pharmaceutically acceptable salt thereof, wherein:
R¹ is a fully saturated C₄₋₇ heterocycle or a 5 to 8 membered bridged-heterocyclic ring system which contain 1 to 2 heteroatoms independently selected from nitrogen and oxygen, said C₄₋₇ heterocycle or a 5 to 8 membered bridged-heterocyclic ring system may be optionally substituted with 1 or 2 substituents independently selected from the group consisting of C₁₋₄ alkyl, halogen, halo-substitutedC₁₋₄ alkyl, hydroxyl and C₁₋₄ alkoxy.

22. The compound of any one of claims 1 to 19, or a pharmaceutically acceptable salt thereof, wherein:
R¹ is a C₁₋₅ alkyl which is optionally substituted with 1 or 3 substituents independently selected from the group consisting of halogen, halo-substitutedC₁₋₄ alkyl, hydroxyl, C₁₋₄ alkoxy and C₃₋₆ cycloalkyl, wherein said C₃₋₆ cycloalkyl is optionally substituted with 1 or 2 substituents independently selected from the group consisting of halogen, halo-substitutedCl₁₋₄ alkyl, hydroxyl and C₁₋₄ alkoxy.

23. The compound of any one of claims 1 to 19, or a pharmaceutically acceptable salt thereof, wherein R¹ is a C₄₋₇ heterocycle, —C₁₋₂ alkyl-C₄₋₇ heterocycle or a 5 to 8 membered bridged-heterocyclic ring system containing 1 to 2 heteroatoms independently selected from nitrogen and oxygen, wherein the C₄₋₇ heterocycle is fully saturated and contains 1 to 2 heteroatoms independently selected from nitrogen and oxygen and at least one of the heteroatoms is oxygen and wherein the C₄₋₇ heterocycle or the 5 to 8 membered bridged-heterocyclic ring system is optionally substituted with 1 or 2 substituents independently selected from the group consisting of C₁₋₄ alkyl, halogen, halo-substitutedC₁₋₄ alkyl, hydroxyl and C₁₋₄ alkoxy; or R¹ is a C₁₋₅ alkyl which is optionally substituted with 1 or 3 substituents independently selected from the group consisting of halogen, halo-substitutedC₁₋₄ alkyl, hydroxy-substitutedC₁₋₄ alkyl, hydroxyl, C₁₋₄ alkoxy and C₃₋₆ cycloalkyl, wherein said C₃₋₆ cycloalkyl is optionally substituted with 1 or 2 substituents independently selected from the group consisting of halogen, halo-substitutedC₁₋₄ alkyl, hydroxyl and C₁₋₄ alkoxy.

24. The compound of any one of claims 1 to 19, or a pharmaceutically acceptable salt thereof, wherein R¹ is a C₄₋₇ heterocycle, —C₁₋₂ alkyl-C₄₋₇ heterocycle or a 5 to 8 membered bridged-heterocyclic ring system containing 1 to 2 heteroatoms independently selected from nitrogen and oxygen, wherein the C₄₋₇ heterocycle is fully saturated and contains 1 to 2 heteroatoms independently selected from nitrogen and oxygen and at least one of heteroatom is oxygen and wherein the C₄₋₇ heterocycle or the 5 to 8 membered bridged-heterocyclic ring system may be optionally substituted with 1 or 2 substituents independently selected from the group consisting of C₁₋₄ alkyl, halogen, halo-substitutedC₁₋₄ alkyl, hydroxyl and C₁₋₄ alkoxy.

25. The compound of any one of claims 1 to 19, or a pharmaceutically acceptable salt thereof, wherein R¹ is a C₁₋₅ alkyl substituted with 1 or 3 substituents independently selected from the group consisting of halo-substitutedC₁₋₄ alkyl, hydroxyl, C₁₋₄ alkoxy and C₄₋₆ cycloalkyl, wherein said C₃₋₆ cycloalkyl is optionally substituted with 1 or 2 substituents independently selected from the group consisting of halogen, halo-substitutedC₁₋₄ alkyl, hydroxyl and C₁₋₄ alkoxy.

26. The compound of any one of claims 1 to 19, or a pharmaceutically acceptable salt thereof, wherein R¹ is a 5 to 8 membered bridged-heterocyclic ring system which contains 1 to 2 heteroatoms independently selected from nitrogen and oxygen, wherein the 5 to 8 membered bridged-heterocyclic ring system is optionally substituted with one or two substituents R^{1a} independently selected from C₁₋₄ alkyl, halogen, halo-substitutedC₁₋₄ alkyl, hydroxyl and C₁₋₄ alkoxy.

27. The compound of claim 26, or a pharmaceutically acceptable salt thereof, wherein R¹ is a 5 to 8 membered bridged-heterocyclic ring system containing one oxygen atom and wherein the 5 to 8 membered bridged-heterocyclic ring is optionally substituted with one or two substituents R^{1a} independently selected from C₁₋₄ alkyl, halogen, halo-substitutedC₁₋₄ alkyl, hydroxyl and C₁₋₄ alkoxy.

28. The compound of claim 27, or a pharmaceutically acceptable salt thereof, wherein R¹ is a 5 to 8 membered bridged-heterocyclic ring system represented by the following formula: 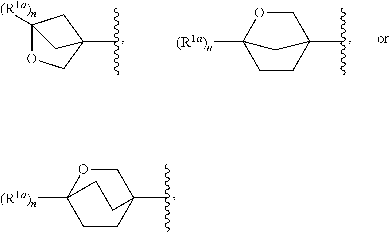
wherein R^{1a} is C₁₋₄ alkyl or halo-substitutedC₁₋₄ alkyl; and n is 0 or 1.

29. The compound of claim 28, or a pharmaceutically acceptable salt thereof, wherein R^{1a} is CH₃ or CH₂F.

30. The compound of any one of claim 1 to 4, or a pharmaceutically acceptable salt thereof, wherein:
R¹ is a fully saturated C₄₋₇ heterocycle or a 5 to 8 membered bridged-heterocyclic ring system which contain 1 to 2 heteroatoms independently selected from nitrogen and oxygen, said C₄₋₇ heterocycle or a 5 to 8 membered bridged-heterocyclic ring system may be optionally substituted with 1 or 2 substituents independently selected from the group consisting of C₁₋₄ alkyl,
halogen, halo-substitutedC₁₋₄ alkyl, hydroxyl and C₁₋₄ alkoxy; and R³ is pyridinyl substituted with 1 or 2 substituents independently selected from and C₁₋₄ alkyl and halo-substitutedC₁₋₄ alkyl.

31. The compound of any one of claims 1-16 and 20-30, or a pharmaceutically acceptable salt thereof, wherein R⁶ is an optionally substituted C₁₋₅ alkyl or an optionally substituted C₃₋₆ cycloalkyl, wherein the C₁₋₅ alkyl is optionally substituted with 1 to 3 substituents independently selected from halogen, hydroxyl and C₁₋₄ alkoxy and the C₃₋₆ cycloalkyl is optionally substituted with 1 to 3 substituents independently selected from halo, C₁₋₄ alky, halo-substitutedC₁₋₄ alkyl and C₁₋₄ alkoxy.

32. The compound of claim 1, wherein the compound is represented by the following formula: 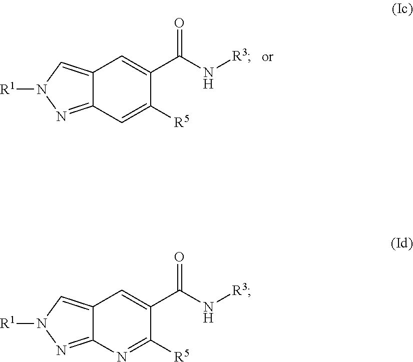
or a pharmaceutically acceptable salt thereof, wherein:
R¹ is —C₁₋₂ alkyl-C₄₋₇ heterocycle or a 5 to 8 membered bridged-heterocyclic ring system containing 1 to 2 heteroatoms independently selected from nitrogen and oxygen, wherein the C₄₋₇ heterocycle is fully saturated and contains 1 to 2 heteroatoms independently selected from nitrogen, sulfur and oxygen and wherein the C₄₋₇ heterocycle and the 5 to 8 membered bridged-heterocyclic ring system is optionally substituted with one or two substituents R^{1a};
R^{1a}, for each occurrence, is independently selected from C₁₋₄ alkyl, halogen, halo-substitutedC₁₋₄ alkyl, hydroxyl and C₁₋₄ alkoxy;
R³ is phenyl, a 5 or 6 membered monocyclic heteroaryl having 1 to 2 heteroatoms independently selected from nitrogen and oxygen, pyridinyl-2 (1H)-one or a 8 to 10 membered bicyclic heteroaryl having 1 to 3 heteroatoms independently selected from nitrogen and oxygen, wherein the monocyclic heteroaryl, pyridinyl-2 (1H)-one or the bicyclic heteroaryl are each optionally substituted with 1 or 2 R⁴;
R⁴, for each occurrence, is independently selected from hydroxyl, halo, halo-substitutedC₁₋₄ alkyl, —NR⁸R⁹, and C₁₋₄ alkyl;
R⁵ is OR⁶; and
R⁶ is an optionally substituted C₁₋₅ alkyl or an optionally substituted C₃₋₆ cycloalkyl, wherein the C₁₋₅ alkyl is optionally substituted with 1 to 3 substituents independently selected from halogen, hydroxyl and C₁₋₄ alkoxy and the C₃₋₆ cycloalkyl is optionally substituted with 1 to 3 substituents independently selected from halo, C₁₋₄ alky, halo-substitutedC₁₋₄ alkyl and C₁₋₄ alkoxy.

33. The compound of claim 32, or a pharmaceutically acceptable salt thereof, wherein
R¹ is —C₁₋₂ alkyl-C₄₋₇ heterocycle or a 5 to 8 membered bridged-heterocyclic ring system containing one oxygen atom, wherein the C₄₋₇ heterocycle contains one oxygen atom and wherein the C₄₋₇ heterocycle and the 5 to 8 membered bridged-heterocyclic ring system is optionally substituted with one substituent R^{1a};
R^{1a} is C₁₋₄ alkyl or halo-substitutedC₁₋₄ alkyl;
R³ is phenyl, a 5 or 6 membered monocyclic heteroaryl having 1 to 2 nitrogen atoms, pyridinyl-2 (1H)-one or a 8 to 10 membered bicyclic heteroaryl having 2 to 3 nitrogen atoms, wherein the monocyclic heteroaryl, pyridinyl-2 (1H)-one or the bicyclic heteroaryl are each optionally substituted with 1 or 2 R⁴;
R⁴, for each occurrence, is independently selected from hydroxyl, halo, C₁₋₄ alkoxy, halo-substitutedC₁₋₄ alkyl, and C₁₋₄ alkyl;
R⁵ is OR⁶; and
R⁶ is an optionally substituted C₁₋₅ alkyl or an optionally substituted C₃₋₆ cycloalkyl, wherein the C₁₋₅ alkyl is optionally substituted with 1 to 3 substituents independently selected from halogen and the C₃₋₆ cycloalkyl is optionally substituted with 1 to 3 substituents independently selected from C₁₋₄ alkyl, halo-substitutedC₁₋₄ alkyl and halogen.

34. The compound of claim 33, or a pharmaceutically acceptable salt thereof, wherein:
R¹ is 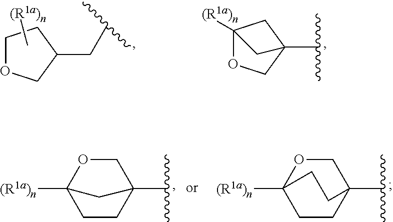
R^{1a} is C₁₋₄ alkyl or halo-substitutedC₁₋₄ alkyl;
n is 0 or 1;
R³ is 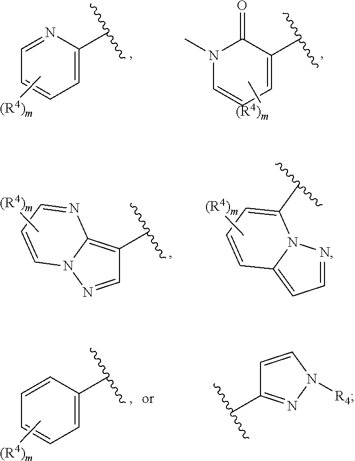
R⁴ is halo, C₁₋₄ alkoxy, C₁₋₄ alkyl or halo-substitutedC₁₋₄ alkyl;
m is 0 or 1;
R⁵ is OR⁶; and
R⁶ is C₁₋₄ alkyl or C₄₋₆ cycloalkyl.

35. The compound of claim 34, wherein R^{1a} is CH₃; and R⁴ is CH₃, F, OMe, or CHF₂; and R⁶ is —CH(CH₃)₂, cyclobutyl, or cyclopentyl.

36. The compound of claim 1, selected from any one of the compounds of Examples 1-140 or a pharmaceutically acceptable salt thereof.

37. A pharmaceutical composition comprising a compound of any one of the preceding claims or a pharmaceutically acceptable salt thereof.

38. The pharmaceutical composition of claim 37, further comprising one or more additional pharmaceutical agent(s).

39. A method of treating an IRAK4 mediated disease in a subject comprising administering to the subject a compound or a pharmaceutically acceptable salt thereof of any one of claims 1 to 36 or a pharmaceutical composition of any one of claims 37 to 38.

40. The method of claim 39, wherein the IRAK4 mediated disease is selected from the group consisting from Rheumatoid Arthritis, Psoriatic arthritis, Osteoarthritis, Systemic Lupus Erythematosus, Lupus nephritis, Ankylosing Spondylitis, Osteoporosis, Systemic sclerosis, Multiple Sclerosis, Psoriasis, Type I diabetes, Type II diabetes, Inflammatory Bowel Disease, Crohn's Disease, Ulcerative Colitis, Hyperimmunoglobulinaemia D, periodic fever syndrome, Cryopyrin-associated periodic syndromes, Schnitzler's syndrome, Systemic juvenile idiopathic arthritis, Adult's onset Still's disease, Gout, Pseudogout, SAPHO syndrome, Castleman's disease, Sepsis, Stroke, Atherosclerosis, Celiac disease, Deficiency of IL-1 Receptor Antagonist, Alzheimer's disease, Parkinson's disease, Multiple Sclerosis and Cancer.

41. The method of claim 39, wherein the IRAK4 mediated disease is selected from the group consisting from is selected from an autoimmune disease, an inflammatory disease, bone diseases, metabolic diseases, neurological and neurodegenerative diseases and/or disorders, cardiovascular diseases, allergies, asthma, hormone-related diseases, Ischemic stroke, Cerebral Ischemia, hypoxia, Traumatic Brain Injury, Chronic Traumatic Encephalopathy, epilepsy, Parkinson's disease, and Amyotrophic Lateral Sclerosis.
